(19) United States
(12) Patent Application Publication
(10) Pub. No.: US 2022/0244244 A1
(43) Pub. Date: Aug. 4, 2022

(54) A GENETIC PHARMACOPEIA FOR COMPREHENSIVE FUNCTIONAL PROFILING OF HUMAN CANCERS

(71) Applicant: Function Oncology, Inc., San Diego, CA (US)

(72) Inventors: Christian SCHMEDT, El Cerrito, CA (US); Srihari C. SAMPATH, San Diego, CA (US); Srinath C. SAMPATH, San Diego, CA (US)

(21) Appl. No.: 17/619,563

(22) PCT Filed: Jun. 18, 2020
(86) PCT No.: PCT/US20/38503

§ 371 Date: Dec. 15, 2021

Related U.S. Application Data
(60) Provisional application No. 62/865,047, filed on Jun. 21, 2019.

(51) Int. Cl. G01N 33/50; C12N 15/10; C12N 5/09; C12N 15/11; C12N 9/22; C12N 15/86

(52) U.S. Cl. CPC G01N 33/5011; C12N 15/1082; C12N 15/1065; G01N 33/5044; C12N 5/0693; C12N 2740/15043; C12N 9/22; C12N 15/86; C12N 2310/20; C12N 2800/80; C12N 15/11

(57) ABSTRACT

Described herein is a genetic pharmacopeia for interrogating individual cancer susceptibilities to available molecularly targeted therapies.

### CROSS-REFERENCE TO RELATED APPILCATIONS

This application claims the benefit of U.S. Provisional Patent Application Ser. No. 62/865,047, filed Jun. 21, 2019, which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Jun. 18, 2020, is named 56322-701_601_SL.txt and is 732,058 bytes in size.

### BACKGROUND OF THE INVENTION

Human cancers are extraordinarily heterogeneous, differing in DNA sequence, epigenomic landscape, RNA expression, and protein levels, resulting in vast combinatorial complexity in cell behavior. Despite impressive advances in our armamentarium of molecularly targeted anti-cancer therapies, the extreme molecular complexity underlying cancer cell behavior has led to dramatic shortfalls in our ability to predict which patients will benefit from any particular therapy. The lack of an effective means of predicting patient response directly leads to cycles of futile therapy, at enormous opportunity cost to patients and economic cost to both patients and healthcare payers.

### SUMMARY

The presently disclosed methods seek to provide a rational and personalized selection of therapeutics by determining which molecularly targeted therapy would be effective for a particular patient's disease. In one aspect, the methods comprise determining the functional susceptibility of a patient's cancer cells to a library of perturbagens which model the action of a library of known oncology drugs. Representative perturbagens include components of a gene editing or silencing system capable of knocking out, or knocking down, the genes encoding for the protein targets of the known oncology drugs. For instance, the perturbagens may include gene modulatory reagents such as guide RNA sequences for CRISPR-based gene editing, or RNAi for gene silencing. Accordingly, an exemplary method of functional susceptibility profiling comprises modifying a patient's cancer cells with a library of gene modulatory reagents capable of knocking down, or knocking out, the function of the genes encoding for protein targets of a library of known oncology drugs. In some methods the functionality of all such genes is knocked down or knocked out such that the susceptibility of a patient's cancer to all available molecularly targeted therapies may be interrogated. The modified cancer cells may be screened by next-generation sequencing to determine the effect of the individual genetic perturbations on the viability of the patient's cancer cells. Oncology drugs associated with the perturbagens that reduce viability of the cancer cells may be selected as a putative therapeutic, allowing for personalized selection of a cancer therapeutic.

In one aspect, provided herein is a method of treating cancer in a subject in need thereof, the method comprising administering to the subject a therapeutic molecule selected from a library of therapeutic molecules; wherein the therapeutic molecule has been selected by a method comprising: modifying cancer cells from the subject to knock down or knock out the function of a plurality of genes, each gene in the plurality of genes encoding for a protein target of a therapeutic molecule in the library of therapeutic molecules, whereby the therapeutic molecule has been selected if knocking down or knocking out the function of the gene that encodes for the protein target of the selected therapeutic molecule impairs cancer cell viability. In some embodiments, the library of therapeutic molecules comprises at least 1, at least 2, at least 3, at least 4, at least 5, at least 10, or at least 20 therapeutic agents of Table 2. In some embodiments, the library of therapeutic molecules comprises at least 1, at least 2, at least 3, at least 4, at least 5, at least 10, or at least 20 therapeutic agents of Table 3. In some embodiments, one or more of the plurality of genes encode for a protein of Table 5B. In some embodiments, one or more of the plurality of genes encode for a protein of Table 5A. In some embodiments, one or more of the plurality of genes encode for a protein of Table 5C. In some embodiments, one or more of the plurality of genes encode for a protein of Table 5D. In some embodiments, one or more of the plurality of genes encode for a protein of Table 4. In some embodiments, one or more of the plurality of genes encode for a protein of Table 3.

In another aspect, provided herein is a method of treating cancer in a subject in need thereof, the method comprising administering to the subject a therapeutic molecule selected from a library of therapeutic molecules; wherein the cancer of the subject has been determined to be susceptible to the selected therapeutic molecule by a method comprising: (a) contacting a sample of cancer cells from the subject with a library of gene modulatory reagents to generate a plurality of modified cancer cells, wherein each modified cancer cell harbors one or more of the gene modulatory reagents, and each gene modulatory reagent is capable of knocking down or knocking out the function of a gene that encodes a protein target of a therapeutic molecule in the library of therapeutic molecules, and (b) sequencing the plurality of modified cancer cells, wherein a gene modulatory reagent that impairs cell viability will have fewer sequence reads than a gene modulatory reagent that does not impair cell viability, and the gene that is knocked down or knocked out by the gene modulatory reagent that impairs cell viability encodes for the protein targeted by the selected therapeutic molecule. In some embodiments, prior to sequencing, one or more of the plurality of modified cancer cells have been propagated. In some embodiments, propagation comprises maintenance of the modified cancer cells in a 2D in vitro culture. In some embodiments, propagation comprises maintenance of the modified cancer cells in a 3D in vitro culture. In some embodiments, propagation comprises maintenance of the modified cancer cells in vivo. In some embodiments, propagation occurs within an animal model. In some embodiments, the animal is a rodent. In some embodiments, the cancer cells are primary cancer cells.

In some embodiments, contacting comprises introducing the one or more gene modulatory reagents into each cancer cell by a viral or non-viral delivery method. In some embodiments, one or more of the gene modulatory reagents in the library are encoded on a viral vector. In some embodiments, the viral vector comprises a lentiviral vector, adenoviral vector, or adeno-associated viral vector. In some embodiments, the non-viral delivery method comprises transposase-mediated transposition.

In some embodiments, the library comprises from about 10 to about 2,000, from about 10 to about 500, from about 10 to about 200, from about 10 to about 150, from about 50 to about 500, from about 50 to about 200, from about 50 to about 2,000, from about 100 to about 2,000, or from about 500 to about 2,000 different gene modulatory reagents. In some embodiments, one or more gene modulatory reagents from the library of gene modulatory reagents comprises a nucleic acid sequence homologous to at least about 15 contiguous nucleotides of a gene encoding a protein of Tables 3-5D. In some embodiments, one or more gene modulatory reagents from the library of gene modulatory reagents comprises a nucleic acid sequence homologous to at least about 15 contiguous nucleotides of a gene encoding a protein of Table 5B. In some embodiments, one or more gene modulatory reagents from the library of gene modulatory reagents comprises a nucleic acid sequence homologous to at least about 15 contiguous nucleotides of a gene encoding a protein of Table 5C. In some embodiments, one or more gene modulatory reagents from the library of gene modulatory reagents comprises a nucleic acid sequence homologous to at least about 15 contiguous nucleotides of a gene encoding a protein of Table 5D. In some embodiments, the homology is at least about 90% sequence homology. In some embodiments, the homology is at least about 90% sequence identity. In some embodiments, the library of therapeutic molecules comprises at least 1, at least 2, at least 3, at least 4, at least 5, at least 10, or at least 20 therapeutic agents of Table 2. In some embodiments, the library of therapeutic molecules comprises at least 1, at least 2, at least 3, at least 4, at least 5, at least 10, or at least 20 therapeutic agents of Table 3.

In some embodiments, the cancer comprises at least one cancer chosen from the group comprising acute lymphoblastic leukemia (ALL), acute myeloid leukemia (AML), adrenocortical carcinoma, AIDS-related cancers, Kaposi sarcoma, AIDS-related lymphoma, primary CNS lymphoma, anal cancer, appendix cancer, astrocytomas, atypical teratoid/rhabdoid tumor, central nervous system cancers, basal cell carcinoma of the skin, bile duct cancer, bladder cancer, bone cancer, brain tumors, breast cancer, bronchial tumors, Burkitt lymphoma, carcinoid tumor, cardiac tumors, central nervous system cancers, embryonal tumors, germ cell tumor, primary CNS lymphoma, cervical cancer, cholangiocarcinoma, chordoma, chronic lymphocytic leukemia (CLL), chronic myelogenous leukemia (CML), chronic myeloproliferative neoplasms, colorectal cancer, craniopharyngioma, cutaneous t-cell lymphoma, ductal carcinoma in situ (DCIS), embryonal tumors, central nervous system cancer, endometrial cancer, ependymoma, esophageal cancer, esthesioneuroblastoma, Ewing sarcoma, extracranial germ cell tumor, extragonadal germ cell tumor, eye cancer, intraocular melanoma, retinoblastoma, fallopian tube cancer, fibrous histiocytoma of bone, osteosarcoma, gallbladder cancer, gastric cancer, gastrointestinal carcinoid tumor, gastrointestinal stromal tumors (GIST), testicular cancer, gestational trophoblastic disease, hairy cell leukemia, head and neck cancer, heart tumors, hepatocellular cancer, Hodgkin lymphoma, hypopharyngeal cancer, intraocular melanoma, islet cell tumors, pancreatic neuroendocrine tumors, kidney cancer, Langerhans cell histiocytosis, laryngeal cancer, leukemia, lip and oral cavity cancer, liver cancer, lung cancer (non-small cell and small cell), lymphoma, male breast cancer, malignant fibrous histiocytoma of bone and osteosarcoma, melanoma, intraocular melanoma, Merkel cell carcinoma, mesothelioma, metastatic cancer, metastatic squamous neck cancer with occult primary, midline tract carcinoma with NUT gene changes, mouth cancer, multiple endocrine neoplasia syndromes, multiple myeloma/plasma cell neoplasms, mycosis fungoides, myelodysplastic syndromes, myelodysplastic/myeloproliferative neoplasms, myelogenous leukemia, chronic (CML), myeloid leukemia, acute (AML), nasal cavity and paranasal sinus cancer, nasopharyngeal cancer, neuroblastoma, non-Hodgkin lymphoma, non-small cell lung cancer, oral cancer, oropharyngeal cancer, ovarian cancer, pancreatic cancer, pancreatic neuroendocrine tumors (islet cell tumors), papillomatosis, paraganglioma, paranasal sinus and nasal cavity cancer, parathyroid cancer, penile cancer, pharyngeal cancer, pheochromocytoma, pituitary tumor, plasma cell neoplasm/multiple myeloma, pleuropulmonary blastoma, primary central nervous system (CNS) lymphoma, primary peritoneal cancer, prostate cancer, rectal cancer, recurrent cancer, renal cell cancer, retinoblastoma, rhabdomyosarcoma, salivary gland cancer, sarcoma, rhabdomyosarcoma, vascular tumors, osteosarcoma, soft tissue sarcoma, uterine sarcoma, Sézary syndrome, skin cancer, small intestine cancer, squamous cell carcinoma of the skin, squamous neck cancer with occult primary, stomach cancer, T-cell lymphoma, testicular cancer, throat cancer, nasopharyngeal cancer, oropharyngeal cancer, hypopharyngeal cancer, thymoma and thymic carcinoma, thyroid cancer, transitional cell cancer of the renal pelvis and ureter, ureter and renal pelvis, urethral cancer, uterine cancer, endometrial, uterine sarcoma, vaginal cancer, vascular tumors, vulvar cancer, Wilms tumor, and other childhood kidney tumors.

In some embodiments, one or more of the gene modulatory reagents each comprise a guide RNA (gRNA) sequence comprising at least about 90% homology to a sequence selected from SEQ ID NOS: 1-2789, 2980-3071. In some embodiments, one or more of the gene modulatory reagents each comprise a guide RNA (gRNA) sequence comprising at least about 90% homology to a sequence selected from SEQ ID NOS: 1526-2789. In some embodiments, one or more of the gene modulatory reagents each comprise a guide RNA (gRNA) sequence comprising at least about 90% homology to a sequence selected from SEQ ID NOS: 2980-3071. In some embodiments, the at least about 90% homology is at least about 90% identity. In some embodiments, one or more of the gene modulatory reagents comprise a guide RNA (gRNA) sequence comprising homology to at least a portion of the gene that encodes a protein target of a therapeutic molecule in the library of therapeutic molecules. In some embodiments, the gRNA comprises homology to about 10 to about 50 contiguous nucleotides of the gene. In some embodiments, the homology is at least about 90% sequence homology. In some embodiments, the homology is at least about 90% sequence identity. In some embodiments, the sample of cancer cells is contacted with an endonuclease. In some embodiments, the endonuclease comprises a Cas9 or Cas12a endonuclease. In some embodiments, the Cas9 or Cas12a endonuclease is selected from S. pyogenes Cas9 (SpCas9), SpCas9 D1135E variant, SpCas9 VRER variant, SpCas9 EQR variant, xCas9, SpCas9-NG, S. aureus Cas9 (SaCas9), Acidaminococcus sp. (AsCpfl), Lachnospiraceae bacterium (LbCpfl), AsCpfl RR variant, LbCpfl RR variant, AsCpfl RVR variant, C. jejuni Cas9 (Cj Cas9), N meningitidis (NmCas9), S. thermophilus (StCas9), T. denticola (TdCas9), and Mad7. In some embodiments, the endonuclease does not comprise a Cas9 or Cas12a endonuclease. In some embodiments, the gRNA is positioned within a vector. In some embodiments, the vector further comprises genetic elements of a virus. In some embodiments, the virus comprises an adenovirus, retrovirus, adeno-associated virus (AAV), pox virus, parvovirus, baculovirus, measles virus, herpes simplex virus (HSV), Moloney Murine Leukemia Virus (MoMLV, MMLV, MuLV, or MLV), Murine Stem cell Virus (MSCV), or human immunodeficiency virus (HIV), or a combination thereof In some embodiments, the vector further comprises an auxiliary nucleic acid sequence. In some embodiments, the auxiliary nucleic acid sequence comprises a sequence encoding a marker, an antibiotic resistance cassette, and surface epitope expression cassette. In some embodiments, the marker is a fluorescent marker. In some embodiments, the auxiliary nucleic acid allows for the selection of cancer cells that have been modified to harbor the one or more gene modulatory reagents.

In some embodiments, one or more of the gene modulatory reagents comprise a short hairpin RNA (shRNA) sequence comprising homology to at least a portion of the gene that encodes a protein target of a therapeutic molecule in the library of therapeutic molecules. In some embodiments, the shRNA comprises homology to about 10 to about 50 contiguous nucleotides of the gene. In some embodiments, the homology is at least about 90% sequence homology. In some embodiments, the homology is at least about 90% sequence identity. In some embodiments, the shRNA is positioned within a vector. In some embodiments, the vector further comprises genetic elements of a virus. In some embodiments, the virus comprises an adenovirus, retrovirus, adeno-associated virus (AAV), pox virus, parvovirus, baculovirus, measles virus, herpes simplex virus (HSV), Moloney Murine Leukemia Virus (MoMLV, MMLV, MuLV, or MLV), Murine Stem cell Virus (MSCV), or human immunodeficiency virus (HIV), or a combination thereof. In some embodiments, the vector further comprises an auxiliary nucleic acid sequence. In some embodiments, the auxiliary nucleic acid sequence comprises a sequence encoding a marker, an antibiotic resistance cassette, or surface epitope expression cassette. In some embodiments, the marker is a fluorescent marker. In some embodiments, the auxiliary nucleic acid allows for the selection of cancer cells that have been modified to harbor the one or more gene modulatory reagents.

In another aspect, provided herein is a method of generating a plurality of modified cancer cells from a subject having cancer, the method comprising delivering a library of gene modulatory reagents to a sample of cancer cells from the subject to generate the plurality of modified cancer cells; wherein each modified cancer cell harbors one or more of the gene modulatory reagents, and each gene modulatory reagent is capable of knocking down or knocking out the function of a gene that encodes a protein target from a library of protein targets. In some embodiments, one or more of the gene modulatory reagents comprises a guide RNA (gRNA) sequence comprising homology to at least a portion of the gene whose function is knocked down or knocked out in the modified cancer cell. In some embodiments, the gRNA comprises homology to about 10 to about 50 contiguous nucleotides of the gene. In some embodiments, the homology is at least about 90% sequence homology. In some embodiments, the homology is at least about 90% sequence identity. In some embodiments, one or more of the gene modulatory reagents each comprise a guide RNA (gRNA) sequence comprising at least about 90% homology to a sequence selected from SEQ ID NOS: 1-2789, 2980-3071. In some embodiments, one or more of the gene modulatory reagents each comprise a guide RNA (gRNA) sequence comprising at least about 90% homology to a sequence selected from SEQ ID NOS: 1526-2789. In some embodiments, one or more of the gene modulatory reagents each comprise a guide RNA (gRNA) sequence comprising at least about 90% homology to a sequence selected from SEQ ID NOS: 2980-3071. In some embodiments, the homology is at least about 90% identity. In some embodiments, the sample of cancer cells is contacted with an endonuclease. In some embodiments, the endonuclease comprises a Cas9 or Cas12a endonuclease. In some embodiments, the Cas9 or Cas12a endonuclease is selected from S. pyogenes Cas9 (SpCas9), SpCas9 D1135E variant, SpCas9 VRER variant, SpCas9 EQR variant, xCas9, SpCas9-NG, S. aureus Cas9 (SaCas9), Acidaminococcus sp. (AsCpfl), Lachnospiraceae bacterium (LbCpfl), AsCpfl RR variant, LbCpfl RR variant, AsCpfl RVR variant, C. jejuni Cas9 (Cj Cas9), N. meningitidis (NmCas9), S. thermophilus (StCas9), T. denticola (TdCas9), and Mad7. In some embodiments, the endonuclease does not comprise a Cas9 or Cas12a endonuclease. In some embodiments, the gRNA is positioned within a vector. In some embodiments, the vector further comprises genetic elements of a virus. In some embodiments, the virus comprises an adenovirus, retrovirus, adeno-associated virus (AAV), pox virus, parvovirus, baculovirus, measles virus, herpes simplex virus (HSV), Moloney Murine Leukemia Virus (MoMLV, MMLV, MuLV, or MLV), Murine Stem cell Virus (MSCV), or human immunodeficiency virus (HIV), or a combination thereof In some embodiments, the vector further comprises an auxiliary nucleic acid sequence. In some embodiments, the auxiliary nucleic acid sequence comprises a sequence encoding a marker, an antibiotic resistance cassette, or surface epitope expression cassette. In some embodiments, the marker is a fluorescent marker. In some embodiments, the auxiliary nucleic acid allows for the selection of cancer cells that have been modified to harbor the one or more gene modulatory reagents.

In some embodiments, one or more of the gene modulatory reagents comprise a short hairpin RNA (shRNA) sequence comprising homology to at least a portion of the gene whose function is knocked down or knocked out in the modified cancer cell. In some embodiments, the shRNA comprises homology to about 10 to about 50 contiguous nucleotides of the gene. In some embodiments, the homology is at least about 90% sequence homology. In some embodiments, the homology is at least about 90% sequence identity. In some embodiments, the shRNA is positioned within a vector. In some embodiments, the vector further comprises genetic elements of a virus. In some embodiments, the virus comprises an adenovirus, retrovirus, adeno-associated virus (AAV), pox virus, parvovirus, baculovirus, measles virus, herpes simplex virus (HSV), Moloney Murine Leukemia Virus (MoMLV, MMLV, MuLV, or MLV), Murine Stem cell Virus (MSCV), or human immunodeficiency virus (HIV), or a combination thereof. In some embodiments, the vector further comprises an auxiliary nucleic acid sequence. In some embodiments, the auxiliary nucleic acid sequence comprises a sequence encoding a marker, an antibiotic resistance cassette, or surface epitope expression cassette. In some embodiments, the marker is a fluorescent marker. In some embodiments, the auxiliary nucleic acid allows for the selection of cancer cells that have been modified to harbor the one or more gene modulatory reagents.

In some embodiments, delivery comprises transposase-mediated transposition. In some embodiments, the sample of cancer cells comprises primary cancer cells. In some embodiments, the sample of cancer cells comprises about 10⁵ to about 10⁸ cells. In some embodiments, the library comprises from about 10 to about 2,000, from about 10 to about 500, from about 10 to about 200, from about 10 to about 150, from about 50 to about 500, from about 50 to about 200, from about 50 to about 2,000, from about 100 to about 2,000, or from about 500 to about 2,000 different gene modulatory reagents. In some embodiments, at least about 90% of the gene modulatory reagents are present in the library in a quantity within about 10% of the average gene modulatory reagent quantity. In some embodiments, the library of protein targets comprises at least 1, at least 2, at least 3, at least 4, at least 5, at least 10, or at least 20 protein targets of Table 5B. In some embodiments, the library of protein targets comprises at least 1, at least 2, at least 3, at least 4, at least 5, at least 10, or at least 20 protein targets of Table 5A. In some embodiments, the library of protein targets comprises at least 1, at least 2, at least 3, at least 4, at least 5, at least 10, or at least 20 protein targets of Table 5C. In some embodiments, the library of protein targets comprises at least 1, at least 2, at least 3, at least 4, at least 5, at least 10, or at least 20 protein targets of Table 5D. In some embodiments, the library of protein targets comprises at least 1, at least 2, at least 3, at least 4, at least 5, at least 10, or at least 20 protein targets of Table 4. In some embodiments, the library of protein targets comprises at least 1, at least 2, at least 3, at least 4, at least 5, at least 10, or at least 20 protein targets of Table 3. In some embodiments, the sample of cancer cells has been processed to preserve cell viability.

In some embodiments, the method further comprises preparing the sample of cancer cells to preserve cell viability prior to and/or after delivery of the library of gene modulatory reagents. In some embodiments, the method further comprises propagating the modified cancer cells. In some embodiments, propagation comprises maintenance of the modified cancer cells in a 2D in vitro culture. In some embodiments, propagation comprises maintenance of the modified cancer cells in a 3D in vitro culture. In some embodiments, propagation comprises maintenance of the modified cancer cells in vivo. In some embodiments, propagation occurs within an animal model. In some embodiments, the animal model is a rodent.

In another aspect, provided herein is a compilation comprising a plurality of modified cancer cells, wherein each modified cancer cell harbors one or more gene modulatory reagents, and each gene modulatory reagent is capable of knocking down or knocking out the function of a gene that encodes a protein target from a library of protein targets. In some embodiments, at least one of the one or more gene modulatory reagents comprises a sequence selected from SEQ ID NOS: 1-2789, 2980-3071. In some embodiments, at least one of the one or more gene modulatory reagents comprises a sequence selected from SEQ ID NOS: 1526-2789. In some embodiments, at least one of the one or more gene modulatory reagents comprises a sequence selected from SEQ ID NOS: 2980-3071. In some embodiments, at least one of the one or more of gene modulatory reagents comprises a sequence at least about 90% homologous to a sequence selected from SEQ ID NOS: 1-2789, 2980-3071. In some embodiments, at least one of the one or more of gene modulatory reagents comprises a sequence at least about 90% homologous to a sequence selected from SEQ ID NOS: 1526-2789. In some embodiments, at least one of the one or more of gene modulatory reagents comprises a sequence at least about 90% homologous to a sequence selected from SEQ ID NOS: 2980-3071. In some embodiments, the homology is 90% identity.

In some embodiments, the library of protein targets comprises at least 1, at least 2, at least 3, at least 4, at least 5, at least 10, or at least 20 protein targets of Table 5B. In some embodiments, the library of protein targets comprises at least 1, at least 2, at least 3, at least 4, at least 5, at least 10, or at least 20 protein targets of Table 5A. In some embodiments, the library of protein targets comprises at least 1, at least 2, at least 3, at least 4, at least 5, at least 10, or at least 20 protein targets of Table 5C. In some embodiments, the library of protein targets comprises at least 1, at least 2, at least 3, at least 4, at least 5, at least 10, or at least 20 protein targets of Table 5D. In some embodiments, the library of protein targets comprises at least 1, at least 2, at least 3, at least 4, at least 5, at least 10, or at least 20 protein targets of Table 4. In some embodiments, the library of protein targets comprises at least 1, at least 2, at least 3, at least 4, at least 5, at least 10, or at least 20 protein targets of Table 3. In some embodiments, one of more of the gene modulatory reagents comprise a guide RNA (gRNA) sequence comprising homology to at least a portion of the gene whose function is knocked down or knocked out in the modified cancer cell. In some embodiments, the gRNA comprises homology to about 10 to about 50 contiguous nucleotides of the gene. In some embodiments, the homology is at least about 90% sequence homology. In some embodiments, the homology is at least about 90% sequence identity.

In some embodiments, the compilation comprises an endonuclease. In some embodiments, the endonuclease comprises a Cas9 or Cas12a endonuclease. In some embodiments, the Cas9 or Cas12a endonuclease is selected from S. pyogenes Cas9 (SpCas9), SpCas9 D1135E variant, SpCas9 VRER variant, SpCas9 EQR variant, xCas9, SpCas9-NG, S. aureus Cas9 (SaCas9), Acidaminococcus sp. (AsCpfl), Lachnospiraceae bacterium (LbCpfl), AsCpfl RR variant, LbCpfl RR variant, AsCpfl RVR variant, C. jejuni Cas9 (CjCas9), N. meningitidis (NmCas9), S. thermophilus (StCas9), T. denticola (TdCas9), and Mad7. In some embodiments, the endonuclease does not comprise a Cas9 or Cas12a endonuclease.

In some embodiments, the gRNA is positioned within a vector. In some embodiments, the vector further comprises genetic elements of a virus. In some embodiments, the virus comprises an adenovirus, retrovirus, adeno-associated virus (AAV), pox virus, parvovirus, baculovirus, measles virus, herpes simplex virus (HSV), Moloney Murine Leukemia Virus (MoMLV, MMLV, MuLV, or MLV), Murine Stem cell Virus (MSCV), or human immunodeficiency virus (HIV), or a combination thereof. In some embodiments, the vector further comprises an auxiliary nucleic acid sequence. In some embodiments, the auxiliary nucleic acid sequence comprises a sequence encoding a marker, an antibiotic resistance cassette, or surface epitope expression cassette. In some embodiments, the marker is a fluorescent marker. In some embodiments, the auxiliary nucleic acid allows for the selection of the modified cancer cells.

In some embodiments, one or more of the gene modulatory reagents comprise a short hairpin RNA (shRNA) sequence comprising homology to at least a portion of the gene whose function is knocked down or knocked out in the modified cancer cell. In some embodiments, the shRNA comprises homology to about 10 to about 50 contiguous nucleotides of the gene. In some embodiments, the homology is at least about 90% sequence homology. In some embodiments, the homology is at least about 90% sequence identity. In some embodiments, the shRNA is positioned within a vector. In some embodiments, the vector further comprises genetic elements of a virus. In some embodiments, the virus comprises an adenovirus, retrovirus, adeno-associated virus (AAV), pox virus, parvovirus, baculovirus, measles virus, herpes simplex virus (HSV), Moloney Murine Leukemia Virus (MoMLV, MMLV, MuLV, or MLV), Murine Stem cell Virus (MSCV), or human immunodeficiency virus (HIV), or a combination thereof. In some embodiments, the vector further comprises an auxiliary nucleic acid sequence. In some embodiments, the auxiliary nucleic acid sequence comprises a sequence encoding a marker, an antibiotic resistance cassette, or surface epitope expression cassette. In some embodiments, the marker is a fluorescent marker. In some embodiments, the auxiliary nucleic acid allows for the selection of the modified cancer cells. In some embodiments, delivering comprising transposase-mediated transposition.

In some embodiments, the modified cancer cells are modified primary cancer cells. In some embodiments, the compilation comprises from about 10 to about 2,000, from about 10 to about 500, from about 10 to about 200, from about 10 to about 150, from about 50 to about 500, from about 50 to about 200, from about 50 to about 2,000, from about 100 to about 2,000, or from about 500 to about 2,000 different gene modulatory reagents. In some embodiments, the compilation comprises from about 10 to about 2,000, from about 10 to about 500, from about 10 to about 200, from about 10 to about 150, from about 50 to about 500, from about 50 to about 200, from about 50 to about 2,000, from about 100 to about 2,000, or from about 500 to about 2,000 different populations of modified cancer cells.

In another aspect, provided herein is a method of evaluating the functional effect of genetically modifying cancer cells from a subject, the method comprising: sequencing a plurality of modified cancer cells, wherein each modified cancer cell harbors one or more gene modulatory reagents, each gene modulatory reagent capable of knocking down or knocking out the function of a gene that encodes a protein target in a library of protein targets; and wherein a gene modulatory reagent that impairs cell viability will have fewer sequence reads than a gene modulatory reagent that does not impair cell viability. In some embodiments, the method comprises determining which gene modulatory regents have fewer than a threshold number of sequence reads. In some embodiments, the threshold number of sequence reads is an expected number of sequence reads if the gene modulatory reagent did not impair cell viability. In some embodiments, the threshold number of sequence reads is an average number of sequence reads for each gene modulatory reagent in the plurality of modified cancer cells.

In some embodiments, the method comprises correlating each gene modulatory reagent that has fewer than the threshold number of sequence reads to its corresponding protein target in the library of protein targets. In some embodiments, the method comprises correlating the corresponding protein target to a therapeutic molecule. In some embodiments, the library of protein targets comprises at least 1, at least 2, at least 3, at least 4, at least 5, at least 10, or at least 20 protein targets of Table 5B. In some embodiments, the library of protein targets comprises at least 1, at least 2, at least 3, at least 4, at least 5, at least 10, or at least 20 protein targets of Table 5A. In some embodiments, the library of protein targets comprises at least 1, at least 2, at least 3, at least 4, at least 5, at least 10, or at least 20 protein targets of Table 5C. In some embodiments, the library of protein targets comprises at least 1, at least 2, at least 3, at least 4, at least 5, at least 10, or at least 20 protein targets of Table 5D. In some embodiments, the library of protein targets comprises at least 1, at least 2, at least 3, at least 4, at least 5, at least 10, or at least 20 protein targets of Table 4. In some embodiments, the library of protein targets comprises at least 1, at least 2, at least 3, at least 4, at least 5, at least 10, or at least 20 protein targets of Table 3.

In some embodiments, one or more of the gene modulatory reagents each comprise a guide RNA (gRNA) sequence comprising at least about 90% homology to a sequence selected from SEQ ID NOS: 1-2789, 2980-3071. In some embodiments, one or more of the gene modulatory reagents each comprise a guide RNA (gRNA) sequence comprising at least about 90% homology to a sequence selected from SEQ ID NOS: 1526-2789. In some embodiments, one or more of the gene modulatory reagents each comprise a guide RNA (gRNA) sequence comprising at least about 90% homology to a sequence selected from SEQ ID NOS: 2980-3071. In some embodiments, the at least about 90% homology is at least about 90% identity.

In another aspect, provided herein is a library comprising a plurality of gene modulatory reagents, each gene modulatory reagent capable of knocking down or knocking out the function of a gene that encodes a protein target from a library of protein targets. In some embodiments, the plurality of gene modulatory reagents is capable of knocking down or knocking out the function of at least about 50% of the genes that encode for the protein targets in the library. In some embodiments, the at least about 50% is at least about 60%. In some embodiments, the at least about 60% is at least about 70%. In some embodiments, the at least about 70% is at least about 80%. In some embodiments, the at least about 80% is at least about 90%. In some embodiments, the library of protein targets comprises all known proteins targeted by known drugs capable of treating a particular disease or condition. In some embodiments, the disease or condition is cancer. In some embodiments, the cancer comprises at least one cancer from the group comprising acute lymphoblastic leukemia (ALL), acute myeloid leukemia (AML), adrenocortical carcinoma, AIDS-related cancers, Kaposi sarcoma, AIDS-related lymphoma, primary CNS lymphoma, anal cancer, appendix cancer, astrocytomas, atypical teratoid/rhabdoid tumor, central nervous system cancers, basal cell carcinoma of the skin, bile duct cancer, bladder cancer, bone cancer, brain tumors, breast cancer, bronchial tumors, Burkitt lymphoma, carcinoid tumor, cardiac tumors, central nervous system cancers, embryonal tumors, germ cell tumor, primary CNS lymphoma, cervical cancer, cholangiocarcinoma, chordoma, chronic lymphocytic leukemia (CLL), chronic myelogenous leukemia (CML), chronic myeloproliferative neoplasms, colorectal cancer, craniopharyngioma, cutaneous t-cell lymphoma, ductal carcinoma in situ (DCIS), embryonal tumors, central nervous system cancer, endometrial cancer, ependymoma, esophageal cancer, esthesioneuroblastoma, Ewing sarcoma, extracranial germ cell tumor, extragonadal germ cell tumor, eye cancer, intraocular melanoma, retinoblastoma, fallopian tube cancer, fibrous histiocytoma of bone, osteosarcoma, gallbladder cancer, gastric cancer, gastrointestinal carcinoid tumor, gastrointestinal stromal tumors (GIST), testicular cancer, gestational trophoblastic disease, hairy cell leukemia, head and neck cancer, heart tumors, hepatocellular cancer, Hodgkin lymphoma, hypopharyngeal cancer, intraocular melanoma, islet cell tumors, pancreatic neuroendocrine tumors, kidney cancer, Langerhans cell histiocytosis, laryngeal cancer, leukemia, lip and oral cavity cancer, liver cancer, lung cancer (non-small cell and small cell), lymphoma, male breast cancer, malignant fibrous histiocytoma of bone and osteosarcoma, melanoma, intraocular melanoma, Merkel cell carcinoma, mesothelioma, metastatic cancer, metastatic squamous neck cancer with occult primary, midline tract carcinoma with NUT gene changes, mouth cancer, multiple endocrine neoplasia syndromes, multiple myeloma/plasma cell neoplasms, mycosis fungoides, myelodysplastic syndromes, myelodysplastic/myeloproliferative neoplasms, myelogenous leukemia, chronic (CML), myeloid leukemia, acute (AML), nasal cavity and paranasal sinus cancer, nasopharyngeal cancer, neuroblastoma, non-Hodgkin lymphoma, non-small cell lung cancer, oral cancer, oropharyngeal cancer, ovarian cancer, pancreatic cancer, pancreatic neuroendocrine tumors (islet cell tumors), papillomatosis, paraganglioma, paranasal sinus and nasal cavity cancer, parathyroid cancer, penile cancer, pharyngeal cancer, pheochromocytoma, pituitary tumor, plasma cell neoplasm/multiple myeloma, pleuropulmonary blastoma, primary central nervous system (CNS) lymphoma, primary peritoneal cancer, prostate cancer, rectal cancer, recurrent cancer, renal cell cancer, retinoblastoma, rhabdomyosarcoma, salivary gland cancer, sarcoma, rhabdomyosarcoma, vascular tumors, osteosarcoma, soft tissue sarcoma, uterine sarcoma, Sézary syndrome, skin cancer, small intestine cancer, squamous cell carcinoma of the skin, squamous neck cancer with occult primary, stomach cancer, T-cell lymphoma, testicular cancer, throat cancer, nasopharyngeal cancer, oropharyngeal cancer, hypopharyngeal cancer, thymoma and thymic carcinoma, thyroid cancer, transitional cell cancer of the renal pelvis and ureter, ureter and renal pelvis, urethral cancer, uterine cancer, endometrial, uterine sarcoma, vaginal cancer, vascular tumors, vulvar cancer, Wilms tumor, and other childhood kidney tumors.

In some embodiments, the known drugs comprise at least 1, at least 2, at least 3, at least 4, at least 5, at least 10, or at least 20 drugs of Table 2. In some embodiments, the known drugs comprise at least 1, at least 2, at least 3, at least 4, at least 5, at least 10, or at least 20 drugs of Table 3. In some embodiments, the library of protein targets comprises at least 1, at least 2, at least 3, at least 4, at least 5, at least 10, or at least 20 protein targets of Table 5B. In some embodiments, the library of protein targets comprises at least 1, at least 2, at least 3, at least 4, at least 5, at least 10, or at least 20 protein targets of Table 5A. In some embodiments, the library of protein targets comprises at least 1, at least 2, at least 3, at least 4, at least 5, at least 10, or at least 20 protein targets of Table 5C. In some embodiments, the library of protein targets comprises at least 1, at least 2, at least 3, at least 4, at least 5, at least 10, or at least 20 protein targets of Table 5D. In some embodiments, the library of protein targets comprises at least 1, at least 2, at least 3, at least 4, at least 5, at least 10, or at least 20 protein targets of Table 4. In some embodiments, the library of protein targets comprises at least 1, at least 2, at least 3, at least 4, at least 5, at least 10, or at least 20 protein targets of Table 3. In some embodiments, one or more of the plurality of gene modulatory reagents each comprise a guide RNA (gRNA) sequence comprising at least about 90% homology to a sequence selected from SEQ ID NOS: 1-2789, 2980-3071. In some embodiments, one or more of the plurality of gene modulatory reagents each comprise a guide RNA (gRNA) sequence comprising at least about 90% homology to a sequence selected from SEQ ID NOS: 1526-2789. In some embodiments, one or more of the plurality of gene modulatory reagents each comprise a guide RNA (gRNA) sequence comprising at least about 90% homology to a sequence selected from SEQ ID NOS: 2980-3071. In some embodiments, the at least about 90% homology is at least about 90% identity.

In some embodiments, the plurality of gene modulatory reagents is capable of knocking down or knocking out the function of about 10 to about 2,000, from about 10 to about 500, from about 10 to about 200, from about 10 to about 150, from about 50 to about 500, from about 50 to about 200, about 50 to about 2,000, or about 100 to about 2,000 genes. In some embodiments, the library comprises about 10 to about 2,000, from about 10 to about 500, from about 10 to about 200, from about 10 to about 150, from about 50 to about 500, from about 50 to about 200, about 50 to about 2,000, or about 100 to about 2,000 gene modulatory reagents.

In some embodiments, at least one of the gene modulatory reagents is capable of knocking out the function of a gene. In some embodiments, at least one of the gene modulatory reagents comprise a gRNA sequence having homology to at least a portion of the gene whose function is knocked out by the gene modulatory reagent. In some embodiments, at least one of the gene modulatory reagents is capable of knocking down the function of a gene. In some embodiments, at least one of the gene modulatory reagents comprise a shRNA sequence having homology to at least a portion of the gene whose function is knocked down by the gene modulatory reagent. In some embodiments, the homology is at least about 90% sequence homology. In some embodiments, the homology is at least about 90% sequence identity. In some embodiments, the at least a portion is at least about 15 contiguous nucleotides.

In some embodiments, at least one of the gene modulatory reagents is positioned within a vector. In some embodiments, the vector comprises an adapter sequence. In some embodiments, the adapter sequence comprises a type IIS restriction enzyme cleavage sites. In some embodiments, the vector further comprises genetic elements of a virus. In some embodiments, the virus comprises an adenovirus, retrovirus, adeno-associated virus (AAV), pox virus, parvovirus, baculovirus, measles virus, herpes simplex virus (HSV), Moloney Murine Leukemia Virus (MoMLV, MMLV, MuLV, or MLV), Murine Stem cell Virus (MSCV), or human immunodeficiency virus (HIV), or a combination thereof In some embodiments, the vector further comprises a sequence encoding a marker, an antibiotic resistance cassette, or surface epitope expression cassette. In some embodiments, the marker is a fluorescent marker.

### BRIEF DESCRIPTION OF THE DRAWINGS

FIG. 1 is a clinical workflow of a cancer functional susceptibility profiling method described herein.

FIG. 2 is a schematic of a CRISPR-based platform for personalized functional genomics.

FIG. 3 is a schematic for identifying cancer therapeutic vulnerabilities in the gene space via CRISPR.

FIG. 4 is a table of the characteristics of a targeted oncology CRISPR library.

FIG. 5 shows distribution of gRNA representation in pooled plasmid DNA (left) and transduced cells (right).

FIG. 6A shows a 3D collagen scaffold containing infected primary tumor cells.

FIG. 6B shows re-isolated cells demonstrating the outgrowth of the small tumor-derived tumoroids/organoids.

FIG. 7 shows expression of B2M, demonstrating loss of B2M protein expression at the precise frequency expected based on the relative abundance of B2M-tarting gRNAs in a gRNA library.

FIG. 8 is a volcano plot of CRISPR library screening in A549 lung carcinoma cells. Core selected genes are shown in dark circles (TOP2A, TUBB, RPL3, TUBG1, PSMB5). Negative control genes are shown in gray circles.

FIG. 9 is a volcano plot of CRISPR library screening in primary PDX-derived human melanoma tumor cells. The known melanoma driver gene BRAF is identified as a therapeutic vulnerability. Negative control genes are shown in gray circles.

### DETAILED DESCRIPTION OF THE INVENTION

Most previous efforts in personalized cancer sensitivity testing have focused on treatment of tumor cells with proposed therapeutic small molecules in vitro. However, numerous studies have demonstrated that key differences exist between the behavior of cancer cells in vitro and their corresponding behavior in vivo, including the response of cancer cells to inhibition of various molecular pathways. Indeed, one of the most clinically successful approaches to date for chemosensitivity testing utilizes engraftment of primary patient-derived cancer cells into mice, followed by in vivo treatment of the animals with drugs. This approach is effective but extraordinarily slow (6-12 months), expensive (cost of goods related to compounds and animals; hands-on time for dosing, analysis), and non-comprehensive (i.e. only a few drugs can be tested). As a result, the approach is intrinsically non-scalable.

Over 300 molecularly targeted therapies are either approved or under study for the treatment of cancer. Each of these drugs (usually a low molecular weight compound, or in some cases an antibody) binds to and, in nearly all cases, inactivates the function of a particular protein target. While it is conceptually appealing to test each of these drugs on an individual patient's cancer cells in order to find effective therapies, this has proven over several decades to be an inherently limited and suboptimal process for a number of reasons: (1) The number of cells required to perform the test limits the number of therapies (drugs) which can be tested. (2) Testing can only be performed in vitro, which differs significantly from the in vivo context in which clinical therapy is performed. (3) Accurate testing depends on knowledge of in vitro drug stability and cellular exposure, which are usually not known. As a result, the data gathered from compound testing in vitro does not reflect achievable in vivo tissue exposure. (4) High cost of goods associated with maintaining validated and updated stocks of all drugs. (5) Testing cannot be performed in a pooled or multiplexed format, raising costs and limiting throughput. These processes are therefore in principle unable to be scaled to commercial levels. (6) Testing cannot be used to identify new targets, i.e. those for which drugs are not already available.

The presently described methods work by pivoting the currently available suite of anti-cancer therapies from ‘drug space’ to ‘gene space’. Specifically, the methods depend on the critical insight that each protein target of an existing therapy can also be inhibited indirectly via mutagenesis of the gene encoding that protein target, e.g. via gene editing. Thus, the ‘drug pharmacopeia’ can instead be represented by a ‘genetic pharmacopeia’. The genetic pharmacopeia can represent an entire targeted therapy landscape (e.g., for the oncology landscape, over 300 therapeutic molecules are represented), in a genetic format. This may be achieved by designing inhibitory genetic elements, for instance sgRNAs (CRISPR) or shRNAs (RNAi) corresponding to the gene or mRNA respectively of the protein target of each potential therapeutic. Accordingly, the genetic pharmacopeia allows for a genetic determination of the functional susceptibility of cancer cells to known oncology drugs, mitigating the shortcomings described above and as shown in Table 1.

TABLE 1
Mitigating the shortcomings of existing methods with a genetic pharmacopeia.

| Shortcoming of existing solutions | Mitigation via Genetic Pharmacopeia |
|---|---|
| The number of cells required to perform the test | Genetic platform allows pooled screening with |
| limits the number of therapies (drugs) which can | highly parallel, NGS-based readout of target |
| be tested | modulation |
| Testing can only be performed in vitro, which | Testing can be performed in vivo, as well as in |
| differs significantly from the in vivo context in | complex in vitro environments mimicking the in |
| which clinical therapy is performed | vivo context (e.g. 3D matrices, engineered niches) |
| Accurate testing depends on knowledge of in | No knowledge needed of pharmacokinetic or |
| vitro drug stability and cellular exposure, which | chemical properties of existing drug entities |
| are usually not known. As a result, the data | |
| gathered from compound testing in vitro does | |
| not reflect achievable in vivo tissue exposure | |
| High cost of goods associated with maintaining | No pharmacologic stocks are required. Required |
| validated and updated stocks of all drugs | consumable reagents are widely available at |
| | commodity pricing |
| Testing cannot be performed in a pooled or | Tests are performed in a pooled, highly |
| multiplexed format, raising costs and limiting | multiplexed format |
| throughput | |
| Testing cannot be used to identify new targets, | Genetic pools can be designed to support novel |
| i.e. those for which drugs are not already | drug discovery, i.e. for protein targets for which |
| available | no current inhibitor is available, in primary |
| | patient-derived cells |

In the same way that a chemical pharmacopeia reduces the vast potential drug space (i.e. all LMW chemical structures) to a size that is useful for the selection of therapies in actual practice, a genetic pharmacopeia reduces the complexity of the human genome to a scale suitable for practical use in personalized diagnostics. The limited availability of patient-derived cells, which are usually derived from scant biopsy or resection specimens, and the limited ability to propagate these cells in culture mandates this reduction in complexity, and makes the use of a genetic pharmacopeia indispensable for diagnostics applications. The use of larger (e.g. whole genome) libraries for personalized medicine is simply not feasible, which until now has precluded the use of these technologies for precision medicine.

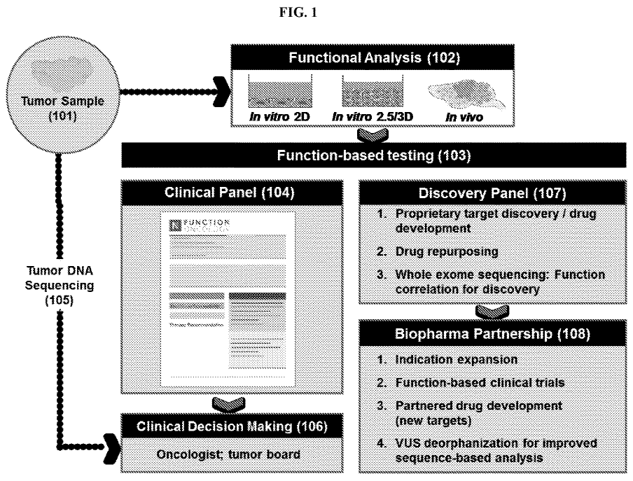

In one aspect, provided herein are methods of determining the susceptibility of a disease or condition to a library of therapeutic agents represented by a library of perturbagens which model the action of those therapeutic agents. A clinical workflow of a functional susceptibility profiling method for a patient with cancer is shown in FIG. 1. In an initial step 101, a sample of primary, patient-derived cancer cells is obtained from the patient. In a subsequent step 102, the cancer cells are contacted with a library of gene modulatory reagents which model the function of a library of cancer drugs having known protein targets by editing (e.g., CRISPR-based methods) and/or silencing (e.g., siRNA) the genes encoding for those protein targets. The resulting modified cancer cells are propagated by in vitro 2D culture, in vitro 2.5D/3D culture, or in vivo. This step may involve use of improved 3D in vitro models of in vivo growth, methods for suppression of stromal cell outgrowth, co-culture with autologous or allogenic immune cells (e.g. T cells), or improved methods for xenograft development in vivo, or any combination thereof. To evaluate the effect of each gene perturbation, in a subsequent step 103, the propagated modified cancer cells are tested, e.g., by next generation sequencing (NGS), to obtain a readout regarding which gene modulatory reagents affect the viability of the patient's cancer cells. This step may involve use of developed internal references to calibrate dropout analysis and correct for sample-to-sample variation. A clinical panel 104 is generated identifying the effective gene modulatory reagents and/or corresponding cancer drugs. A clinician, such as an oncologist, or a group of clinicians, such as a tumor board, evaluate the clinical panel 104 and make a clinical decision 106 regarding a course of treatment for the patient. To assist with the clinical decision 106, the unmodified tumor itself may be subjected to DNA sequencing 105.

In another aspect, the methods described herein facilitate the generation of a discovery panel 107, which may include newly discovered drug targets, e.g., to assist with drug development; newly discovered use(s) of a known drug (drug repurposing); and/or the functional correlation to the discoveries based on whole exome sequencing. These discoveries may be partnered with Biopharmaceutical companies 108 to assist with expansion of drug indications for known drugs; function-based clinical trials of known drugs; development of drugs against newly discovered targets; and/or improvement of sequence-based analyses via deorphanization of variants of unknown significance (VUS).

The method described in FIG. 1 may further comprise designing the library of gene modulatory reagents used in the functional analysis step 102. The design may involve defining the full targeted pharmacologic landscape by generating a list of all targeted drugs (drug library) for cancer. As an example, the drug library comprises at least one of the cancer drugs of Table 2, e.g., at least about 5, 10, 20, 50, 100, 150, 200, 250, 300, 400, 500, 1000, or 1500 of the drugs listed in Table 2. As another example, the drug library comprises at least one of the cancer drugs of Table -3. In some cases, the drug library comprises a plurality of cancer drugs of Table 3, e.g., at least about 5, 10, 20, 50, 100, 150, 200, 250, 300, 400, 500, 1000, or 1500 of the drugs listed in Table 3. The drug library may comprise all of the targeted drugs for a particular type of cancer. As used herein, “all targeted drugs” may refer to at least about 90%, 95%, or 100% of all FDA-approved drugs for a particular indication, e.g., cancer in general or a particular type of cancer. All targeted drugs may also include investigational drugs, such as drugs undergoing regulatory review, but have not yet been approved, and drugs used in clinical trials or pre-clinical testing.

The method described in FIG. 1 may further comprise determining the protein and associated gene targets of the drugs in a drug library, such as the drug library comprising one or more cancer drugs of Tables 2-3, e.g., a drug of Table 2. This requires that a target is known or proposed for each drug included in the analysis. In the case of non-specific inhibitors, such as multi-kinase inhibitors, the targets may include multiple gene targets. As a non-limiting example, the library comprises at least one of the targets of Tables 4-6B, 6D. In some cases, the library comprises a plurality of targets of Tables 4-6B, 6D, e.g., at least about 5, 10, 20, 50, 100, 150, 200, 250, or 300 of the targets listed in Tables 4-6B, 6D.

The library of gene modulatory reagents used in the functional analysis shown in FIG. 1 may be designed by selecting reagents that target the genes of the target library, e.g., the reagents target the genes encoding for one or more targets of Tables 4-6B, 6D, e.g., a target from Table 6D. Reagents may be selected that have been validated for efficacy in inhibiting the target, thus providing a more “compact” library. In an exemplary embodiment, the library comprises at least one nucleic acid comprising a sequence selected from SEQ ID NOS: 1-2789, 2980-3071. In some cases, the library comprises a plurality of nucleic acid sequences selected from SEQ ID NOS: 1-2789, 2980-3071. In an exemplary embodiment, the library comprises at least one nucleic acid comprising a sequence selected from SEQ ID NOS: 1526-2789. In some cases, the library comprises a plurality of nucleic acid sequences selected from SEQ ID NOS: 1526-2789. In an exemplary embodiment, the library comprises at least one nucleic acid comprising a sequence selected from SEQ ID NOS: 2980-3071. In some cases, the library comprises a plurality of nucleic acid sequences selected from SEQ ID NOS: 2980-3071. In some CRISPR-based methods, the library comprises a control gRNA sequence, e.g., a non-cutting control sequence that does not have a target in the human genome and/or a cutting sequence that targets a non-genetic region of the human genome. For example, the library may comprise one or more of the sequences of SEQ ID NOS: 2790-2971 (Table 6C). The library of reagents may be constructed in a format compatible with use in cells, e.g., primary (directly patient-derived) cancer cells. This step may involve the use of novel viral vector systems, the use of non-viral methods for reagent delivery to the cells, or the use of novel gene editing agents (e.g., non-Cas9 CRISPR nucleases), or any combination thereof

Accordingly, an exemplary method of the present disclosure may comprise one or more of the following steps: (1) Defining the full targeted pharmacologic landscape by generating a list of all targeted drugs for a disease or condition (drug library). (2) Determining the protein targets of these drugs, and the genes encoding those protein targets (genetic pharmacopeia). (3) Designing a library of gene modulatory reagents to target the genes encoding these proteins. (4) Constructing the library as well as any needed gene silencing/editing agents in a format compatible with use in cells, e.g., primary cancer cells. (5) Delivering the library and any needed gene silencing/editing agents into cells, e.g., primary, patient-derived cancer cells. (6) Propagating the edited cells. (7) Obtaining a readout of the effect of each perturbation, e.g., by next generation sequencing (NGS)-based methods. (8) Interpreting the resulting barcode distributions to determine the effect of individual perturbations on the viability of the patient's diseased cells. Although the methods have been exemplified with regard to personalized cancer treatment, these methods are also suitable for treatment of non-cancer based diseases or conditions.

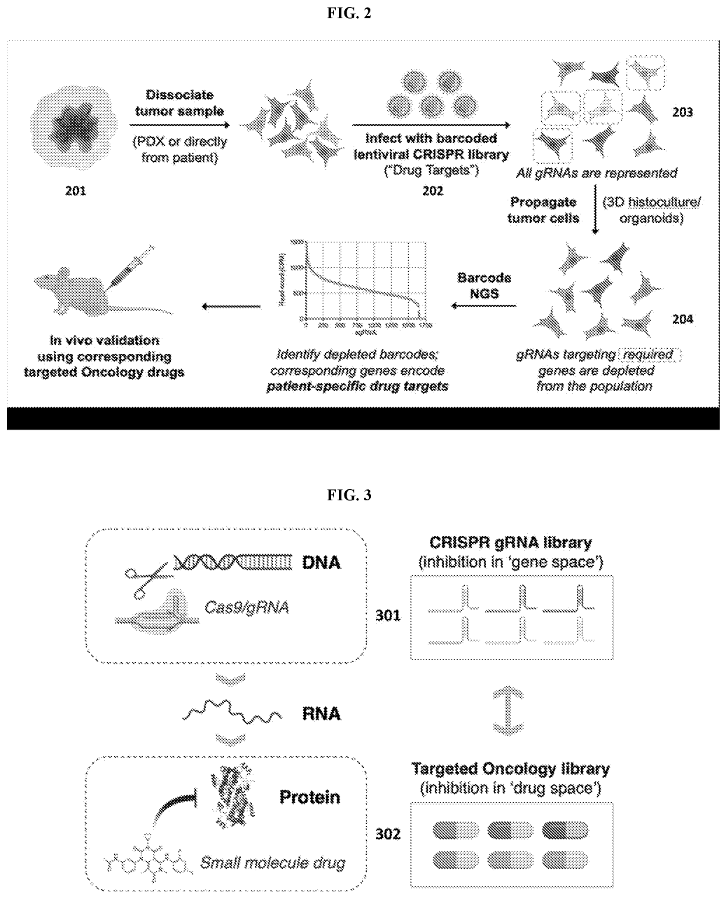

A non-limiting exemplary generic flowchart for the identification of patient-specific tumor therapeutic vulnerabilities utilizing function genomics described herein is shown in FIG. 2. Patient-derived samples (201), either obtained directly from the patient or after passage in mice (PDX), are dissociated (202) and infected with a gRNA library corresponding to the desired therapeutic drug collection (203). Cells are viably maintained in vitro, for instance using 3D and/or organoid approaches, allowing gRNA which target essential tumor regulators to be gradually depleted from the population (“drop-out”) (204). Next-generation sequencing is performed to identify depleted barcodes corresponding to genes depleted from the population and encoding for patient-specific drug targets (205). Oncology drugs corresponding to the patient-specific drug targets are validated in vivo (206). As represented by the schematic in FIG. 3, this approach leverages the insight that the effect of each clinically used targeted oncology drug (302) can be modeled by CRISPR-mediated mutation of the corresponding gene encoding the drug target (301).

In the present description, certain specific details are set forth in order to provide a thorough understanding of various embodiments. However, one skilled in the art will understand that the embodiments provided may be practiced without these details. Unless the context requires otherwise, throughout the description and claims which follow, the word “comprise” and variations thereof, such as, “comprises” and “comprising,” are to be construed in an open, inclusive sense, that is, as “including, but not limited to.” As used in this description and the appended claims, the singular forms “a,” “an,” and “the” include plural referents unless the context clearly dictates otherwise. It should also be noted that the term “or” includes “and/or” unless the context clearly dictates otherwise. Further, headings provided herein are for convenience only and do not interpret the scope or meaning of the claimed embodiments.

The terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J Mol Biol. 1990 Oct. 5; 215(3):403-10; Nucleic Acids Res. 1997 Sep. 1; 25(17):3389-402). Percent homology of sequences can be determined using the most recent version of BLAST, as of the filing date of this application. Percent identity of sequences can be determined using the most recent version of BLAST, as of the filing date of this application.

### Targeted Pharmacologic Landscape

In one aspect, provided herein is a pharmacologic landscape comprising a library of therapeutic agents having known protein targets, referred to as a drug library. The drug library may include low molecular weight drugs (e.g., having a molecule weight less than about 1 kDa) and biologic drugs (e.g., proteins such as antibodies). The drug library may comprise drugs suitable for a patient's particular disease or condition, such as cancer or an autoimmune disease. In various embodiments, the drug library includes FDA-approved therapeutic agents and as such may be expanded as new drugs are developed. The drug library may include all or nearly all of the targeted drugs treating a particular class of disease, e.g., the drug library includes at least about 50%, at least about 60%, at least about 70%, at least about 80%, at least about 90%, or 100% of known FDA-approved drugs for a particular disease class having a known protein target. Also provided herein are focused libraries for investigational therapies (e.g., those in Phase I-III clinical testing), and libraries of a particular target classes of interest (e.g., G-protein coupled receptors, kinases, etc.).

Drug Library for Cancer

In certain embodiments, a drug library is designed comprising two or more therapies shown to be efficacious for, and/or have received FDA approval for, treating cancer. In some embodiments, the drug library comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, or at least 300 therapeutic agents. In some embodiments, the drug library comprises up to about 100, up to about 200, up to about 300, up to about 400, up to about 500, or up to about 1000 therapeutic agents. One or more of the therapeutic agents may be selected from Table 2. One or more of the therapeutic agents may be selected from Table 3.

In certain embodiments, a drug library is designed comprising two or more cancer therapeutics specific for a certain type of cancer. As non-limiting examples, the drug library comprises two or more cancer therapeutics shown to be efficacious for, and/or have received FDA approved for, melanoma, thyroid, colorectal, endometrial, lung, pancreatic, breast, genitourinary, gastrointestinal, ovarian, or head and neck cancer, or any cancer listed herein or known in the art. In some embodiments, the cancer-specific drug library comprises at least 1, at least 2, at least 3, at least 4, at least 5, at least 6, at least 7, at least 8, at least 9, or at least 10 therapeutic agents. In some embodiments, the cancer-specific drug library comprises up to about 10, 20, 30, 40, 50, 60, 70, 80, 90, or 100 therapeutic agents. One or more of the therapeutic agents may be selected from Table 2. One or more of the therapeutic agents may be selected from Table 3.

In certain embodiments, the drug library comprises at least one cancer therapeutic agent chosen from Table 2. The drug library may include at least 2, at least 3, at least 4, at least 5, at least 6, at least 7, at least 8, at least 9, at least 10, at least 15, at least 20, at least 25, at least 30, at least 35, at least 40, at least 45, at least 50, at least 60, at least 70, at least 80, at least 90, at least 100, at least 120, at least 140, at least 160, at least 180, at least 200, at least 250, at least 300, at least 350, at least 400, at least 450, at least 500, at least 600, at least 700, at least 800, or at least 900 therapeutic agents chosen from Table 2. The drug library may comprise the at least one cancer therapeutic agent chosen from Table 2, and one or more additional FDA-approved therapeutic agent(s) for cancer. The drug library may comprise at least about 50%, at least about 60%, at least about 70%, at least about 80%, at least about 90%, or 100% of known FDA-approved molecularly targeted cancer drugs. The drug library may comprise the at least one cancer therapeutic agent chosen from Table 2, and one or more additional therapeutic agent(s) for cancer that is undergoing FDA-approval and/or is the subject of any current or completed clinical trial.

In certain embodiments, the drug library comprises at least one cancer therapeutic agent chosen from Table 3. The drug library may include at least 2, at least 3, at least 4, at least 5, at least 6, at least 7, at least 8, at least 9, at least 10, at least 15, at least 20, at least 25, at least 30, at least 35, at least 40, at least 45, at least 50, at least 60, at least 70, at least 80, at least 90, at least 100, at least 120, at least 140, at least 160, at least 180, at least 200, at least 250, at least 300, at least 350, at least 400, at least 450, at least 500, at least 600, at least 700, at least 800, or at least 900 therapeutic agents chosen from Table 3. The drug library may comprise the at least one cancer therapeutic agent chosen from Table 3, and one or more additional FDA-approved therapeutic agent(s) for cancer. The drug library may comprise at least about 50%, at least about 60%, at least about 70%, at least about 80%, at least about 90%, or 100% of known FDA-approved molecularly targeted cancer drugs. The drug library may comprise the at least one cancer therapeutic agent chosen from Table 3, and one or more additional therapeutic agent(s) for cancer that is undergoing FDA-approval and/or is the subject of any current or completed clinical trial.

Gene Target Libraries

Further provided herein is a library of genetic targets comprising the genes encoding the proteins targeted by the therapeutic agents in the drug library. For therapeutic agents that are non-specific inhibitors, such as multi-kinase inhibitors, the targets may include multiple gene targets. The number of targeted genes must be significantly smaller than the “whole genome,” generating a compact library amenable to both in vitro and in vivo analysis. Non-limiting examples of targeted genes are shown in Table 4. Non-limiting examples of targeted genes for oncology are shown in Tables 5A-6B, 6D. The targeted genes described herein may comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, or at least 300 genes from Table 5A. The targeted genes described herein may comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, or at least 300 genes from Table 5B. The targeted genes described herein may comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, or at least 300 genes from Table 5C. The targeted genes described herein may comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, or all of the genes from Table 5D. The targeted genes described herein may comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, or at least 300 genes from Table 6A. The targeted genes described herein may comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, or at least 300 genes from Table 6B. The targeted genes described herein may comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, or all of the genes from Table 6D. In some embodiments, the library comprises one or more genes to validate successful gene editing. A non-limiting example utilized in experiments described herein is the B2M gene.

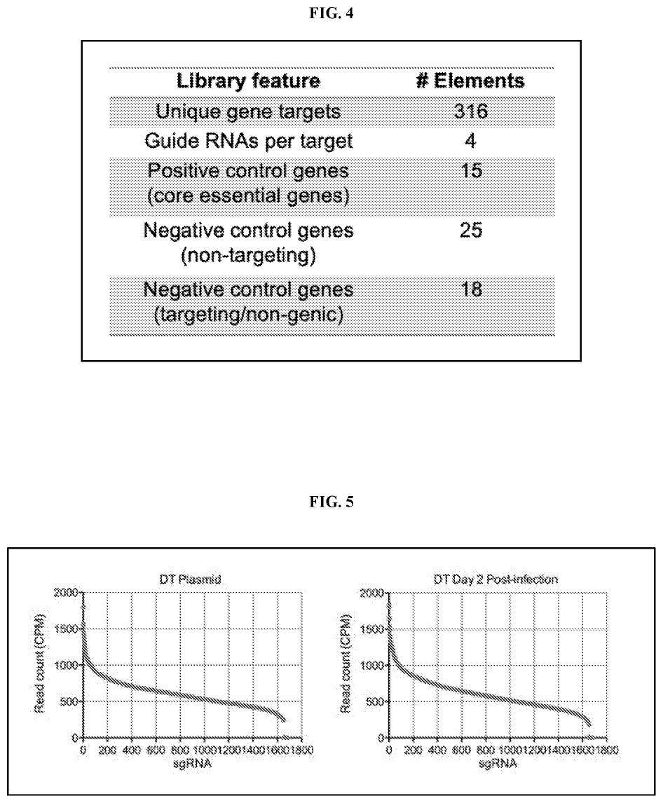

A non-limiting exemplary gene target library was constructed as further described in the examples and characterized in FIG. 4 as targeting 316 unique genes. The genes targeted by the library include those listed in Table 5C. Accordingly, provided herein is a library targeting one or more of the genes of Table 5C, e.g., at least about 1, 2, 3, 4, 5, 6, 7, 8, 9, 10, 11, 12, 13, 14, 15, 20, 25, 30, 35, 40, 45, 50, 60, 70, 80, 90, 100, 120, 140, 160, 180, 200, 220, 240, 260, 280, 300, 310, or all of the genes of Table 5C.

Another non-limiting exemplary gene target library was constructed that targets 23 unique genes, as further described in the examples. The genes targeted by the library include those listed in Table 5D and B2M. Accordingly, provided herein is a library targeting one or more of the genes of Table 5D, e.g., at least about 1, 2, 3, 4, 5, 6, 7, 8, 9, 10, 11, 12, 13, 14, 15, 16, 17, 18, 19, 20, 21, 22, or all of the genes of Table 5D. In some cases the gene target library comprises a gene for validation purposes, such as B2M.

### Genetic Pharmacopeia

In one aspect, provided herein is a library of genetic elements which represent a collection of existing drugs for a particular disease or condition. These genetic elements are capable of modifying a patient's cells to mimic the effect of the existing drugs on the patient, allowing for personalized comprehensive functional profiling. The profiling may be performed in a pooled screening format to allow for screening of the effects of the modifications in parallel. Such highly parallel functional genomics methodology is utilized in preclinical biology, but has not been applicable to personalized therapeutic sensitivity profiling. Additionally, this approach enables comprehensive assessment of the impact of therapeutic manipulations in an in vivo testing paradigm, of critical importance for the reasons previously indicated herein.

Accordingly, disclosed herein are methods for the design, construction, and use of a genetic pharmacopeia comprising a plurality of gene modulatory reagents capable of modifying a patient's cells to knock out, or knock down, function of genes encoding for protein targets of a collection of existing drugs. In some embodiments, a genetic pharmacopeia is designed using publicly available tools, e.g., publicly available methods and reagents for gene editing or gene silencing. In some embodiments, a subset of these reagents will work poorly, most will be acceptable, and a minority will demonstrate exceptional performance. Pre-selection of reagents that have been validated to work well will be advantageous both with regard to efficiency of delivery and production of a more “compact” library, both of which reduce the number of patient-derived cells needed and increase the quality of data produced. In some embodiments, the design includes selection of the most efficacious or advantageous modulatory mechanism (e.g., CRISPR, RNAi). For CRISPR-based methods, the design comprises selection of the most advantageous RNA-guided endonuclease (e.g., Cas9 vs. Cas12a vs. Mad7). The design may also include selection of the most efficacious guide or seed sequences. The design may also include multiple gene modulatory reagents expressed from a single vector as a single or multiple transcriptional units. For instance, multiplexed gRNAs may be constructed for use with a Cas12 based nuclease (e.g., Cpfl) to generate a highly compact library. The design may also include elements in the library that allow for the identification, selection, or enrichment of transduced cells (e.g., fluorescent markers, antibiotic resistance cassettes, surface epitope expression cassettes).

The genetic pharmacopeia may be constructed in a format that is compatible with use in patient derived cells, e.g., primary cancer cells. In some embodiments, a viral delivery method is chosen for introduction of the gene modulatory reagent (e.g., guide or seed sequence). Non-limiting examples of viruses include lentivirus, adenovirus, adeno-associated virus, and other viruses disclosed herein. In some embodiments, a non-viral delivery method is selected. As a non-limiting example, the delivery method is transposase-mediated transposition. The library may be constructed using a combination of gene synthesis and pooled molecular cloning techniques. The library may be subject to quality control analysis to ensure full and approximately equal representation of the desired sequences. In some embodiments of a viral delivery method, pooled high-titer virus is prepared. In other embodiments, the virus is delivered in an array to facilitate an arrayed screening format.

Library of Gene Modulatory Reagents

In one aspect, provided herein are libraries comprising a plurality of gene modulatory reagents, each gene modulatory reagent capable of knocking down or knocking out the function of a gene that encodes a protein target from a library of protein targets. The plurality of gene modulatory reagents may be capable of knocking down or knocking out the function of at least about 50%, at least about 60%, at least about 70%, at least about 80%, at least about 90%, or at least about 95% of the genes that encode for the protein targets in the library. In some cases, the library of protein targets comprises all known proteins targeted by known drugs capable of treating a particular disease or condition. An exemplary disease or condition is cancer, e.g., a cancer disclosed herein or otherwise known in the art.

In some embodiments, the library of gene modulatory reagents is capable of knocking down or knocking out the function of one or more genes encoding protein targets selected from Table 5B. In some cases, the protein targets comprise at least 1, at least 2, at least 3, at least 4, at least 5, at least 10, or at least 20 protein targets of Table 5B. In some embodiments, the library of gene modulatory reagents is capable of knocking down or knocking out the function of one or more genes encoding protein targets selected from Table 5A. In some cases, the protein targets comprise at least 1, at least 2, at least 3, at least 4, at least 5, at least 10, or at least 20 protein targets of Table 5A. In some embodiments, the library of gene modulatory reagents is capable of knocking down or knocking out the function of one or more genes encoding protein targets selected from Table 5C. In some cases, the protein targets comprise at least 1, at least 2, at least 3, at least 4, at least 5, at least 10, or at least 20 protein targets of Table 5C. In some embodiments, the library of gene modulatory reagents is capable of knocking down or knocking out the function of one or more genes encoding protein targets selected from Table 5D. In some cases, the protein targets comprise at least 1, at least 2, at least 3, at least 4, at least 5, at least 10, or at least 20 protein targets of Table 5D. In some cases, the library of gene modulatory reagents is capable of knocking down or knocking out the function of one or more genes encoding for protein targets of one or more known drugs selected from Table 2-3. In some cases, the one or more known drugs comprises at least 1, at least 2, at least 3, at least 4, at least 5, at least 10, or at least 20 drugs of Table 2-3. In some cases, the library of gene modulatory reagents is capable of knocking down or knocking out the function of one or more genes encoding for protein targets of one or more known drugs selected from Table 2. In some cases, the one or more known drugs comprises at least 1, at least 2, at least 3, at least 4, at least 5, at least 10, or at least 20 drugs of Table 2. The plurality of gene modulatory reagents may be capable of knocking down or knocking out the function of about 10 to about 2,000, from about 10 to about 500, from about 10 to about 200, from about 10 to about 150, from about 50 to about 500, from about 50 to about 200, about 50 to about 2,000, or about 100 to about 2,000 genes. The library may comprise about 10 to about 2,000, from about 10 to about 500, from about 10 to about 200, from about 10 to about 150, from about 50 to about 500, from about 50 to about 200, about 50 to about 2,000, or about 100 to about 2,000 gene modulatory reagents.

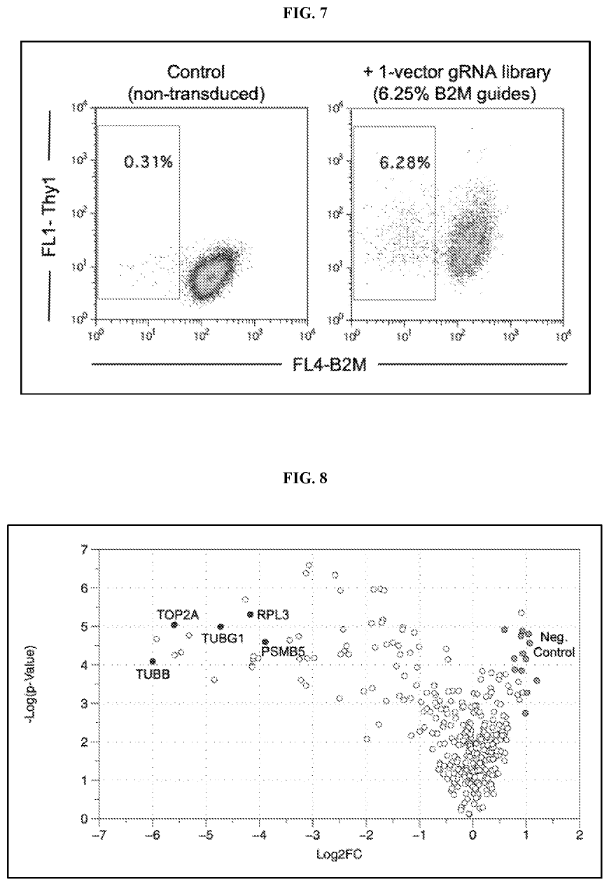

At least one of the gene modulatory reagents may be capable of knocking out the function of a gene. For instance, the at least one gene modulatory reagent is part of a CRISPR-based gene editing system. In some cases, one or more of the plurality of gene modulatory reagents each comprise a gRNA sequence comprising at least about 90% homology or identity to a sequence selected from SEQ ID NOS: 1-2789, 2980-3071. In some cases, one or more of the plurality of gene modulatory reagents each comprise a gRNA sequence comprising at least about 90% homology or identity to a sequence selected from SEQ ID NOS: 1526-2789. In some cases, one or more of the plurality of gene modulatory reagents each comprise a gRNA sequence comprising at least about 90% homology or identity to a sequence selected from SEQ ID NOS: 2980-3071. In some cases, at least one of the gene modulatory reagents comprise a gRNA sequence having homology to at least a portion of the gene whose function is knocked out by the gene modulatory reagent. In some embodiments, the gene modulatory reagents comprise one or more control sequences. As a non-limiting example, the sequence is a gRNA control that does not have a target in the human genome. As another non-limiting example, the sequence is a gRNA control that targets a non-genetic region of the human genome. For instance, the library may comprise one or more of the sequences of SEQ ID NOS: 2790-2971 (Table 6C). The inclusion of targeting (e.g., CTRL-hg38 of Table 6C) and non-targeting (e.g., CTRL-non sequences of Table 6C) control gRNAs enables an estimate of the impact of dsDNA breaks in innocuous genome locations. In some embodiments, the gene modulatory reagents comprise a gRNA that targets a gene for validation of successful gene editing. For instance, as described in the examples and FIG. 7, gRNAs may be included that target the cell surface marker B2M at 6.25% of all gRNAs in the focused library (SEQ ID NOS: 2960-3071 and 2890-2905), enabling the validation of successful CRISPR editing in the population by flow cytometry.

At least one of the gene modulatory reagents may be capable of knocking down the function of a gene. For instance, the at least one gene modulatory reagent comprises an shRNA sequence having homology to at least a portion of the gene whose function is knocked down by the gene modulatory reagent. The homology may be at least about 90% sequence homology or identity. The at least a portion may be at least about 15 contiguous nucleotides.

Non-limiting exemplary libraries of gene modulatory reagents were prepared and characterized (FIG. 4). One library was constructed for CRISPR-based gene editing, targeting 316 unique genes, with 4 guide RNAs per target. The guide RNAs utilized are listed in Table 6B. The library also included the control guide RNAs of Table 6C. Another library of gene modulatory reagents was constructed for CRISPR-based gene editing, targeting 23 unique genes, with 4 guide RNAs per target. The guide RNAs utilized in the later library are listed in Table 6D. The library also included guide RNAs of Table 6C having SEQ ID NOS: 2890-2905 and 2960-2979. This later library has a smaller size, which enables screening to be performed with smaller cell numbers, such as with primary cancer cells.

In some embodiments, a library of gene modulatory reagents comprises one or more gene modulatory reagents that target a gene of Table 5D. In some embodiments, the library comprises one or more gene modulatory reagents that target at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, or all of the gene targets of Table 5D. In some embodiments, the library comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 55, at least 60, at least 65, at least 70, at least 75, at least 80, at least 85, at least 90, or all of the gRNA of Table 6D.

In some embodiments, one or more of the gene modulatory reagents is designed to knock out or knock down the function of a positive control gene, such as a core essential gene for the cell. Such reagents may serve as a positive control for library functionality. In some embodiments, one or more of the gene modulatory reagents is designed to knock out or knock down the function of a non-targeting gene and/or a targeting and non-genic gene. Such gene modulatory reagents may serve as negative controls. Non-limiting control gene modulatory reagents are provided in Table 6C.

In some embodiments, one or more of the gene modulatory reagents is positioned within a vector. The vector may comprise an adapter sequence. The adapter sequence may comprise a type IIS restriction enzyme cleavage site, which may allow for GoldenGate assembly cloning. The adapter sequence may comprise homology arms compatible with a destination vector allowing for cloning by overhang homology based methods, such as Gibson assembly. The vector may also comprise genetic elements of a virus. Non-limiting examples of viruses include adenovirus, retrovirus, adeno-associated virus (AAV), pox virus, parvovirus, baculovirus, measles virus, herpes simplex virus (HSV), Moloney Murine Leukemia Virus (MoMLV, MMLV, MuLV, or MLV), Murine Stem cell Virus (MSCV), and human immunodeficiency virus (HIV). The vector may also comprise a sequence encoding a marker, an antibiotic resistance cassette, or surface epitope expression cassette, or a combination thereof. The marker may be a fluorescent marker.

CRISPR

In one aspect, provided is a library comprising a plurality of gene modulatory reagents, wherein each modulatory reagent comprises a guide RNA (gRNA) homologous to a target gene. The target gene may encode for a protein targeted by a known therapeutic agent (e.g., a therapeutic agent from Tables 2-3). Non-limiting examples of target genes are listed in Tables 4-6B, 6D. In some embodiments, one or more of the gRNAs comprise a sequence at least about 85%, 90%, 95%, or 100% homologous to at least about 10, 15, or 20 contiguous nucleobases of a target gene. In some embodiments, one or more of the gRNAs comprise a sequence at least about 85%, 90%, 95%, or 100% homologous to at least about 10, 15, or 20 contiguous nucleobases of a target gene chosen from Tables 4-6B, 6D. In some embodiments, the library comprises one or a plurality of sequences selected from SEQ ID NOS: 1-2789, 2980-3071. In some embodiments, the library comprises one or a plurality of sequences having at least about 85%, 90%, 95%, or 100% homology to a sequence selected from SEQ ID NOS: 1-2789, 2980-3071. In some embodiments, the library comprises one or a plurality of sequences selected from SEQ ID NOS: 1526-2789. In some embodiments, the library comprises one or a plurality of sequences having at least about 85%, 90%, 95%, or 100% homology to a sequence selected from SEQ ID NOS: 1526-2789. In some embodiments, the library comprises one or a plurality of sequences selected from SEQ ID NOS: 2790-2959. In some embodiments, the library comprises one or a plurality of sequences having at least about 85%, 90%, 95%, or 100% homology to a sequence selected from SEQ ID NOS: 2790-2959. In some embodiments, the library comprises one or a plurality of sequences selected from SEQ ID NOS: 1526-2790. In some embodiments, the library comprises one or a plurality of sequences having at least about 85%, 90%, 95%, or 100% homology to a sequence selected from SEQ ID NOS: 1526-2790. In some embodiments, the library comprises one or a plurality of sequences selected from SEQ ID NOS: 2980-3071. In some embodiments, the library comprises one or a plurality of sequences having at least about 85%, 90%, 95%, or 100% homology to a sequence selected from SEQ ID NOS: 2980-3071. The library may comprise from about 10 to about 2,000, from about 10 to about 500, from about 10 to about 200, from about 10 to about 150, from about 50 to about 500, from about 50 to about 200, from about 50 to about 2,000, from about 100 to about 2,000, from about 200 to about 2,000, or from about 500 to about 2,000 different gRNA sequences. In some embodiments, one or more of the gRNA sequences is encoded on a vector.

In some embodiments, the library further comprises an RNA-guided endonuclease such as Cas9, Cas12, Cas12a (or Cpfl or Mad7), Cas12b (or C2c1 or Cpf2), Cas12c (C2c3), Cas12d (or CasY), Cas12e (or CasX), Cas13, Cas13a (or C2c2), Cas13b (or C2c6), Cas13c (or C2c7), Cas13d (or Casrx), Cas14, Cas14a, Cas14b, Cas14c, Cas1, Cas1B, Cas2, Cas3, Cas4, Cas5, Cas5e (CasD), Cash, Cas6e, Cas6f, Cas7, Cas8a, Cas8al, Cas8a2, Cas8b, Cas8c, Csnl, Csx12, Cas10, Cas10d, Cas10, Cas10d, CasF, CasG, CasH, Csy1, Csy2, Csy3, Csel (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cul966, or derivative thereof, variant thereof, fragment thereof, or any combination thereof. In some embodiments, the endonuclease is of the Cas9 or Cas12a family, which may include, but is not limited to, S. pyogenes Cas9 (SpCas9), SpCas9 D1135E variant, SpCas9 VRER variant, SpCas9 EQR variant, xCas9, SpCas9-NG, S. aureus Cas9 (SaCas9), Acidaminococcus sp. (AsCpfl), Lachnospiraceae bacterium (LbCpfl), AsCpfl RR variant, LbCpfl RR variant, AsCpfl RVR variant, C. jejuni Cas9 (Cj Cas9), N. meningitidis (NmCas9), S. thermophilus (StCas9), T. denticola (TdCas9), and Mad7. Additionally, other RNA-guided endonucleases that are suitable for the library disclosed herein include zinc finger nucleases (ZFN), transcription activator-like effector nucleases (TALEN), meganucleases, RNA-binding proteins (RBP), recombinases, flippases, transposases, Argonaute (Ago) proteins (e.g., prokaryotic Argonaute (pAgo), archaeal Argonaute (aAgo), and eukaryotic Argonaute (eAgo)), and any functional fragment thereof, and any combination thereof.

In some cases, the gRNA and/or endonuclease is encoded on a vector. In some cases, a vector comprising gRNA and/or endonuclease comprises one or more features of a viral genome. As a non-limiting example, the viral vector includes retroviral vector, adenoviral vector, adeno-associated viral vector (AAV), pox vectors, parvoviral vectors, baculovirus vectors, measles viral vectors, or herpes simplex virus vector (HSV). In some instances, the retroviral vector includes gamma-retroviral vector, such as a vector derived from the Moloney Murine Leukemia Virus (MoMLV, MMLV, MuLV, or MLV) or the Murine Stem cell Virus (MSCV) genome. In some instances, the retroviral vector comprises lentiviral vectors such as those derived from the human immunodeficiency virus (HIV) genome. In some instances, AAV vector comprises AAV1, AAV2, AAV4, AAV5, AAV6, AAV7, AAV8, or AAV9 serotype. In some instances, the viral vector is a chimeric viral vector, comprising viral portions from two or more viruses. In additional instances, the viral vector is a recombinant viral vector.

In some embodiments, the vector comprises a marker for selection, e.g., an antibiotic resistance cassette or surface epitope expression cassette. In some embodiments, the gene modulatory reagent and endonuclease are encoded by separate vectors. As a non-limiting example, the endonuclease is delivered via adenovirus, while the gRNA is delivered by lentivirus. In some embodiments, the endonuclease coding sequence may be split between two vectors. For instance, this method may be employed when constructing large endonucleases such as Cas9. In some embodiments, the gene modulatory reagent is encoded by a viral vector and the endonuclease is provided as a ribonuclear protein complex transfected into target cells, for instance using lipid or electroporation techniques.

RNAi

In one aspect, provided is a library comprising a plurality of gene modulatory reagents, wherein one or more of the modulatory reagents comprise a short hairpin RNA (shRNA) complementary to a target mRNA of a protein targeted by a known therapeutic agent (e.g., a therapeutic agent chosen from Tables 2-3). Non-limiting examples of target proteins include those encoded by the genes listed in Tables 4-6B, 6D. In some embodiments, one or more of the shRNA each comprise a sequence at least about 85%, 90%, 95%, or 100% complementary to at least about 10, 15, or 20 contiguous nucleobases of a target mRNA. In some embodiments, one or more of the shRNA each comprise a sequence at least about 85%, 90%, 95%, or 100% complementary to at least about 10, 15, or 20 contiguous nucleobases of a target mRNA encoding for a protein selected from Tables 4-6B, 6D. The library may comprise from about 10 to about 2,000, from about 50 to about 2,000, from about 10 to about 500, from about 10 to about 200, from about 10 to about 150, from about 50 to about 500, from about 50 to about 200, from about 50 to about 2,000, from about 100 to about 2,000, or from about 500 to about 2,000 different shRNA sequences.

### Genetic Modification and Cell Propagation

In some aspects of the disclosure, a library comprising a plurality of gene modulatory reagents is delivered to a sample of cells from a subject having a disease or condition to generate a plurality of modified cells. In exemplary embodiments, the subject has cancer and the sample of cells comprise primary cancer cells. For some embodiments involving cancer cells, tumor samples are processed in a manner that preserves cancer cell viability, while maximizing cellular yield. Non-limiting examples of delivery methods include viral methods (e.g., lentivirus, adenovirus, or adeno-associated virus) as well as non-viral methods (e.g., transposase-mediated transposition employing transposons such as piggybac or sleeping beauty, or integrases such as phi31). In some embodiments, delivery of viral particles to the cells is performed in a manner that ensures equal and adequate representation of clones, while minimizing multiplicity of infection. In particular, the number of times each clone is presented within the population (“representation”) may be a crucial factor which determines the power of the eventual analysis to sensitively and specifically detect changes in barcode abundance following in vitro or in in vivo propagation.

Methods of Genetic Modification

An exemplary method for generating a plurality of modified cancer cells from a subject comprises: delivering a library of gene modulatory reagents to a sample of cancer cells from the subject to generate the plurality of modified cancer cells, wherein each modified cancer cell harbors one or more of the gene modulatory reagents, and each gene modulatory reagent is capable of knocking down or knocking out the function of a gene that encodes a protein target from a library of protein targets.

In some embodiments, the method for generating the plurality of modified cancer cells comprises a CRISPR/endonuclease-based gene editing system. For instance, one or more of the gene modulatory reagents comprises a gRNA sequence comprising homology to at least a portion of the gene whose function is knocked out in the modified cancer cell. The gRNA may comprise homology to about 10 to about 50 contiguous nucleotides of the gene. The homology may be at least about 90% sequence homology or identity. In some embodiments, one or more of the gene modulatory reagents each comprise a gRNA sequence comprising at least about 90% homology or identity to a sequence selected from SEQ ID NOS: 1-2789, 2980-3071. In some embodiments, one or more of the gene modulatory reagents each comprise a gRNA sequence comprising at least about 90% homology or identity to a sequence selected from SEQ ID NOS: 1526-2789. In some embodiments, one or more of the gene modulatory reagents each comprise a gRNA sequence comprising at least about 90% homology or identity to a sequence selected from SEQ ID NOS: 2790-2959. In some embodiments, one or more of the gene modulatory reagents each comprise a gRNA sequence comprising at least about 90% homology or identity to a sequence selected from SEQ ID NOS: 2980-3071. The gRNA may be positioned within a vector, e.g., for viral delivery as discussed herein.

The method for generating modified cancer cells may further comprise contacting the cancer cells with an endonuclease. The endonuclease may comprise a Cas9 or Cas12a endonuclease. Non-limiting examples of Cas9 or Cas12a endonucleases include S. pyogenes Cas9 (SpCas9), SpCas9 D1135E variant, SpCas9 VRER variant, SpCas9 EQR variant, xCas9, SpCas9-NG, S. aureus Cas9 (SaCas9), Acidaminococcus sp. (AsCpfl), Lachnospiraceae bacterium (LbCpfl), AsCpfl RR variant, LbCpfl RR variant, AsCpfl RVR variant, C. jejuni Cas9 (CjCas9), N. meningitidis (NmCas9), S. thermophilus (StCas9), T. denticola (TdCas9), and Mad7. In some cases, the endonuclease does not comprise a Cas9 or Cas12a endonuclease.

In some embodiments, the method for generating the plurality of modified cancer cells comprises an RNA interference (RNAi) gene silencing system. For instance, each gene modulatory reagent comprises a shRNA sequence targeting mRNA encoding for a protein target from the library of protein targets. The shRNA may have homology to about 10 to about 50 contiguous nucleotides of the gene. The homology may be at least about 90% sequence homology or identity. The shRNA may be positioned within a vector, e.g., for viral delivery as discussed herein.

In some embodiments, the library of gene modulatory reagents comprises from about 10 to about 2,000, from about 10 to about 500, from about 10 to about 200, from about 10 to about 150, from about 50 to about 500, from about 50 to about 200, from about 50 to about 2,000, from about 100 to about 2,000, or from about 500 to about 2,000 different gene modulatory reagents. In some cases, at least about 90% of the gene modulatory reagents are present in the library in a quantity within about 10% of the average gene modulatory reagent quantity.

In some embodiments, the library of protein targets comprises at least 1, at least 2, at least 3, at least 4, at least 5, at least 10, or at least 20 protein targets of Table 5B. In some embodiments, the library of protein targets comprises at least 1, at least 2, at least 3, at least 4, at least 5, at least 10, or at least 20 protein targets of Table 5A. In some embodiments, the library of protein targets comprises at least 1, at least 2, at least 3, at least 4, at least 5, at least 10, or at least 20 protein targets of Table 5C. In some embodiments, the library of protein targets comprises at least 1, at least 2, at least 3, at least 4, at least 5, at least 10, or at least 20 protein targets of Table 5D.

In some embodiments, the sample of cancer cells comprises primary cancer cells. The sample of cancer cells may comprise about 10⁵ to about 10⁸ cells. The sample of cancer cells may have been processed to preserve cell viability. The method may thus further comprise preparing the sample of cancer cells to preserve cell viability prior to and/or after delivery of the library of gene modulatory reagents. The method may also further comprise propagating the modified cancer cells. Propagation may comprise maintenance of the modified cancer cells in a 2D in vitro culture. Propagation may comprise maintenance of the modified cancer cells in a 3D in vitro culture. Propagation may comprise maintenance of the modified cancer cells in vivo. In some cases, propagation occurs within an animal model, e.g., in a rodent.

CRISPR Gene Editing

In some embodiments, a sample of cells is modified using a CRISPR-based gene editing method. The gene editing method may comprise contacting the sample of cells with a plurality of gRNA sequences, wherein one or more of the gRNAs have sequence homology to a target gene encoding a protein targeted by a therapeutic agent. Non-limiting examples of target genes are provided in Tables 4-6B, 6D. Non-limiting examples of therapeutic agents are provided in Tables 2-3. In some embodiments, the sample of cells is contacted with at least one or a plurality of gRNA sequences chosen from SEQ ID NOS: 1-2789, 2980-3071. In some embodiments, the sample of cells is contacted with at least one or a plurality of gRNA sequences, each having at least about 85% homology to a sequence chosen from SEQ ID NOS: 1-2789, 2980-3071. In some embodiments, the sample of cells is contacted with at least one or a plurality of gRNA sequences chosen from SEQ ID NOS: 2980-3071. In some embodiments, the sample of cells is contacted with at least one or a plurality of gRNA sequences, each having at least about 85% homology to a sequence chosen from SEQ ID NOS: 2980-3071. In some embodiments, the sample of cells is contacted with at least one or a plurality of gRNA sequences chosen from SEQ ID NOS: 1526-2789. In some embodiments, the sample of cells is contacted with at least one or a plurality of gRNA sequences, each having at least about 85% homology to a sequence chosen from SEQ ID NOS: 1526-2789. In some embodiments, the sample of cells is contacted with at least one or a plurality of gRNA sequences chosen from SEQ ID NOS: 1526-2959. In some embodiments, the sample of cells is contacted with at least one or a plurality of gRNA sequences, each having at least about 85% homology to a sequence chosen from SEQ ID NOS: 1526-2959. In some embodiments, the sample of cells is contacted with at least one or a plurality of gRNA sequences chosen from SEQ ID NOS: 2790-2959. In some embodiments, the sample of cells is contacted with at least one or a plurality of gRNA sequences, each having at least about 85% homology to a sequence chosen from SEQ ID NOS: 2790-2959. The sample of cells is also contacted with an RNA-guided endonuclease, e.g., Cas9, Cas12, Cas12a (or Cpfl or Mad7), Cas12b (or C2c1 or Cpf2), Cas12c (C2c3), Cas12d (or CasY), Cas12e (or CasX), Cas13, Cas13a (or C2c2), Cas13b (or C2c6), Cas13c (or C2c7), Cas13d (or Casrx), Cas14, Cas14a, Cas14b, Cas14c, Cas1, Cas1B, Cas2, Cas3, Cas4, Cas5, Cas5e (CasD), Cash, Cas6e, Cas6f, Cas7, Cas8a, Cas8a1, Cas8a2, Cas8b, Cas8c, Csn1, Csx12, Cas10, Cas10d, Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmrl , Cmr3, Cmr4, Cmr5, Cmr6, Csbl, Csb2, Csb3, Csx17, Csx14, Csx10, Csx16, CsaX, Csx3, Csxl, Csx15, Csf1, Csf2, Csf3, Csf4, Cul966, zinc finger nucleases (ZFN), transcription activator-like effector nucleases (TALEN), meganucleases, RNA-binding proteins (RBP), recombinases, flippases, transposases, Argonaute (Ago) proteins (e.g., prokaryotic Argonaute (pAgo), archaeal Argonaute (aAgo), or eukaryotic Argonaute (eAgo)), or derivative thereof, variant thereof, fragment thereof, or combination thereof.

RNAi

In some embodiments, a sample of cells is modified using an RNAi method. In some embodiments, the sample of cells is contacted with a plurality of shRNA sequences, each shRNA sequence complementary to a target mRNA of a protein targeted by a therapeutic agent. Non-limiting examples of target proteins include those encoded by the genes listed in Tables 4-6B, 6D. Non-limiting examples of therapeutic agents are provided in Tables 2-3.

Compilation of Modified Cancer Cells

Further provided herein are compilations of modified cancer cells. An exemplary compilation comprises a plurality of modified cancer cells, wherein each modified cancer cell harbors one or more gene modulatory reagents, and each gene modulatory reagent is capable of knocking down or knocking out the function of a gene that encodes a protein target from a library of protein targets. In some embodiments, the library of protein targets comprises at least 1, at least 2, at least 3, at least 4, at least 5, at least 10, or at least 20 protein targets of Table 5B. In some embodiments, the library of protein targets comprises at least 1, at least 2, at least 3, at least 4, at least 5, at least 10, or at least 20 protein targets of Table 5C. In some embodiments, the library of protein targets comprises at least 1, at least 2, at least 3, at least 4, at least 5, at least 10, or at least 20 protein targets of Table 5D. In some embodiments, the library of protein targets comprises at least 1, at least 2, at least 3, at least 4, at least 5, at least 10, or at least 20 protein targets of Table 5A. In some embodiments, the library of protein targets comprises at least 1, at least 2, at least 3, at least 4, at least 5, at least 10, or at least 20 protein targets of Table 3. In some embodiments, the library of protein targets comprises at least 1, at least 2, at least 3, at least 4, at least 5, at least 10, or at least 20 protein targets of Table 4. In some embodiments, the modified cancer cells are modified primary cancer cells. The modified cancer cells may comprise from about 10 to about 2,000, from about 50 to about 2,000, from about 10 to about 500, from about 10 to about 200, from about 10 to about 150, from about 50 to about 500, from about 50 to about 200, from about 100 to about 2,000, or from about 500 to about 2,000 different populations of modified cancer cells.

The modified cancer cells may comprise from about 10 to about 2,000, from about 10 to about 500, from about 10 to about 200, from about 10 to about 150, from about 50 to about 500, from about 50 to about 200, from about 50 to about 2,000, from about 100 to about 2,000, or from about 500 to about 2,000 different gene modulatory reagents. At least one of the one or more gene modulatory reagents may comprise a sequence selected from SEQ ID NOS: 1-2789, 2980-3071. At least one of the one or more of gene modulatory reagents may comprise a sequence at least about 90% homologous or identical to a sequence selected from SEQ ID NOS: 1-2789, 2980-3071. At least one of the one or more gene modulatory reagents may comprise a sequence selected from SEQ ID NOS: 2980-3071. At least one of the one or more of gene modulatory reagents may comprise a sequence at least about 90% homologous or identical to a sequence selected from SEQ ID NOS: 2980-3071. At least one of the one or more gene modulatory reagents may comprise a sequence selected from SEQ ID NOS: 1526-2789. At least one of the one or more of gene modulatory reagents may comprise a sequence at least about 90% homologous or identical to a sequence selected from SEQ ID NOS: 1526-2789. At least one of the one or more gene modulatory reagents may comprise a sequence selected from SEQ ID NOS: 1526-2959. At least one of the one or more of gene modulatory reagents may comprise a sequence at least about 90% homologous or identical to a sequence selected from SEQ ID NOS: 1526-2959. At least one of the one or more gene modulatory reagents may comprise a sequence selected from SEQ ID NOS: 2790-2959. At least one of the one or more of gene modulatory reagents may comprise a sequence at least about 90% homologous or identical to a sequence selected from SEQ ID NOS: 2790-2959.

The modified cancer cells may have been modified by gene editing using a CRISPR-based method. As such, the gene modulatory reagents harbored by the modified cancer cells may comprise a gRNA sequence comprising homology to at least a portion of the gene whose function is knocked down or knocked out in the modified cancer cell. In some cases, the gRNA comprises homology to about 10 to about 50 contiguous nucleotides of the gene. The homology may be at least about 90% sequence homology or identity. The shRNA may be positioned within a vector, e.g., for viral delivery as discussed herein.

The modified cancer cells may also comprise an endonuclease, for instance, where the cells are modified using a gene editing system such as CRISPR. The endonuclease may comprise a Cas9 or Cas12a endonuclease. Non-limiting examples of Cas9 or Cas12a endonuclease include S. pyogenes Cas9 (SpCas9), SpCas9 D1135E variant, SpCas9 VRER variant, SpCas9 EQR variant, xCas9, SpCas9-NG, S. aureus Cas9 (SaCas9), Acidaminococcus sp. (AsCpfl), Lachnospiraceae bacterium (LbCpfl), AsCpfl RR variant, LbCpfl RR variant, AsCpfl RVR variant, C. jejuni Cas9 (CjCas9), N. meningitidis (NmCas9), S. thermophilus (StCas9), T. denticola (TdCas9), and Mad7. The endonuclease may not comprise a Cas9 or Cas12a endonuclease.

The modified cancer cells may have been modified by gene silencing using shRNA gene modulatory reagents. Therefore, one or more of the gene modulatory reagents may comprise an shRNA sequence comprising homology to at least a portion of the gene whose function is knocked down in the modified cancer cell. The shRNA may comprise homology to about 10 to about 50 contiguous nucleotides of the gene. The homology may be at least about 90% sequence homology or identity. The shRNA may be positioned within a vector, e.g., for viral delivery as discussed herein.

Cell Propagation

In one aspect, provided herein are methods of propagating the plurality of genetically modified cells. For example, the genetically modified cells are modified using a CRISPR gene editing system or RNAi as described herein. The cells may be modified from primary cancer cells. In some embodiments, the plurality of modified cells is propagated in 2D format in vitro, 3D format in vitro, or in vivo. Non-limiting examples of the 3D in vitro format could include propagating cells embedded in sponge matrices (e.g., collagen-based), scaffolds, extracellular matrix (ECM) conditions such as basement membrane extract or Matrigel, in suspension, in organoid culture, or in microfluidic platforms. Exemplary materials constituting 3D in vitro format for cell propagation include collagen, gelatin, elastin, fibronectin, laminin, vitronectin, poly-lysine, poly-L-ornithine, silicone, polysaccharide polymers such as alginate, agar, dextran, carrageenan, chitosan, pectin, cellulose, gellan gum, xanthan gum, pullulan, glycosaminoglycan and any fragmented or derivative forms, hyaluronic acid, heparan, heparin, dermatan, chondroitin, or any hydrogel or biocompatible polymer. For in vitro approaches with cancer cells, the cancer cells are maintained under conditions that both support bulk cell survival while allowing selective pressure from induced mutations. For in vivo approaches, a propagation technique is selected which maximizes engraftment efficiency and survival. In some embodiments, in vivo cell propagation can include patient derived xenograft via either heterotopic implantation or orthotopic implantation. Additionally, for in vivo approaches, modified cancer cells may be implanted orthotopically (e.g., within the pancreas, for a pancreatic-origin tumor) or ectopically (e.g., subcutaneously, for a pancreatic origin tumor).

### Screening Methods

In one aspect, provided are methods of evaluating a sample of cells for the presence, absence, and/or quantity of a nucleic acid sequence from the genetic pharmacopeia. The power of the genetic pharmacopeia becomes evident in the ability to read out effects on cell growth directly via ‘barcode’ counting of modified cells (e.g., transduced cancer cells). Cells harboring a gRNA or shRNA impairing cell viability will be less represented in the overall population (i.e. will ‘dropout’); this manifests as less frequent appearance of the gRNA/shRNA sequence itself within the overall population of guide/shRNA sequences. The method may employ next-generation sequencing (NGS), which is well-established, cost effective, commercial scale, robust, highly quantitative, and highly amenable to multiplexed analysis.

Sequencing can be performed with any appropriate sequencing technology, including but not limited to, single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis. Sequencing methods also include next-generation sequencing, e.g., modern sequencing technologies such as Illumina sequencing (e.g., Solexa), Roche 454 sequencing, Ion torrent sequencing, and SOLiD sequencing. In some cases, next-generation sequencing involves high-throughput sequencing methods. Additional sequencing methods available to one of skill in the art may also be employed.

The resulting barcode distributions are interpreted to determine the effect of individual perturbations on the viability of a subject's cells. In some implementations, raw sequencing read counts are interpreted and remapped back into ‘drug space’. For instance, in the hypothetical case described above, if a particular gRNA was found to be less prevalent than expected within the population, this would suggest that the protein encoded by the gene target of the gRNA is required for the survival or proliferation of the patient's cancer cells. As such, the drug targeting that protein (identified in step 1 above) is suggested to be a potentially higher value therapeutic for the patient.

An exemplary method of evaluating the functional effect of genetically modifying cancer cells from a subject comprises: sequencing a plurality of modified cancer cells, wherein each modified cancer cell harbors one or more gene modulatory reagents, each gene modulatory reagent capable of knocking down or knocking out the function of a gene that encodes a protein target in a library of protein targets; and wherein a gene modulatory reagent that impairs cell viability will have fewer sequence reads than a gene modulatory reagent that does not impair cell viability. The method may further comprise determining which gene modulatory regents have fewer than a threshold number of sequence reads. The threshold number of sequence reads may be an expected number of sequence reads if the gene modulatory reagent did not impair cell viability. In some cases the threshold number of sequence reads is an average number of sequence reads for each gene modulatory reagent in the plurality of modified cancer cells. In some embodiments, the method further comprises correlating each gene modulatory reagent that has fewer than the threshold number of sequence reads to its corresponding protein target in the library of protein targets. The method may then also comprise correlating the corresponding protein target to a therapeutic molecule. The library of protein targets may comprise at least 1, at least 2, at least 3, at least 4, at least 5, at least 10, or at least 20 protein targets of Table 5B. The library of protein targets may comprise at least 1, at least 2, at least 3, at least 4, at least 5, at least 10, or at least 20 protein targets of Table 5C. The library of protein targets may comprise at least 1, at least 2, at least 3, at least 4, at least 5, at least 10, or at least 20 protein targets of Table 5D. The library of protein targets may comprise at least 1, at least 2, at least 3, at least 4, at least 5, at least 10, or at least 20 protein targets of Table 5A. The library of protein targets may comprise at least 1, at least 2, at least 3, at least 4, at least 5, at least 10, or at least 20 protein targets of Table 3. The library of protein targets may comprise at least 1, at least 2, at least 3, at least 4, at least 5, at least 10, or at least 20 protein targets of Table 4. At least one of the one or more of the gene modulatory reagents may comprise a gRNA sequence comprising at least about 90% homology or identity to a sequence selected from SEQ ID NOS: 1-2789, 2980-3071. At least one of the one or more of the gene modulatory reagents may comprise a gRNA sequence comprising at least about 90% homology or identity to a sequence selected from SEQ ID NOS: 2980-3071. At least one of the one or more of the gene modulatory reagents may comprise a gRNA sequence comprising at least about 90% homology or identity to a sequence selected from SEQ ID NOS: 1526-2789.

### Methods of Treatment

Further provided herein are methods of treating a subject having a disease or condition, wherein the subject has been determined to be susceptible to a therapeutic agent using a method described herein. In some cases, the disease or condition is cancer. Non-limiting examples of cancer include acute lymphoblastic leukemia (ALL), acute myeloid leukemia (AML), adrenocortical carcinoma, AIDS-related cancers, Kaposi sarcoma, AIDS-related lymphoma, primary CNS lymphoma, anal cancer, appendix cancer, astrocytomas, atypical teratoid/rhabdoid tumor, central nervous system cancers, basal cell carcinoma of the skin, bile duct cancer, bladder cancer, bone cancer, brain tumors, breast cancer, bronchial tumors, Burkitt lymphoma, carcinoid tumor, cardiac tumors, central nervous system cancers, embryonal tumors, germ cell tumor, primary CNS lymphoma, cervical cancer, cholangiocarcinoma, chordoma, chronic lymphocytic leukemia (CLL), chronic myelogenous leukemia (CML), chronic myeloproliferative neoplasms, colorectal cancer, craniopharyngioma, cutaneous t-cell lymphoma, ductal carcinoma in situ (DCIS), embryonal tumors, central nervous system cancer, endometrial cancer, ependymoma, esophageal cancer, esthesioneuroblastoma, Ewing sarcoma, extracranial germ cell tumor, extragonadal germ cell tumor, eye cancer, intraocular melanoma, retinoblastoma, fallopian tube cancer, fibrous histiocytoma of bone, osteosarcoma, gallbladder cancer, gastric cancer, gastrointestinal carcinoid tumor, gastrointestinal stromal tumors (GIST), testicular cancer, gestational trophoblastic disease, hairy cell leukemia, head and neck cancer, heart tumors, hepatocellular cancer, Hodgkin lymphoma, hypopharyngeal cancer, intraocular melanoma, islet cell tumors, pancreatic neuroendocrine tumors, kidney cancer, Langerhans cell histiocytosis, laryngeal cancer, leukemia, lip and oral cavity cancer, liver cancer, lung cancer (non-small cell and small cell), lymphoma, male breast cancer, malignant fibrous histiocytoma of bone and osteosarcoma, melanoma, intraocular melanoma, Merkel cell carcinoma, mesothelioma, metastatic cancer, metastatic squamous neck cancer with occult primary, midline tract carcinoma with NUT gene changes, mouth cancer, multiple endocrine neoplasia syndromes, multiple myeloma/plasma cell neoplasms, mycosis fungoides, myelodysplastic syndromes, myelodysplastic/myeloproliferative neoplasms, myelogenous leukemia, chronic (CML), myeloid leukemia, acute (AML), nasal cavity and paranasal sinus cancer, nasopharyngeal cancer, neuroblastoma, non-Hodgkin lymphoma, non-small cell lung cancer, oral cancer, oropharyngeal cancer, ovarian cancer, pancreatic cancer, pancreatic neuroendocrine tumors (islet cell tumors), papillomatosis, paraganglioma, paranasal sinus and nasal cavity cancer, parathyroid cancer, penile cancer, pharyngeal cancer, pheochromocytoma, pituitary tumor, plasma cell neoplasm/multiple myeloma, pleuropulmonary blastoma, primary central nervous system (CNS) lymphoma, primary peritoneal cancer, prostate cancer, rectal cancer, recurrent cancer, renal cell cancer, retinoblastoma, rhabdomyosarcoma, salivary gland cancer, sarcoma, rhabdomyosarcoma, vascular tumors, osteosarcoma, soft tissue sarcoma, uterine sarcoma, Sézary syndrome, skin cancer, small intestine cancer, squamous cell carcinoma of the skin, squamous neck cancer with occult primary, stomach cancer, T-cell lymphoma, testicular cancer, throat cancer, nasopharyngeal cancer, oropharyngeal cancer, hypopharyngeal cancer, thymoma and thymic carcinoma, thyroid cancer, transitional cell cancer of the renal pelvis and ureter, ureter and renal pelvis, urethral cancer, uterine cancer, endometrial, uterine sarcoma, vaginal cancer, vascular tumors, vulvar cancer, and Wilms tumor and other childhood kidney tumors. In some embodiments, the therapeutic agent is selected from Table 2A. In some embodiments, the therapeutic agent is selected from Table 2B. In some embodiments, the therapeutic agent is selected from Table 3.

A non-limiting example of a method for treating cancer in a subject comprises: administering to the subject a therapeutic molecule selected from a library of therapeutic molecules, wherein the therapeutic molecule has been selected by a method comprising: modifying cancer cells from the subject to knock down or knock out the function of a plurality of genes, each gene in the plurality of genes encoding for a protein target of a therapeutic molecule in the library of therapeutic molecules, whereby the therapeutic molecule has been selected if knocking down or knocking out the function of the gene that encodes for the protein target of the selected therapeutic molecule impairs cancer cell viability. The library of therapeutic molecules may comprise at least 1, at least 2, at least 3, at least 4, at least 5, at least 10, or at least 20 therapeutic agents of Table 2. The library of therapeutic molecules may comprise at least 1, at least 2, at least 3, at least 4, at least 5, at least 10, or at least 20 therapeutic agents of Table 3. The one or more of the plurality of genes may encode for a protein of Table 5B. The one or more of the plurality of genes may encode for a protein of Table 5A. The one or more of the plurality of genes may encode for a protein of Table 5C. The one or more of the plurality of genes may encode for a protein of Table 5D. The one or more of the plurality of genes may encode for a protein of Table 3. The one or more of the plurality of genes may encode for a protein of Table 4.

Another exemplary method for treating cancer comprises: administering to the subject a therapeutic molecule selected from a library of therapeutic molecules; wherein the cancer of the subject has been determined to be susceptible to the selected therapeutic molecule by a method comprising: (a) contacting a sample of cancer cells from the subject with a library of gene modulatory reagents to generate a plurality of modified cancer cells, wherein each modified cancer cell harbors one or more of the gene modulatory reagents, and each gene modulatory reagent is capable of knocking down or knocking out the function of a gene that encodes a protein target of a therapeutic molecule in the library of therapeutic molecules, and (b) sequencing the plurality of modified cancer cells, wherein a gene modulatory reagent that impairs cell viability will have fewer sequence reads than a gene modulatory reagent that does not impair cell viability, and the gene that is knocked down or knocked out by the gene modulatory reagent that impairs cell viability encodes for the protein targeted by the selected therapeutic molecule. In some cases, prior to sequencing, the plurality of modified cancer cells has been propagated. Propagation may comprise maintenance of the modified cancer cells in a 2D in vitro culture. Propagation may comprise maintenance of the modified cancer cells in a 3D in vitro culture. Propagation may comprise maintenance of the modified cancer cells in vivo. Propagation may occur within an animal model, e.g., where the animal is a rodent.

In some embodiments, the cancer cells contacted with the library of gene modulatory reagents are primary cancer cells. Contacting may comprise introducing the one or more gene modulatory reagents into each cancer cell by a viral or non-viral delivery method. Each of the gene modulatory reagents in the library may be encoded on a viral vector. In non-limiting embodiments, the viral vector comprises a lentiviral vector, adenoviral vector, or adeno-associated viral vector. An exemplary non-viral delivery method comprises transposase-mediated transposition.

In some embodiments, the library of therapeutic molecules comprises at least 1, at least 2, at least 3, at least 4, at least 5, at least 10, or at least 20 therapeutic agents of Table 2. In some embodiments, the library of therapeutic molecules comprises at least 1, at least 2, at least 3, at least 4, at least 5, at least 10, or at least 20 therapeutic agents of Table 3. In some embodiments, the library of gene modulatory reagents comprises from about 10 to about 2,000, from about 10 to about 500, from about 10 to about 200, from about 10 to about 150, from about 50 to about 500, from about 50 to about 200, from about 50 to about 2,000, from about 100 to about 2,000, or from about 500 to about 2,000 different gene modulatory reagents. In some embodiments, one or more gene modulatory reagents from the library of gene modulatory reagents comprises a nucleic acid sequence homologous to at least about 15 contiguous nucleotides of a gene encoding a protein of Table 5B. In some embodiments, one or more gene modulatory reagents from the library of gene modulatory reagents comprises a nucleic acid sequence homologous to at least about 15 contiguous nucleotides of a gene encoding a protein of Table 5C. In some embodiments, one or more gene modulatory reagents from the library of gene modulatory reagents comprises a nucleic acid sequence homologous to at least about 15 contiguous nucleotides of a gene encoding a protein of Table 5A. In some embodiments, one or more gene modulatory reagents from the library of gene modulatory reagents comprises a nucleic acid sequence homologous to at least about 15 contiguous nucleotides of a gene encoding a protein of Table 5D. In some embodiments, one or more gene modulatory reagents from the library of gene modulatory reagents comprises a nucleic acid sequence homologous to at least about 15 contiguous nucleotides of a gene encoding a protein of Table 3. In some embodiments, one or more gene modulatory reagents from the library of gene modulatory reagents comprises a nucleic acid sequence homologous to at least about 15 contiguous nucleotides of a gene encoding a protein of Table 4. The homology may be least about 90% sequence homology or identity.

In some cases, one or more of the gene modulatory reagents each comprise a gRNA sequence comprising at least about 90% homology or identity to a sequence selected from SEQ ID NOS: 1-1525. In some cases, one or more of the gene modulatory reagents each comprise a gRNA sequence comprising at least about 90% homology or identity to a sequence selected from SEQ ID NOS: 1-2789, 2980-3071. In some cases, one or more of the gene modulatory reagents each comprise a gRNA sequence comprising at least about 90% homology or identity to a sequence selected from SEQ ID NOS: 2980-3071. In some cases, one or more of the gene modulatory reagents each comprise a gRNA sequence comprising at least about 90% homology or identity to a sequence selected from SEQ ID NOS: 1526-2789. In some cases, one or more of the gene modulatory reagents each comprise a gRNA sequence comprising at least about 90% homology or identity to a sequence selected from SEQ ID NOS: 2790-2959. In some embodiments, each gene modulatory reagent comprises a gRNA sequence comprising homology to at least a portion of the gene that encodes a protein target of a therapeutic molecule in the library of therapeutic molecules. The gRNA may comprise homology to about 10 to about 50 contiguous nucleotides of the gene. The homology may be at least about 90% sequence homology or identity. The gRNA may be positioned within a vector, e.g., for viral delivery as discussed herein.

The method of determining susceptibility to the selected therapeutic molecule may further comprise contacting the cells with an endonuclease. In some embodiments, the endonuclease comprises a Cas9 or Cas12a endonuclease. Non-limiting examples of Cas9 or Cas12a endonucleases include S. pyogenes Cas9 (SpCas9), SpCas9 D1135E variant, SpCas9 VRER variant, SpCas9 EQR variant, xCas9, SpCas9-NG, S. aureus Cas9 (SaCas9), Acidaminococcus sp. (AsCpfl), Lachnospiraceae bacterium (LbCpfl), AsCpfl RR variant, LbCpfl RR variant, AsCpfl RVR variant, C. jejuni Cas9 (CjCas9), N. meningitidis (NmCas9), S. thermophilus (StCas9), T. denticola (TdCas9), and Mad7. In some embodiments, the endonuclease does not comprise a Cas9 or Cas12a endonuclease.

In some embodiments, the gene modulatory reagents comprise a shRNA sequence comprising homology to at least a portion of the gene that encodes a protein target of a therapeutic molecule in the library of therapeutic molecules. The shRNA may comprise homology to about 10 to about 50 contiguous nucleotides of the gene. The homology may be at least about 90% sequence homology or identity. The shRNA may be positioned within a vector, e.g., for viral delivery as discussed herein.

### Further Embodiments

(1) A method of treating cancer in a subject in need thereof, the method comprising administering to the subject a therapeutic molecule selected from a library of therapeutic molecules; wherein the therapeutic molecule has been selected by a method comprising: modifying cancer cells from the subject to knock down or knock out the function of a plurality of genes, each gene in the plurality of genes encoding for a protein target of a therapeutic molecule in the library of therapeutic molecules, whereby the therapeutic molecule has been selected if knocking down or knocking out the function of the gene that encodes for the protein target of the selected therapeutic molecule impairs cancer cell viability.

(2) The method of embodiment 1, wherein the library of therapeutic molecules comprises at least 1, at least 2, at least 3, at least 4, at least 5, at least 10, or at least 20 therapeutic agents of Tables 2-3.

(3) The method of embodiment 1 or embodiment 2, wherein one or more of the plurality of genes encode for a protein of Table 3-5D.

(4) A method of treating cancer in a subject in need thereof, the method comprising administering to the subject a therapeutic molecule selected from a library of therapeutic molecules; wherein the cancer of the subject has been determined to be susceptible to the selected therapeutic molecule by a method comprising:
(a) contacting a sample of cancer cells from the subject with a library of gene modulatory reagents to generate a plurality of modified cancer cells, wherein each modified cancer cell harbors one or more of the gene modulatory reagents, and each gene modulatory reagent is capable of knocking down or knocking out the function of a gene that encodes a protein target of a therapeutic molecule in the library of therapeutic molecules, and
(b) sequencing the plurality of modified cancer cells, wherein a gene modulatory reagent that impairs cell viability will have fewer sequence reads than a gene modulatory reagent that does not impair cell viability, and the gene that is knocked down or knocked out by the gene modulatory reagent that impairs cell viability encodes for the protein targeted by the selected therapeutic molecule.

(5) The method of embodiment 4, wherein prior to sequencing, one or more of the plurality of modified cancer cells have been propagated.

(6) The method of embodiment 5, wherein propagation comprises maintenance of the modified cancer cells in a 2D in vitro culture.

(7) The method of embodiment 5, wherein propagation comprises maintenance of the modified cancer cells in a 3D in vitro culture.

(8) The method of embodiment 5, wherein propagation comprises maintenance of the modified cancer cells in vivo.

(9) The method of embodiment 8, wherein propagation occurs within an animal model.

(10) The method of embodiment 9, wherein the animal is a rodent.

(11) The method of any one of embodiments 4-10, wherein the cancer cells are primary cancer cells.

(12) The method of any one of embodiments 4-11, wherein contacting comprises introducing the one or more gene modulatory reagents into each cancer cell by a viral or non-viral delivery method.

(13) The method of embodiment 12, wherein one or more of the gene modulatory reagents in the library are encoded on a viral vector.

(14) The method of embodiment 13, wherein the viral vector comprises a lentiviral vector, adenoviral vector, or adeno-associated viral vector.

(15) The method of embodiment 12, wherein the non-viral delivery method comprises transposase-mediated transposition.

(16) The method of any one of embodiments 4-15, wherein the library comprises from about 10 to about 2,000, from about 10 to about 500, from about 10 to about 200, from about 10 to about 150, from about 50 to about 500, from about 50 to about 200, from about 50 to about 2,000, from about 100 to about 2,000, or from about 500 to about 2,000 different gene modulatory reagents.

(17) The method of any one of embodiments 4-16, wherein one or more gene modulatory reagents from the library of gene modulatory reagents comprises a nucleic acid sequence homologous to at least about 15 contiguous nucleotides of a gene encoding a protein of Tables 3-5D.

(18) The method of embodiment 17, wherein the homology is at least about 90% sequence homology.

(19) The method of embodiment 18, wherein the homology is at least about 90% sequence identity.

(20) The method of any one of embodiments 4-19, wherein the library of therapeutic molecules comprises at least 1, at least 2, at least 3, at least 4, at least 5, at least 10, or at least 20 therapeutic agents of Tables 2-3.

(21) The method of any one of embodiments 4-20, wherein the cancer comprises at least one cancer chosen from the group comprising acute lymphoblastic leukemia (ALL), acute myeloid leukemia (AML), adrenocortical carcinoma, AIDS-related cancers, Kaposi sarcoma, AIDS-related lymphoma, primary CNS lymphoma, anal cancer, appendix cancer, astrocytomas, atypical teratoid/rhabdoid tumor, central nervous system cancers, basal cell carcinoma of the skin, bile duct cancer, bladder cancer, bone cancer, brain tumors, breast cancer, bronchial tumors, Burkitt lymphoma, carcinoid tumor, cardiac tumors, central nervous system cancers, embryonal tumors, germ cell tumor, primary CNS lymphoma, cervical cancer, cholangiocarcinoma, chordoma, chronic lymphocytic leukemia (CLL), chronic myelogenous leukemia (CML), chronic myeloproliferative neoplasms, colorectal cancer, craniopharyngioma, cutaneous t-cell lymphoma, ductal carcinoma in situ (DCIS), embryonal tumors, central nervous system cancer, endometrial cancer, ependymoma, esophageal cancer, esthesioneuroblastoma, Ewing sarcoma, extracranial germ cell tumor, extragonadal germ cell tumor, eye cancer, intraocular melanoma, retinoblastoma, fallopian tube cancer, fibrous histiocytoma of bone, osteosarcoma, gallbladder cancer, gastric cancer, gastrointestinal carcinoid tumor, gastrointestinal stromal tumors (GIST), testicular cancer, gestational trophoblastic disease, hairy cell leukemia, head and neck cancer, heart tumors, hepatocellular cancer, Hodgkin lymphoma, hypopharyngeal cancer, intraocular melanoma, islet cell tumors, pancreatic neuroendocrine tumors, kidney cancer, Langerhans cell histiocytosis, laryngeal cancer, leukemia, lip and oral cavity cancer, liver cancer, lung cancer (non-small cell and small cell), lymphoma, male breast cancer, malignant fibrous histiocytoma of bone and osteosarcoma, melanoma, intraocular melanoma, Merkel cell carcinoma, mesothelioma, metastatic cancer, metastatic squamous neck cancer with occult primary, midline tract carcinoma with NUT gene changes, mouth cancer, multiple endocrine neoplasia syndromes, multiple myeloma/plasma cell neoplasms, mycosis fungoides, myelodysplastic syndromes, myelodysplastic/myeloproliferative neoplasms, myelogenous leukemia, chronic (CML), myeloid leukemia, acute (AML), nasal cavity and paranasal sinus cancer, nasopharyngeal cancer, neuroblastoma, non-Hodgkin lymphoma, non-small cell lung cancer, oral cancer, oropharyngeal cancer, ovarian cancer, pancreatic cancer, pancreatic neuroendocrine tumors (islet cell tumors), papillomatosis, paraganglioma, paranasal sinus and nasal cavity cancer, parathyroid cancer, penile cancer, pharyngeal cancer, pheochromocytoma, pituitary tumor, plasma cell neoplasm/multiple myeloma, pleuropulmonary blastoma, primary central nervous system (CNS) lymphoma, primary peritoneal cancer, prostate cancer, rectal cancer, recurrent cancer, renal cell cancer, retinoblastoma, rhabdomyosarcoma, salivary gland cancer, sarcoma, rhabdomyosarcoma, vascular tumors, osteosarcoma, soft tissue sarcoma, uterine sarcoma, Sézary syndrome, skin cancer, small intestine cancer, squamous cell carcinoma of the skin, squamous neck cancer with occult primary, stomach cancer, T-cell lymphoma, testicular cancer, throat cancer, nasopharyngeal cancer, oropharyngeal cancer, hypopharyngeal cancer, thymoma and thymic carcinoma, thyroid cancer, transitional cell cancer of the renal pelvis and ureter, ureter and renal pelvis, urethral cancer, uterine cancer, endometrial, uterine sarcoma, vaginal cancer, vascular tumors, vulvar cancer, Wilms tumor, and other childhood kidney tumors.

(22) The method of any one of embodiments 4-21, wherein one or more of the gene modulatory reagents each comprise a guide RNA (gRNA) sequence comprising at least about 90% homology to a sequence selected from SEQ ID NOS: 1-1525, SEQ ID NOS: 1-2789, SEQ ID NOS: 1526-2789, and/or SEQ ID NOS: 2980-3071.

(23) The method of embodiment 22, wherein the at least about 90% homology is at least about 90% identity.

(24) The method of any one of embodiments 4-23, wherein one or more of the gene modulatory reagents comprise a guide RNA (gRNA) sequence comprising homology to at least a portion of the gene that encodes a protein target of a therapeutic molecule in the library of therapeutic molecules.

(25) The method of embodiment 24, wherein the gRNA comprises homology to about 10 to about 50 contiguous nucleotides of the gene.

(26) The method of embodiment 24 or embodiment 25, wherein the homology is at least about 90% sequence homology.

(27) The method of embodiment 26, wherein the homology is at least about 90% sequence identity.

(28) The method of any one of embodiments 4-27, wherein the sample of cancer cells is contacted with an endonuclease.

(29) The method of embodiment 28, wherein the endonuclease comprises a Cas9 or Cas12a endonuclease.

(30) The method of embodiment 29, wherein the Cas9 or Cas12a endonuclease is selected from S. pyogenes Cas9 (SpCas9), SpCas9 D1135E variant, SpCas9 VRER variant, SpCas9 EQR variant, xCas9, SpCas9-NG, S. aureus Cas9 (SaCas9), Acidaminococcus sp. (AsCpfl), Lachnospiraceae bacterium (LbCpfl), AsCpfl RR variant, LbCpfl RR variant, AsCpfl RVR variant, C. jejuni Cas9 (CjCas9), N. meningitidis (NmCas9), S. thermophilus (StCas9), T. denticola (TdCas9), and Mad7.

(31) The method of embodiment 28, wherein the endonuclease does not comprise a Cas9 or Cas12a endonuclease.

(32) The method of any one of embodiments 24-31, wherein the gRNA is positioned within a vector.

(33) The method of embodiment 32, wherein the vector further comprises genetic elements of a virus.

(34) The method of embodiment 33, wherein the virus comprises an adenovirus, retrovirus, adeno-associated virus (AAV), pox virus, parvovirus, baculovirus, measles virus, herpes simplex virus (HSV), Moloney Murine Leukemia Virus (MoMLV, MMLV, MuLV, or MLV), Murine Stem cell Virus (MSCV), or human immunodeficiency virus (HIV), or a combination thereof.

(35) The method of any one of embodiments 32-34, wherein the vector further comprises an auxiliary nucleic acid sequence.

(36) The method of embodiment 35, wherein the auxiliary nucleic acid sequence comprises a sequence encoding a marker, an antibiotic resistance cassette, and surface epitope expression cassette.

(37) The method of embodiment 36, wherein the marker is a fluorescent marker.

(38) The method of any one of embodiments 35-37, wherein the auxiliary nucleic acid allows for the selection of cancer cells that have been modified to harbor the one or more gene modulatory reagents.

(39) The method of any one of embodiments 4-23, wherein one or more of the gene modulatory reagents comprise a short hairpin RNA (shRNA) sequence comprising homology to at least a portion of the gene that encodes a protein target of a therapeutic molecule in the library of therapeutic molecules.

(40) The method of embodiment 39, wherein the shRNA comprises homology to about 10 to about 50 contiguous nucleotides of the gene.

(41) The method of embodiment 39 or embodiment 40, wherein the homology is at least about 90% sequence homology.

(42) The method of embodiment 41, wherein the homology is at least about 90% sequence identity.

(43) The method of any one of embodiments 39-42, wherein the shRNA is positioned within a vector.

(44) The method of embodiment 43, wherein the vector further comprises genetic elements of a virus.

(45) The method of embodiment 44, wherein the virus comprises an adenovirus, retrovirus, adeno-associated virus (AAV), pox virus, parvovirus, baculovirus, measles virus, herpes simplex virus (HSV), Moloney Murine Leukemia Virus (MoMLV, MMLV, MuLV, or MLV), Murine Stem cell Virus (MSCV), or human immunodeficiency virus (HIV), or a combination thereof.

(46) The method of any one of embodiments 43-45, wherein the vector further comprises an auxiliary nucleic acid sequence.

(47) The method of embodiment 46, wherein the auxiliary nucleic acid sequence comprises a sequence encoding a marker, an antibiotic resistance cassette, or surface epitope expression cassette.

(48) The method of embodiment 47, wherein the marker is a fluorescent marker.

(49) The method of any one of embodiments 46-48, wherein the auxiliary nucleic acid allows for the selection of cancer cells that have been modified to harbor the one or more gene modulatory reagents.

(50) A method of generating a plurality of modified cancer cells from a subject having cancer, the method comprising delivering a library of gene modulatory reagents to a sample of cancer cells from the subject to generate the plurality of modified cancer cells; wherein each modified cancer cell harbors one or more of the gene modulatory reagents, and each gene modulatory reagent is capable of knocking down or knocking out the function of a gene that encodes a protein target from a library of protein targets.

(51) The method of embodiment 50, wherein one or more of the gene modulatory reagents comprises a guide RNA (gRNA) sequence comprising homology to at least a portion of the gene whose function is knocked down or knocked out in the modified cancer cell.

(52) The method of embodiment 51, wherein the gRNA comprises homology to about 10 to about 50 contiguous nucleotides of the gene.

(53) The method of embodiment 51 or embodiment 52, wherein the homology is at least about 90% sequence homology.

(54) The method of embodiment 53, wherein the homology is at least about 90% sequence identity.

(55) The method of embodiment 50, wherein one or more of the gene modulatory reagents each comprise a guide RNA (gRNA) sequence comprising at least about 90% homology to a sequence selected from SEQ ID NOS: 1-1525, SEQ ID NOS: 1-2789, SEQ ID NOS: 1526-2789, and/or SEQ ID NOS: 2980-3071.

(56) The method of embodiment 55, wherein the homology is at least about 90% identity.

(57) The method of any one of embodiments 50-56, wherein the sample of cancer cells is contacted with an endonuclease.

(58) The method of embodiment 57, wherein the endonuclease comprises a Cas9 or Cas12a endonuclease.

(59) The method of embodiment 58, wherein the Cas9 or Cas12a endonuclease is selected from S. pyogenes Cas9 (SpCas9), SpCas9 D1135E variant, SpCas9 VRER variant, SpCas9 EQR variant, xCas9, SpCas9-NG, S. aureus Cas9 (SaCas9), Acidaminococcus sp. (AsCpfl), Lachnospiraceae bacterium (LbCpfl), AsCpfl RR variant, LbCpfl RR variant, AsCpfl RVR variant, C. jejuni Cas9 (CjCas9), N. meningitidis (NmCas9), S. thermophilus (StCas9), T. denticola (TdCas9), and Mad7.

(60) The method of embodiment 57, wherein the endonuclease does not comprise a Cas9 or Cas12a endonuclease.

(61) The method of any one of embodiments 51-56, wherein the gRNA is positioned within a vector.

(62) The method of embodiment 61, wherein the vector further comprises genetic elements of a virus.

(63) The method of embodiment 62, wherein the virus comprises an adenovirus, retrovirus, adeno-associated virus (AAV), pox virus, parvovirus, baculovirus, measles virus, herpes simplex virus (HSV), Moloney Murine Leukemia Virus (MoMLV, MMLV, MuLV, or MLV), Murine Stem cell Virus (MSCV), or human immunodeficiency virus (HIV), or a combination thereof.

(64) The method of any one of embodiments 61-63, wherein the vector further comprises an auxiliary nucleic acid sequence.

(65) The method of embodiment 64, wherein the auxiliary nucleic acid sequence comprises a sequence encoding a marker, an antibiotic resistance cassette, or surface epitope expression cassette.

(66) The method of embodiment 65, wherein the marker is a fluorescent marker.

(67) The method of any one of embodiments 64-66, wherein the auxiliary nucleic acid allows for the selection of cancer cells that have been modified to harbor the one or more gene modulatory reagents.

(68) The method of embodiment 50, wherein one or more of the gene modulatory reagents comprise a short hairpin RNA (shRNA) sequence comprising homology to at least a portion of the gene whose function is knocked down or knocked out in the modified cancer cell.

(69) The method of embodiment 68, wherein the shRNA comprises homology to about 10 to about 50 contiguous nucleotides of the gene.

(70) The method of embodiment 68 or embodiment 69, wherein the homology is at least about 90% sequence homology.

(71) The method of embodiment 70, wherein the homology is at least about 90% sequence identity.

(72) The method of any one of embodiments 68-71, wherein the shRNA is positioned within a vector.

(73) The method of embodiment 72, wherein the vector further comprises genetic elements of a virus.

(74) The method of embodiment 73, wherein the virus comprises an adenovirus, retrovirus, adeno-associated virus (AAV), pox virus, parvovirus, baculovirus, measles virus, herpes simplex virus (HSV), Moloney Murine Leukemia Virus (MoMLV, MMLV, MuLV, or MLV), Murine Stem cell Virus (MSCV), or human immunodeficiency virus (HIV), or a combination thereof.

(75) The method of any one of embodiments 72-74, wherein the vector further comprises an auxiliary nucleic acid sequence.

(76) The method of embodiment 75, wherein the auxiliary nucleic acid sequence comprises a sequence encoding a marker, an antibiotic resistance cassette, or surface epitope expression cassette.

(77) The method of embodiment 76, wherein the marker is a fluorescent marker.

(78) The method of any one of embodiments 75-77, wherein the auxiliary nucleic acid allows for the selection of cancer cells that have been modified to harbor the one or more gene modulatory reagents.

(79) The method of embodiment 50, wherein delivering comprising transposase-mediated transposition.

(80) The method of any one of embodiments 50-79, wherein the sample of cancer cells comprises primary cancer cells.

(81) The method of any one of embodiments 50-80, wherein the sample of cancer cells comprises about 10⁵ to about 10⁸ cells.

(82) The method of any one of embodiments 50-81, wherein the library comprises from about 10 to about 2,000, from about 10 to about 500, from about 10 to about 200, from about 10 to about 150, from about 50 to about 500, from about 50 to about 200, from about 50 to about 2,000, from about 100 to about 2,000, or from about 500 to about 2,000 different gene modulatory reagents.

(83) The method of any one of embodiments 50-82, wherein at least about 90% of the gene modulatory reagents are present in the library in a quantity within about 10% of the average gene modulatory reagent quantity.

(84) The method of any one of embodiments 50-83, wherein the library of protein targets comprises at least 1, at least 2, at least 3, at least 4, at least 5, at least 10, or at least 20 protein targets of Tables 3-5D.

(85) The method of any one of embodiments 50-84, wherein the sample of cancer cells has been processed to preserve cell viability.

(86) The method of any one of embodiments 50-85, further comprising preparing the sample of cancer cells to preserve cell viability prior to and/or after delivery of the library of gene modulatory reagents.

(87) The method of any one of embodiments 50-86, further comprising propagating the modified cancer cells.

(88) The method of embodiment 87, wherein propagation comprises maintenance of the modified cancer cells in a 2D in vitro culture.

(89) The method of embodiment 87, wherein propagation comprises maintenance of the modified cancer cells in a 3D in vitro culture.

(90) The method of embodiment 87, wherein propagation comprises maintenance of the modified cancer cells in vivo.

(91) The method of embodiment 90, wherein propagation occurs within an animal model.

(92) The method of embodiment 91, wherein the animal model is a rodent.

(93) A compilation comprising a plurality of modified cancer cells, wherein each modified cancer cell harbors one or more gene modulatory reagents, and each gene modulatory reagent is capable of knocking down or knocking out the function of a gene that encodes a protein target from a library of protein targets.

(94) The compilation of embodiment 93, wherein at least one of the one or more gene modulatory reagents comprises a sequence selected from SEQ ID NOS: 1-1525, SEQ ID NOS: 1-2789, SEQ ID NOS: 1526-2789, and/or SEQ ID NOS: 2980-3071.

(95) The compilation of embodiment 93, wherein at least one of the one or more of gene modulatory reagents comprises a sequence at least about 90% homologous to a sequence selected from SEQ ID NOS: 1-1525, SEQ ID NOS: 1-2789, SEQ ID NOS: 1526-2789, and/or SEQ ID NOS: 2980-3071.

(96) The compilation of embodiment 95, wherein the homology is 90% identity.

(97) The compilation of any of embodiments 93-96, wherein the library of protein targets comprises at least 1, at least 2, at least 3, at least 4, at least 5, at least 10, or at least 20 protein targets of Tables 3-5D.

(98) The compilation of any of embodiments 93-97, wherein one of more of the gene modulatory reagents comprise a guide RNA (gRNA) sequence comprising homology to at least a portion of the gene whose function is knocked down or knocked out in the modified cancer cell.

(99) The compilation of embodiment 98, wherein the gRNA comprises homology to about 10 to about 50 contiguous nucleotides of the gene.

(100) The compilation of embodiment 98 or embodiment 99, wherein the homology is at least about 90% sequence homology.

(101) The compilation of embodiment 100, wherein the homology is at least about 90% sequence identity.

(102) The compilation of any one of embodiments 93-101, further comprising an endonuclease.

(103) The compilation of embodiment 102, wherein the endonuclease comprises a Cas9 or Cas12a endonuclease.

(104) The method of embodiment 103, wherein the Cas9 or Cas12a endonuclease is selected from S. pyogenes Cas9 (SpCas9), SpCas9 D1135E variant, SpCas9 VRER variant, SpCas9 EQR variant, xCas9, SpCas9-NG, S. aureus Cas9 (SaCas9), Acidaminococcus sp. (AsCpfl), Lachnospiraceae bacterium (LbCpfl), AsCpfl RR variant, LbCpfl RR variant, AsCpfl RVR variant, C. jejuni Cas9 (CjCas9), N. meningitidis (NmCas9), S. thermophilus (StCas9), T. denticola (TdCas9), and Mad7.

(105) The compilation of embodiment 102, wherein the endonuclease does not comprise a Cas9 or Cas12a endonuclease.

(106) The compilation of any one of embodiments 98-105, wherein the gRNA is positioned within a vector.

(107) The compilation of embodiment 106, wherein the vector further comprises genetic elements of a virus.

(108) The compilation of embodiment 107, wherein the virus comprises an adenovirus, retrovirus, adeno-associated virus (AAV), pox virus, parvovirus, baculovirus, measles virus, herpes simplex virus (HSV), Moloney Murine Leukemia Virus (MoMLV, MMLV, MuLV, or MLV), Murine Stem cell Virus (MSCV), or human immunodeficiency virus (HIV), or a combination thereof.

(109) The compilation of any one of embodiments 106-108, wherein the vector further comprises an auxiliary nucleic acid sequence.

(110) The compilation of embodiment 109, wherein the auxiliary nucleic acid sequence comprises a sequence encoding a marker, an antibiotic resistance cassette, or surface epitope expression cassette.

(111) The compilation of embodiment 110, wherein the marker is a fluorescent marker.

(112) The compilation of any one of embodiments 109-111, wherein the auxiliary nucleic acid allows for the selection of the modified cancer cells.

(113) The compilation of embodiment 93, wherein one or more of the gene modulatory reagents comprise a short hairpin RNA (shRNA) sequence comprising homology to at least a portion of the gene whose function is knocked down or knocked out in the modified cancer cell.

(114) The compilation of embodiment 113, wherein the shRNA comprises homology to about 10 to about 50 contiguous nucleotides of the gene.

(115) The compilation of embodiment 113 or embodiment 114, wherein the homology is at least about 90% sequence homology.

(116) The compilation of embodiment 115, wherein the homology is at least about 90% sequence identity.

(117) The compilation of any one of embodiments 113-116, wherein the shRNA is positioned within a vector.

(118) The compilation of embodiment 117, wherein the vector further comprises genetic elements of a virus.

(119) The compilation of embodiment 118, wherein the virus comprises an adenovirus, retrovirus, adeno-associated virus (AAV), pox virus, parvovirus, baculovirus, measles virus, herpes simplex virus (HSV), Moloney Murine Leukemia Virus (MoMLV, MMLV, MuLV, or MLV), Murine Stem cell Virus (MSCV), or human immunodeficiency virus (HIV), or a combination thereof.

(120) The compilation of any one of embodiments 117-119, wherein the vector further comprises an auxiliary nucleic acid sequence.

(121) The compilation of embodiment 120, wherein the auxiliary nucleic acid sequence comprises a sequence encoding a marker, an antibiotic resistance cassette, or surface epitope expression cassette.

(122) The compilation of embodiment 121, wherein the marker is a fluorescent marker.

(123) The compilation of any one of embodiments 120-122, wherein the auxiliary nucleic acid allows for the selection of the modified cancer cells.

(124) The compilation of any one of embodiments 93-105, wherein delivering comprising transposase-mediated transposition.

(125) The compilation of any one of embodiments 93-124, wherein the modified cancer cells are modified primary cancer cells.

(126) The compilation of any one of embodiments 93-125, comprising from about 10 to about 2,000, from about 10 to about 500, from about 10 to about 200, from about 10 to about 150, from about 50 to about 500, from about 50 to about 200, from about 50 to about 2,000, from about 100 to about 2,000, or from about 500 to about 2,000 different gene modulatory reagents.

(127) The compilation of any one of embodiments 93-126, comprising from about 10 to about 2,000, from about 10 to about 500, from about 10 to about 200, from about 10 to about 150, from about 50 to about 500, from about 50 to about 200, from about 50 to about 2,000, from about 100 to about 2,000, or from about 500 to about 2,000 different populations of modified cancer cells.

(128) A method of evaluating the functional effect of genetically modifying cancer cells from a subject, the method comprising: sequencing a plurality of modified cancer cells, wherein each modified cancer cell harbors one or more gene modulatory reagents, each gene modulatory reagent capable of knocking down or knocking out the function of a gene that encodes a protein target in a library of protein targets; and wherein a gene modulatory reagent that impairs cell viability will have fewer sequence reads than a gene modulatory reagent that does not impair cell viability.

(129) The method of embodiment 128, further comprising determining which gene modulatory regents have fewer than a threshold number of sequence reads.

(130) The method of embodiment 129, wherein the threshold number of sequence reads is an expected number of sequence reads if the gene modulatory reagent did not impair cell viability.

(131) The method of embodiment 129, wherein the threshold number of sequence reads is an average number of sequence reads for each gene modulatory reagent in the plurality of modified cancer cells.

(132) The method of any one of embodiments 128-131, further comprising correlating each gene modulatory reagent that has fewer than the threshold number of sequence reads to its corresponding protein target in the library of protein targets.

(133) The method of embodiment 132, further comprising correlating the corresponding protein target to a therapeutic molecule.

(134) The method of any one of embodiments 128-133, wherein the library of protein targets comprises at least 1, at least 2, at least 3, at least 4, at least 5, at least 10, or at least 20 protein targets of Tables 3-5D.

(135) The method of any one of embodiments 128-134, wherein one or more of the gene modulatory reagents each comprise a guide RNA (gRNA) sequence comprising at least about 90% homology to a sequence selected from SEQ ID NOS: 1-1525, SEQ ID NOS: 1-2789, SEQ ID NOS: 1526-2789, and/or SEQ ID NOS: 2980-3071.

(136) The method of embodiment 135, wherein the at least about 90% homology is at least about 90% identity.

(137) A library comprising a plurality of gene modulatory reagents, each gene modulatory reagent capable of knocking down or knocking out the function of a gene that encodes a protein target from a library of protein targets.

(138) The library of embodiment 137, wherein the plurality of gene modulatory reagents is capable of knocking down or knocking out the function of at least about 50% of the genes that encode for the protein targets in the library.

(139) The library of embodiment 138, wherein the at least about 50% is at least about 60%.

(140) The library of embodiment 139, wherein the at least about 60% is at least about 70%.

(141) The library of embodiment 140, wherein the at least about 70% is at least about 80%.

(142) The library of embodiment 141, wherein the at least about 80% is at least about 90%.

(143) The library of any one of embodiments 137-142, wherein the library of protein targets comprises all known proteins targeted by known drugs capable of treating a particular disease or condition.

(144) The library of embodiment 143, wherein the disease or condition is cancer.

(145) The library of embodiment 144, wherein the cancer comprises at least one cancer from the group comprising acute lymphoblastic leukemia (ALL), acute myeloid leukemia (AML), adrenocortical carcinoma, AIDS-related cancers, Kaposi sarcoma, AIDS-related lymphoma, primary CNS lymphoma, anal cancer, appendix cancer, astrocytomas, atypical teratoid/rhabdoid tumor, central nervous system cancers, basal cell carcinoma of the skin, bile duct cancer, bladder cancer, bone cancer, brain tumors, breast cancer, bronchial tumors, Burkitt lymphoma, carcinoid tumor, cardiac tumors, central nervous system cancers, embryonal tumors, germ cell tumor, primary CNS lymphoma, cervical cancer, cholangiocarcinoma, chordoma, chronic lymphocytic leukemia (CLL), chronic myelogenous leukemia (CML), chronic myeloproliferative neoplasms, colorectal cancer, craniopharyngioma, cutaneous t-cell lymphoma, ductal carcinoma in situ (DCIS), embryonal tumors, central nervous system cancer, endometrial cancer, ependymoma, esophageal cancer, esthesioneuroblastoma, Ewing sarcoma, extracranial germ cell tumor, extragonadal germ cell tumor, eye cancer, intraocular melanoma, retinoblastoma, fallopian tube cancer, fibrous histiocytoma of bone, osteosarcoma, gallbladder cancer, gastric cancer, gastrointestinal carcinoid tumor, gastrointestinal stromal tumors (GIST), testicular cancer, gestational trophoblastic disease, hairy cell leukemia, head and neck cancer, heart tumors, hepatocellular cancer, Hodgkin lymphoma, hypopharyngeal cancer, intraocular melanoma, islet cell tumors, pancreatic neuroendocrine tumors, kidney cancer, Langerhans cell histiocytosis, laryngeal cancer, leukemia, lip and oral cavity cancer, liver cancer, lung cancer (non-small cell and small cell), lymphoma, male breast cancer, malignant fibrous histiocytoma of bone and osteosarcoma, melanoma, intraocular melanoma, Merkel cell carcinoma, mesothelioma, metastatic cancer, metastatic squamous neck cancer with occult primary, midline tract carcinoma with NUT gene changes, mouth cancer, multiple endocrine neoplasia syndromes, multiple myeloma/plasma cell neoplasms, mycosis fungoides, myelodysplastic syndromes, myelodysplastic/myeloproliferative neoplasms, myelogenous leukemia, chronic (CML), myeloid leukemia, acute (AML), nasal cavity and paranasal sinus cancer, nasopharyngeal cancer, neuroblastoma, non-Hodgkin lymphoma, non-small cell lung cancer, oral cancer, oropharyngeal cancer, ovarian cancer, pancreatic cancer, pancreatic neuroendocrine tumors (islet cell tumors), papillomatosis, paraganglioma, paranasal sinus and nasal cavity cancer, parathyroid cancer, penile cancer, pharyngeal cancer, pheochromocytoma, pituitary tumor, plasma cell neoplasm/multiple myeloma, pleuropulmonary blastoma, primary central nervous system (CNS) lymphoma, primary peritoneal cancer, prostate cancer, rectal cancer, recurrent cancer, renal cell cancer, retinoblastoma, rhabdomyosarcoma, salivary gland cancer, sarcoma, rhabdomyosarcoma, vascular tumors, osteosarcoma, soft tissue sarcoma, uterine sarcoma, Sézary syndrome, skin cancer, small intestine cancer, squamous cell carcinoma of the skin, squamous neck cancer with occult primary, stomach cancer, T-cell lymphoma, testicular cancer, throat cancer, nasopharyngeal cancer, oropharyngeal cancer, hypopharyngeal cancer, thymoma and thymic carcinoma, thyroid cancer, transitional cell cancer of the renal pelvis and ureter, ureter and renal pelvis, urethral cancer, uterine cancer, endometrial, uterine sarcoma, vaginal cancer, vascular tumors, vulvar cancer, Wilms tumor, and other childhood kidney tumors.

(146) The library of any one of embodiments 143-145, wherein the known drugs comprise at least 1, at least 2, at least 3, at least 4, at least 5, at least 10, or at least 20 drugs of Tables 2-3.

(147) The library of any one of embodiments 137-146, wherein the library of protein targets comprises at least 1, at least 2, at least 3, at least 4, at least 5, at least 10, or at least 20 protein targets of Tables 3-5D.

(148) The library of any one of embodiments 137-147, wherein one or more of the plurality of gene modulatory reagents each comprise a guide RNA (gRNA) sequence comprising at least about 90% homology to a sequence selected from SEQ ID NOS: 1-1525, SEQ ID NOS: 1-2789, SEQ ID NOS: 1526-2789, or SEQ ID NOS: 2980-3071.

(149) The library of embodiment 148, wherein the at least about 90% homology is at least about 90% identity.

(150) The library of any one of embodiments 137-149, wherein the plurality of gene modulatory reagents is capable of knocking down or knocking out the function of about 10 to about 2,000, from about 10 to about 500, from about 10 to about 200, from about 10 to about 150, from about 50 to about 500, from about 50 to about 200, about 50 to about 2,000, or about 100 to about 2,000 genes.

(151) The library of any one of embodiments 137-150, wherein the library comprises about 10 to about 2,000, from about 10 to about 500, from about 10 to about 200, from about 10 to about 150, from about 50 to about 500, from about 50 to about 200, about 50 to about 2,000, or about 100 to about 2,000 gene modulatory reagents.

(152) The library of any one of embodiments 137-151, wherein at least one of the gene modulatory reagents is capable of knocking out the function of a gene.

(153) The library of embodiment 152, wherein at least one of the gene modulatory reagents comprise a gRNA sequence having homology to at least a portion of the gene whose function is knocked out by the gene modulatory reagent.

(154) The library of any one of embodiments 137-147, wherein at least one of the gene modulatory reagents is capable of knocking down the function of a gene.

(155) The library of embodiment 154, wherein at least one of the gene modulatory reagents comprise a shRNA sequence having homology to at least a portion of the gene whose function is knocked down by the gene modulatory reagent.

(156) The library of embodiment 153 or embodiment 155, wherein the homology is at least about 90% sequence homology.

(157) The library of embodiment 156, wherein the homology is at least about 90% sequence identity.

(158) The library of embodiment 155 or embodiment 156, wherein the at least a portion is at least about 15 contiguous nucleotides.

(159) The library of any one of embodiments 137-158, wherein at least one of the gene modulatory reagents is positioned within a vector.

(160) The library of embodiment 159, wherein the vector comprises an adapter sequence.

(161) The library of embodiment 160, wherein the adapter sequence comprises a type IIS restriction enzyme cleavage sites.

(162) The library of any one of embodiments 159-161, wherein the vector further comprises genetic elements of a virus.

(163) The library of embodiment 162, wherein the virus comprises an adenovirus, retrovirus, adeno-associated virus (AAV), pox virus, parvovirus, baculovirus, measles virus, herpes simplex virus (HSV), Moloney Murine Leukemia Virus (MoMLV, MMLV, MuLV, or MLV), Murine Stem cell Virus (MSCV), or human immunodeficiency virus (HIV), or a combination thereof.

(164) The library of any one of embodiments 159-163, wherein the vector further comprises a sequence encoding a marker, an antibiotic resistance cassette, or surface epitope expression cassette.

(165) The library of embodiment 164, wherein the marker is a fluorescent marker.

### Tables

Tables 2-3 provide exemplary therapeutic agents, one or more of which may be a member of a drug library described herein.

TABLE 2
Exemplary cancer therapeutic agents.

681640
(5Z)-7-oxozeaenol
17-AAG
4EGI-1
5-fluorouracil
6-bromoindirubin-3′-oxime
7-ethyl-10-hydroxycamptothecin
Abarelix
Abemaciclib
Abexinostat
Abiraterone
abiraterone acetate
Abitrexate
ABT-199
ABT-263
ABT-737
ABT-869
ABT-888
AC220
AC55649
acalabrutinib
ado-trastuzumab emtansine
Adriamycin
adriamycin PFS
Adrucil
AEB071
AEE788, NVP-AEE788
AEW541
Afatinib
afatinib dimaleate
Aflibercept
Afuresertib
AG013736
AG014699
AG-014699
AHPN
AKT inhibitor VIII
aldesleukin
alectinib
alemtuzumab
alisertib
alitretinoin
all-trans retinoic acid
alpelisib
alvocidib
AM-580
amatuximab
AMG 208
AMG 337
AMG 595
AMG 780
AMG-706
AMG900
AMN107
amonafide
amuvatinib
anastrozole
anti-mesothelin iCasp9M28z CAR-
transduced autologous T lymphocytes
AP26113
apalutamide
apatinib
apatorsen
apicidin
apitolisib
APO866
arimidex
armala
aromasin
ARQ 736
ARQ-197
ASP3026
AT101
AT13148
AT13387
AT-406
AT7867
AT9283
atezolizumab
ATRA
AV 203
AV-951
avelumab
avicin D
AVL-292
axicabtagene ciloleucel
axitinib
AZ191
AZ628
AZ960
azacitidine
AZD0530
AZD1152-HQPA
AZD1208
AZD1480
AZD1775, MK1775
AZD2014
AZD2171
AZD-2281
AZD4547
AZD5069
AZD5363
AZD6244
AZD6482
AZD7451
AZD7762
AZD8055
AZD8186
AZD8330
AZD8931
bafetinib
barasertib
bax channel blocker
BAY 1187982
BAY1125976
BAY1143572
BAY-43-9006
BAY-73-4506
Bazedoxifene
BCL-LZH-4
belimumab
belinostat
bevacizumab
bexarotene
bextra
BEZ235
BGB-283
BGJ398, NVP-BGJ398
BGT226, NVP-BGT226
BI 836845
BI 847325
BI-2536
BIBF 1120
BIBR-1532
BIBW2992
bicalutamide
BIIB022
BIND-014
binimetinib
BIO
birabresib
BIRB-796
birinapant
BIX-01294
blinatumomab
BLZ945
BMN-673
BMS-195614
BMS-270394
BMS-345541
BMS-354825
BMS-387032
BMS-536924
BMS-582664
BMS-599626, AC480
BMS-690514, EVRI
BMS-708163
BMS-754807
BMS-777607
BMS-907351
BMS-911543
bortezomib
bosutinib
BRD0667
BRD3547
BRD4658
BRD4770
BRD6430
BRD6929
BRD9047
BRD9876
BRD-K11533227
BRD-K29313308
BRD-K51490254
BRD-K55478147
BRD-K61166597
BRD-K69840642
BRD-K81491172
BRD-K85133207
BRD-K88742110
brentuximab vedotin
briciclib
briciclib sodium
brigatinib
brilanestrant
brivanib
brontictuzumab
buparlisib
BVD-523
BX-795
BXL-628
BYL719
BYL-719
C6-ceramide
cabazitaxel
cabozantinib
CAL-101
camptosar
camptothecin
canakinumab
canertinib
capecitabine
carfilzomib
casodex
CAY10618
CBB-1007
CC-223
CCI-779
CD-1530
CD-437
CDK9 inhibitor 14
cediranib
cemiplimab-rwlc
cenisertib
CEP-701
ceritinib
cerubidine
cetuximab
CF102
CGP60474
Ch-55
Chembridge cat# 7667791
CHIR-265
CHIR-99021
CHR-2797
CHS-828
CI-1033
CI-1040
CI-994
ciclopirox olamine
Cimetidine
citarinostat
cixutumumab
clofarabine
clolar
cMet CAR-mRNA Electroporated
autologous T lymphocytes
cobimetinib
cometriq
compazine
compound 7d-cis
copanlisib
copanlisib hydrochloride
copper Cu 64-DOTA-trastuzumab
CP-358774
CPI-444
crenolanib
crizotinib
CS 7017, RS5444
CT 99021
CT53518
CT99021
CX-4945
cyanoquinoline 11
cyclopamine
CYT-387
dabrafenib
dabrafenib mesylate
dacomitinib
dactolisib
dalantercept
dalotuzumab
danusertib
daraprim
daratumumab
dasatinib
daunorubicin
daunorubicin hydrochloride
daunoxome
Debio 0932
Debio 1347, CH5183284
decadron
decitabine
defactinib
degarelix
denileukin diftitox
denintuzumab mafodotin
denosumab
depatuxizumab
depatuxizumab mafodotin
dexamethasone
dezapelisib
dinaciclib
dinutuximab
DMOT4039A
docetaxel
dociparstat sodium
doramapimod
dovitinib
doxil
doxorubicin
doxorubicin hydrochloride
DPD
duligotuzumab
durvalumab
dusigitumab
duvelisib
E7080
efudex
EGF816
EHT 1864
EHT 5372
elesclomol
ellence
elocalcitol
elotuzumab
emactuzumab
embelin
EMD 1214063, MSC2156119J
emibetuzumab
enasidenib
enasidenib mesylate
encorafenib
ENMD-0276
ENMD-2076
enobosarm
ensituximab
entinostat
entolimod
entospletinib
entrectinib
enzalutamide
enzastaurin
epacadostat
epidaza
epirubicin
epitinib
Eplerenone
EPZ004777 analog
EPZ-5676
EPZ-6438, E7438
erdafitinib
eribulin mesylate
erlotinib
erlotinib hydrochloride
estramustine
estramustine phosphate
estybon
etimoxir
etirinotecan pegol
Etomidate
etoposide
etoposide/etoposide phosphate
eulexin, apimid
everolimus
evista, keoxifene
EX-527
EXEL-2880
exemestane
fareston
faslodex
faz053
fedratinib
femara
ficlatuzumab
figitumumab
filanesib
fingolimod
firmagon
FK866
flavopiridol
fluorouracil
fluoxymesterone
flutamide
foretinib
fostamatinib
fruquintinib
FTY720
fulvestrant
futuximab
galeterone
galunisertib
gandotinib
ganetespib
ganitumab
GANT-61
GDC-0449
GDC-0623
GDC-0879
GDC-0941
gedatolisib
gefitinib
geldanamycin
gemcitabine
gemtuzumab ozogamicin
genasense
gilenya
gilteritinib
glasdegib
glasdegib maleate
glesatinib
glycooptimized trastuzumab-GEX
GMX-1778
GNF4877
goserelin
gossypol
GSK089
GSK1059615
GSK1070916
GSK1120212 (Trametanib)
GSK1210151A
GSK1363089
GSK2118436 (Dabrafenib)
GSK2256098
GSK-2636771
GSK269962A
GSK2879552
GSK3326595
GSK461364
GSK525762A
GSK-626616
GU 17
guadecitabine
GW 441756
GW 5074
GW2016
GW572016
GW786034
GW-843682X
HGS1036, FP-1039, GSK3052230
HKI-272
HLI-373
HMN-214
hycamptin
hycamtin
hydroxydaunorubicin
hydroxyurea
I-BET
I-BET151
ibritumomab tiuxetan
ibrutinib
IC-87114
icotinib
idamycin
idarubicin
idasanutlin
idelalisib
ilorasertib
imatinib
imatinib mesylate
imgatuzumab
imiquimod
INCB018424
INCB028060, INC280
INCB052793
INCB-18424
Indisulam
indoximod
inebilizumab
Infinity compound 1
iniparib
INK-1117
inotuzumab ozogamicin
interferon alfa-2b, recombinant
iobenguane I 131
ipafricept
ipatasertib
ipilimumab
irinotecan
irinotecan liposome
irinotecan trihydrochloride
isoliquiritigenin
ISOX
istiratumab
istradefylline
istubal
itacitinib
IV-2
ivosidenib
ixabepilone
ixazomib citrate
jakavi
JNJ-26854165
JNK Inhibitor VIII
JQ1
JTP-74057
JW74
Ketoconazole
Ki8751
KU-0059436
KU-0060648
KU-0063794
KW 2449
KW-6002
KX01
KX2-391
L-685458
lanreotide
lanreotide acetate
lapatinib
lapatinib ditosylate
larotrectinib sulfate
LB-100
LBH-589
LBW242
LE-135
lenalidomide
lenvatinib
lenvatinib mesylate
leptomycin b
lestaurtinib
letrozole
leucovorin
leuprolide
leuprolide acetate
leurocristine
LFM-A13/DDE-28
LGK974
linifanib
linsitinib
liposomal daunorubicin
liposomal doxorubicin
LJM716
lomeguatrib
loprox
lorlatinib
LOXO-101
lucitanib
luminespib
lupron
luspatercept
lutetium Lu 177-dotatate
LY-2157299
LY2510924
LY2874455
LY317615
LY450139
manumycin a
margetuximab
marinopyrrole a
maritoclax
masitinib
masivet
MDX-1105, BMS-936559
MEDI-3617
mercaptopurine
Merck60
methotrexate
methyltestosterone
Metyrapone
Metyrapone
MGCD-265
MH2075
midostaurin
mipsagargin
mirin
mirvetuximab soravtansine
Mitotane
mitoxantrone
MK-0457
MK-0752
MK-1775
MK-2206
MK-2461
MK5108
MK-8353, SCH900353
ML204
MLN2238
MLN-2480
MLN-4924
MLN518
MLN8237
MLN9708
MM_V_GSK_2d1
MM-111
MM-151
mocetinostat
modotuximab
mogamulizumab-kpkc
momelotinib
motesanib
motolimod
moxetumomab pasudotox-tdfk
MRK 003
MRK-560
MST-312
mubritinib
muparfostat
N9-isopropylolomoucine
naquotinib
navicixizumab
navitoclax
navoximod
necitumumab
nelarabine
neratinib
neratinib maleate
nesvacumab
N-hexanoyl-D-sphingosine
niclocide
niclosamide
nilandron, anandron
nilotinib
nilutamide
nimotuzumab
nintedanib
niraparib
niraparib tosylate monohydrate
nirogacestat
nivolumab
NMS-1286937
novantrone
novonex
NSC23766
NSC303580
NSC652287
NSC718781
NSC74859
NU-7441
nutlin-3
Nutlin-3a
NVP-ADW742
NVP-BEZ235
NVP-BGJ-398
NVP-BSK805
NVP-BYL-719
NVP-LDE225
NVP-TAE684
O-6-benzylguanine
obatoclax
obatoclax mesylate
obinutuzumab
oblimerson
ofatumumab
olaparib
olaratumab
olmutinib
omacetaxine mepesuccinate
omipalisib
ON-01910
onalespib
onartuzumab
Oncovin
Onivyde
OPB-31121
orantinib
OSI-027
OSI-774
OSI-906
OSI-930
osimertinib
ostarine
paclitaxel
pacritinib
palbociclib
pandacostat
panitumumab
panobinostat
patidegib
patritumab
pazopanib
pazopanib hydrochloride
PCI-32765
PD 153035
PD0325901
PD-0332991
PD-173074
PD318088
peginterferon alfa-2b
pelitinib
pembrolizumab
pemetrexed disodium
pentostatin
pertuzumab
pevonedistat
PF-01367338
PF-02341066
PF-03814735
PF-04217903
PF-184
PF-2341066
PF-4691502
PF-4708671
PF477736
PF-562271
PF-573228
PHA665752
PHA-793887
PI-103
pictilisib
pifithrin-mu
PIK-93
pilaralisib
PIM447
pimasertib
pluripotin
PLX3397, PLX108-01
PLX-4032
PLX4720
PLX-4720
PLX7486
pomalidomide
ponatinib
ponatinib hydrochloride
porfimer
poziotinib
pralatrexate
prexasertib
prochlorperazine
prochlorperazine dimaleate
PRT062070
PSMA ADC
purmorphamine
PWT33597
PX-12
PX-866
PXD-101
pyrimethamine
quizartinib
QW-BI-011
R-406
R428
rabusertib
radium 223 dichloride
RAF265
ralimetinib
raloxifene
ramucirumab
rapamune
rapamycin
rebastinib tosylate
refametinib
REGN1400
REGN421
regorafenib
relugolix
remetinostat
reparixin
Repligen 136
resminostat
retaspimycin
revatio
RG-108
RG7204
Ribavirin
ribociclib
ricolinostat
ridaforolimus (deforolimus)
rigosertib
rilotumumab
rimiducid
RITA
rituximab
rituximab and hyaluronidase human
rituximab/hyaluronidase human
RO-3306
RO4929097, R4733
RO5126766, CH5126766
RO5185426
RO5212054, PLX3603
rociletinib
romidepsin
roniciclib
rosomidnar
rucaparib
Rucaparib
rucaparib camsylate
ruxolitinib
ruxolitinib phosphate
S3I-201
SAHA
salermide
sapanisertib
SAR125844
SAR245409
SAR302503
SAR3419
SAR405838, MI-773
SAR650984
saracatinib
SB 216763
SB-225002
SB-431542
SB-505124
SB-525334
SB-743921
SC144
SCH 530348
SCH727965
SCH772984
SCH-79797
seliciclib
selinexor
selumetinib
semagacestat
SEN0014196
serdemetan
seribantumab
SF1126
SGC0946
SGX-523
sildenafil
silmitasertib
siltuximab
sipuleucel-T
sirolimus
sitagliptin
sitravatinib
SJ-172550
skepinone-L
SKI-606
SL 0101-1
SL 0101-1
SM-406
SN-38
SNS-032
SNS-314
SNX-2112
SNX-5422
sonidegib
sophoretin, quercetin
sorafenib
sorafenib tosylate
sotatercept
sotrastaurin
SP600125
Spironolactone
Spironolactone
SRT-1720
stelazine
STI571
streptozocin
SU11248
SU11274
sulfatinib
sunitinib
sunitinib malate
sutent
SZ4TA2
tagraxofusp-erzs
TAK-733
TAK901
taladegib
talazoparib
tamoxifen
tamoxifen citrate
tandutinib
tanespimycin
tanespymicin
tarextumab
targretin
TAS-119
taselisib
tasocitinib
taxol
taxotere
telatinib
telisotuzumab
temsirolimus
teniposide
TEW-7197
TG100-115
TG-101348
TGX-221
thalidomide
theliatinib
THM-I-91
tipifarnib
tipifarnib-P1
tipifarnib-P2
tisagenlecleucel
tisagenlecleucel-T
tivantinib
tivozanib
TL-32711
tocilizumab
tofacitinib
tofacitinib citrate
toposar, etopophos
topotecan
topotecan hydrochloride
toremifene
tosedostat
tositumomab
tositumomab and iodine i 131 tositumomab
tovetumab
tozasertib
trametinib
trastuzumab
trastuzumab emtansine
trebananib
trelstar
tretinoin
trifluoperazine
triptorelin
TSR-011
tubastatinA
tucatinib
tucidinostat
TW-37
tyverb
ublituximab
ulocuplumab
umbralisibtosylate
UNC0321
UNC0638
UNC0642
uprosertib
valdecoxib
valodex
valrubicin
valstar
vandetanib
vanucizumab
vargatef
vatalanib
veliparib
vemurafenib
vemurarinib
venetoclax
VER-155008
vesanoid
viagra
vinblastine sulfate
vincristine
vincristine sulfate
vinorelbine tartrate
vintafolide
vismodegib
VM-26
volasertib
volitinib
vorapaxar
vorinostat
voxtalisib
VS-4718
vumon
VX-680
VX-803
WH-4-025
WZ4002
WZ8040
X-396
X-82
XAV 939
XL019
XL184
XL228
XL281, BMS-908662
XL647, KD019
XL765
XL820
XL880
XL888
XMT-1522
Xtandi
YK 4-279
YM155
YM-155
zactima
zarnestra
ZD-1839
ZD6474
zebularine
zibotentan
ziv-aflibercept
ZM-447439
zoladex
ZSTK474
ZW25
zytiga

TABLE 3
Exemplary cancer therapeutic agents with associated targets.

| Target Gene | |
|---|---|
| Symbol | Drug Names (Development, Generic or Trade Name) |
| ABL1 | nilotinib (e.g., Tasigna ®, AMN107); ponatinib (e.g., Iclusig ®); cenisertib; AT9283; |
| | dasatinib (e.g., BMS-354825, Sprycel ®); bafetinib; bosutinib (e.g., Bosulif ®, SKI-606); |
| | imatinib (e.g., Gleevec ®); XL228saracatinib (AZD0530); regorafenib (e.g., Stivarga ®); |
| | KW 2449; imatinib mesylate (e.g., STI571) |
| ABL2 | dasatinib (e.g., BMS-354825, Sprycel ®) |
| ACPP | sipuleucel-T |
| (ACP3) | |
| ADA | pentostatin |
| ADORA2A | CPI-444; istradefylline (e.g., KW-6002) |
| ADORA3 | CF102 |
| AGXT | O-6-benzylguanine |
| AKT1 | cenisertib; AT13148; AZD5363; BAY1125976; ipatasertib; afuresertib; uprosertib; MK- |
| | 2206; AT7867; gefitinib (e.g., ZD-1839; Iressa ®); AKT inhibitor VIII |
| AKT2 | cenisertib; AT13148; AZD5363; BAY1125976; ipatasertib; afuresertib; uprosertib; MK- |
| | 2206; AT7867; AKT inhibitor VIII |
| AKT3 | cenisertib; AT13148; AZD5363; BAY1125976; ipatasertib; afuresertib; uprosertib; MK- |
| | 2206; AT7867; AKT inhibitor VIII |
| ALK | dalantercept; brigatinib (e.g., Alunbrig ®); gilteritinib (e.g., Xospata ®); ASP3026; alectinib |
| | (e.g., Alecensa ®); ceritinib (e.g., Zykadia ®); crizotinib (e.g., Xalkori ®); lorlatinib (e.g., |
| | Lorbrena ®); entrectinib; TSR-011; X-396 (ensartinib); AP26113; NVP-TAE684; PF- |
| | 2341066 |
| ANGPT1 | AMG 780; trebananib |
| ANGPT2 | AMG 780; MEDI-3617; nesvacumab; vanucizumab; trebananib |
| ANPEP | tosedostat; CHR-2797 |
| APH1A | MK0752; MRK 003; RO4929097; semagacestat; LY450139; L-685458; BMS-708163 |
| APH1B | MK0752; MRK 003; BMS-708163 |
| AR | enobosarm (Ostarine ®); nilutamide (e.g., Nilandron ®, Anandron ®); bicalutamide (e.g., |
| | Casodex ®); flutamide (e.g., Eulexin ®, Apimid); enzalutamide (e.g., Xtandi ®); galeterone; |
| | fluoxymesterone; methyltestosterone |
| ARAF | AZ628; MLN-2480 |
| ATR | VX-803 |
| AURKA | cenisertib; AT9283; ENMD-2076; MK5108; alisertib; PF-03814735; TAK901; TAS-119; |
| | ilorasertib; AMG900; BI 847325; danusertib; SNS-314; SNS 314; MLN8237; KW 2449; |
| | tozasertib; VX-680; MK-0457 |
| AURKB | cenisertib; AT9283; barasertib; GSK1070916; PF-03814735; ilorasertib; AMG900; BI |
| | 847325; danusertib; SNS-314; SNS 314; tozasertib; azd1152-HQPA; SL 0101-1; VX-680; |
| | MK-0457; BX-795; ZM-447439 |
| AURKC | GSK1070916; SNS 314; tozasertib; VX-680; MK-0457; BX-795 |
| AXL | gilteritinib; glesatinib; sitravatinib; R428 |
| B4GALNT1 | dinutuximab (e.g., Unituxin ®) |
| BAX | Bax channel blocker; BRD3547; gossypol |
| BCL2 | navitoclax; AT101; venetoclax (e.g., Venclexta ®); obatoclax; oblimerson (e.g., |
| | Genasense ®); rosomidnar; docetaxel (e.g., Taxotere ®); gossypol; TW-37; ABT-737; ABT- |
| | 199; Infinity compound 1; ABT-263; obatoclax mesylate |
| BCL2L1 | navitoclax; BCL-LZH-4; obatoclax; TW-37; SZ4TA2; ABT-737; ABT-263; obatoclax |
| | mesylate |
| BCL2L2 | navitoclax; ABT-737; ABT-263 |
| BIRC5 | YM155 |
| BLK | dasatinib (e.g., BMS-354825, Sprycel ®) |
| BMX | dasatanib (e.g., BMS-354825, Sprycel ®) |
| BRAF | ARQ 736; BGB-283; (dabrafenib, e.g., Tafinlar ®); vemurafenib (e.g., Zelboraf ®); |
| | RAF265; RO5212054, PLX3603; sorafenib (e.g., Nexavar ®, BAY-43-9006); regorafenib |
| | (e.g., Stivarga ®); encorafenib; MLN2480; RO5126766, CH5126766; XL281, BMS- |
| | 908662; AZ628; sorafenib tosylate; dabrafenib mesylate (e.g., GSK2118436); PLX-4720; |
| | MLN-2480; PLX-4032; RG7204; RO5185426; GDC-0879; CHIR-265 |
| BRD2 | birabresib; GSK525762A; I-BET; GSK1210151A; I-BET151; JQ1 |
| BRD3 | birabresib; GSK525762A; I-BET; GSK1210151A; I-BET151; JQ1 |
| BRD4 | birabresib; GSK525762A; I-BET; GSK1210151A; I-BET151; JQ1 |
| BTK | acalabrutinib; cenisertib; AVL-292; ibrutinib (e.g., Imbruvica ®, PCI-32765) dasatinib (e.g., |
| | Sprycel ® BMS-354825); LFM-A13/DDE-28 |
| CCND1 | briciclib; briciclib sodium |
| CCND2 | briciclib; briciclib sodium |
| CCND3 | briciclib; briciclib sodium |
| CD19 | inebilizumab; blinatumomab (e.g., Blincyto ®); SAR3419; denintuzumab mafodotin; |
| | tisagenlecleucel-T |
| CD274 | MDX-1105, BMS-936559; durvalumab; (e.g., Imfinzi ®); atezolizumab (e.g., Tecentriq ®); |
| | avelumab (e.g., Bavencio); FAZ053 |
| CD38 | daratumumab (e.g., Darzalex ®), HuMax-CD38; SAR650984 |
| CDK1 | alvocidib; roniciclib; dinaciclib; CGP60474; RO-3306; SCH727965; PHA-793887; |
| | flavopiridol; N9-isopropylolomoucine |
| CDK2 | alvocidib; roniciclib; dinaciclib; seliciclib; CGP60474; SCH727965; SNS-032; BMS- |
| | 387032; PHA-793887; flavopiridol |
| CDK4 | alvocidib; roniciclib; ribociclib (e.g., Kisqali ®); abemaciclib; palbociclib (e.g., Ibrance ®); |
| | PHA-793887; flavopiridol; PD-0332991 |
| CDK5 | alvocidib; dinaciclib; CGP60474; SCH727965; PHA-793887; N9-isopropylolomoucine |
| CDK6 | alvocidib; ribociclib (e.g., Kisqali ®); abemaciclib; palbociclib (e.g., Ibrance ®); |
| | flavopiridol; PD-0332991 |
| CDK7 | alvocidib; roniciclib; seliciclib; CGP60474; SNS-032; BMS-387032; PHA-793887 |
| CDK9 | alvocidib; roniciclib; dinaciclib; seliciclib; BAY1143572; CGP60474; SCH727965; CDK9 |
| | inhibitor 14; SNS-032; BMS-387032; PHA-793887 |
| CHD1 | epirubicin |
| CHEK1 | prexasertib; 681640; AZD7762; PF477736 |
| CHEK2 | rabusertib; AZD7762; PF477736 |
| CPT1A | etimoxir |
| CRBN | thalidomide; lenalidomide; pomalidomide |
| CRTC1 | AZD8055; sapanisertib; OSI-027; NVP-BEZ235 |
| CRTC2 | AZD8055; sapanisertib; OSI-027; NVP-BEZ235 |
| CSF1R | PLX3397, PLX108-01; PLX7486; emactuzumab; BLZ945; sunitinib malate (e.g., Sutent ®, |
| | SU11248); linifanib; ABT-869; pazopanib |
| CSNK2A1 | silmitasertib; CX-4945 |
| CSNK2A2 | silmitasertib; CX-4945 |
| CXCR1 | reparixin |
| CXCR2 | AZD5069; reparixin; SB-225002 |
| CXCR4 | ulocuplumab; LY2510924; dociparstat sodium |
| CYP17A1 | abiraterone acetate (e.g., Zytiga ®) |
| CYP19A1 | letrozole (e.g., Femara ®); exemestane (e.g., Aromasin ®); anastrozole (e.g., Arimidex ®) |
| CYP11B1 | Metyrapone; Mitotane; Ketoconazole; Spironolactone; Cimetidine |
| CYP11B2 | Eplerenone; Etomidate; Metyrapone; Spironolactone |
| DDR2 | regorafenib (e.g., Sitravatinib ®) |
| DHFR | methotrexate; pemetrexed disodium; pralatrexate; abitrexate |
| DHH | glasdegib; vismodegib (e.g., Erivedge ®) |
| DHX9 | YK 4-279 |
| DNMT1 | guadecitabine; azacitidine; decitabine; zebularine; RG-108 |
| DOT1L | EPZ-5676; EPZ004777 analog; SGC0946 |
| DPP4 | sitagliptin |
| DRD2 | prochlorperazine; prochlorperazine dimaleate (e.g., Compazine ®); trifluoperazine (e.g., |
| | Stelazine ®) |
| DYRK1A | EHT 5372; GNF4877; AZ191 |
| DYRK1B | EHT 5372; GNF4877; AZ191 |
| DYRK2 | GSK-626616 |
| DYRK3 | GSK-626616 |
| DYRK4 | GSK-626616 |
| EDNRA | Zibotentan |
| EGFR | AEE788, NVP-AEE788; brigatinib (e.g., Alunbrig ®); naquotinib; vandetanib (e.g., |
| | Zactima ®, Caprelsa ®); osimertinib (e.g., Tagrisso ®); BGB-283; afatinib (e.g., Gilotrif ™, |
| | Tomtovok ®, BIBW2992); icotinib; canertinib; rociletinib; EGF816; olmutinib; epitinib; |
| | theliatinib; erlotinib (e.g., Tarceva ®); XL647, KD019; gefitinib (e.g., Iressa ®); AZD8931; |
| | BMS-599626, AC480; modotuximab; depatuxizumab; panitumumab (e.g., Vectibix ®); |
| | nimotuzumab; necitumumab (e.g., Portrazza ™); cetuximab (e.g., Erbitux ®); |
| | duligotuzumab; MM-151; imgatuzumab; futuximab; depatuxizumab mafodotin; AMG 595; |
| | aldesleukin (e.g., Proleukin ®); lapatinib (e.g., Tykerb ®); osimertinib (e.g., Tagrisso ®); |
| | AP26113; dacomitinib; erlotinib hydrochloride; lapatinib ditosylate; cyanoquinoline 11; |
| | GW572016; GW2016; Tyverb; PD 153035; CI-1033; ZD-1839; CP-358774; OSI-774; |
| | NSC718781; WZ4002; WZ8040; neratinib; HKI-272 |
| EHMT1 | UNC0638; UNC0642 |
| EHMT2 | BIX-01294; QW-BI-011; BRD4770; UNC0321; UNC0638; UNC0642 |
| EIF4E | 4EGI-1; Ribavirin |
| EPHA2 | regorafenib; dasatinib (e.g., BMS-354825, Sprycel ®); vandetanib |
| EPHB4 | sitravatinib; XL647, KD019; vandetanib |
| ERBB2 | tucatinib; BMS-690514, EVRI; AEE788, NVP-AEE788; afatinib (e.g., Gilotrif ™, |
| | Tomtovok ®, BIBW2992); canertinib; lapatinib (e.g., Tykerb ®); neratinib (e.g., Nerlynx ®); |
| | mubritinib; XL647, KD019; glycooptimized trastuzumab-GEX; margetuximab; MM-111; |
| | pertuzumab (e.g., Perjeta ®, Omnitarg ®); trastuzumab (e.g., Herceptin ®); ado trastuzumab |
| | emtansine (e.g., Kadcyla ®); XMT-1522; ZW25; copper Cu 64-DOTA-trastuzumab; |
| | aldesleukin (e.g., Proleukin ®); dacomitinib; lapatinib ditosylate; GW572016; GW2016; |
| | Tyverb; CI-1033; erlotinib (e.g., Tarceva ®); CP-358774; OSI-774; NSC718781; HKI-272; |
| | AZD8931; vandetanib |
| ERBB3 | afatinib (e.g., Gilotrif ™); AV 203; LJM716; duligotuzumab; MM-111; seribantumab; |
| | istiratumab; REGN1400; patritumab; dacomitinib; AZD8931; vandetanib |
| ERBB4 | pelitinib; poziotinib; dacomitinib; afatinib; (e.g., Gilotrif ™); vandetanib |
| ESR1 | fulvestrant (e.g., Faslodex ®); tamoxifen; tamoxifen citrate (e.g., Nolvadex ®, Istubal, |
| | Valodex, Soltamox ™); raloxifene (e.g., Evista ®, Keoxifene); toremifene (e.g., Fareston ®); |
| | brilanestrant; galeterone; fluoxymesterone; estramustine |
| ESR2 | fulvestrant (e.g., Faslodex ®); tamoxifen; tamoxifen citrate (e.g., Nolvadex ®, Istubal, |
| | Valodex, Soltamox ™); raloxifene (e.g., Evista ®), Keoxifene); toremifene (e.g., Fareston ®); |
| | brilanestrant; galeterone; estramustine; estramustine phosphate |
| EZH2 | EPZ-6438, E7438; MM_V_GSK_2d1; QW-BI-011; BRD4770 |
| F2R | SCH-79797; vorapaxar; SCH-530348 |
| FCGR1A | porfimer |
| FGF1 | muparfostat; pazopanib hydrochloride |
| FGF2 | muparfostat |
| FGFR1 | masitinib; ponatinib (e.g., Iclusig ®); AZD4547; BGJ398, NVP-BGJ398; nintedanib (e.g., |
| | Vargatef ®, BIBF 1120); Debio 1347, CH5183284; lucitanib; sulfatinib; LY2874455; |
| | dovitinib; XL228; erdafitinib; orantinib; HGS1036, FP-1039, GSK3052230; sorafenib |
| | tosylate; regorafenib; PD-173074; lenvatinib; pazopanib |
| FGFR2 | masitinib; ponatinib (e.g., Iclusig ®); AZD4547; BGJ398, NVP-BGJ398; nintedanib (e.g., |
| | Vargatef ®, BIBF 1120); Debio 1347, CH5183284; lucitanib; sulfatinib; LY2874455; |
| | dovitinib; XL228; erdafitinib; BAY 1187982; regorafenib; Ki8751 |
| FGFR3 | masitinib; ponatinib (e.g., Iclusig ®); AZD4547; BGJ398, NVP-BGJ398; nintedanib (e.g., |
| | Vargatef ®, BIBF 1120); Debio 1347, CH5183284; lucitanib; sulfatinib; LY2874455; |
| | dovitinib; XL228; erdafitinib; ENMD-2076; NVP-BGJ-398; pazopanib hydrochloride; |
| | masivet; PD-173074; pazopanib |
| FGFR4 | erdafitinib; NVP-BGJ-398; nintedanib |
| FGR | dasatinib (e.g., BMS-354825, Sprycel ®) |
| FKBP1A | Rimiducid |
| FLT1 | ilorasertib; axitinib (e.g., Inlyta ®); motesanib; regorafenib (e.g., Stivarga ®, BAY-73-4506); |
| | nintedanib (e.g., Vargatef ®, BIBF 1120); lucitanib; pazopanib (e.g., Votrient ®, |
| | GW786034, Armala ™); fruquintinib; tivozanib; glesatinib; sitravatinib; sorafenib tosylate; |
| | sunitinib malate; pazopanib hydrochloride; sunitinib (e.g., Sutent ®, SU11248); MGCD- |
| | 265; cediranib; AZD2171; linifanib; ABT-869 |
| FLT3 | quizartinib; ponatinib (e.g., Iclusig ®); cenisertib; gilteritinib; sorafenib (e.g., Nexavar ®, |
| | BAY-43-9006); lestaurtinib; crenolanib; ENMD-0276; tandutinib; amuvatinib; midostaurin |
| | (e.g., Rydapt ®); PLX3397, PLX108-01; sunitinib malate (e.g., Sutent ®, SU11248); |
| | cabozantinib (e.g., Cabometyx ® (tablet), Cometriq ® (capsule)); tozasertib; AZD1152- |
| | HQPA; sorafenib tosylate; KW 2449; XL184; BMS-907351; MLN518; CT53518; AC220; |
| | linifanib; ABT-869; CEP-701 |
| FLT4 | ilorasertib; axitinib (e.g., Inlyta ®); motesanib; regorafenib (e.g., Stivarga ®, BAY-73-4506; |
| | nintedanib (e.g., Vargatef ®, BIBF 1120); lucitanib; pazopanib (e.g., Votrient ®, |
| | GW786034, Armala); fruquintinib; tivozanib; glesatinib; sitravatinib; telatinib; sorafenib |
| | tosylate; sunitinib malate (e.g., Sutent ®, SU11248); pazopanib hydrochloride; sorafenib |
| | (e.g., BAY-43-9006, Nexavar ®); AG013736; lenvatinib; E7080; MGCD-265; cediranib; |
| | AZD2171 |
| FNTA | tipifarnib; Zarnestra; tipifarnib-P2; tipifarnib-P1; manumycin A |
| FOLH1 | PSMA ADC; mipsagargin; BIND-014 |
| FOLR1 | mirvetuximab soravtansine; vintafolide |
| FOLR2 | vintafolide |
| FOLR3 | vintafolide |
| FRK | regorafenib; dasatinib (e.g., BMS-354825, Sprycel ®) |
| FYN | dasatinib (e.g., BMS-354825, Sprycel ®) |
| FZD8 | ipafricept |
| GART | pemetrexed disodium |
| GNRH1 | Degarelix (e.g., Firmagon ®); leuprolide (e.g., Lupron ®); triptorelin (e.g., Trelstar ®); |
| | goserelin (e.g., Zoladex ®); relugolix |
| GNRHR | goserelin; leuprolide acetate; abarelix; degarelix |
| GSK3A | CT 99021; CHIR-99021; CT99021; SB 216763 |
| GSK3B | CT 99021; BIO; 6-bromoindirubin-3′-oxime; 6BIO; CHIR-99021; CT99021; JW74; BRD- |
| | K81491172; SB 216763 |
| HCK | dasatinib (e.g., BMS-354825, Sprycel ®); bosutinib |
| HDAC1 | resminostat; citarinostat; abexinostat; romidepsin (e.g., Istodax); tucidinostat; Epidaza ®; |
| | panobinostat (e.g., Farydak ®, Faridak); mocetinostat; vorinostat (e.g., Zolinza); entinostat; |
| | belinostat (e.g., Beleodaq ®); remetinostat; THM-I-91; LBH-589; Merck60; BRD6929; |
| | BRD-K11533227; PXD-101; pandacostat; CI-994; BRD-K61166597; apicidin; SAHA; |
| | BRD-K85133207 |
| HDAC10 | vorinostat (e.g., Zolinza ®) |
| HDAC11 | vorinostat (e.g., Zolinza ®) |
| HDAC2 | resminostat; citarinostat; abexinostat; romidepsin (e.g., Istodax ®); tucidinostat; Epidaza ®; |
| | panobinostat (e.g., Farydak ®, Faridak); mocetinostat; vorinostat (e.g., Zolinza ®); |
| | entinostat; belinostat (e.g., Beleodaq ®); remetinostat; romidepsin; THM-I-91; LBH-589; |
| | Merck60; BRD6929; BRD-K11533227; PXD-101; pandacostat; CI-994; BRD-K61166597; |
| | apicidin; SAHA |
| HDAC3 | resminostat; citarinostat; abexinostat; romidepsin (e.g., Istodax ®); tucidinostat; Epidaza ®; |
| | panobinostat (e.g., Farydak ®, Faridak); mocetinostat; vorinostat (e.g., Zolinza ®); |
| | entinostat; belinostat (e.g., Beleodaq); remetinostat; THM-I-91; LBH-589; PXD-101; BRD- |
| | K29313308; pandacostat; Repligen 136; CI-994; apicidin; SAHA |
| HDAC4 | vorinostat (e.g., Zolinza ®); belinostat; panobinostat; romidepsin |
| HDAC5 | vorinostat (e.g., Zolinza ®); belinostat; panobinostat; romidepsin |
| HDAC6 | resminostat; citarinostat; abexinostat; romidepsin (e.g., Istodax ®); tucidinostat; Epidaza ®; |
| | panobinostat (e.g., Farydak ®, Faridak); mocetinostat; vorinostat (e.g., Zolinza ®); |
| | entinostat; belinostat (e.g., Beleodaq ®); remetinostat; ricolinostat; tubastatin A; THM-I-91; |
| | LBH-589; PXD-101; pandacostat; BRD-K51490254; CI-994; BRD-K55478147; apicidin; |
| | ISOX; BRD-K69840642; SAHA |
| HDAC7 | vorinostat (e.g., Zolinza ®); belinostat; panobinostat; romidepsi |
| HDAC8 | vorinostat (e.g., Zolinza ®); panobinostat; THM-I-91; LBH-589; belinostat; PXD-101; |
| | pandacostat; BRD-K51490254; CI-994; apicidin; entinostat; SAHA; BRD-K88742110; |
| | romidepsin |
| HDAC9 | vorinostat (e.g., Zolinza ®); belinostat; panobinostat; romidepsin |
| HPRT1 | mercaptopurine |
| HPSE | muparfostat |
| HSP90AA1 | tanespimycin; onalespib; luminespib; Debio 0932; retaspimycin; SNX-5422; ganetespib; |
| | XL888; geldanamycin; AT13387; SNX-2112; tanespymicin; 17-AAG |
| HSPA1A | elesclomol; VER-155008; pifithrin-mu; NSC303580 |
| HSPA1B | elesclomol; VER-155008; pifithrin-mu; NSC303580 |
| HSPB1 | apatorsen |
| IDH1 | ivosidenib; ML204; MH2075 |
| IDH2 | enasidenib (e.g., Idhifa ®) |
| IDO1 | epacadostat; navoximod; indoximod |
| IFNAR1 | interferon alfa-2b, recombinant; peginterferon alfa-2b |
| IFNAR2 | interferon alfa-2b, recombinant; peginterferon alfa-2b |
| IGF1R | BMS-536924; BMS-754807; linsitinib; XL228; ganitumab; BI 836845; BIIB022; |
| | figitumumab; cixutumumab; dusigitumab; dalotuzumab; istiratumab; AEW541; NVP- |
| | ADW742; OSI-906 |
| IHH | glasdegib; vismodegib (e.g., Erivedge ®) |
| IKBKB | PF-184; BMS-345541 |
| IL1B | canakinumab (e.g., Ilaris ®) |
| IL2RA | aldesleukin; denileukin diftitox |
| IL2RB | aldesleukin; denileukin diftitox |
| IL2RG | aldesleukin; denileukin diftitox |
| IL6 | Siltuximab (e.g., Sylvant ®) |
| IL6R | Tocilizumab (e.g., Actemra ®) |
| IL6ST | Bazedoxifene; SC144 |
| INHA | sotatercept |
| INHBA | sotatercept |
| INHBB | sotatercept |
| INHBC | sotatercept |
| INHBE | sotatercept |
| ITK | pazopanib hydrochloride; pazopanib |
| JAK1 | PRT062070; itacitinib; INCB052793; AZD1480; momelotinib; ruxolitinib (e.g., Jakafi ®, |
| | Jakavi, INCB018424); ruxolitinib phosphate; BIO; 6-bromoindirubin-3′-oxime (6BIO); |
| | CYT-387; INCB-18424 |
| JAK2 | AZD1480; momelotinib; ruxolitinib (e.g., Jakafi ®, INCB018424, Jakavi); AT9283; BMS- |
| | 911543; lestaurtinib; fedratinib; pacritinib; XL019; gandotinib; AZD1152-HQPA; AZ960; |
| | ruxolitinib phosphate; NVP-BSK805; BIO; 6-bromoindirubin-3′-oxime (6BIO); TG- |
| | 101348; SAR302503; CYT-387; CEP-701; INCB-18424 |
| JAK3 | tofacitinib citrate (e.g., Xeljanz ®); PRT062070; tasocitinib; BIO; 6-bromoindirubin-3′- |
| | oxime (6BIO) |
| KDM1A | GSK2879552; CBB-1007 |
| KDR | linifanib; ENMD-2076; foretinib; sulfatinib; vatalanib; orantinib; X-82; XL647, KD019; |
| | ilorasertib; axitinib (e.g., Inlyta ®); motesanib; regorafenib (e.g., Stivarga ®, BAY-73-4506); |
| | nintedanib (e.g., Vargatef ®, BIBF 1120); lucitanib; pazopanib (e.g., Votrient ®, armala, |
| | GW786034); fruquintinib; tivozanib; glesatinib; sitravatinib; AEE788, NVP-AEE788; |
| | ponatinib (e.g., Iclusig ®); cediranib; vandetanib (e.g., Zactima ™, Caprelsa ®, ZD6474); |
| | sorafenib (e.g., Nexavar ®, BAY-43-9006); brivanib; BMS-690514, EVRI; rebastinib |
| | tosylate; lenvatinib (e.g., Lenvima ®); midostaurin (e.g., Rydapt ®); RAF265; sunitinib (e.g., |
| | Sutent ®, SU11248); cabozantinib (e.g., Cabometyx ® (tablet), Cometriq ® (capsule)); |
| | XL820; apatinib; telatinib; ramucirumab (e.g., Cyramza ®); sorafenib tosylate; sunitinib |
| | malate; pazopanib hydrochloride; thalidomide; XL880; EXEL-2880; GSK1363089; |
| | GSK089; OSI-930; BMS-582664; AG013736; E7080; Ki8751; XL184; BMS-907351; |
| | AV-951; BRD4658; MGCD-265; AZD2171; CHIR-265; ABT-869 |
| KIF11 | filanesib; SB-743921 |
| KIT | masitinib; axitinib (e.g., Inlyta ®); motesanib; cenisertib; telatinib; regorafenib (e.g., |
| | Stivarga ®, BAY-73-4506); dasatinib (e.g., BMS-354825, Sprycel ®); pazopanib (e.g., |
| | Votrient ®, GW786034, armala); sitravatinib; tandutinib; amuvatinib; midostaurin (e.g., |
| | Rydapt ®); PLX3397, PLX108-01; imatinib (e.g., Gleevec ®); sunitinib malate (e.g., |
| | Sutent ®, SU11248); cabozantinib (e.g., Cabometyx ® (tablet), Cometriq ® (capsule)); |
| | XL820; sorafenib (e.g., Nexavar ®); midostaurin; sorafenib tosylate; pazopanib |
| | hydrochloride; sorafenib (e.g., BAY-43-9006, Nexavar ®); Ki8751; cabozantinib (e.g., |
| | Cometriq ®); XL184; BMS-907351; Masivet; nilotinib (e.g., AMN107, Tasigna ®); |
| | MLN518; CT53518; AMG-706; imatinib; lenvatinib; ponatinib |
| KRAS | BGB-283 |
| LAP3 | tosedostat; CHR-2797 |
| LCK | dasatinib (e.g., BMS-354825, Sprycel ®); pazopanib |
| LDLR | porfimer |
| LHCGR | goserelin |
| LIMK1 | dabrafenib |
| LYN | bafetinib; cenisertib; dasatinib (e.g., BMS-354825, Sprycel ®); bosutinib; ponatinib |
| MAPIA | estramustine |
| MAP2 | estramustine |
| MAP2K1 | pimasertib; selumetinib; AZD8330; refametinib; BI 847325; CI-1040; GDC-0623; |
| | cobimetinib (e.g., Cotellic ®); trametinib (e.g., Mekinist ®, GSK1120212); binimetinib; |
| | PD0325901, RO5126766, CH5126766; TAK-733; PD318088; GSK1120212; JTP-74057; |
| | AZD6244 |
| MAP2K2 | pimasertib; selumetinib; AZD8330; refametinib; BI 847325; CI-1040; GDC-0623; |
| | cobimetinib (e.g., Cotellic ®); trametinib (e.g., Mekinist ®); binimetinib; PD0325901; |
| | RO5126766, CH5126766; TAK-733; PD318088; GSK1120212; JTP-74057; AZD6244; |
| | GSK1120212 |
| MAP3K7 | (5Z)-7-Oxozeaenol |
| MAP3K8 | cyanoquinoline 11 |
| MAPK1 | BVD-523; ralimetinib; MK-8353, SCH900353; N-hexanoyl-D-sphingosine; C6-ceramide; |
| | SCH772984 |
| MAPK11 | regorafenib |
| MAPK14 | BIRB-796; doramapimod; skepinone-L; BIRB 0796 |
| MAPK3 | pluripotin; SCH772984 |
| MAPK8 | SP600125; BIRB 0796; JNK Inhibitor VIII |
| MAPK9 | SP600125 |
| MCL1 | navitoclax; obatoclax; marinopyrrole a; maritoclax; obatoclax mesylate |
| MDM2 | SAR405838, MI-773; idasanutlin; nutlin-3; RITA; NSC652287; HLI-373; DPD; JNJ- |
| | 26854165; serdemetan; SJ-172550; Nutlin-3a |
| MET | sitravatinib; AMG 208; AMG 337; tivantinib; BMS-777607; EMD 1214063, |
| | MSC2156119J; foretinib; volitinib; INCB028060, INC280; glesatinib; MK-2461; |
| | amuvatinib; crizotinib (e.g., Xalkori ®); PF-04217903; SAR125844; cabozantinib (e.g., |
| | Cabometyx ® (tablet), Cometriq ® (capsule)); rilotumumab; ficlatuzumab; telisotuzumab; |
| | emibetuzumab; onartuzumab; cMet CAR-mRNA Electroporated autologous T |
| | lymphocytes; MGCD-265; PHA665752; SU11274; XL880; EXEL-2880; GSK1363089; |
| | GSK089; SGX-523; OSI-930; ARQ-197; XL184; BMS-907351; PF-2341066; PF- |
| | 02341066 |
| MGMT | Lomeguatrib |
| MRE11 | Mirin |
| MS4A1 | obinutuzumab (e.g., Gazyvaro ®, Gazyva ®); rituximab (e.g., Rituxan ®, Mabthera ®); |
| | ibritumomab tiuxetan (e.g., Zevalin ®); ublituximab; ofatumumab (e.g., Arzerra ®, HuMax- |
| | CD20); rituximab/hyaluronidase human (e.g., Rituxan Hycela); rituximab (e.g., Rituxan ®, |
| | Mabthera); tositumomab (e.g., Bexxar ®); tositumomab and Iodine I 131 tositumomab; |
| | ofatumumab; obinutuzumab |
| MSLN | DMOT4039A; anti-mesothelin iCasp9M28z CAR-transduced autologous T lymphocytes; |
| | amatuximab |
| MST1R | Glesatinib |
| MTOR | ridaforolimus (deforolimus); sirolimus (e.g., Rapamune ®); AZD2014; AZD8055; |
| | dactolisib; BGT226, NVP-BGT226; CC-223; temsirolimus (e.g., Torisel ®); apitolisib; |
| | sapanisertib; OSI-027; PF-4691502; PI-103; gedatolisib; PWT33597; everolimus (e.g., |
| | Afinitor ®); SF1126; voxtalisib; BEZ235; GSK1059615; CCI-779; NVP-BEZ235; KU- |
| | 0063794; XL765; SAR245409; rapamycin |
| MUC5AC | ensituximab |
| NAE1 | pevonedistat; MLN-4924 |
| NAMPT | GMX-1778; CHS-828; APO866; FK866; BRD0667; CAY10618 |
| NBN | Rucaparib; AG014699; PF-01367338; AG-014699 |
| NCSTN | MK-0752; RO4929097; semagacestat; LY450139; L-685458 |
| NEK11 | dabrafenib |
| NOTCH1 | brontictuzumab; MK0752; tarextumab; nirogacestat; REGN421; RO4929097, R4733 |
| NOTCH2 | MK0752; tarextumab; nirogacestat; REGN421; RO4929097, R4733 |
| NOTCH3 | MK0752; tarextumab; nirogacestat; REGN421; RO4929097, R4733 |
| NOTCH4 | MK0752; tarextumab; nirogacestat; REGN421; RO4929097, R4733 |
| NPEPPS | tosedostat; CHR-2797 |
| NR3C1 | fluoxymesterone; avicin D; dexamethasone (e.g., Decadron ®) |
| NRAS | BGB-283 |
| NTRK1 | AZD7451; LOXO-101; sitravatinib; PLX7486; entrectinib; TSR-011; lestaurtinib; |
| | regorafenib; CEP-701; GW 441756 |
| NTRK2 | AZD7451; LOXO-101; sitravatinib; PLX7486; entrectinib; TSR-011 |
| NTRK3 | AZD7451; LOXO-101; sitravatinib; PLX7486; entrectinib; TSR-011 |
| PARP1 | veliparib; rucaparib (e.g., Rubraca ®); olaparib (e.g., Lynparza ®); talazoparib; iniparib; |
| | niraparib (e.g., Zejula ®); rucaparib camsylate; AZD-2281; KU-0059436; NVP-LDE225; |
| | BRD6430; ABT-888; BMN-673 |
| PARP2 | veliparib; rucaparib (e.g., Rubraca ®); olaparib (e.g., Lynparza ®); talazoparib; iniparib; |
| | niraparib (e.g., Zejula ®); rucaparib camsylate; AZD-2281; KU-0059436; NVP-LDE225; |
| | BRD6430; ABT-888; BMN-673 |
| PARP3 | veliparib; rucaparib (e.g., Rubraca ®); olaparib (e.g., Lynparza ®); talazoparib; iniparib; |
| | niraparib (e.g., Zejula ®); rucaparib camsylate |
| PDE5A | Sildenafil (e.g., Viagra ®, Revatio ®) |
| PDGFRA | ilorasertib; motesanib; nintedanib (e.g., Vargatef ®, BIBF 1120); pazopanib (e.g., Votrient); |
| | sitravatinib; tandutinib; imatinib (e.g., Gleevec); X-82; crenolanib; amuvatinib; midostaurin |
| | (e.g., Rydapt ®); olaratumab (e.g., Lartruvo ®); tovetumab; sorafenib (e.g., Nexavar ®); |
| | sunitinib malate; pazopanib hydrochloride; regorafenib; Ki8751; masitinib (e.g., Masivet ®); |
| | AMG-706; axitinib |
| PDGFRB | ilorasertib; motesanib; nintedanib (e.g., Vargatef ®, BIBF 1120); pazopanib (e.g., |
| | Votrient ®, GW786034, armala); sitravatinib; tandutinib; imatinib (e.g., Gleevec ®); X-82; |
| | linifanib; axitinib (e.g., Inlyta ®); sorafenib (e.g., Nexavar ®); telatinib; regorafenib (e.g., |
| | Stivarga ®, BAY-43-9006); sunitinib (e.g., Sutent ®, SU11248); orantinib; XL820; |
| | midostaurin; sorafenib tosylate; sunitinib malate; pazopanib hydrochloride; dasatinib (e.g., |
| | BMS-354825, Sprycel ®); masitinib (e.g., Masivet ®); MLN518; CT53518; ABT-869; |
| | AMG-706 |
| PGF | ziv-aflibercept (e.g., Zaltrap ®) |
| PIGF | ziv-aflibercept (e.g., Zaltrap ®) |
| PIK3CA | copanlisib; dactolisib; BGT226, NVP-BGT226; buparlisib; alpelisib; pictilisib; apitolisib; |
| | omipalisib; GSK2636771; PF-4691502; PI-103; gedatolisib; PWT33597; PX-866; SF1126; |
| | pilaralisib; voxtalisib; sophoretin, quercetin; taselisib; INK-1117; BEZ235; GDC0941; |
| | NVP-BYL-719; GSK1059615; NVP-BEZ235; BYL-719; XL765; SAR245409 |
| PIK3CB | AZD8186; BEZ235; GDC0941; TGX-221; GSK1059615; NVP-BEZ235; GSK-2636771; |
| | AZD6482; PI-103; XL765; SAR245409 |
| PIK3CD | Idelalisib (e.g., Zydelig ®); dezapelisib; umbralisib tosylate; duvelisib; GDC0941; |
| | GSK1059615; BEZ235; NVP-BEZ235; CAL-101; TG100-115; PI-103; XL765; |
| | SAR245409; IC-87114 |
| PIK3CG | AZD8186; duvelisib; GDC0941; GSK1059615; BEZ235; NVP-BEZ235; PIK-93; |
| | ZSTK474; TG100-115; PI-103; XL765; SAR245409 |
| PIM1 | AZD1208; PIM447 |
| PIM2 | AZD1208; PIM447 |
| PIM3 | AZD1208; PIM447; SL 0101-1 |
| PLK1 | volasertib; BI 2536; GSK461364; NMS-1286937; rigosertib (e.g., Estybon ®, ON-01910); |
| | LFM-A13/DDE-28; Novonex; HMN-214; GW-843682X |
| PLK2 | BI-2536; LFM-A13/DDE-28 |
| PLK3 | BI-2536; LFM-A13/DDE-28; GW-843682X |
| POLA1 | clofarabine (e.g., Clolar ®); nelarabine |
| PORCN | LGK974 |
| PPARG | CS 7017, RS5444 |
| PPP2CA | LB-100; N-hexanoyl-D-sphingosine; C6-ceramide |
| PRKCA | sophoretin, quercetin; enzastaurin; midostaurin (e.g., Rydapt ®) |
| PRKCB | sophoretin, quercetin; enzastaurin; sotrastaurin; AEB071; LY317615 |
| PRKCE | sophoretin, quercetin; enzastaurin |
| PRKCG | sophoretin, quercetin; enzastaurin |
| PRKCI | Gossypol |
| PRKDC | KU-0060648; PI-103; NU-7441 |
| PRLR | Fluoxymesterone |
| PSEN1 | MK-0752; RO4929097; semagacestat; LY450139; L-685458 |
| PRMT5 | GSK3326595 |
| PSEN1 | MRK-560 |
| PSENEN | MK0752; MRK 003; RO4929097; semagacestat; LY450139; L-685458; BMS-708163 |
| PSMB1 | bortezomib (e.g., Velcade ®); carfilzomib |
| PSMB10 | Carfilzomib |
| PSMB2 | bortezomib; carfilzomib |
| PSMB5 | bortezomib (e.g., Velcade ®); carfilzomib; MLN2238; MLN9708 |
| PSMB8 | Carfilzomib |
| PSMB9 | Carfilzomib |
| PSMD1 | Bortezomib |
| PSMD2 | Bortezomib |
| PTCH1 | vismodegib (e.g., Erivedge ®) |
| PTGS2 | valdecoxib (e.g., Bextra ®) |
| PTK2 | masitinib; GSK2256098; VS-4718; defactinib; PF-573228; PF-562271 |
| PTK6 | Vandetanib |
| RAC1 | NSC23766; EHT 1864 |
| RAD50 | Mirin |
| RAF1 | sorafenib (e.g., Nexavar ®); regorafenib (e.g., Stivarga ®, BAY-73-4506); encorafenib; |
| | MLN2480; RO5126766, CH5126766; XL281, BMS-908662; RAF265; AZ628; GW 5074; |
| | sorafenib tosylate; dabrafenib |
| RARA | alitretinoin; AM-580; BMS-195614; ATRA; all-trans retinoic acid; tretinoin (e.g., |
| | Vesanoid ®); Ch-55 |
| RARB | alitretinoin; LE-135; AM-580; BMS-195614; ATRA; all-trans retinoic acid; tretinoin (e.g., |
| | Vesanoid ®); Ch-55; AC55649 |
| RARG | alitretinoin; BMS-270394; AM-580; CD-1530; CD-437; AHPN; BMS-195614; ATRA; all- |
| | trans retinoic acid; tretinoin (e.g., Vesanoid ®); Ch-55 |
| RBM39 | Indisulam |
| RET | motesanib; vandetanib (e.g., Zactima ™, Caprelsa ®); sorafenib (e.g., Nexavar ®); |
| | regorafenib (e.g., Stivarga ®, BAY-73-4506); sitravatinib; amuvatinib; sunitinib (e.g., |
| | Sutent ®, SU11248); cabozantinib (e.g., Cabometyx ® (tablet), Cometriq ® (capsule)); |
| | AZD1152-HQPA; lestaurtinib; sorafenib tosylate; AMG-706; CEP-701; lenvatinib |
| RICTOR | OSI-027 |
| ROCK1 | GSK269962A |
| ROCK2 | GSK269962A |
| ROS1 | crizotinib (e.g., Xalkori ®); lorlatinib; entrectinib |
| RPL3 | omacetaxine mepesuccinate |
| RPS6KB1 | AT13148; PF-4708671 |
| RPTOR | OSI-027 |
| RRM1 | gemcitabine; Hydroxyurea; ciclopirox olamine (e.g., Loprox ®); clofarabine (e.g., Clolar ®) |
| RXRA | alitretinoin; bexarotene; targretin |
| RXRB | alitretinoin; bexarotene; targretin |
| RXRG | alitretinoin; bexarotene; targretin |
| S1PR1 | fingolimod (e.g., FTY720, Gilenya ®) |
| SH2B3 | pazopanib hydrochloride |
| SHH | glasdegib; vismodegib (e.g., Erivedge) |
| SIK1 | dabrafenib |
| SIRT1 | salermide; isoliquiritigenin; GU 17; SRT-1720; EX-527; SEN0014196 |
| SLAMF7 | elotuzumab (e.g., Empliciti ®) |
| SLC2A2 | streptozocin |
| SMO | vismodegib (e.g., Erivedge ®); patidegib; sonidegib (e.g., Odomzo ®); taladegib; |
| | Cyclopamine; Vismodegib; GANT-61; purmorphamine; GDC-0449 |
| SRC | saracatinib; ilorasertib; dasatinib (e.g., BMS-354825, Sprycel ®); KX2-391; XL228; WH-4- |
| | 025; AZD0530; KX01; bosutinib (e.g., SKI-606, Bosulif ®); vandetanib |
| SSTR2 | lanreotide |
| SSTR5 | lanreotide |
| STAT3 | OPB-31121; pyrimethamine (e.g., Daraprim ®); niclosamide (e.g., Niclocide ®); S3I-201; |
| | NSC74859 |
| SYK | entospletinib; PRT062070; fostamatinib; R-406 |
| TBK1 | momelotinib; BX-795 |
| TEK | glesatinib; cabozantinib (e.g., Cabometyx ® (tablet), Cometriq ® (capsule)); vandetanib; |
| | regorafenib; XL184; BMS-907351; MGCD-265 |
| TERT | BIBR-1532; MST-312 |
| TGFB1 | luspatercept |
| TGFBR1 | galunisertib; TEW-7197; SB-525334; SB-431542; LY-2157299; SB-505124 |
| TLR5 | Entolimod |
| TLR7 | Imiquimod |
| TLR8 | motolimod; imiquimod |
| TNF | thalidomide; lenalidomide |
| TNFRSF8 | brentuximab vedotin (e.g., Adcetris ®) |
| TNFSF11 | denosumab (e.g., Xgeva) |
| TNFSF13B | belimumab (e.g., Benlysta) |
| TNKS | Chembridge cat# 7667791; XAV 939 |
| TOP1 | etirinotecan pegol; irinotecan (e.g., Camptosar ®); irinotecan liposome (e.g., Onivyde ®); |
| | camptothecin; irinotecan; topotecan hydrochloride; SN-38; 7-ethyl-10- |
| | hydroxycamptothecin; topotecan (e.g., Hycamtin ®); irinotecan trihydrochloride |
| TOP1MT | irinotecan; topotecan hydrochloride |
| TOP2A | mitoxantrone (e.g., Novantrone ®); daunorubicin (e.g., Cerubidine ®); liposomal |
| | daunorubicin (e.g., DaunoXome ®); doxorubicin (e.g., Adriamycin PFS ®); liposomal |
| | doxorubicin (e.g., Doxil ®); epirubicin (e.g., Ellence ®); idarubicin (e.g., Idamycin ®); |
| | valrubicin (e.g., Valstar); etoposide/etoposide phosphate (e.g., Toposar, Etopophos ®); |
| | teniposide (e.g., Vumon ®, VM-26); amonafide; hydroxydaunorubicin |
| TOP2B | mitoxantrone (e.g., Novantrone ®); daunorubicin (e.g., Cerubidine ®); liposomal |
| | daunorubicin (e.g., DaunoXome ®); doxorubicin (e.g., Adriamycin PFS ®); liposomal |
| | doxorubicin (e.g., Doxil ®); epirubicin (e.g., Ellence ®); idarubicin (e.g., Idamycin ®); |
| | valrubicin (e.g., Valstar); etoposide/etoposide phosphate (e.g., Toposar, Etopophos ®); |
| | teniposide (e.g., Vumon ®, VM-26); amonafide |
| TUBA1A | vinblastine sulfate (e.g., Oncovin ®) |
| TUBA4A | vincristine sulfate (e.g., Oncovin ®); paclitaxel (e.g., Taxol ®); docetaxel (e.g., Taxotere ®); |
| | cabazitaxel; eribulin mesylate |
| TUBB | vincristine sulfate (e.g., Oncovin ®); vinblastine sulfate; vinorelbine tartrate |
| TUBB1 | paclitaxel (e.g., Taxol ®); docetaxel (e.g., Taxotere ®); cabazitaxel; eribulin mesylate; |
| | vincristine (e.g., Oncovin ®); leurocristine |
| TUBB3 | Ixabepilone |
| TUBD1 | vinblastine sulfate |
| TUBE1 | vinblastine sulfate |
| TUBG1 | vinblastine sulfate |
| TXN | PX-12; IV-2 |
| TYK2 | INCB-18424 |
| TYMS | leucovorin; gemcitabine; capecitabine; pemetrexed disodium; pralatrexate; fluorouracil |
| | (e.g., Adrucil ®, Efudex ®) |
| VDR | BXL-628; elocalcitol |
| VEGFA | bevacizumab (e.g., Avastin ®); navicixizumab; vanucizumab; muparfostat; ziv-aflibercept |
| | (e.g., Zaltrap ®); vandetanib |
| VEGFB | ziv-aflibercept (e.g., Zaltrap ®); muparfostat; bevacizumab (e.g., Avastin ®) |
| WEE1 | AZD1775, MK1775; 681640; MK-1775; BRD9876 |
| XIAP | birinapant; embelin; LBW242; SM-406; AT-406; TL-32711 |
| XPO1 | selinexor; leptomycin B; compound 7d-cis; BRD9047 |
| YES1 | dasatinib (e.g., BMS-354825, Sprycel ®) |

Tables 4-5C provide exemplary protein targets of known therapeutic agents, one or more of which may be useful in a target library described herein.

TABLE 4
Gene targets of therapeutic agents.

| Gene | | | | |
|---|---|---|---|---|
| Symbol | Ensembl ID | Gene Name | Chr | Position |
| ABAT | ENSG00000183044 | 4-aminobutyrate aminotransferase | 16 | 8674596-8784575 |
| ABCA1 | ENSG00000165029 | ATP binding cassette subfamily A member 1 | 9 | 104781006-104928155 |
| ABCC1 | ENSG00000103222 | ATP binding cassette subfamily C member 1 | 16 | 15949577-16143074 |
| ABCC2 | ENSG00000023839 | ATP binding cassette subfamily C member 2 | 10 | 99782640-99852594 |
| ABCC8 | ENSG00000006071 | ATP binding cassette subfamily C member 8 | 11 | 17392498-17476879 |
| ABL1 | ENSG00000097007 | ABL proto-oncogene 1, non-receptor tyrosine | 9 | 130713016-130887675 |
| | | kinase | | |
| ABL2 | ENSG00000143322 | ABL proto-oncogene 2, non-receptor tyrosine | 1 | 179099330-179229684 |
| | | kinase | | |
| ACAA1 | ENSG00000060971 | Acetyl-CoA acyltransferase 1 | 3 | 38103129-38137242 |
| ACE | ENSG00000159640 | Angiotensin I converting enzyme | 17 | 63477061-63498380 |
| ACE2 | ENSG00000130234 | Angiotensin I converting enzyme 2 | X | 15561033-15602148 |
| ACHE | ENSG00000087085 | Acetylcholinesterase (Cartwright blood group) | 7 | 100889994-100896974 |
| ACP3 | ENSG00000014257 | Acid phosphatase 3 | 3 | 132317369-132368298 |
| ADA | ENSG00000196839 | Adenosine deaminase | 20 | 44619522-44652233 |
| ADH1A | ENSG00000187758 | Alcohol dehydrogenase 1A (class I), alpha | 4 | 99276369-99291003 |
| | | polypeptide | | |
| ADH1B | ENSG00000196616 | Alcohol dehydrogenase 1B (class I), beta | 4 | 99304971-99352760 |
| | | polypeptide | | |
| ADH1C | ENSG00000248144 | Alcohol dehydrogenase 1C (class I), gamma | 4 | 99336497-99352746 |
| | | polypeptide | | |
| ADK | ENSG00000156110 | Adenosine kinase | 10 | 74151202-74709963 |
| ADORA1 | ENSG00000163485 | Adenosine A1 receptor | 1 | 203090654-203167405 |
| ADORA2A | ENSG00000128271 | Adenosine A2a receptor | 22 | 24417879-24442357 |
| ADORA2B | ENSG00000170425 | Adenosine A2b receptor | 17 | 15944917-15975746 |
| ADORA3 | ENSG00000282608 | Adenosine A3 receptor | 1 | 111499429-111503633 |
| ADRA1A | ENSG00000120907 | Adrenoceptor alpha 1A | 8 | 26748150-26867278 |
| ADRA1B | ENSG00000170214 | Adrenoceptor alpha 1B | 5 | 159865080-159973012 |
| ADRA1D | ENSG00000171873 | Adrenoceptor alpha 1D | 20 | 4220630-4249287 |
| ADRA2A | ENSG00000150594 | Adrenoceptor alpha 2A | 10 | 111077029-111080907 |
| ADRA2C | ENSG00000184160 | Adrenoceptor alpha 2C | 4 | 3766348-3768526 |
| ADRB1 | ENSG00000043591 | Adrenoceptor beta 1 | 10 | 114043866-114046904 |
| ADRB2 | ENSG00000169252 | Adrenoceptor beta 2 | 5 | 148825245-148828687 |
| ADRB3 | ENSG00000188778 | Adrenoceptor beta 3 | 8 | 37962990-37966599 |
| AGTR1 | ENSG00000144891 | Angiotensin II receptor type 1 | 3 | 148697784-148743008 |
| AGXT | ENSG00000172482 | Alanine--glyoxylate and serine--pyruvate | 2 | 240868824-240880502 |
| | | aminotransferase | | |
| AKR1C2 | ENSG00000151632 | Aldo-keto reductase family 1 member C2 | 10 | 4987400-5018031 |
| AKR1D1 | ENSG00000122787 | Aldo-keto reductase family 1 member D1 | 7 | 138002324-138118305 |
| AKT1 | ENSG00000142208 | AKT serine/threonine kinase 1 | 14 | 104769349-104795751 |
| AKT2 | ENSG00000105221 | AKT serine/threonine kinase 2 | 19 | 40230317-40285536 |
| AKT3 | ENSG00000117020 | AKT serine/threonine kinase 3 | 1 | 243488233-243851079 |
| ALAD | ENSG00000148218 | Aminolevulinate dehydratase | 9 | 113386312-113401290 |
| ALDH2 | ENSG00000111275 | Aldehyde dehydrogenase 2 family member | 12 | 111766887-111817532 |
| ALK | ENSG00000171094 | ALK receptor tyrosine kinase | 2 | 29192774-29921586 |
| ALOX5 | ENSG00000012779 | Arachidonate 5-lipoxygenase | 10 | 45374176-45446119 |
| ALPG | ENSG00000163286 | Alkaline phosphatase, germ cell | 2 | 232406844-232410714 |
| AMY2A | ENSG00000243480 | Amylase alpha 2A | 1 | 103616811-103625780 |
| ANGPT1 | ENSG00000154188 | Angiopoietin 1 | 8 | 107249482-107498055 |
| ANGPT2 | ENSG00000091879 | Angiopoietin 2 | 8 | 6499632-6563409 |
| ANO1 | ENSG00000131620 | Anoctamin 1 | 11 | 70078302-70189528 |
| ANPEP | ENSG00000166825 | Alanyl aminopeptidase, membrane | 15 | 89784895-89815401 |
| ANXA1 | ENSG00000135046 | Annexin A1 | 9 | 73151865-73170393 |
| AOC3 | ENSG00000131471 | Amine oxidase copper containing 3 | 17 | 42851184-42858130 |
| APEX1 | ENSG00000100823 | Apurinic/apyrimidinic endodeoxyribonuclease 1 | 14 | 20455191-20457772 |
| APH1A | ENSG00000117362 | Aph-1 homolog A, gamma-secretase subunit | 1 | 150265399-150269580 |
| APH1B | ENSG00000138613 | Aph-1 homolog B, gamma-secretase subunit | 15 | 63276018-63309126 |
| APP | ENSG00000142192 | Amyloid beta precursor protein | 21 | 25880550-26171128 |
| AR | ENSG00000169083 | Androgen receptor | X | 67544021-67730619 |
| ARAF | ENSG00000078061 | A-Raf proto-oncogene, serine/threonine kinase | X | 47561100-47571920 |
| ASIC1 | ENSG00000110881 | Acid sensing ion channel subunit 1 | 12 | 50057548-50083611 |
| ATIC | ENSG00000138363 | 5-aminoimidazole-4-carboxamide ribonucleotide | 2 | 215311956-215349773 |
| | | formyltransferase/IMP cyclohydrolase | | |
| ATP1A1 | ENSG00000163399 | ATPase Na+/K+ transporting subunit alpha 1 | 1 | 116372668-116410261 |
| ATP2C1 | ENSG00000017260 | ATPase secretory pathway Ca2+ transporting 1 | 3 | 130850595-131016712 |
| ATP4A | ENSG00000105675 | ATPase H+/K+ transporting subunit alpha | 19 | 35550031-35563658 |
| ATP6V1B2 | ENSG00000147416 | ATPase H+ transporting V1 subunit B2 | 8 | 20197381-20226819 |
| ATR | ENSG00000175054 | ATR serine/threonine kinase | 3 | 142449007-142578733 |
| AURKA | ENSG00000087586 | Aurora kinase A | 20 | 56369389-56392337 |
| AURKB | ENSG00000178999 | Aurora kinase B | 17 | 8204733-8210600 |
| AURKC | ENSG00000105146 | Aurora kinase C | 19 | 57230802-57235548 |
| AVPR1A | ENSG00000166148 | Arginine vasopressin receptor 1A | 12 | 63142759-63151201 |
| AVPR1B | ENSG00000198049 | Arginine vasopressin receptor 1B | 1 | 206106936-206117699 |
| AVPR2 | ENSG00000126895 | Arginine vasopressin receptor 2 | X | 153902531-153907166 |
| AXL | ENSG00000167601 | AXL receptor tyrosine kinase | 19 | 41219223-41261766 |
| B4GALNT1 | ENSG00000135454 | Beta-1,4-N-acetyl-galactosaminyltransferase 1 | 12 | 57623409-57633239 |
| BAX | ENSG00000087088 | BCL2 associated X, apoptosis regulator | 19 | 48954815-48961798 |
| BCHE | ENSG00000114200 | Butyrylcholinesterase | 3 | 165772904-165837462 |
| BCL2 | ENSG00000171791 | BCL2 apoptosis regulator | 18 | 63123346-63320128 |
| BCL2L1 | ENSG00000171552 | BCL2 like 1 | 20 | 31664452-31723989 |
| BCL2L2 | ENSG00000129473 | BCL2 like 2 | 14 | 23298790-23311751 |
| BCR | ENSG00000186716 | BCR activator of RhoGEF and GTPase | 22 | 23179704-23318037 |
| BDKRB2 | ENSG00000168398 | Bradykinin receptor B2 | 14 | 96204679-96244166 |
| BGLAP | ENSG00000242252 | Bone gamma-carboxyglutamate protein | 1 | 156242184-156243317 |
| BIRC5 | ENSG00000089685 | Baculoviral IAP repeat containing 5 | 17 | 78214186-78225636 |
| BLK | ENSG00000136573 | BLK proto-oncogene, Src family tyrosine kinase | 8 | 11486894-11564599 |
| BLVRB | ENSG00000090013 | Biliverdin reductase B | 19 | 40447765-40465764 |
| BMX | ENSG00000102010 | BMX non-receptor tyrosine kinase | X | 15464246-15556529 |
| BRAF | ENSG00000157764 | B-Raf proto-oncogene, serine/threonine kinase | 7 | 140719327-140924928 |
| BRD2 | ENSG00000204256 | Bromodomain containing 2 | 6 | 32968594-32981505 |
| BRD3 | ENSG00000169925 | Bromodomain containing 3 | 9 | 134030305-134068535 |
| BRD4 | ENSG00000141867 | Bromodomain containing 4 | 19 | 15235519-15332545 |
| BTK | ENSG00000010671 | Bruton tyrosine kinase | X | 101349447-101390796 |
| C1R | ENSG00000159403 | Complement C1r | 12 | 7080214-7092540 |
| C1S | ENSG00000182326 | Complement C1s | 12 | 6988259-7071032 |
| C3 | ENSG00000125730 | Complement C3 | 19 | 6677704-6730562 |
| C4A | ENSG00000244731 | Complement C4A (Rodgers blood group) | 6 | 31982057-32002681 |
| C4B | ENSG00000224389 | Complement C4B (Chido blood group) | 6 | 32014795-32035418 |
| C5 | ENSG00000106804 | Complement C5 | 9 | 120952335-121050275 |
| CA1 | ENSG00000133742 | Carbonic anhydrase 1 | 8 | 85327608-85379014 |
| CA14 | ENSG00000118298 | Carbonic anhydrase 14 | 1 | 150257251-150265078 |
| CA2 | ENSG00000104267 | Carbonic anhydrase 2 | 8 | 85463968-85481493 |
| CA3 | ENSG00000164879 | Carbonic anhydrase 3 | 8 | 85373436-85449040 |
| CA4 | ENSG00000167434 | Carbonic anhydrase 4 | 17 | 60149942-60170899 |
| CA7 | ENSG00000168748 | Carbonic anhydrase 7 | 16 | 66844414-66854153 |
| CACNA1A | ENSG00000141837 | Calcium voltage-gated channel subunit alpha1 A | 19 | 13206442-13633025 |
| CACNA1B | ENSG00000148408 | Calcium voltage-gated channel subunit alpha1 B | 9 | 137877782-138124624 |
| CACNA1C | ENSG00000151067 | Calcium voltage-gated channel subunit alpha1 C | 12 | 1970786-2697950 |
| CACNA1D | ENSG00000157388 | Calcium voltage-gated channel subunit alpha1 D | 3 | 53328963-53813733 |
| CACNA1F | ENSG00000102001 | Calcium voltage-gated channel subunit alpha1 F | X | 49205063-49233371 |
| CACNA1G | ENSG00000006283 | Calcium voltage-gated channel subunit alpha1 G | 17 | 50560715-50627474 |
| CACNA1H | ENSG00000196557 | Calcium voltage-gated channel subunit alpha1 H | 16 | 1153106-1221771 |
| CACNA1I | ENSGOOOOO100346 | Calcium voltage-gated channel subunit alpha1 I | 22 | 39570753-39689735 |
| CACNA1S | ENSG00000081248 | Calcium voltage-gated channel subunit alpha1 S | 1 | 201039512-201112451 |
| CACNA2D1 | ENSG00000153956 | Calcium voltage-gated channel auxiliary subunit | 7 | 81946444-82443777 |
| | | alpha2delta 1 | | |
| CACNA2D2 | ENSG00000007402 | Calcium voltage-gated channel auxiliary subunit | 3 | 50362799-50504244 |
| | | alpha2delta 2 | | |
| CACNB1 | ENSG00000067191 | Calcium voltage-gated channel auxiliary subunit | 17 | 39173453-39197703 |
| | | beta 1 | | |
| CACNB2 | ENSG00000165995 | Calcium voltage-gated channel auxiliary subunit | 10 | 18140424-18543557 |
| | | beta 2 | | |
| CACNB3 | ENSG00000167535 | Calcium voltage-gated channel auxiliary subunit | 12 | 48813794-48828941 |
| | | beta 3 | | |
| CACNB4 | ENSG00000182389 | Calcium voltage-gated channel auxiliary subunit | 2 | 151832768-152099475 |
| | | beta 4 | | |
| CACNG1 | ENSG00000108878 | Calcium voltage-gated channel auxiliary subunit | 17 | 67044554-67056797 |
| | | gamma 1 | | |
| CALY | ENSG00000130643 | Calcyon neuron specific vesicular protein | 10 | 133324072-133336935 |
| CAMLG | ENSG00000164615 | Calcium modulating ligand | 5 | 134738548-134752157 |
| CARTPT | ENSG00000164326 | CART prepropeptide | 5 | 71719275-71721048 |
| CASR | ENSG00000036828 | Calcium sensing receptor | 3 | 122183668-122291629 |
| CAT | ENSG00000121691 | Catalase | 11 | 34438934-34472060 |
| CCKAR | ENSG00000163394 | Cholecystokinin A receptor | 4 | 26481396-26490484 |
| CCKBR | ENSG00000110148 | Cholecystokinin B receptor | 11 | 6259806-6272127 |
| CCL2 | ENSG00000108691 | C-C motif chemokine ligand 2 | 17 | 34255218-34257203 |
| CCND1 | ENSG00000110092 | Cyclin D1 | 11 | 69641156-69654474 |
| CCND2 | ENSG00000118971 | Cyclin D2 | 12 | 4273762-4305353 |
| CCND3 | ENSG00000112576 | Cyclin D3 | 6 | 41934934-42050357 |
| CCR5 | ENSG00000160791 | C-C motif chemokine receptor 5 | 3 | 46370854-46376206 |
| | | (gene/pseudogene) | | |
| CD19 | ENSG00000177455 | CD19 molecule | 16 | 28931965-28939342 |
| CD2 | ENSG00000116824 | CD2 molecule | 1 | 116754430-116769229 |
| CD247 | ENSG00000198821 | CD247 molecule | 1 | 167430640-167518610 |
| CD274 | ENSG00000120217 | CD274 molecule | 9 | 5450503-5470566 |
| CD33 | ENSG00000105383 | CD33 molecule | 19 | 51225064-51243860 |
| CD38 | ENSG00000004468 | CD38 molecule | 4 | 15778275-15853232 |
| CD3D | ENSG00000167286 | CD3d molecule | 11 | 118338954-118342744 |
| CD3E | ENSG00000198851 | CD3e molecule | 11 | 118304730-118316175 |
| CD3G | ENSG00000160654 | CD3g molecule | 11 | 118344344-118355161 |
| CD4 | ENSG00000010610 | CD4 molecule | 12 | 6786858-6820799 |
| CD44 | ENSG00000026508 | CD44 molecule (Indian blood group) | 11 | 35138870-35232402 |
| CD52 | ENSG00000169442 | CD52 molecule | 1 | 26317958-26320523 |
| CD80 | ENSG00000121594 | CD80 molecule | 3 | 119524293-119559614 |
| CD86 | ENSG00000114013 | CD86 molecule | 3 | 122055362-122121139 |
| CDK1 | ENSG00000170312 | Cyclin dependent kinase 1 | 10 | 60778331-60794852 |
| CDK2 | ENSG00000123374 | Cyclin dependent kinase 2 | 12 | 55966781-55972789 |
| CDK4 | ENSG00000135446 | Cyclin dependent kinase 4 | 12 | 57747727-57756013 |
| CDK5 | ENSG00000164885 | Cyclin dependent kinase 5 | 7 | 151053815-151057897 |
| CDK6 | ENSG00000105810 | Cyclin dependent kinase 6 | 7 | 92604921-92836573 |
| CDK7 | ENSG00000134058 | Cyclin dependent kinase 7 | 5 | 69234795-69277430 |
| CDK9 | ENSG00000136807 | Cyclin dependent kinase 9 | 9 | 127785679-127790792 |
| CES1 | ENSG00000198848 | Carboxylesterase 1 | 16 | 55802851-55833337 |
| CFTR | ENSG00000001626 | CF transmembrane conductance regulator | 7 | 117287120-117715971 |
| CHD1 | ENSG00000153922 | Chromodomain helicase DNA binding protein 1 | 5 | 98853985-98928957 |
| CHEK1 | ENSG00000149554 | Checkpoint kinase 1 | 11 | 125625665-125676255 |
| CHEK2 | ENSG00000183765 | Checkpoint kinase 2 | 22 | 28687743-28742422 |
| CHRM1 | ENSG00000168539 | Cholinergic receptor muscarinic 1 | 11 | 62908679-62921807 |
| CHRM2 | ENSG00000181072 | Cholinergic receptor muscarinic 2 | 7 | 136868669-137020255 |
| CHRM3 | ENSG00000133019 | Cholinergic receptor muscarinic 3 | 1 | 239386565-239915452 |
| CHRM4 | ENSG00000180720 | Cholinergic receptor muscarinic 4 | 11 | 46385098-46386608 |
| CHRM5 | ENSG00000184984 | Cholinergic receptor muscarinic 5 | 15 | 33968497-34067458 |
| CHRNA10 | ENSG00000129749 | Cholinergic receptor nicotinic alpha 10 subunit | 11 | 3665587-3671384 |
| CHRNA2 | ENSG00000120903 | Cholinergic receptor nicotinic alpha 2 subunit | 8 | 27459756-27479883 |
| CHRNA3 | ENSG00000080644 | Cholinergic receptor nicotinic alpha 3 subunit | 15 | 78593052-78621295 |
| CHRNA4 | ENSG00000101204 | Cholinergic receptor nicotinic alpha 4 subunit | 20 | 63343223-63378401 |
| CHRNA7 | ENSG00000175344 | Cholinergic receptor nicotinic alpha 7 subunit | 15 | 31923438-32173018 |
| CHRNB2 | ENSG00000160716 | Cholinergic receptor nicotinic beta 2 subunit | 1 | 154567778-154580013 |
| CHRNB4 | ENSG00000117971 | Cholinergic receptor nicotinic beta 4 subunit | 15 | 78624111-78727754 |
| CKB | ENSG00000166165 | Creatine kinase B | 14 | 103519667-103522833 |
| CKM | ENSG00000104879 | Creatine kinase, M-type | 19 | 45306413-45322875 |
| CKMT1A | ENSG00000223572 | Creatine kinase, mitochondrial 1A | 15 | 43692886-43699222 |
| CKMT1B | ENSG00000237289 | Creatine kinase, mitochondrial 1B | 15 | 43593054-43604901 |
| CKMT2 | ENSG00000131730 | Creatine kinase, mitochondrial 2 | 5 | 81233320-81266398 |
| CLCN2 | ENSG00000114859 | Chloride voltage-gated channel 2 | 3 | 184346185-184361650 |
| CNR1 | ENSG00000118432 | Cannabinoid receptor 1 | 6 | 88139864-88166359 |
| CNR2 | ENSG00000188822 | Cannabinoid receptor 2 | 1 | 23870515-23913362 |
| COMT | ENSG00000093010 | Catechol-O-methyltransferase | 22 | 19941733-19969975 |
| CPS1 | ENSG00000021826 | Carbamoyl-phosphate synthase 1 | 2 | 210477682-210679107 |
| CPT1A | ENSG00000110090 | Carnitine palmitoyltransferase 1A | 11 | 68754620-68844410 |
| CPT2 | ENSG00000157184 | Carnitine palmitoyltransferase 2 | 1 | 53196792-53214197 |
| CRBN | ENSG00000113851 | Cereblon | 3 | 3144628-3179727 |
| CRHR1 | ENSG00000120088 | Corticotropin releasing hormone receptor 1 | 17 | 45784280-45835828 |
| CRTC1 | ENSG00000105662 | CREB regulated transcription coactivator 1 | 19 | 18683678-18782333 |
| CRTC2 | ENSG00000160741 | CREB regulated transcription coactivator 2 | 1 | 153947669-153958615 |
| CSF1R | ENSG00000182578 | Colony stimulating factor 1 receptor | 5 | 150053291-150113372 |
| CSF2RA | ENSG00000198223 | Colony stimulating factor 2 receptor subunit | X | 1268800-1310381 |
| | | alpha | | |
| CSF2RB | ENSG00000100368 | Colony stimulating factor 2 receptor subunit beta | 22 | 36913628-36940439 |
| CSF3R | ENSG00000119535 | Colony stimulating factor 3 receptor | 1 | 36466043-36483278 |
| CSNK2A1 | ENSG00000101266 | Casein kinase 2 alpha 1 | 20 | 472498-543835 |
| CSNK2A2 | ENSG00000070770 | Casein kinase 2 alpha 2 | 16 | 58157907-58198106 |
| CTLA4 | ENSG00000163599 | Cytotoxic T-lymphocyte associated protein 4 | 2 | 203867771-203873965 |
| CXCR1 | ENSG00000163464 | C-X-C motif chemokine receptor 1 | 2 | 218162841-218166962 |
| CXCR2 | ENSG00000180871 | C-X-C motif chemokine receptor 2 | 2 | 218125289-218137251 |
| CXCR4 | ENSG00000121966 | C-X-C motif chemokine receptor 4 | 2 | 136114349-136118149 |
| CYP11A1 | ENSG00000140459 | Cytochrome P450 family 11 subfamily A | 15 | 74337759-74367646 |
| | | member 1 | | |
| CYP11B1 | ENSG00000160882 | Cytochrome P450 family 11 subfamily B | 8 | 142872356-142879846 |
| | | member 1 | | |
| CYP11B2 | ENSG00000179142 | Cytochrome P450 family 11 subfamily B | 8 | 142910558-142917862 |
| | | member 2 | | |
| CYP17A1 | ENSG00000148795 | Cytochrome P450 family 17 subfamily A | 10 | 102830531-102837472 |
| | | member 1 | | |
| CYP19A1 | ENSG00000137869 | Cytochrome P450 family 19 subfamily A | 15 | 51208057-51338601 |
| | | member 1 | | |
| CYP51A1 | ENSG00000001630 | Cytochrome P450 family 51 subfamily A | 7 | 92112153-92134803 |
| | | member 1 | | |
| CYSLTR1 | ENSG00000173198 | Cysteinyl leukotriene receptor 1 | X | 78271468-78327691 |
| CYSLTR2 | ENSG00000152207 | Cysteinyl leukotriene receptor 2 | 13 | 48653711-48711226 |
| DBH | ENSG00000123454 | Dopamine beta-hydroxylase | 9 | 133636363-133659329 |
| DCK | ENSG00000156136 | Deoxycytidine kinase | 4 | 70992538-71030914 |
| DDC | ENSG00000132437 | Dopa decarboxylase | 7 | 50458436-50565405 |
| DDR2 | ENSG00000162733 | Discoidin domain receptor tyrosine kinase 2 | 1 | 162631373-162787405 |
| DHFR | ENSG00000228716 | Dihydrofolate reductase | 5 | 80626226-80654983 |
| DHH | ENSG00000139549 | Desert hedgehog signaling molecule | 12 | 49086656-49094801 |
| DHODH | ENSG00000102967 | Dihydroorotate dehydrogenase (quinone) | 16 | 72008588-72027664 |
| DHX9 | ENSG00000135829 | DExH-box helicase 9 | 1 | 182839347-182887982 |
| DNMT1 | ENSG00000130816 | DNA methyltransferase 1 | 19 | 10133345-10231286 |
| DOT1L | ENSG00000104885 | DOT1 like histone lysine methyltransferase | 19 | 2163933-2232578 |
| DPEP1 | ENSG00000015413 | Dipeptidase 1 | 16 | 89613308-89638456 |
| DPP4 | ENSG00000197635 | Dipeptidyl peptidase 4 | 2 | 161992245-162074215 |
| DRD1 | ENSG00000184845 | Dopamine receptor D1 | 5 | 175440036-175444182 |
| DRD2 | ENSG00000149295 | Dopamine receptor D2 | 11 | 113409605-113475691 |
| DRD3 | ENSG00000151577 | Dopamine receptor D3 | 3 | 114127580-114199407 |
| DRD4 | ENSG00000069696 | Dopamine receptor D4 | 11 | 637269-640706 |
| DRD5 | ENSG00000169676 | Dopamine receptor D5 | 4 | 9781634-9784009 |
| DYRK1A | ENSG00000157540 | Dual specificity tyrosine phosphorylation | 21 | 37365573-37526358 |
| | | regulated kinase 1A | | |
| DYRK1B | ENSG00000105204 | Dual specificity tyrosine phosphorylation | 19 | 39825350-39834201 |
| | | regulated kinase 1B | | |
| DYRK2 | ENSG00000127334 | Dual specificity tyrosine phosphorylation | 12 | 67648338-67665406 |
| | | regulated kinase 2 | | |
| DYRK3 | ENSG00000143479 | Dual specificity tyrosine phosphorylation | 1 | 206635536-206684419 |
| | | regulated kinase 3 | | |
| DYRK4 | ENSG00000010219 | Dual specificity tyrosine phosphorylation | 12 | 4562204-4615302 |
| | | regulated kinase 4 | | |
| EDNRA | ENSG00000151617 | Endothelin receptor type A | 4 | 147480917-147544954 |
| EDNRB | ENSG00000136160 | Endothelin receptor type B | 13 | 77895481-77975529 |
| EGF | ENSG00000138798 | Epidermal growth factor | 4 | 109912883-110013766 |
| EGFR | ENSG00000146648 | Epidermal growth factor receptor | 7 | 55019021-55211628 |
| EHMT1 | ENSG00000181090 | Euchromatic histone lysine methyltransferase 1 | 9 | 137618992-137870016 |
| EHMT2 | ENSG00000204371 | Euchromatic histone lysine methyltransferase 2 | 6 | 31879759-31897687 |
| EIF4E | ENSG00000151247 | Eukaryotic translation initiation factor 4E | 4 | 98871684-98930637 |
| ELANE | ENSG00000197561 | Elastase, neutrophil expressed | 19 | 851014-856247 |
| EPHA2 | ENSG00000142627 | EPH receptor A2 | 1 | 16124337-16156069 |
| EPHB4 | ENSG00000196411 | EPH receptor B4 | 7 | 100802565-100827523 |
| EPOR | ENSG00000187266 | Erythropoietin receptor | 19 | 11377207-11384342 |
| ERBB2 | ENSG00000141736 | Erb-b2 receptor tyrosine kinase 2 | 17 | 39687914-39730426 |
| ERBB3 | ENSG00000065361 | Erb-b2 receptor tyrosine kinase 3 | 12 | 56076799-56103505 |
| ERBB4 | ENSG00000178568 | Erb-b2 receptor tyrosine kinase 4 | 2 | 211375717-212538841 |
| ESRI | ENSG00000091831 | Estrogen receptor 1 | 6 | 151656691-152129619 |
| ESR2 | ENSG00000140009 | Estrogen receptor 2 | 14 | 64084232-64338112 |
| ESRRG | ENSG00000196482 | Estrogen related receptor gamma | 1 | 216503246-217137755 |
| EZH2 | ENSG00000106462 | Enhancer of zeste 2 polycomb repressive | 7 | 148807383-148884321 |
| | | complex 2 subunit | | |
| F10 | ENSG00000126218 | Coagulation factor X | 13 | 113122799-113149529 |
| F11 | ENSG00000088926 | Coagulation factor XI | 4 | 186266189-186289681 |
| F12 | ENSG00000131187 | Coagulation factor XII | 5 | 177402140-177409576 |
| F2 | ENSG00000180210 | Coagulation factor II, thrombin | 11 | 46719196-46739506 |
| F2R | ENSG00000181104 | Coagulation factor II thrombin receptor | 5 | 76716126-76735770 |
| F3 | ENSG00000117525 | Coagulation factor III, tissue factor | 1 | 94529173-94541759 |
| F5 | ENSG00000198734 | Coagulation factor V | 1 | 169511951-169586588 |
| F7 | ENSG00000057593 | Coagulation factor VII | 13 | 113105788-113120685 |
| F8 | ENSG00000185010 | Coagulation factor VIII | X | 154835788-155026940 |
| F9 | ENSG00000101981 | Coagulation factor IX | X | 139530739-139563459 |
| FADS1 | ENSG00000149485 | Fatty acid desaturase 1 | 11 | 61799627-61829318 |
| FADS2 | ENSG00000134824 | Fatty acid desaturase 2 | 11 | 61792980-61867354 |
| FASN | ENSG00000169710 | Fatty acid synthase | 17 | 82078338-82098294 |
| FCER1A | ENSG00000179639 | Fc fragment of IgE receptor Ia | 1 | 159289714-159308224 |
| FCER1G | ENSG00000158869 | Fc fragment of IgE receptor Ig | 1 | 161215234-161220699 |
| FCGR1A | ENSG00000150337 | Fc fragment of IgG receptor Ia | 1 | 149782671-149792518 |
| FCGR1B | ENSG00000198019 | Fc fragment of IgG receptor Ib | 1 | 121087345-121097161 |
| FCGR2A | ENSG00000143226 | Fc fragment of IgG receptor IIa | 1 | 161505430-161524013 |
| FCGR2B | ENSG00000072694 | Fc fragment of IgG receptor IIb | 1 | 161663147-161678654 |
| FCGR3A | ENSG00000203747 | Fc fragment of IgG receptor IIIa | 1 | 161541759-161550737 |
| FCGR3B | ENSG00000162747 | Fc fragment of IgG receptor IIIb | 1 | 161623196-161631963 |
| FDPS | ENSG00000160752 | Famesyl diphosphate synthase | 1 | 155308748-155320666 |
| FFAR1 | ENSG00000126266 | Free fatty acid receptor 1 | 19 | 35351552-35353862 |
| FGA | ENSG00000171560 | Fibrinogen alpha chain | 4 | 154583128-154590745 |
| FGF1 | ENSG00000113578 | Fibroblast growth factor 1 | 5 | 142592178-142698070 |
| FGF2 | ENSG00000138685 | Fibroblast growth factor 2 | 4 | 122826708-122898236 |
| FGFR1 | ENSG00000077782 | Fibroblast growth factor receptor 1 | 8 | 38400215-38468834 |
| FGFR2 | ENSG00000066468 | Fibroblast growth factor receptor 2 | 10 | 121478332-121598458 |
| FGFR3 | ENSG00000068078 | Fibroblast growth factor receptor 3 | 4 | 1793293-1808872 |
| FGFR4 | ENSG00000160867 | Fibroblast growth factor receptor 4 | 5 | 177086905-177098144 |
| FGR | ENSG00000000938 | FGR proto-oncogene, Src family tyrosine kinase | 1 | 27612064-27635185 |
| FKBP1A | ENSG00000088832 | FKBP prolyl isomerase 1A | 20 | 1368978-1393172 |
| FLT1 | ENSG00000102755 | Fms related receptor tyrosine kinase 1 | 13 | 28300346-28495145 |
| FLT3 | ENSG00000122025 | Fms related receptor tyrosine kinase 3 | 13 | 28003274-28100592 |
| FLT4 | ENSG00000037280 | Fms related receptor tyrosine kinase 4 | 5 | 180601506-180649624 |
| FN1 | ENSG00000115414 | Fibronectin 1 | 2 | 215360440-215436073 |
| FNTA | ENSG00000168522 | Famesyltransferase, CAAX box, alpha | 8 | 43034194-43085788 |
| FOLH1 | ENSG00000086205 | Folate hydrolase 1 | 11 | 49145092-49208638 |
| FOLR1 | ENSG00000110195 | Folate receptor alpha | 11 | 72189558-72196323 |
| FOLR2 | ENSG00000165457 | Folate receptor beta | 11 | 72216601-72221950 |
| FOLR3 | ENSG00000110203 | Folate receptor gamma | 11 | 72114869-72139892 |
| FRK | ENSG00000111816 | Fyn related Src family tyrosine kinase | 6 | 115931149-116060891 |
| FSHR | ENSG00000170820 | Follicle stimulating hormone receptor | 2 | 48962157-49154527 |
| FTH1 | ENSG00000167996 | Ferritin heavy chain 1 | 11 | 61959718-61967634 |
| FTL | ENSG00000087086 | Ferritin light chain | 19 | 48965309-48966879 |
| FXYD2 | ENSG00000137731 | FXYD domain containing ion transport regulator | 11 | 117800844-117828698 |
| | | 2 | | |
| FYN | ENSG00000010810 | FYN proto-oncogene, Src family tyrosine kinase | 6 | 111660332-111873452 |
| FZD8 | ENSG00000177283 | Frizzled class receptor 8 | 10 | 35638249-35642278 |
| GAA | ENSG00000171298 | Glucosidase alpha, acid | 17 | 80101556-80119881 |
| GABBR1 | ENSG00000204681 | Gamma-aminobutyric acid type B receptor | 6 | 29555629-29633976 |
| | | subunit 1 | | |
| GABBR2 | ENSG00000136928 | Gamma-aminobutyric acid type B receptor | 9 | 98288109-98708935 |
| | | subunit 2 | | |
| GABRA1 | ENSG00000022355 | Gamma-aminobutyric acid type A receptor | 5 | 161847063-161899981 |
| | | subunit alpha1 | | |
| GABRA2 | ENSG00000151834 | Gamma-aminobutyric acid type A receptor | 4 | 46248427-46475230 |
| | | subunit alpha2 | | |
| GABRA3 | ENSG00000011677 | Gamma-aminobutyric acid type A receptor | X | 152166234-152451315 |
| | | subunit alpha3 | | |
| GABRA4 | ENSG00000109158 | Gamma-aminobutyric acid type A receptor | 4 | 46918900-46993581 |
| | | subunit alpha4 | | |
| GABRA5 | ENSG00000186297 | Gamma-aminobutyric acid type A receptor | 15 | 26866911-26949208 |
| | | subunit alpha5 | | |
| GABRA6 | ENSG00000145863 | Gamma-aminobutyric acid type A receptor | 5 | 161547063-161702593 |
| | | subunit alpha6 | | |
| GABRB1 | ENSG00000163288 | Gamma-aminobutyric acid type A receptor | 4 | 46993723-47426447 |
| | | subunit beta1 | | |
| GABRB2 | ENSG00000145864 | Gamma-aminobutyric acid type A receptor | 5 | 161288429-161549044 |
| | | subunit beta2 | | |
| GABRB3 | ENSG00000166206 | Gamma-aminobutyric acid type A receptor | 15 | 26543546-26939539 |
| | | subunit beta3 | | |
| GABRD | ENSG00000187730 | Gamma-aminobutyric acid type A receptor | 1 | 2019329-2030758 |
| | | subunit delta | | |
| GABRE | ENSG00000102287 | Gamma-aminobutyric acid type A receptor | X | 151953124-151974680 |
| | | subunit epsilon | | |
| GABRG1 | ENSG00000163285 | Gamma-aminobutyric acid type A receptor | 4 | 46035769-46124054 |
| | | subunit gamma1 | | |
| GABRG2 | ENSG00000113327 | Gamma-aminobutyric acid type A receptor | 5 | 162000057-162162977 |
| | | subunit gamma2 | | |
| GABRG3 | ENSG00000182256 | Gamma-aminobutyric acid type A receptor | 15 | 26971181-27541984 |
| | | subunit gamma3 | | |
| GABRP | ENSG00000094755 | Gamma-aminobutyric acid type A receptor | 5 | 170763350-170814047 |
| | | subunit pi | | |
| GABRQ | ENSG00000268089 | Gamma-aminobutyric acid type A receptor | X | 152637895-152657542 |
| | | subunit theta | | |
| GABRR1 | ENSG00000146276 | Gamma-aminobutyric acid type A receptor | 6 | 89177504-89231278 |
| | | subunit rho1 | | |
| GABRR2 | ENSG00000111886 | Gamma-aminobutyric acid type A receptor | 6 | 89254464-89315299 |
| | | subunit rho2 | | |
| GABRR3 | ENSG00000183185 | Gamma-aminobutyric acid type A receptor | 3 | 97986673-98035304 |
| | | subunit rho3 (gene/pseudogene) | | |
| GAMT | ENSG00000130005 | Guanidinoacetate N-methyltransferase | 19 | 1397026-1401570 |
| GANAB | ENSG00000089597 | Glucosidase II alpha subunit | 11 | 62624826-62646726 |
| GANC | ENSG00000214013 | Glucosidase alpha, neutral C | 15 | 42273233-42356935 |
| GART | ENSG00000159131 | Phosphoribosylglycinamide formyltransferase, | 21 | 33503931-33543491 |
| | | phosphoribosylglycinamide synthetase, | | |
| | | phosphoribosylaminoimidazole synthetase | | |
| GCGR | ENSG00000215644 | Glucagon receptor | 17 | 81804132-81814008 |
| GGCX | ENSG00000115486 | Gamma-glutamyl carboxylase | 2 | 85544720-85561547 |
| GGPS1 | ENSG00000152904 | Geranylgeranyl diphosphate synthase 1 | 1 | 235327350-235344532 |
| GHR | ENSG00000112964 | Growth hormone receptor | 5 | 42423439-42721878 |
| GHRHR | ENSG00000106128 | Growth hormone releasing hormone receptor | 7 | 30938669-30993254 |
| GLP1R | ENSG00000112164 | Glucagon like peptide 1 receptor | 6 | 39048781-39091303 |
| GLP2R | ENSG00000065325 | Glucagon like peptide 2 receptor | 17 | 9822206-9892099 |
| GLRA1 | ENSG00000145888 | Glycine receptor alpha 1 | 5 | 151822513-151924851 |
| GLRA2 | ENSG00000101958 | Glycine receptor alpha 2 | X | 14529298-14731812 |
| GLRA3 | ENSG00000145451 | Glycine receptor alpha 3 | 4 | 174636920-174829247 |
| GLRB | ENSG00000109738 | Glycine receptor beta | 4 | 157076125-157172090 |
| GNRH1 | ENSG00000147437 | Gonadotropin releasing hormone 1 | 8 | 25419258-25424654 |
| GNRHR | ENSG00000109163 | Gonadotropin releasing hormone receptor | 4 | 67737118-67754388 |
| GRIA1 | ENSG00000155511 | Glutamate ionotropic receptor AMPA type | 5 | 153489615-153813869 |
| | | subunit 1 | | |
| GRIK1 | ENSG00000171189 | Glutamate ionotropic receptor kainate type | 21 | 29536933-29940033 |
| | | subunit 1 | | |
| GRIN1 | ENSG00000176884 | Glutamate ionotropic receptor NMDA type | 9 | 137139154-137168756 |
| | | subunit 1 | | |
| GRIN2A | ENSG00000183454 | Glutamate ionotropic receptor NMDA type | 16 | 9753404-10182754 |
| | | subunit 2A | | |
| GRIN2B | ENSG00000273079 | Glutamate ionotropic receptor NMDA type | 12 | 13437942-13981957 |
| | | subunit 2B | | |
| GRIN2C | ENSG00000161509 | Glutamate ionotropic receptor NMDA type | 17 | 74842023-74861504 |
| | | subunit 2C | | |
| GRIN2D | ENSG00000105464 | Glutamate ionotropic receptor NMDA type | 19 | 48394875-48444931 |
| | | subunit 2D | | |
| GRIN3A | ENSG00000198785 | Glutamate ionotropic receptor NMDA type | 9 | 101569352-101738647 |
| | | subunit 3A | | |
| GRIN3B | ENSG00000116032 | Glutamate ionotropic receptor NMDA type | 19 | 1000419-1009732 |
| | | subunit 3B | | |
| GRM5 | ENSG00000168959 | Glutamate metabotropic receptor 5 | 11 | 88504576-89065945 |
| GSK3A | ENSG00000105723 | Glycogen synthase kinase 3 alpha | 19 | 42230190-42242625 |
| GSK3B | ENSG00000082701 | Glycogen synthase kinase 3 beta | 3 | 119821321-120094447 |
| GSR | ENSG00000104687 | Glutathione-disulfide reductase | 8 | 30678066-30727846 |
| GSS | ENSG00000100983 | Glutathione synthetase | 20 | 34928432-34956027 |
| GUCY1A2 | ENSG00000152402 | Guanylate cyclase 1 soluble subunit alpha 2 | 11 | 106674019-107018476 |
| GUCY2C | ENSG00000070019 | Guanylate cyclase 2C | 12 | 14612632-14696599 |
| HBA1 | ENSG00000206172 | Hemoglobin subunit alpha 1 | 16 | 176680-177522 |
| HBA2 | ENSG00000188536 | Hemoglobin subunit alpha 2 | 16 | 172876-173710 |
| HBB | ENSG00000244734 | Hemoglobin subunit beta | 11 | 5225464-5229395 |
| HCAR2 | ENSG00000182782 | Hydroxycarboxylic acid receptor 2 | 12 | 122701293-122703357 |
| HCAR3 | ENSG00000255398 | Hydroxycarboxylic acid receptor 3 | 12 | 122714756-122716811 |
| HCK | ENSG00000101336 | HCK proto-oncogene, Src family tyrosine kinase | 20 | 32052188-32101856 |
| HCRTR1 | ENSG00000121764 | Hypocretin receptor 1 | 1 | 31617686-31632518 |
| HCRTR2 | ENSG00000137252 | Hypocretin receptor 2 | 6 | 55106460-55282620 |
| HDAC1 | ENSG00000116478 | Histone deacetylase 1 | 1 | 32292083-32333635 |
| HDAC10 | ENSG00000100429 | Histone deacetylase 10 | 22 | 50245183-50251405 |
| HDAC11 | ENSG00000163517 | Histone deacetylase 11 | 3 | 13479724-13506424 |
| HDAC2 | ENSG00000196591 | Histone deacetylase 2 | 6 | 113933028-114011308 |
| HDAC3 | ENSG00000171720 | Histone deacetylase 3 | 5 | 141620876-141636849 |
| HDAC4 | ENSG00000068024 | Histone deacetylase 4 | 2 | 239048168-239401654 |
| HDAC5 | ENSG00000108840 | Histone deacetylase 5 | 17 | 44076746-44123702 |
| HDAC6 | ENSG00000094631 | Histone deacetylase 6 | X | 48801377-48824982 |
| HDAC7 | ENSG00000061273 | Histone deacetylase 7 | 12 | 47782722-47833132 |
| HDAC8 | ENSG00000147099 | Histone deacetylase 8 | X | 72329516-72573101 |
| HDAC9 | ENSG00000048052 | Histone deacetylase 9 | 7 | 18086949-19002416 |
| HMGCR | ENSG00000113161 | 3-hydroxy-3-methylglutaryl-CoA reductase | 5 | 75336329-75362101 |
| HMMR | ENSG00000072571 | Hyaluronan mediated motility receptor | 5 | 163460203-163491941 |
| HPD | ENSG00000158104 | 4-hydroxyphenylpyruvate dioxygenase | 12 | 121839527-121863596 |
| HPRT1 | ENSG00000165704 | Hypoxanthine phosphoribosyltransferase 1 | X | 134460165-134520513 |
| HPSE | ENSG00000173083 | Heparanase | 4 | 83292461-83335153 |
| HRH1 | ENSG00000196639 | Histamine receptor H1 | 3 | 11137093-11263557 |
| HRH2 | ENSG00000113749 | Histamine receptor H2 | 5 | 175658030-175710756 |
| HRH3 | ENSG00000101180 | Histamine receptor H3 | 20 | 62214960-62220278 |
| HSD3B1 | ENSG00000203857 | Hydroxy-delta-5-steroid dehydrogenase, 3 beta- | 1 | 119507198-119515054 |
| | | and steroid delta-isomerase 1 | | |
| HSD3B2 | ENSG00000203859 | Hydroxy-delta-5-steroid dehydrogenase, 3 beta- | 1 | 119414931-119423035 |
| | | and steroid delta-isomerase 2 | | |
| HSP90AA1 | ENSG00000080824 | Heat shock protein 90 alpha family class A | 14 | 102080742-102139699 |
| | | member 1 | | |
| HSPA1A | ENSG00000204389 | Heat shock protein family A (Hsp70) member 1A | 6 | 31815543-31817946 |
| HSPA1B | ENSG00000204388 | Heat shock protein family A (Hsp70) member 1B | 6 | 31827738-31830254 |
| HSPB1 | ENSG00000106211 | Heat shock protein family B (small) member 1 | 7 | 76302673-76304295 |
| HTR1A | ENSG00000178394 | 5-hydroxytryptamine receptor 1A | 5 | 63957874-63962507 |
| HTR1B | ENSG00000135312 | 5-hydroxytryptamine receptor 1B | 6 | 77460924-77463491 |
| HTR1D | ENSG00000179546 | 5-hydroxytryptamine receptor 1D | 1 | 23191895-23194729 |
| HTR1E | ENSG00000168830 | 5-hydroxytryptamine receptor 1E | 6 | 86937528-87016679 |
| HTR1F | ENSG00000179097 | 5-hydroxytryptamine receptor 1F | 3 | 87990696-87993835 |
| HTR2A | ENSG00000102468 | 5-hydroxytryptamine receptor 2A | 13 | 46831550-46897076 |
| HTR2B | ENSG00000135914 | 5-hydroxytryptamine receptor 2B | 2 | 231108230-231125042 |
| HTR2C | ENSG00000147246 | 5-hydroxytryptamine receptor 2C | X | 114584078-114910061 |
| HTR3A | ENSG00000166736 | 5-hydroxytryptamine receptor 3A | 11 | 113974881-113990313 |
| HTR3B | ENSG00000149305 | 5-hydroxytryptamine receptor 3B | 11 | 113904677-113949078 |
| HTR3C | ENSG00000178084 | 5-hydroxytryptamine receptor 3C | 3 | 184053047-184060673 |
| HTR3D | ENSG00000186090 | 5-hydroxytryptamine receptor 3D | 3 | 184031544-184039369 |
| HTR3E | ENSG00000186038 | 5-hydroxytryptamine receptor 3E | 3 | 184097064-184106995 |
| HTR4 | ENSG00000164270 | 5-hydroxytryptamine receptor 4 | 5 | 148451032-148677235 |
| HTR6 | ENSG00000158748 | 5-hydroxytryptamine receptor 6 | 1 | 19664875-19680966 |
| HTR7 | ENSG00000148680 | 5-hydroxytryptamine receptor 7 | 10 | 90740823-90858039 |
| ICAM1 | ENSG00000090339 | Intercellular adhesion molecule 1 | 19 | 10271093-10286615 |
| IDE | ENSG00000119912 | Insulin degrading enzyme | 10 | 92451684-92574093 |
| IDH1 | ENSG00000138413 | Isocitrate dehydrogenase (NADP(+)) 1 | 2 | 208236227-208266074 |
| IDH2 | ENSG00000182054 | Isocitrate dehydrogenase (NADP(+)) 2 | 15 | 90083045-90102477 |
| IDO1 | ENSG00000131203 | Indoleamine 2,3-dioxygenase 1 | 8 | 39902275-39928790 |
| IFNAR1 | ENSG00000142166 | Interferon alpha and beta receptor subunit 1 | 21 | 33324429-33359864 |
| IFNAR2 | ENSG00000159110 | Interferon alpha and beta receptor subunit 2 | 21 | 33229901-33265675 |
| IFNG | ENSG00000111537 | Interferon gamma | 12 | 68154768-68159740 |
| IFNGR1 | ENSG00000027697 | Interferon gamma receptor 1 | 6 | 137197485-137219449 |
| IFNGR2 | ENSG00000159128 | Interferon gamma receptor 2 | 21 | 33402896-33479348 |
| IGF1R | ENSG00000140443 | Insulin like growth factor 1 receptor | 15 | 98648539-98964530 |
| IHH | ENSG00000163501 | Indian hedgehog signaling molecule | 2 | 219054424-219060921 |
| IKBKB | ENSG00000104365 | Inhibitor of nuclear factor kappa B kinase subunit | 8 | 42271302-42332653 |
| | | beta | | |
| IL11RA | ENSG00000137070 | Interleukin 11 receptor subunit alpha | 9 | 34650702-34661902 |
| IL12B | ENSG00000113302 | Interleukin 12B | 5 | 159314780-159330487 |
| IL17A | ENSG00000112115 | Interleukin 17A | 6 | 52186375-52190638 |
| IL1B | ENSG00000125538 | Interleukin 1 beta | 2 | 112829751-112836816 |
| IL1R1 | ENSG00000115594 | Interleukin 1 receptor type 1 | 2 | 102064544-102179874 |
| IL23A | ENSG00000110944 | Interleukin 23 subunit alpha | 12 | 56334174-56340410 |
| IL2RA | ENSG00000134460 | Interleukin 2 receptor subunit alpha | 10 | 6010689-6062370 |
| IL2RB | ENSG00000100385 | Interleukin 2 receptor subunit beta | 22 | 37125843-37175054 |
| IL2RG | ENSG00000147168 | Interleukin 2 receptor subunit gamma | X | 71107404-71112108 |
| IL3RA | ENSG00000185291 | Interleukin 3 receptor subunit alpha | X | 1336616-1382689 |
| IL5 | ENSG00000113525 | Interleukin 5 | 5 | 132541445-132556838 |
| IL6 | ENSG00000136244 | Interleukin 6 | 7 | 22725884-22732002 |
| IL6R | ENSG00000160712 | Interleukin 6 receptor | 1 | 154405193-154469450 |
| IL6ST | ENSG00000134352 | Interleukin 6 signal transducer | 5 | 55935095-55995022 |
| IMPDH1 | ENSG00000106348 | Inosine monophosphate dehydrogenase 1 | 7 | 128392277-128410252 |
| IMPDH2 | ENSG00000178035 | Inosine monophosphate dehydrogenase 2 | 3 | 49024325-49029408 |
| INHA | ENSG00000123999 | Inhibin subunit alpha | 2 | 219569162-219575711 |
| INHBA | ENSG00000122641 | Inhibin subunit beta A | 7 | 41667168-41705834 |
| INHBB | ENSG00000163083 | Inhibin subunit beta B | 2 | 120346136-120351803 |
| INHBC | ENSG00000175189 | Inhibin subunit beta C | 12 | 57434784-57452062 |
| INHBE | ENSG00000139269 | Inhibin subunit beta E | 12 | 57452323-57459280 |
| INSR | ENSG00000171105 | Insulin receptor | 19 | 7112255-7294414 |
| ITGA2B | ENSG00000005961 | Integrin subunit alpha 2b | 17 | 44372180-44389649 |
| ITGA4 | ENSG00000115232 | Integrin subunit alpha 4 | 2 | 181457202-181538940 |
| ITGAL | ENSG00000005844 | Integrin subunit alpha L | 16 | 30472719-30523185 |
| ITGAV | ENSG00000138448 | Integrin subunit alpha V | 2 | 186590056-186680901 |
| ITGB1 | ENSG00000150093 | Integrin subunit beta 1 | 10 | 32900318-33005792 |
| ITGB3 | ENSG00000259207 | Integrin subunit beta 3 | 17 | 47253827-47313743 |
| ITGB7 | ENSG00000139626 | Integrin subunit beta 7 | 12 | 53191323-53207282 |
| ITK | ENSG00000113263 | IL2 inducible T cell kinase | 5 | 157142933-157255185 |
| JAK1 | ENSG00000162434 | Janus kinase 1 | 1 | 64833229-65067754 |
| JAK2 | ENSG00000096968 | Janus kinase 2 | 9 | 4984390-5129948 |
| JAK3 | ENSG00000105639 | Janus kinase 3 | 19 | 17824780-17848071 |
| JUN | ENSG00000177606 | Jun proto-oncogene, AP-1 transcription factor | 1 | 58780791-58784047 |
| | | subunit | | |
| KCNA1 | ENSG00000111262 | Potassium voltage-gated channel subfamily A | 12 | 4909905-4918256 |
| | | member 1 | | |
| KCNA10 | ENSG00000143105 | Potassium voltage-gated channel subfamily A | 1 | 110517217-110519175 |
| | | member 10 | | |
| KCNA2 | ENSG00000177301 | Potassium voltage-gated channel subfamily A | 1 | 110519837-110631474 |
| | | member 2 | | |
| KCNA3 | ENSG00000177272 | Potassium voltage-gated channel subfamily A | 1 | 110672465-110674940 |
| | | member 3 | | |
| KCNA4 | ENSG00000182255 | Potassium voltage-gated channel subfamily A | 11 | 30009730-30017030 |
| | | member 4 | | |
| KCNA5 | ENSG00000130037 | Potassium voltage-gated channel subfamily A | 12 | 5043879-5046788 |
| | | member 5 | | |
| KCNA6 | ENSG00000151079 | Potassium voltage-gated channel subfamily A | 12 | 4809176-4813412 |
| | | member 6 | | |
| KCNA7 | ENSG00000104848 | Potassium voltage-gated channel subfamily A | 19 | 49067418-49072941 |
| | | member 7 | | |
| KCNB1 | ENSG00000158445 | Potassium voltage-gated channel subfamily B | 20 | 49293394-49484297 |
| | | member 1 | | |
| KCNB2 | ENSG00000182674 | Potassium voltage-gated channel subfamily B | 8 | 72537225-72938349 |
| | | member 2 | | |
| KCNC1 | ENSG00000129159 | Potassium voltage-gated channel subfamily C | 11 | 17734774-17856804 |
| | | member 1 | | |
| KCNC2 | ENSG00000166006 | Potassium voltage-gated channel subfamily C | 12 | 75040077-75209839 |
| | | member 2 | | |
| KCNC3 | ENSG00000131398 | Potassium voltage-gated channel subfamily C | 19 | 50311937-50333515 |
| | | member 3 | | |
| KCND1 | ENSG00000102057 | Potassium voltage-gated channel subfamily D | X | 48961378-48971844 |
| | | member 1 | | |
| KCND2 | ENSG00000184408 | Potassium voltage-gated channel subfamily D | 7 | 120273175-120750337 |
| | | member 2 | | |
| KCND3 | ENSG00000171385 | Potassium voltage-gated channel subfamily D | 1 | 111770662-111989155 |
| | | member 3 | | |
| KCNE1 | ENSG00000180509 | Potassium voltage-gated channel subfamily E | 21 | 34446688-34512210 |
| | | regulatory subunit 1 | | |
| KCNH2 | ENSG00000055118 | Potassium voltage-gated channel subfamily H | 7 | 150944961-150978321 |
| | | member 2 | | |
| KCNJ1 | ENSG00000151704 | Potassium inwardly rectifying channel subfamily | 11 | 128836315-128867373 |
| | | J member 1 | | |
| KCNJ11 | ENSG00000187486 | Potassium inwardly rectifying channel subfamily | 11 | 17385859-17389331 |
| | | J member 11 | | |
| KCNJ12 | ENSG00000184185 | Potassium inwardly rectifying channel subfamily | 17 | 21376357-21419870 |
| | | J member 12 | | |
| KCNJ8 | ENSG00000121361 | Potassium inwardly rectifying channel subfamily | 12 | 21764955-21775600 |
| | | J member 8 | | |
| KCNK2 | ENSG00000082482 | Potassium two pore domain channel subfamily K | 1 | 215005775-215237093 |
| | | member 2 | | |
| KCNK3 | ENSG00000171303 | Potassium two pore domain channel subfamily K | 2 | 26692690-26733420 |
| | | member 3 | | |
| KCNK9 | ENSG00000169427 | Potassium two pore domain channel subfamily K | 8 | 139600838-139704109 |
| | | member 9 | | |
| KCNMA1 | ENSG00000156113 | Potassium calcium-activated channel subfamily | 10 | 76869601-77638369 |
| | | M alpha 1 | | |
| KCNN4 | ENSG00000104783 | Potassium calcium-activated channel subfamily | 19 | 43766533-43781257 |
| | | N member 4 | | |
| KCNQ1 | ENSG00000053918 | Potassium voltage-gated channel subfamily Q | 11 | 2444684-2849105 |
| | | member 1 | | |
| KCNQ2 | ENSG00000075043 | Potassium voltage-gated channel subfamily Q | 20 | 63400210-63472677 |
| | | member 2 | | |
| KCNQ3 | ENSG00000184156 | Potassium voltage-gated channel subfamily Q | 8 | 132120859-132481095 |
| | | member 3 | | |
| KDM1A | ENSG00000004487 | Lysine demethylase 1A | 1 | 23019443-23083689 |
| KDR | ENSG00000128052 | Kinase insert domain receptor | 4 | 55078481-55125595 |
| KEAP1 | ENSG00000079999 | Kelch like ECH associated protein 1 | 19 | 10486125-10503558 |
| KIF11 | ENSG00000138160 | Kinesin family member 11 | 10 | 92593130-92655395 |
| KIT | ENSG00000157404 | KIT proto-oncogene, receptor tyrosine kinase | 4 | 54657918-54740715 |
| KLKB1 | ENSG00000164344 | Kallikrein B1 | 4 | 186208979-186258471 |
| KRAS | ENSG00000133703 | KRAS proto-oncogene, GTPase | 12 | 25205246-25250936 |
| LAP3 | ENSG00000002549 | Leucine aminopeptidase 3 | 4 | 17577192-17607972 |
| LCK | ENSG00000182866 | LCK proto-oncogene, Src family tyrosine kinase | 1 | 32251239-32286165 |
| LDLR | ENSG00000130164 | Low density lipoprotein receptor | 19 | 11089462-11133820 |
| LEPR | ENSG00000116678 | Leptin receptor | 1 | 65420652-65641559 |
| LHCGR | ENSG00000138039 | Luteinizing hormone/choriogonadotropin | 2 | 48686774-48755730 |
| | | receptor | | |
| LIMK1 | ENSG00000106683 | LIM domain kinase 1 | 7 | 74082933-74122525 |
| LIPF | ENSG00000182333 | Lipase F, gastric type | 10 | 88664441-88678814 |
| LPL | ENSG00000175445 | Lipoprotein lipase | 8 | 19901717-19967259 |
| LYN | ENSG00000254087 | LYN proto-oncogene, Src family tyrosine kinase | 8 | 55879835-56014169 |
| M6PR | ENSG00000003056 | Mannose-6-phosphate receptor, cation dependent | 12 | 8940361-8949761 |
| MAOA | ENSG00000189221 | Monoamine oxidase A | X | 43654907-43746824 |
| MAOB | ENSG00000069535 | Monoamine oxidase B | X | 43766610-43882450 |
| MAP1A | ENSG00000166963 | Microtubule associated protein 1A | 15 | 43510958-43531620 |
| MAP2 | ENSG00000078018 | Microtubule associated protein 2 | 2 | 209424058-209734118 |
| MAP2K1 | ENSG00000169032 | Mitogen-activated protein kinase kinase 1 | 15 | 66386837-66491544 |
| MAP2K2 | ENSG00000126934 | Mitogen-activated protein kinase kinase 2 | 19 | 4090321-4124122 |
| MAP3K7 | ENSG00000135341 | Mitogen-activated protein kinase kinase kinase 7 | 6 | 90513573-90587072 |
| MAP3K8 | ENSG00000107968 | Mitogen-activated protein kinase kinase kinase 8 | 10 | 30434021-30461833 |
| MAP4 | ENSG00000047849 | Microtubule associated protein 4 | 3 | 47850690-48089272 |
| MAPK1 | ENSG00000100030 | Mitogen-activated protein kinase 1 | 22 | 21759657-21867680 |
| MAPK11 | ENSG00000185386 | Mitogen-activated protein kinase 11 | 22 | 50263713-50270767 |
| MAPK14 | ENSG00000112062 | Mitogen-activated protein kinase 14 | 6 | 36027677-36111236 |
| MAPK3 | ENSG00000102882 | Mitogen-activated protein kinase 3 | 16 | 30114105-30123506 |
| MAPK8 | ENSG00000107643 | Mitogen-activated protein kinase 8 | 10 | 48306639-48439360 |
| MAPK9 | ENSG00000050748 | Mitogen-activated protein kinase 9 | 5 | 180233143-180292099 |
| MAPT | ENSG00000186868 | Microtubule associated protein tau | 17 | 45894382-46028334 |
| MC2R | ENSG00000185231 | Melanocortin 2 receptor | 18 | 13882044-13915707 |
| MCL1 | ENSG00000143384 | MCL1 apoptosis regulator, BCL2 family member | 1 | 150574551-150579738 |
| MDM2 | ENSG00000135679 | MDM2 proto-oncogene | 12 | 68808177-68850686 |
| MET | ENSG00000105976 | MET proto-oncogene, receptor tyrosine kinase | 7 | 116672196-116798386 |
| METAP2 | ENSG00000111142 | Methionyl aminopeptidase 2 | 12 | 95473520-95515839 |
| MGAM | ENSG00000257335 | Maltase-glucoamylase | 7 | 141907813-142106747 |
| MGMT | ENSG00000170430 | O-6-methylguanine-DNA methyltransferase | 10 | 129467190-129770983 |
| MME | ENSG00000196549 | Membrane metalloendopeptidase | 3 | 155024124-155183729 |
| MMP1 | ENSG00000196611 | Matrix metallopeptidase 1 | 11 | 102789919-102798160 |
| MMP10 | ENSG00000166670 | Matrix metallopeptidase 10 | 11 | 102770502-102780628 |
| MMP11 | ENSG00000099953 | Matrix metallopeptidase 11 | 22 | 23768226-23784316 |
| MMP12 | ENSG00000262406 | Matrix metallopeptidase 12 | 11 | 102862736-102874982 |
| MMP13 | ENSG00000137745 | Matrix metallopeptidase 13 | 11 | 102942995-102955732 |
| MMP14 | ENSG00000157227 | Matrix metallopeptidase 14 | 14 | 22836560-22849027 |
| MMP15 | ENSG00000102996 | Matrix metallopeptidase 15 | 16 | 58025754-58046901 |
| MMP16 | ENSG00000156103 | Matrix metallopeptidase 16 | 8 | 88032011-88328025 |
| MMP17 | ENSG00000198598 | Matrix metallopeptidase 17 | 12 | 131828393-131851783 |
| MMP19 | ENSG00000123342 | Matrix metallopeptidase 19 | 12 | 55835433-55842966 |
| MMP2 | ENSG00000087245 | Matrix metallopeptidase 2 | 16 | 55389700-55506691 |
| MMP20 | ENSG00000137674 | Matrix metallopeptidase 20 | 11 | 102576832-102625332 |
| MMP21 | ENSG00000154485 | Matrix metallopeptidase 21 | 10 | 125753580-125775821 |
| MMP23B | ENSG00000189409 | Matrix metallopeptidase 23B | 1 | 1632163-1635263 |
| MMP24 | ENSG00000125966 | Matrix metallopeptidase 24 | 20 | 35226690-35276998 |
| MMP25 | ENSG00000008516 | Matrix metallopeptidase 25 | 16 | 3046561-3060726 |
| MMP26 | ENSG00000167346 | Matrix metallopeptidase 26 | 11 | 4704927-4992429 |
| MMP27 | ENSG00000137675 | Matrix metallopeptidase 27 | 11 | 102691487-102705769 |
| MMP28 | ENSG00000271447 | Matrix metallopeptidase 28 | 17 | 35756249-35795707 |
| MMP3 | ENSG00000149968 | Matrix metallopeptidase 3 | 11 | 102835801-102843609 |
| MMP7 | ENSG00000137673 | Matrix metallopeptidase 7 | 11 | 102520508-102530750 |
| MMP8 | ENSG00000118113 | Matrix metallopeptidase 8 | 11 | 102711796-102727050 |
| MMP9 | ENSG00000100985 | Matrix metallopeptidase 9 | 20 | 46008908-46016561 |
| MPL | ENSG00000117400 | MPL proto-oncogene, thrombopoietin receptor | 1 | 43337818-43354466 |
| MR1 | ENSG00000153029 | Major histocompatibility complex, class I-related | 1 | 181033425-181061938 |
| MS4A1 | ENSG00000156738 | Membrane spanning 4-domains A1 | 11 | 60455846-60470752 |
| MS4A2 | ENSG00000149534 | Membrane spanning 4-domains A2 | 11 | 60088261-60098467 |
| MSLN | ENSG00000102854 | Mesothelin | 16 | 760734-768865 |
| MST1R | ENSG00000164078 | Macrophage stimulating 1 receptor | 3 | 49887002-49903873 |
| MTNR1A | ENSG00000168412 | Melatonin receptor 1A | 4 | 186533655-186555567 |
| MTNR1B | ENSG00000134640 | Melatonin receptor 1B | 11 | 92969720-92985066 |
| MTOR | ENSG00000198793 | Mechanistic target of rapamycin kinase | 1 | 11106535-11262551 |
| MTR | ENSG00000116984 | 5-methyltetrahydrofolate-homocysteine | 1 | 236795292-236921278 |
| | | methyltransferase | | |
| MTTP | ENSG00000138823 | Microsomal triglyceride transfer protein | 4 | 99563761-99623999 |
| MUC5AC | ENSG00000215182 | Mucin 5AC, oligomeric mucus/gel-forming | 11 | 1157953-1201138 |
| MUTT | ENSG00000146085 | Methylmalonyl-CoA mutase | 6 | 49430360-49463191 |
| NAE1 | ENSG00000159593 | NEDD8 activating enzyme E1 subunit 1 | 16 | 66802875-66873256 |
| NAMPT | ENSG00000105835 | Nicotinamide phosphoribosyltransferase | 7 | 106248298-106286326 |
| NBN | ENSG00000104320 | Nibrin | 8 | 89933331-90003228 |
| NCSTN | ENSG00000162736 | Nicastrin | 1 | 160343316-160358952 |
| NEK11 | ENSG00000114670 | NIMA related kinase 11 | 3 | 131026850-131350465 |
| NFKB1 | ENSG00000109320 | Nuclear factor kappa B subunit 1 | 4 | 102501331-102617302 |
| NISCH | ENSG00000010322 | Nischarin | 3 | 52455118-52493068 |
| NNMT | ENSG00000166741 | Nicotinamide N-methyltransferase | 11 | 114257787-114313285 |
| NOS2 | ENSG00000007171 | Nitric oxide synthase 2 | 17 | 27756766-27800529 |
| NOS3 | ENSG00000164867 | Nitric oxide synthase 3 | 7 | 150991017-151014588 |
| NOTCH1 | ENSG00000148400 | Notch receptor 1 | 9 | 136494433-136546048 |
| NOTCH2 | ENSG00000134250 | Notch receptor 2 | 1 | 119911553-120100779 |
| NOTCH3 | ENSG00000074181 | Notch receptor 3 | 19 | 15159038-15200995 |
| NOTCH4 | ENSG00000204301 | Notch receptor 4 | 6 | 32194843-32224067 |
| NOXO1 | ENSG00000196408 | NADPH oxidase organizer 1 | 16 | 1978917-1984192 |
| NPC1L1 | ENSG00000015520 | NPC1 like intracellular cholesterol transporter 1 | 7 | 44512535-44541315 |
| NPEPPS | ENSG00000141279 | Aminopeptidase puromycin sensitive | 17 | 47522942-47623276 |
| NPR1 | ENSG00000169418 | Natriuretic peptide receptor 1 | 1 | 153678688-153693992 |
| NPR2 | ENSG00000159899 | Natriuretic peptide receptor 2 | 9 | 35792154-35809732 |
| NR1H4 | ENSG00000012504 | Nuclear receptor subfamily 1 group H member 4 | 12 | 100473708-100564414 |
| NR1I2 | ENSG00000144852 | Nuclear receptor subfamily 1 group I member 2 | 3 | 119780484-119818485 |
| NR3C1 | ENSG00000113580 | Nuclear receptor subfamily 3 group C member 1 | 5 | 143277931-143435512 |
| NR3C2 | ENSG00000151623 | Nuclear receptor subfamily 3 group C member 2 | 4 | 148078762-148444698 |
| NRAS | ENSG00000213281 | NRAS proto-oncogene, GTPase | 1 | 114704469-114716771 |
| NTRK1 | ENSG00000198400 | Neurotrophic receptor tyrosine kinase 1 | 1 | 156815640-156881850 |
| NTRK2 | ENSG00000148053 | Neurotrophic receptor tyrosine kinase 2 | 9 | 84668551-85027070 |
| NTRK3 | ENSG00000140538 | Neurotrophic receptor tyrosine kinase 3 | 15 | 87859751-88256768 |
| NTSR2 | ENSG00000169006 | Neurotensin receptor 2 | 2 | 11658178-11670195 |
| ODC1 | ENSG00000115758 | Ornithine decarboxylase 1 | 2 | 10439968-10448327 |
| OPRD1 | ENSG00000116329 | Opioid receptor delta 1 | 1 | 28812142-28871267 |
| OPRK1 | ENSG00000082556 | Opioid receptor kappa 1 | 8 | 53225716-53251697 |
| OPRM1 | ENSG00000112038 | Opioid receptor mu 1 | 6 | 154010496-154246867 |
| OXTR | ENSG00000180914 | Oxytocin receptor | 3 | 8750408-8769628 |
| P2RX4 | ENSG00000135124 | Purinergic receptor P2X 4 | 12 | 121209857-121234106 |
| P2RY12 | ENSG00000169313 | Purinergic receptor P2Y12 | 3 | 151336843-151384753 |
| P2RY2 | ENSG00000175591 | Purinergic receptor P2Y2 | 11 | 73218298-73236352 |
| PAH | ENSG00000171759 | Phenylalanine hydroxylase | 12 | 102836889-102958410 |
| PARP1 | ENSG00000143799 | Poly(ADP-ribose) polymerase 1 | 1 | 226360691-226408093 |
| PARP2 | ENSG00000129484 | Poly(ADP-ribose) polymerase 2 | 14 | 20343582-20357905 |
| PARP3 | ENSG00000041880 | Poly(ADP-ribose) polymerase family member 3 | 3 | 51942345-51948867 |
| PCSK9 | ENSG00000169174 | Proprotein convertase subtilisin/kexin type 9 | 1 | 55039447-55064852 |
| PDCD1 | ENSG00000188389 | Programmed cell death 1 | 2 | 241849884-241858894 |
| PDE10A | ENSG00000112541 | Phosphodiesterase 10A | 6 | 165327287-165988078 |
| PDE3A | ENSG00000172572 | Phosphodiesterase 3A | 12 | 20369245-20684381 |
| PDE4A | ENSG00000065989 | Phosphodiesterase 4A | 19 | 10416773-10469630 |
| PDE4B | ENSG00000184588 | Phosphodiesterase 4B | 1 | 65792514-66374579 |
| PDE4C | ENSG00000105650 | Phosphodiesterase 4C | 19 | 18208652-18248200 |
| PDE4D | ENSG00000113448 | Phosphodiesterase 4D | 5 | 58969038-60522120 |
| PDE5A | ENSG00000138735 | Phosphodiesterase 5A | 4 | 119494397-119628804 |
| PDE7A | ENSG00000205268 | Phosphodiesterase 7A | 8 | 65714334-65842322 |
| PDE7B | ENSG00000171408 | Phosphodiesterase 7B | 6 | 135851701-136195574 |
| PDGFRA | ENSG00000134853 | Platelet derived growth factor receptor alpha | 4 | 54229280-54298245 |
| PDGFRB | ENSG00000113721 | Platelet derived growth factor receptor beta | 5 | 150113839-150155872 |
| PDXK | ENSG00000160209 | Pyridoxal kinase | 21 | 43719094-43762307 |
| PGF | ENSG00000119630 | Placental growth factor | 14 | 74941834-74955626 |
| PGR | ENSG00000082175 | Progesterone receptor | 11 | 101029624-101129813 |
| PIGF | ENSG00000151665 | Phosphatidylinositol glycan anchor biosynthesis | 2 | 46580937-46617055 |
| | | class F | | |
| PIK3CA | ENSG00000121879 | Phosphatidylinositol-4,5-bisphosphate 3-kinase | 3 | 179148114-179240093 |
| | | catalytic subunit alpha | | |
| PIK3CB | ENSG00000051382 | Phosphatidylinositol-4,5-bisphosphate 3-kinase | 3 | 138652698-138834928 |
| | | catalytic subunit beta | | |
| PIK3CD | ENSG00000171608 | Phosphatidylinositol-4,5-bisphosphate 3-kinase | 1 | 9651731-9729114 |
| | | catalytic subunit delta | | |
| PIK3CG | ENSG00000105851 | Phosphatidylinositol-4,5-bisphosphate 3-kinase | 7 | 106865278-106908980 |
| | | catalytic subunit gamma | | |
| PIM1 | ENSG00000137193 | Pim-1 proto-oncogene, serine/threonine kinase | 6 | 37170152-37175428 |
| PIM2 | ENSG00000102096 | Pim-2 proto-oncogene, serine/threonine kinase | X | 48913182-48919024 |
| PIM3 | ENSG00000198355 | Pim-3 proto-oncogene, serine/threonine kinase | 22 | 49960768-49964072 |
| PLA2G1B | ENSG00000170890 | Phospholipase A2 group IB | 12 | 120322115-120327779 |
| PLA2G2A | ENSG00000188257 | Phospholipase A2 group IIA | 1 | 19975431-19980416 |
| PLA2G4A | ENSG00000116711 | Phospholipase A2 group IVA | 1 | 186828949-186988981 |
| PLA2G6 | ENSG00000184381 | Phospholipase A2 group VI | 22 | 38111495-38214778 |
| PLAT | ENSG00000104368 | Plasminogen activator, tissue type | 8 | 42174718-42207676 |
| PLAU | ENSG00000122861 | Plasminogen activator, urokinase | 10 | 73909177-73917496 |
| PLAUR | ENSG00000011422 | Plasminogen activator, urokinase receptor | 19 | 43646095-43670547 |
| PLCL1 | ENSG00000115896 | Phospholipase C like 1 (inactive) | 2 | 197804593-198572581 |
| PLG | ENSG00000122194 | Plasminogen | 6 | 160702238-160753315 |
| PLIN3 | ENSG00000105355 | Perilipin 3 | 19 | 4838341-4867694 |
| PLK1 | ENSG00000166851 | Polo like kinase 1 | 16 | 23677656-23690367 |
| PLK2 | ENSG00000145632 | Polo like kinase 2 | 5 | 58453982-58460139 |
| PLK3 | ENSG00000173846 | Polo like kinase 3 | 1 | 44800377-44805990 |
| PNLIP | ENSG00000175535 | Pancreatic lipase | 10 | 116545931-116567855 |
| PNP | ENSG00000198805 | Purine nucleoside phosphorylase | 14 | 20468954-20477089 |
| POLA1 | ENSG00000101868 | DNA polymerase alpha 1, catalytic subunit | X | 24693909-24996986 |
| POLB | ENSG00000070501 | DNA polymerase beta | 8 | 42338454-42371808 |
| POLE | ENSG00000177084 | DNA polymerase epsilon, catalytic subunit | 12 | 132623753-132687376 |
| POLE2 | ENSG00000100479 | DNA polymerase epsilon 2, accessory subunit | 14 | 49643555-49688422 |
| POLE3 | ENSG00000148229 | DNA polymerase epsilon 3, accessory subunit | 9 | 113407235-113410675 |
| POLE4 | ENSG00000115350 | DNA polymerase epsilon 4, accessory subunit | 2 | 74958643-74970128 |
| PORCN | ENSG00000102312 | Porcupine O-acyltransferase | X | 48508962-48520814 |
| PPARA | ENSG00000186951 | Peroxisome proliferator activated receptor alpha | 22 | 46150521-46243756 |
| PPARD | ENSG00000112033 | Peroxisome proliferator activated receptor delta | 6 | 35342558-35428191 |
| PPARG | ENSG00000132170 | Peroxisome proliferator activated receptor | 3 | 12287368-12434356 |
| | | gamma | | |
| PPP2CA | ENSG00000113575 | Protein phosphatase 2 catalytic subunit alpha | 5 | 134194332-134226073 |
| PPP3R2 | ENSG00000188386 | Protein phosphatase 3 regulatory subunit B, beta | 9 | 101591604-101595021 |
| PRDX5 | ENSG00000126432 | Peroxiredoxin 5 | 11 | 64318121-64321811 |
| PRKAA1 | ENSG00000132356 | Protein kinase AMP-activated catalytic subunit | 5 | 40759379-40798374 |
| | | alpha 1 | | |
| PRKAB1 | ENSG00000111725 | Protein kinase AMP-activated non-catalytic | 12 | 119667864-119681624 |
| | | subunit beta 1 | | |
| PRKCA | ENSG00000154229 | Protein kinase C alpha | 17 | 66302613-66810743 |
| PRKCB | ENSG00000166501 | Protein kinase C beta | 16 | 23835983-24220611 |
| PRKCE | ENSG00000171132 | Protein kinase C epsilon | 2 | 45651345-46187990 |
| PRKCG | ENSG00000126583 | Protein kinase C gamma | 19 | 53879190-53907652 |
| PRKCI | ENSG00000163558 | Protein kinase C iota | 3 | 170222424-170305977 |
| PRKDC | ENSG00000253729 | Protein kinase, DNA-activated, catalytic subunit | 8 | 47773111-47960178 |
| PRLR | ENSG00000113494 | Prolactin receptor | 5 | 35048756-35230487 |
| PRMT5 | ENSG00000100462 | Protein arginine methyltransferase 5 | 14 | 22920525-22929408 |
| PROC | ENSG00000115718 | Protein C, inactivator of coagulation factors Va | 2 | 127418427-127429242 |
| | | and Villa | | |
| PROS1 | ENSG00000184500 | Protein S | 3 | 93873051-93980003 |
| PSEN1 | ENSG00000080815 | Presenilin 1 | 14 | 73136418-73223691 |
| PSENEN | ENSG00000205155 | Presenilin enhancer, gamma-secretase subunit | 19 | 35745600-35747519 |
| PSMB1 | ENSG00000008018 | Proteasome 20S subunit beta 1 | 6 | 170535120-170553307 |
| PSMB10 | ENSG00000205220 | Proteasome 20S subunit beta 10 | 16 | 67934506-67936864 |
| PSMB2 | ENSG00000126067 | Proteasome 20S subunit beta 2 | 1 | 35599541-35641526 |
| PSMB5 | ENSG00000100804 | Proteasome 20S subunit beta 5 | 14 | 23016543-23035230 |
| PSMB8 | ENSG00000204264 | Proteasome 20S subunit beta 8 | 6 | 32840717-32844679 |
| PSMB9 | ENSG00000240065 | Proteasome 20S subunit beta 9 | 6 | 32844136-32859851 |
| PSMD1 | ENSG00000173692 | Proteasome 26S subunit, non-ATPase 1 | 2 | 231056864-231172827 |
| PSMD2 | ENSG00000175166 | Proteasome 26S subunit, non-ATPase 2 | 3 | 184299198-184309050 |
| PTCH1 | ENSG00000185920 | Patched 1 | 9 | 95442980-95517057 |
| PTGER1 | ENSG00000160951 | Prostaglandin E receptor 1 | 19 | 14472466-14475354 |
| PTGER2 | ENSG00000125384 | Prostaglandin E receptor 2 | 14 | 52314305-52328598 |
| PTGER3 | ENSG00000050628 | Prostaglandin E receptor 3 | 1 | 70852353-71047808 |
| PTGER4 | ENSG00000171522 | Prostaglandin E receptor 4 | 5 | 40679915-40693735 |
| PTGFR | ENSG00000122420 | Prostaglandin F receptor | 1 | 78303884-78540701 |
| PTGIR | ENSG00000160013 | Prostaglandin I2 receptor | 19 | 46620468-46625089 |
| PTGIS | ENSG00000124212 | Prostaglandin I2 synthase | 20 | 49503874-49568137 |
| PTGS1 | ENSG00000095303 | Prostaglandin-endoperoxide synthase 1 | 9 | 122370530-122395703 |
| PTGS2 | ENSG00000073756 | Prostaglandin-endoperoxide synthase 2 | 1 | 186671791-186680423 |
| PTH2R | ENSG00000144407 | Parathyroid hormone 2 receptor | 2 | 208359714-208854503 |
| PTK2 | ENSG00000169398 | Protein tyrosine kinase 2 | 8 | 140657900-141002216 |
| PTK6 | ENSG00000101213 | Protein tyrosine kinase 6 | 20 | 63528001-63537376 |
| QPRT | ENSG00000103485 | Quinolinate phosphoribosyltransferase | 16 | 29663279-29698699 |
| RAC1 | ENSG00000136238 | Rac family small GTPase 1 | 7 | 6374527-6403967 |
| RAD50 | ENSG00000113522 | RAD50 double strand break repair protein | 5 | 132556019-132646349 |
| RAF1 | ENSG00000132155 | Raf-1 proto-oncogene, serine/threonine kinase | 3 | 12583601-12664226 |
| RAMP1 | ENSG00000132329 | Receptor activity modifying protein 1 | 2 | 237858893-237912106 |
| RAMP2 | ENSG00000131477 | Receptor activity modifying protein 2 | 17 | 42758447-42763041 |
| RAMP3 | ENSG00000122679 | Receptor activity modifying protein 3 | 7 | 45157791-45186302 |
| RARA | ENSG00000131759 | Retinoic acid receptor alpha | 17 | 40309180-40357643 |
| RARB | ENSG00000077092 | Retinoic acid receptor beta | 3 | 25174332-25597932 |
| RARG | ENSG00000172819 | Retinoic acid receptor gamma | 12 | 53210567-53232980 |
| RBM39 | ENSG00000131051 | RNA binding motif protein 39 | 20 | 35701347-35742312 |
| REN | ENSG00000143839 | Renin | 1 | 204154819-204190324 |
| RET | ENSG00000165731 | Ret proto-oncogene | 10 | 43077064-43130351 |
| RFK | ENSG00000135002 | Riboflavin kinase | 9 | 76385526-76394517 |
| RICTOR | ENSG00000164327 | RPTOR independent companion of MTOR | 5 | 38937920-39074399 |
| | | complex 2 | | |
| ROCK1 | ENSG00000067900 | Rho associated coiled-coil containing protein | 18 | 20946906-21111813 |
| | | kinase 1 | | |
| ROCK2 | ENSG00000134318 | Rho associated coiled-coil containing protein | 2 | 11179759-11348330 |
| | | kinase 2 | | |
| ROS1 | ENSG00000047936 | ROS proto-oncogene 1, receptor tyrosine kinase | 6 | 117288300-117425855 |
| RPL3 | ENSG00000100316 | Ribosomal protein L3 | 22 | 39312882-39320389 |
| RPS6KB1 | ENSG00000108443 | Ribosomal protein S6 kinase B1 | 17 | 59893046-59950574 |
| RPTOR | ENSG00000141564 | Regulatory associated protein of MTOR complex | 17 | 80544819-80966371 |
| | | 1 | | |
| RRM1 | ENSG00000167325 | Ribonucleotide reductase catalytic subunit M1 | 11 | 4094707-4138932 |
| RRM2 | ENSG00000171848 | Ribonucleotide reductase regulatory subunit M2 | 2 | 10120698-10211725 |
| RRM2B | ENSG00000048392 | Ribonucleotide reductase regulatory TP53 | 8 | 102204502-102238961 |
| | | inducible subunit M2B | | |
| RXRA | ENSG00000186350 | Retinoid X receptor alpha | 9 | 134317098-134440585 |
| RXRB | ENSG00000204231 | Retinoid X receptor beta | 6 | 33193588-33200688 |
| RXRG | ENSG00000143171 | Retinoid X receptor gamma | 1 | 165400922-165445355 |
| RYR1 | ENSG00000196218 | Ryanodine receptor 1 | 19 | 38433691-38587564 |
| RYR2 | ENSG00000198626 | Ryanodine receptor 2 | 1 | 237042184-237833988 |
| S1PR1 | ENSG00000170989 | Sphingosine-1-phosphate receptor 1 | 1 | 101236865-101243713 |
| S1PR5 | ENSG00000180739 | Sphingosine-1-phosphate receptor 5 | 19 | 10512742-10517931 |
| SCN10A | ENSG00000185313 | Sodium voltage-gated channel alpha subunit 10 | 3 | 38696802-38816286 |
| SCN11A | ENSG00000168356 | Sodium voltage-gated channel alpha subunit 11 | 3 | 38845767-39051941 |
| SCN1A | ENSG00000144285 | Sodium voltage-gated channel alpha subunit 1 | 2 | 165984641-166149214 |
| SCN1B | ENSG00000105711 | Sodium voltage-gated channel beta subunit 1 | 19 | 35030470-35040449 |
| SCN2A | ENSG00000136531 | Sodium voltage-gated channel alpha subunit 2 | 2 | 165194993-165392310 |
| SCN2B | ENSG00000149575 | Sodium voltage-gated channel beta subunit 2 | 11 | 118162806-118176639 |
| SCN3A | ENSG00000153253 | Sodium voltage-gated channel alpha subunit 3 | 2 | 165087522-165204067 |
| SCN3B | ENSG00000166257 | Sodium voltage-gated channel beta subunit 3 | 11 | 123629187-123655244 |
| SCN4A | ENSG00000007314 | Sodium voltage-gated channel alpha subunit 4 | 17 | 63938554-63972918 |
| SCN4B | ENSG00000177098 | Sodium voltage-gated channel beta subunit 4 | 11 | 118133377-118152888 |
| SCN5A | ENSG00000183873 | Sodium voltage-gated channel alpha subunit 5 | 3 | 38548057-38649687 |
| SCN9A | ENSG00000169432 | Sodium voltage-gated channel alpha subunit 9 | 2 | 166195185-166376001 |
| SCNN1A | ENSG00000111319 | Sodium channel epithelial 1 subunit alpha | 12 | 6346843-6377730 |
| SCNN1B | ENSG00000168447 | Sodium channel epithelial 1 subunit beta | 16 | 23278231-23381294 |
| SCNN1D | ENSG00000162572 | Sodium channel epithelial 1 subunit delta | 1 | 1280436-1292029 |
| SCNN1G | ENSG00000166828 | Sodium channel epithelial 1 subunit gamma | 16 | 23182745-23216883 |
| SCTR | ENSG00000080293 | Secretin receptor | 2 | 119439843-119525301 |
| SDHD | ENSG00000204370 | Succinate dehydrogenase complex subunit D | 11 | 112086824-112120016 |
| SERPINB2 | ENSG00000197632 | Serpin family B member 2 | 18 | 63871692-63903888 |
| SERPINC1 | ENSG00000117601 | Serpin family C member 1 | 1 | 173903804-173917378 |
| SERPIND1 | ENSG00000099937 | Serpin family D member 1 | 22 | 20774113-20787720 |
| SERPINE1 | ENSG00000106366 | Serpin family E member 1 | 7 | 101127104-101139247 |
| SH2B3 | ENSG00000111252 | SH2B adaptor protein 3 | 12 | 111405923-111451623 |
| SHH | ENSG00000164690 | Sonic hedgehog signaling molecule | 7 | 155799980-155812463 |
| SHMT1 | ENSG00000176974 | Serine hydroxymethyltransferase 1 | 17 | 18327860-18363563 |
| SI | ENSG00000090402 | Sucrase-isomaltase | 3 | 164978898-165078496 |
| SIGMAR1 | ENSG00000147955 | Sigma non-opioid intracellular receptor 1 | 9 | 34634722-34637809 |
| SIK1 | ENSG00000142178 | Salt inducible kinase 1 | 21 | 43414483-43427131 |
| SIRT1 | ENSG00000096717 | Sirtuin 1 | 10 | 67884656-67918390 |
| SIRT5 | ENSG00000124523 | Sirtuin 5 | 6 | 13574529-13615158 |
| SLAMF7 | ENSG00000026751 | SLAM family member 7 | 1 | 160739057-160754821 |
| SLC12A1 | ENSG00000074803 | Solute carrier family 12 member 1 | 15 | 48178438-48304078 |
| SLC12A2 | ENSG00000064651 | Solute carrier family 12 member 2 | 5 | 128083766-128189677 |
| SLC12A3 | ENSG00000070915 | Solute carrier family 12 member 3 | 16 | 56865207-56915850 |
| SLC12A4 | ENSG00000124067 | Solute carrier family 12 member 4 | 16 | 67943474-67969601 |
| SLC12A5 | ENSG00000124140 | Solute carrier family 12 member 5 | 20 | 46021690-46060150 |
| SLC18A1 | ENSG00000036565 | Solute carrier family 18 member A1 | 8 | 20144855-20183206 |
| SLC18A2 | ENSG00000165646 | Solute carrier family 18 member A2 | 10 | 117241093-117279430 |
| SLC22A11 | ENSG00000168065 | Solute carrier family 22 member 11 | 11 | 64555690-64572875 |
| SLC22A12 | ENSG00000197891 | Solute carrier family 22 member 12 | 11 | 64590641-64602353 |
| SLC22A6 | ENSG00000197901 | Solute carrier family 22 member 6 | 11 | 62936385-62984983 |
| SLC22A8 | ENSG00000149452 | Solute carrier family 22 member 8 | 11 | 62989154-63015841 |
| SLC25A4 | ENSG00000151729 | Solute carrier family 25 member 4 | 4 | 185143266-185150382 |
| SLC25A5 | ENSG00000005022 | Solute carrier family 25 member 5 | X | 119468422-119471396 |
| SLC25A6 | ENSG00000169100 | Solute carrier family 25 member 6 | X | 1386152-1392113 |
| SLC2A2 | ENSG00000163581 | Solute carrier family 2 member 2 | 3 | 170996347-171026743 |
| SLC52A2 | ENSG00000185803 | Solute carrier family 52 member 2 | 8 | 144354135-144361272 |
| SLC5A2 | ENSG00000140675 | Solute carrier family 5 member 2 | 16 | 31483002-31490860 |
| SLC6A1 | ENSG00000157103 | Solute carrier family 6 member 1 | 3 | 10992186-11039247 |
| SLC6A2 | ENSG00000103546 | Solute carrier family 6 member 2 | 16 | 55655604-55706192 |
| SLC6A3 | ENSG00000142319 | Solute carrier family 6 member 3 | 5 | 1392794-1445440 |
| SLC6A4 | ENSG00000108576 | Solute carrier family 6 member 4 | 17 | 30194319-30236002 |
| SLC6A8 | ENSG00000130821 | Solute carrier family 6 member 8 | X | 153687926-153696588 |
| SLC7A11 | ENSG00000151012 | Solute carrier family 7 member 11 | 4 | 138164097-138242349 |
| SLC8A1 | ENSG00000183023 | Solute carrier family 8 member A1 | 2 | 40097270-40611053 |
| SLCO2B1 | ENSG00000137491 | Solute carrier organic anion transporter family | 11 | 75100563-75206549 |
| | | member 2B1 | | |
| SMO | ENSG00000128602 | Smoothened, frizzled class receptor | 7 | 129188633-129213545 |
| SMOX | ENSG00000088826 | Spermine oxidase | 20 | 4120980-4187747 |
| SMS | ENSG00000102172 | Spermine synthase | X | 21940709-21994837 |
| SNAP25 | ENSG00000132639 | Synaptosome associated protein 25 | 20 | 10218830-10307418 |
| SOAT1 | ENSG00000057252 | Sterol O-acyltransferase 1 | 1 | 179293714-179358680 |
| SQLE | ENSG00000104549 | Squalene epoxidase | 8 | 124998497-125022283 |
| SRC | ENSG00000197122 | SRC proto-oncogene, non-receptor tyrosine | 20 | 37344685-37406050 |
| | | kinase | | |
| SRD5A1 | ENSG00000145545 | Steroid 5 alpha-reductase 1 | 5 | 6633427-6674386 |
| SRD5A2 | ENSG00000277893 | Steroid 5 alpha-reductase 2 | 2 | 31522480-31580938 |
| SSTR1 | ENSG00000139874 | Somatostatin receptor 1 | 14 | 38207904-38213067 |
| SSTR2 | ENSG00000180616 | Somatostatin receptor 2 | 17 | 73165010-73176633 |
| SSTR5 | ENSG00000162009 | Somatostatin receptor 5 | 16 | 1078781-1080142 |
| STAT3 | ENSG00000168610 | Signal transducer and activator of transcription 3 | 17 | 42313324-42388568 |
| SV2A | ENSG00000159164 | Synaptic vesicle glycoprotein 2A | 1 | 149903318-149917844 |
| SYK | ENSG00000165025 | Spleen associated tyrosine kinase | 9 | 90801787-90898549 |
| SYT2 | ENSG00000143858 | Synaptotagmin 2 | 1 | 202590596-202710454 |
| TAAR1 | ENSG00000146399 | Trace amine associated receptor 1 | 6 | 132644898-132646051 |
| TACR1 | ENSG00000115353 | Tachykinin receptor 1 | 2 | 75046463-75199520 |
| TBK1 | ENSG00000183735 | TANK binding kinase 1 | 12 | 64452092-64502114 |
| TBXA2R | ENSG00000006638 | Thromboxane A2 receptor | 19 | 3594507-3606875 |
| TBXAS1 | ENSG00000059377 | Thromboxane A synthase 1 | 7 | 139777051-140020325 |
| TEK | ENSG00000120156 | TEK receptor tyrosine kinase | 9 | 27109141-27230174 |
| TERT | ENSG00000164362 | Telomerase reverse transcriptase | 5 | 1253147-1295068 |
| TFPI | ENSG00000003436 | Tissue factor pathway inhibitor | 2 | 187464230-187565760 |
| TGFB1 | ENSG00000105329 | Transforming growth factor beta 1 | 19 | 41301587-41353922 |
| TGFBR1 | ENSG00000106799 | Transforming growth factor beta receptor 1 | 9 | 99104038-99154192 |
| TH | ENSG00000180176 | Tyrosine hydroxylase | 11 | 2163929-2171815 |
| THRA | ENSG00000126351 | Thyroid hormone receptor alpha | 17 | 40058290-40093867 |
| THRB | ENSG00000151090 | Thyroid hormone receptor beta | 3 | 24117153-24495756 |
| TLR2 | ENSG00000137462 | Toll like receptor 2 | 4 | 153684070-153705702 |
| TLR5 | ENSG00000187554 | Toll like receptor 5 | 1 | 223109404-223143248 |
| TLR7 | ENSG00000196664 | Toll like receptor 7 | X | 12867072-12890361 |
| TLR8 | ENSG00000101916 | Toll like receptor 8 | X | 12906620-12923169 |
| TLR9 | ENSG00000239732 | Toll like receptor 9 | 3 | 52221080-52226163 |
| TNF | ENSG00000232810 | Tumor necrosis factor | 6 | 31575565-31578336 |
| TNFRSF8 | ENSG00000120949 | TNF receptor superfamily member 8 | 1 | 12063303-12144207 |
| TNFSF11 | ENSG00000120659 | TNF superfamily member 11 | 13 | 42562736-42608013 |
| TNFSF13B | ENSG00000102524 | TNF superfamily member 13b | 13 | 108251240-108308484 |
| TNKS | ENSG00000173273 | Tankyrase | 8 | 9555912-9782346 |
| TNNC1 | ENSG00000114854 | Troponin C1, slow skeletal and cardiac type | 3 | 52451100-52454041 |
| TOP1 | ENSG00000198900 | DNA topoisomerase I | 20 | 41028822-41124487 |
| TOP1MT | ENSG00000184428 | DNA topoisomerase I mitochondrial | 8 | 143304384-143359979 |
| TOP2A | ENSG00000131747 | DNA topoisomerase II alpha | 17 | 40388525-40417896 |
| TOP2B | ENSG00000077097 | DNA topoisomerase II beta | 3 | 25597905-25664907 |
| TPH1 | ENSG00000129167 | Tryptophan hydroxylase 1 | 11 | 18017564-18042426 |
| TPH2 | ENSG00000139287 | Tryptophan hydroxylase 2 | 12 | 71938845-72186618 |
| TPK1 | ENSG00000196511 | Thiamin pyrophosphokinase 1 | 7 | 144451941-144836395 |
| TPMT | ENSG00000137364 | Thiopurine S-methyltransferase | 6 | 18128311-18155077 |
| TPO | ENSG00000115705 | Thyroid peroxidase | 2 | 1374066-1543711 |
| TRHR | ENSG00000174417 | Thyrotropin releasing hormone receptor | 8 | 109086585-109121565 |
| TRPA1 | ENSG00000104321 | Transient receptor potential cation channel | 8 | 72019917-72075584 |
| | | subfamily A member 1 | | |
| TRPM8 | ENSG00000144481 | Transient receptor potential cation channel | 2 | 233917373-234019522 |
| | | subfamily M member 8 | | |
| TRPV1 | ENSG00000196689 | Transient receptor potential cation channel | 17 | 3565444-3609411 |
| | | subfamily V member 1 | | |
| TRPV3 | ENSG00000167723 | Transient receptor potential cation channel | 17 | 3510502-3557812 |
| | | subfamily V member 3 | | |
| TSHR | ENSG00000165409 | Thyroid stimulating hormone receptor | 14 | 80954989-81146302 |
| TSPO | ENSG00000100300 | Translocator protein | 22 | 43151547-43163242 |
| TUBA1A | ENSG00000167552 | Tubulin alpha 1a | 12 | 49184795-49189080 |
| TUBA4A | ENSG00000127824 | Tubulin alpha 4a | 2 | 219249710-219278170 |
| TUBB | ENSG00000196230 | Tubulin beta class I | 6 | 30720352-30725426 |
| TUBB1 | ENSG00000101162 | Tubulin beta 1 class VI | 20 | 59019429-59026654 |
| TUBB3 | ENSG00000258947 | Tubulin beta 3 class III | 16 | 89921392-89938761 |
| TUBB4B | ENSG00000188229 | Tubulin beta 4B class IVb | 9 | 137241287-137243707 |
| TUBD1 | ENSG00000108423 | Tubulin delta 1 | 17 | 59859479-59892945 |
| TUBE1 | ENSG00000074935 | Tubulin epsilon 1 | 6 | 112070663-112087529 |
| TUBG1 | ENSG00000131462 | Tubulin gamma 1 | 17 | 42609683-42615238 |
| TXN | ENSG00000136810 | Thioredoxin | 9 | 110243810-110256507 |
| TXNRD1 | ENSG00000198431 | Thioredoxin reductase 1 | 12 | 104215779-104350307 |
| TYK2 | ENSG00000105397 | Tyrosine kinase 2 | 19 | 10350529-10380572 |
| TYMS | ENSG00000176890 | Thymidylate synthetase | 18 | 657653-673578 |
| TYR | ENSG00000077498 | Tyrosinase | 11 | 89177875-89295759 |
| UGCG | ENSG00000148154 | UDP-glucose ceramide glucosyltransferase | 9 | 111896814-111935369 |
| VAMP1 | ENSG00000139190 | Vesicle associated membrane protein 1 | 12 | 6462237-6470987 |
| VAMP2 | ENSG00000220205 | Vesicle associated membrane protein 2 | 17 | 8159149-8163546 |
| VDR | ENSG00000111424 | Vitamin D receptor | 12 | 47841537-47943048 |
| VEGFA | ENSG00000112715 | Vascular endothelial growth factor A | 6 | 43770184-43786487 |
| VEGFB | ENSG00000173511 | Vascular endothelial growth factor B | 11 | 64234538-64238793 |
| VKORC1 | ENSG00000167397 | Vitamin K epoxide reductase complex subunit 1 | 16 | 31090842-31095980 |
| VKORC1L1 | ENSG00000196715 | Vitamin K epoxide reductase complex subunit 1 | 7 | 65873074-65959563 |
| | | like 1 | | |
| VWF | ENSG00000110799 | Von Willebrand factor | 12 | 5948874-6124770 |
| WEE1 | ENSG00000166483 | WEE1 G2 checkpoint kinase | 11 | 9573670-9593457 |
| XDH | ENSG00000158125 | Xanthine dehydrogenase | 2 | 31334321-31414742 |
| XIAP | ENSG00000101966 | X-linked inhibitor of apoptosis | X | 123859724-123913979 |
| XPO1 | ENSG00000082898 | Exportin 1 | 2 | 61477849-61538626 |
| YES1 | ENSG00000176105 | YES proto-oncogene 1, Src family tyrosine | 18 | 721588-812546 |
| | | kinase | | |

TABLE 5A
Gene targets of cancer therapeutic agents.

ABL1
ABL2
ACPP (ACP3)
ACVR1B
ADA
ADORA2A
ADORA3
AGXT
AKT1
AKT2
AKT3
ALK
AMER1 (FAM123B)
ANGPT1
ANGPT2
ANPEP
APC
APH1A
APH1B
AR
ARAF
ARFRP1
ARID1A
ARID1B
ARID2
ASXL1
ATM
ATR
ATRX
AURKA
AURKB
AURKC
AXIN1
AXL
B4GALNT1
BAP1
BARD1
BAX
BCL2
BCL2L1
BCL2L2
BCL6
BCORL1
BIRC5
BLK
BLM
BMX
BRAF
BRCA1
BRCA2
BRD2
BRD3
BRD4
BRIP1
BTG1
BTK
C11orf30 (EMSY)
CARD11
CAXX
CBFB
CBL
CCND1
CCND2
CCND3
CCNE1
CD19
CD274
CD38
CD79A
CD79B
CDC73
CDH1
CDK1
CDK12
CDK2
CDK4
CDK5
CDK6
CDK7
CDK8
CDK9
CDKN1A
CDKN1B
CDKN2A
CDKN2B
CDKN2C
CEBPA
CHD1
CHD2
CHD4
CHEK1
CHEK2
CIC
CPT1A
CRBN
CREBBP
CRKL
CRLF2
CRTC1
CRTC2
CSF1R
CSNK2A1
CSNK2A2
CTCF
CTNNA1
CTNNB1
CUL3
CXCR1
CXCR2
CXCR4
CYLD
CYP11B1
CYP11B2
CYP17A1
CYP19A1
DDR2
DHFR
DHH
DHX9
DICER
DNMT1
DNMT3A
DOTIL
DPP4
DRD2
DYRK1A
DYRK1B
DYRK2
DYRK3
DYRK4
EDNRA
EGFR
EHMT1
EHMT2
EIF4E
EP300
EPHA2
EPHA3
EPHA5
EPHA7
EPHB1
EPHB4
ERBB2
ERBB3
ERBB4
ERG
ERRF11
ESR1
ESR2
EZH2
F2R
FAM46C
FANCA
FANCC
FANCD2
FANCE
FANCF
FANCG
FANCL
FAS
FAT1
FBXW7
FCGR1A
FGF1
FGF10
FGF14
FGF19
FGF2
FGF23
FGF3
FGF4
FGF6
FGFR1
FGFR2
FGFR3
FGFR4
FGR
FH
FKBP1A
FLCN
FLT1
FLT3
FLT4
FNTA
FOLH1
FOLR1
FOLR2
FOLR3
FOXL2
FOXP1
FRK
FRS2
FUBP1
FYN
FZD8
GABRA6
GART
GATA1
GATA2
GATA3
GATA4
GATA6
GID4 (C17ORF39)
GLI1
GNA11
GNA13
GNAQ
GNAS
GNRH1
GNRHR
GPR124
GRIN2A
GRM3
GSK3A
GSK3B
H3F3A
HCK
HDAC1
HDAC10
HDAC11
HDAC2
HDAC3
HDAC4
HDAC5
HDAC6
HDAC7
HDAC8
HDAC9
HGF
HNF1A
HPRT1
HPSE
HRAS
HSD3B1
HSP90AA1
HSPA1A
HSPA1B
HSPB1
IDH1
IDH2
IDO1
IFNAR1
IFNAR2
IGF1R
IGF2
IHH
IKBKB
IKBKE
IKZF1
IL1B
IL2RA
IL2RB
IL2RG
IL6
IL6R
IL6ST
IL7R
INHA
INHBA
INHBB
INHBC
INHBE
INPP4B
IRF2
IRF4
IRS2
ITK
JAK1
JAK2
JAK3
JUN
KAT6A (MYST3)
KDM1A
KDM5A
KDM5C
KDM6A
KDR
KEAP1
KEL
KIF11
KIT
KLHL6
KMT2A (MLL)
KMT2C (MLL3)
KMT2D (MLL2)
KRAS
LAP3
LCK
LDLR
LHCGR
LIMK1
LMO1
LRP1B
LYN
LZTR1
MAGI2
MAP1A
MAP2
MAP2K1
MAP2K1 (MEK1)
MAP2K2
MAP2K2 (MEK2)
MAP2K4 (MEK4)
MAP3K1
MAP3K7
MAP3K8
MAPK1
MAPK11
MAPK14
MAPK3
MAPK8
MAPK9
MCL1
MDM2
MDM4
MED12
MEF2B
MEN1
MET
MGMT
MITF
MLH1
MPL
MRE11
MRE11A
MS4A1
MSH2
MSH6
MSLN
MST1R
MTOR
MUC5AC
MUTYH
MYC
MYCL (MYCL1)
MYCN
MYD88
NAE1
NAMPT
NBN
NCSTN
NEK11
NF1
NF2
NFE2L2
NFKBIA
NKX2-I
NOTCH1
NOTCH2
NOTCH3
NOTCH4
NPEPPS
NPM1
NR3C1
NRAS
NSD1
NTRK1
NTRK2
NTRK3
NUP93
PAK3
PALB2
PARK2
PARP1
PARP2
PARP3
PAX5
PBRM1
PDCD1LG2
PDE5A
PDGFRA
PDGFRB
PDK1
PGF
PIGF
PIK3C2B
PIK3CA
PIK3CB
PIK3CD
PIK3CG
PIK3R1
PIK3R2
PIM1
PIM2
PIM3
PLCG2
PLK1
PLK2
PLK3
PMS2
POLA1
POLD1
POLE
PORCN
PPARG
PPP2CA
PPP2R1A
PRDM1
PREX2
PRKAR1A
PRKCA
PRKCB
PRKCE
PRKCG
PRKCI
PRKDC
PRLR
PRMT5
PRSS8
PSEN1
PSENEN
PSMB1
PSMB10
PSMB2
PSMB5
PSMB8
PSMB9
PSMD1
PSMD2
PTCH1
PTEN
PTGS2
PTK2
PTK6
PTPN11
QKI
RAC1
RAD50
RAD51
RAD51B
RAD51C
RAD51D
RAD54L
RAF1
RANBP2
RARA
RARB
RARG
RB1
RBM10
RBM39
RET
RICTOR
RNF43
ROCK1
ROCK2
ROS1
RPL3
RPS6KB1
RPTOR
RRM1
RUNX1
RUNX1T1
RXRA
RXRB
RXRG
S1PR1
SDHA
SDHB
SDHC
SDHD
SETD2
SF3B1
SH2B3
SHH
SIK1
SIRT1
SLAMF7
SLC2A2
SLIT2
SMAD2
SMAD3
SMAD4
SMARCA4
SMARCB1
SMO
SNCAIP
SOCS1
SOX10
SOX2
SOX9
SPEN
SPOP
SPTA1
SRC
SSTR2
SSTR5
STAG2
STAT3
STAT4
STK11
SUFU
SYK
TAF1
TBK1
TBX3
TEK
TERC
TERT
TET2
TGFB1
TGFBR1
TGFBR2
TLR5
TLR7
TLR8
TMB
TNF
TNFAIP3
TNFRSF14
TNFRSF8
TNFSF11
TNFSF13B
TNKS
TOP1
TOP1MT
TOP2A
TOP2B
TP53
TSC1
TSC2
TSHR
TUBA1A
TUBA4A
TUBB
TUBB1
TUBB3
TUBD1
TUBE1
TUBG1
TXN
TYK2
TYMS
U2AF1
VDR
VEGFA
VEGFB
VHL
WEE1
WISP3
WT1
XIAP
XPO1
YES1
ZBTB2
ZNF217
ZNF703

TABLE 5B
Gene targets of cancer therapeutic agents.

ABL1
ABL2
ACPP (ACP3)
ADA
ADORA2A
ADORA3
AGXT
AKT1
AKT2
AKT3
ALK
ANGPT1
ANGPT2
ANPEP
APH1A
APH1B
AR
ARAF
ATR
AURKA
AURKB
AURKC
AXL
B4GALNT1
BAX
BCL2
BCL2L1
BCL2L2
BIRC5
BLK
BMX
BRAF
BRD2
BRD3
BRD4
BTK
CCND1
CCND2
CCND3
CD19
CD274
CD38
CDK1
CDK2
CDK4
CDK5
CDK6
CDK7
CDK9
CHD1
CHEK1
CHEK2
CPT1A
CRBN
CRTC1
CRTC2
CSF1R
CSNK2A1
CSNK2A2
CXCR1
CXCR2
CXCR4
CYP17A1
CYP19A1
DDR2
DHFR
DHH
DHX9
DNMT1
DOT1L
DPP4
DRD2
EDNRA
EGFR
EHMT1
EHMT2
EPHA2
EPHB4
ERBB2
ERBB3
ERBB4
ESR1
ESR2
EZH2
F2R
FCGR1A
FGF1
FGF2
FGFR1
FGFR2
FGFR3
FGFR4
FGR
FKBP1A
FLT1
FLT3
FLT4
FNTA
FOLH1
FOLR1
FOLR2
FOLR3
FRK
FYN
FZD8
GART
GNRH1
GNRHR
GSK3A
GSK3B
HCK
HDAC1
HDAC10
HDAC11
HDAC2
HDAC3
HDAC4
HDAC5
HDAC6
HDAC7
HDAC8
HDAC9
HPRT1
HPSE
HSP90AA1
HSPA1A
HSPA1B
HSPB1
IDH1
IDH2
IDO1
IFNAR1
IFNAR2
IGF1R
IHH
IKBKB
IL1B
IL2RA
IL2RB
IL2RG
IL6
IL6R
INHA
INHBA
INHBB
INHBC
INHBE
ITK
JAK1
JAK2
JAK3
KDM1A
KDR
KIF11
KIT
KRAS
LAP3
LCK
LDLR
LHCGR
LIMK1
LYN
MAP1A
MAP2
MAP2K1
MAP2K2
MAP3K7
MAP3K8
MAPK1
MAPK11
MAPK14
MAPK3
MAPK8
MAPK9
MCL1
MDM2
MET
MGMT
MRE11
MS4A1
MSLN
MST1R
MTOR
MUC5AC
NAE1
NAMPT
NCSTN
NEK11
NOTCH1
NOTCH2
NOTCH3
NOTCH4
NPEPPS
NR3C1
NRAS
NTRK1
NTRK2
NTRK3
PARP1
PARP2
PARP3
PDE5A
PDGFRA
PDGFRB
PGF
PIGF
PIK3CA
PIK3CB
PIK3CD
PIK3CG
PIM3
PLK1
PLK2
PLK3
POLA1
PORCN
PPARG
PPP2CA
PRKCA
PRKCB
PRKCE
PRKCG
PRKCI
PRKDC
PRLR
PSEN1
PSENEN
PSMB1
PSMB10
PSMB2
PSMB5
PSMB8
PSMB9
PSMD1
PSMD2
PTCH1
PTGS2
PTK2
PTK6
RAC1
RAD50
RAF1
RARA
RARB
RARG
RET
RICTOR
ROCK1
ROCK2
ROS1
RPL3
RPS6KB1
RPTOR
RRM1
RXRA
RXRB
RXRG
S1PR1
SH2B3
SHH
SIK1
SIRT1
SLAMF7
SLC2A2
SMO
SRC
SSTR2
SSTR5
STAT3
SYK
TBK1
TEK
TERT
TGFB1
TGFBR1
TLR5
TLR7
TLR8
TNF
TNFRSF8
TNFSF11
TNFSF13B
TNKS
TOP1
TOP1MT
TOP2A
TOP2B
TUBA1A
TUBA4A
TUBB
TUBB1
TUBB3
TUBD1
TUBE1
TUBG1
TXN
TYK2
TYMS
VDR
VEGFA
VEGFB
WEE1
XIAP
XPO1
YES1

TABLE 5C
Gene targets of cancer therapeutic agents.

ABL1
ABL2
ACPP (ACP3)
ADA
ADORA2A
ADORA3
AGXT
AKT1
AKT2
AKT3
ALK
ANGPT1
ANGPT2
ANPEP
APH1A
APH1B
AR
ARAF
ATR
AURKA
AURKB
AURKC
AXL
B4GALNT1
BAX
BCL2
BCL2L1
BCL2L2
BIRC5
BLK
BMX
BRAF
BRD2
BRD3
BRD4
BTK
CCND1
CCND2
CCND3
CD19
CD274
CD38
CDK1
CDK2
CDK4
CDK5
CDK6
CDK7
CDK9
CHD1
CHEK1
CHEK2
CPT1A
CRBN
CRTC1
CRTC2
CSF1R
CSNK2A1
CSNK2A2
CXCR1
CXCR2
CXCR4
CYP17A1
CYP19A1
DDR2
DHFR
DHH
DHX9
DNMT1
DOT1L
DPP4
DRD2
DYRK1A
DYRK1B
DYRK2
DYRK3
DYRK4
EDNRA
EGFR
EHMT1
EHMT2
EIF4E
EPHA2
EPHB4
ERBB2
ERBB3
ERBB4
ESR1
ESR2
EZH2
F2R
FCGR1A
FGF1
FGF2
FGFR1
FGFR2
FGFR3
FGFR4
FGR
FKBP1A
FLT1
FLT3
FLT4
FNTA
FOLH1
FOLR1
FOLR2
FOLR3
FRK
FYN
FZD8
GART
GNRH1
GNRHR
GSK3A
GSK3B
HCK
HDAC1
HDAC10
HDAC11
HDAC2
HDAC3
HDAC4
HDAC5
HDAC6
HDAC7
HDAC8
HDAC9
HPRT1
HPSE
HSP90AA1
HSPA1A
HSPA1B
HSPB1
IDH1
IDH2
IDO1
IFNAR1
IFNAR2
IGF1R
IHH
IKBKB
IL1B
IL2RA
IL2RB
IL2RG
IL6
IL6R
IL6ST
INHA
INHBA
INHBB
INHBC
INHBE
ITK
JAK1
JAK2
JAK3
KDM1A
KDR
KIF11
KIT
KRAS
LAP3
LCK
LDLR
LHCGR
LIMK1
LYN
MAP1A
MAP2
MAP2K1
MAP2K2
MAP3K7
MAP3K8
MAPK1
MAPK11
MAPK14
MAPK3
MAPK8
MAPK9
MCL1
MDM2
MET
MGMT
MS4A1
MSLN
MST1R
MTOR
MUC5AC
NAE1
NAMPT
NBN
NCSTN
NEK11
NOTCH1
NOTCH2
NOTCH3
NOTCH4
NPEPPS
NR3C1
NRAS
NTRK1
NTRK2
NTRK3
PARP1
PARP2
PARP3
PDE5A
PDGFRA
PDGFRB
PGF
PIGF
PIK3CA
PIK3CB
PIK3CD
PIK3CG
PIM1
PIM2
PIM3
PLK1
PLK2
PLK3
POLA1
PORCN
PPARG
PPP2CA
PRKCA
PRKCB
PRKCE
PRKCG
PRKCI
PRKDC
PRLR
PRMT5
PSEN1
PSENEN
PSMB1
PSMB10
PSMB2
PSMB5
PSMB8
PSMB9
PSMD1
PSMD2
PTCH1
PTGS2
PTK2
PTK6
RAC1
RAD50
RAF1
RARA
RARB
RARG
RBM39
RET
RICTOR
ROCK1
ROCK2
ROS1
RPL3
RPS6KB1
RPTOR
RRM1
RXRA
RXRB
RXRG
S1PR1
SH2B3
SHH
SIK1
SIRT1
SLAMF7
SLC2A2
SMO
SRC
SSTR2
SSTR5
STAT3
SYK
TBK1
TEK
TERT
TGFB1
TGFBR1
TLR5
TLR7
TLR8
TNF
TNFRSF8
TNFSF11
TNFSF13B
TNKS
TOP1
TOP1MT
TOP2A
TOP2B
TUBA1A
TUBA4A
TUBB
TUBB1
TUBB3
TUBD1
TUBE1
TUBG1
TXN
TYK2
TYMS
VDR
VEGFA
VEGFB
WEE1
XIAP
XPO1
YES1

TABLE 5D
Gene targets of cancer therapeutic agents.

BIRC5
BRAF
CDK4
CDK6
CYP11B1
CYP11B2
EGFR
FLT3
GART
JAK1
JAK2
KIF11
KIT
MAP2K1
MAP2K2
MDM2
MET
MTOR
PIK3CA
PSMB5
RPL3
TOP2A
TUBG1

Tables 6A-6C provide lists of gene modulatory reagents, one or more of which may be used in a method of cell editing described herein.

TABLE 6A
Library of gene modulatory reagents.

| Target | | SEQ | | |
|---|---|---|---|---|
| Gene | | ID | | |
| Symbol | Transcript | NO. | Sequences | gRNA coordinates |
| ABL1 | ENST00000372348.6 | 1 | GGACACAGGCCCATGGTACC | chr9:130854923-130854946_− |
| | | 2 | ATCATTCAACGGTGGCCGAC | chr9:130862814-130862837_+ |
| | | 3 | CATCACGCCAGTCAACAGTC | chr9:130854891-130854914_+ |
| | | 4 | ATCTCAGCGAGATGGACCTC | chr9:130855023-130855046_− |
| | | 5 | AAGAAGGAATCATCGAGGCA | chr9:130714400-130714423_+ |
| ABL2 | ENST00000502732.5 | 6 | CCTCAGCCCCGCGGGATCCG; | chr1:179229326-179229349_− |
| | | 7 | TTACCATGAAGCACAAACTT | chr1:179121669-179121692_− |
| | | 8 | GGTGAAAAGCTACGAGTCCT | chr1:179126650-179126673_− |
| | | 9 | GGAGTACGCGAGAGCAGGGA | chr1:179229393-179229416_− |
| | | 10 | GAGTACGCGAGAGCAGGGAT | chr1:179229392-179229415_− |
| ACPP | ENST00000336375.9 | 11 | GCAGCCCTGTTTCCCCCAGA | chr3:132332248-132332271_+ |
| (ACP3) | | 12 | CCTACTCTGGCAGCCCATCC | chr3:132332292-132332315_+ |
| | | 13 | GAAAGAGGAACTGTGTGCAC | chr3:132332311-132332334_− |
| | | 14 | AGTCACAAACTTCAACTCCT | chr3:132317550-132317573_− |
| | | 15 | CCAGATGCTGACACCTTCTG | chr3:132332261-132332284_− |
| ADA | ENST00000372874.8 | 16 | GAGACTTCGGGGTCAAGGCC | chr20:44625599-44625622_− |
| | | 17 | CAGGCTTGATGGATCCGTCT | chr20:44636248-44636271_+ |
| | | 18 | AAACCATCTTATACTATGGC | chr20:44636225-44636248_− |
| | | 19 | CCAAAGTGGAGCCAATCCCC | chr20:44626466-44626489_− |
| | | 20 | CTCCCAGCTAACACAGCAGA | chr20:44629130-44629153_− |
| ADORA2A | ENST00000611543.4 | 21 | GAACGTCACCAACTACTTTG | chr22:24433517-24433540_+ |
| | | 22 | TTGCCATCCGCATCCCGCTC | chr22:24433714-24433737_+ |
| | | 23 | CATGGCCACAGACGACAGGC | chr22:24433384-24433407_− |
| | | 24 | TGGGCATGGCCACAGACGAC | chr22:24433388-24433411_− |
| | | 25 | AGCACACCAGCACATTGCCC | chr22:24433466-24433489_− |
| ADORA3 | ENST00000241356.4 | 26 | TGGGCATCTTGCCTTCCCAG | chr1:111503347-111503370_+ |
| | | 27 | GACAGAGCAGTGCTGTTGTT | chr1:111503328-111503351_+ |
| | | 28 | AATAGAAGGTGGTGGTCTGC | chr1:111503206-111503229_+ |
| | | 29 | CAAGGACATGATGGAGGCGT | chr1:111503051-111503074_+ |
| | | 30 | GTCCTTGCTGGCCATCGCTG | chr1:111503035-111503058_− |
| AGXT | ENST00000307503.3 | 31 | GGACCCCCCTTTACATGGAC | chr2:240873022-240873045_+ |
| | | 32 | CAACCTGCCTCCTCGCATCA | chr2:240868957-240868980_+ |
| | | 33 | CCCCCGGCTGCCATGATGCG | chr2:240868967-240868990_− |
| | | 34 | TCCAGTACGTGTTCCAGACC | chr2:240869194-240869217_+ |
| | | 35 | CATTTGGGGGCAGCGAGCCG | chr2:240869321-240869344_+ |
| AKT1 | ENST00000349310.7 | 36 | TGGCACCTTCATTGGCTACA | chr14:104780144-104780167_− |
| | | 37 | GGCTCACCCAGTGACAACTC | chr14:104775697-104775720_− |
| | | 38 | GCCGTCAGCCACAGTCTGGA | chr14:104775759-104775782_+ |
| | | 39 | CGACGTGGCTATTGTGAAGG | chr14:104792615-104792638_− |
| | | 40 | GGAGGAGATGGACTTCCGGT | chr14:104775719-104775742_− |
| AKT2 | ENST00000392038.6 | 41 | ATGACAAAGGTGTTGGGTCG | chr19:40255220-40255243_+ |
| | | 42 | CTGGTGCGGGAGAAGGCCAC | chr19:40241986-40242009_− |
| | | 43 | CAGGAAGTACCGTGGCCTCC | chr19:40257016-40257039_+ |
| | | 44 | GTCCATGGGGTCCTCGCCTG | chr19:40242611-40242634_+ |
| | | 45 | TGTCTGTCATCAAAGAAGGC | chr19:40265234-40265257_− |
| AKT3 | ENST00000366539.5 | 46 | TTTGACTATTTGAAACTACT | chr1:243637707-243637730_− |
| | | 47 | TTACCATTGTGAAAGAAGGT | chr1:243843137-243843160_− |
| | | 48 | GATGTTACCATTGTGAAAGA | chr1:243843141-243843164_− |
| | | 49 | TCTCTATAACAGTAGTCCAC | chr1:243664802-243664825_+ |
| | | 50 | TCCCCTCAACAACTTTTCAG | chr1:243695593-243695616_− |
| ALK | ENST00000389048.7 | 51 | GACCTGCCATTGAGGAGTGT | chr2:29320787-29320810_+ |
| | | 52 | CCCCTCCACTGCATGACCTC | chr2:29694949-29694972_− |
| | | 53 | GTCCAGAGCTAGCGAGCCGC | chr2:29920377-29920400_+ |
| | | 54 | TCAGCGAGCTGTTCAGTTGG | chr2:29920128-29920151_− |
| | | 55 | ATTCCAGGGCCACTCGAAAT | chr2:29383797-29383820_+ |
| ANGPT1 | ENST00000517746.5 | 56 | CTGCCATTCTGACTCACATA | chr8:107497504-107497527_− |
| | | 57 | ACAGTGGGAGAAGATATAAC | chr8:107497453-107497476_− |
| | | 58 | TCGTCAAACATATATAATCC | chr8:107322009-107322032_− |
| | | 59 | GAGAAATCCGGTTCCACGTG | chr8:107497322-107497345_+ |
| | | 60 | GCACCCTATGTGAGTCAGAA | chr8:107497501-107497524_+ |
| ANGPT2 | ENST00000325203.9 | 61 | GGCAGTTGTCCATCTCTGGC | chr8:6562774-6562797_+ |
| | | 62 | CGGAAGAGCATGGACAGCAT | chr8:6562845-6562868_− |
| | | 63 | AGCAATATCAGGTCCAGCAT | chr8:6562817-6562840_− |
| | | 64 | AAGCAATATCAGGTCCAGCA | chr8:6562818-6562841_− |
| | | 65 | CAGGAGGAAAGTGTAGCTGC | chr8:6562793-6562816_+ |
| ANPEP | ENST00000300060.6 | 66 | CCACGCTTTACTTTGGTCCA | chr15:89806370-89806393_+ |
| | | 67 | CCCGCTGTCCACACCCGCCT | chr15:89803702-89803725_− |
| | | 68 | CGCCGGCGTTGAAGTCTGGC | chr15:89804374-89804397_+ |
| | | 69 | TTGAACTCGGCCTTCATGGC | chr15:89805376-89805399_+ |
| | | 70 | CTCAGTCTTGTCAATGTCGG | chr15:89806121-89806144_+ |
| APH1A | ENST00000369109.7 | 71 | GCCATCTGGCGGATGGAGAT | chr1:150267720-150267743_+ |
| | | 72 | TGACCGACCGGTCAGATGCC | chr1:150268042-150268065_− |
| | | 73 | TCTTGGTCCATGTGACCGAC | chr1:150268054-150268077_− |
| | | 74 | ACCCATCTCCATCCGCCAGA | chr1:150267721-150267744_− |
| | | 75 | TTAGCATCGCTGAGTGAGGA | chr1:150267750-150267773_− |
| APH1B | ENST00000261879.9 | 76 | ATCAGCAGATATTTCTGTGT | chr15:63279242-63279265_− |
| | | 77 | CTCTTGCCATGAACCAAACA | chr15:63279196-63279219_− |
| | | 78 | TCTTGCCATGAACCAAACAA | chr15:63279195-63279218_− |
| | | 79 | TAGGCCAGCAGTCGCATAGA | chr15:63286603-63286626_− |
| | | 80 | ATAGGCCAGCAGTCGCATAG | chr15:63286604-63286627_− |
| AR | ENST00000374690.8 | 81 | TAGAGGCCCCACAGGCTACC | chrX:67545448-67545471_+ |
| | | 82 | GCAGCTGAGTCATCCTCGTC | chrX:67545602-67545625_− |
| | | 83 | GCCCATCGTAGAGGCCCCAC | chrX:67545440-67545463_+ |
| | | 84 | CAGCAGGGACAACGTGGATG | chrX:67545624-67545647_− |
| | | 85 | TCCAGGACCAGGTAGCCTGT | chrX:67545455-67545478_− |
| ARAF | ENST00000377045.8 | 86 | TGGAGCGGATGCGCTGTAGG | chrX:47566695-47566718_− |
| | | 87 | ACAAAATTGTGCATGGTCAG | chrX:47564878-47564901_− |
| | | 88 | ACAGACTGTGGGGACCTTGG | chrX:47565090-47565113_− |
| | | 89 | GTGGAGCGGATGCGCTGTAG | chrX:47566696-47566719_− |
| | | 90 | GTGGTCTACCGACTCATCAA | chrX:47563306-47563329_+ |
| ATR | ENST00000350721.8 | 91 | CAAGAAGAATATTCCTTGAG | chr3:142556507-142556530_− |
| | | 92 | CGCACGTCAGCATTCTGGCA | chr3:142550228-142550251_+ |
| | | 93 | CAACTTGTCTGTACTCTTCA | chr3:142556058-142556081_− |
| | | 94 | TCCAGAGACAGATGCTGACT | chr3:142553316-142553339_+ |
| | | 95 | ACATGTCCGTGTTCAGAGAA | chr3:142556004-142556027_+ |
| AURKA | ENST00000395913.7 | 96 | GTGCTTGCAAAGGAATGCGC | chr20:56386391-56386414_+ |
| | | 97 | ATTACCTGTAAATAGTGGCC | chr20:56386445-56386468_− |
| | | 98 | ATGCGCTGGGAAGAATTTGA | chr20:56386405-56386428_+ |
| | | 99 | TGCTTGCAAAGGAATGCGCT | chr20:56386392-56386415_+ |
| | | 100 | TGAGTCACGAGAACACGTTT | chr20:56386489-56386512_+ |
| AURKB | ENST00000585124.5 | 101 | TCCCCCTTTCTCTCTAAGGA | chr17:8210220-8210243_− |
| | | 102 | AACTCCTACCCCTGGCCCTA | chr17:8210186-8210209_− |
| | | 103 | GGGCCATCCTTAGAGAGAAA | chr17:8210217-8210240_+ |
| | | 104 | CTCCATCACCTTCTGGCCAG | chr17:8207599-8207622_+ |
| | | 105 | GGACATTGGAGCGGCTCATG | chr17:8207746-8207769_+ |
| AURKC | ENST00000302804.11 | 106 | AATCGGGCGTCCCCTGGGCA | chr19:57232059-57232082_+ |
| | | 107 | ATCGGGCGTCCCCTGGGCAA | chr19:57232060-57232083_+ |
| | | 108 | TTTGAAATCGGGCGTCCCCT | chr19:57232054-57232077_+ |
| | | 109 | CAGTCGATGACTTTGAAATC | chr19:57232043-57232066_+ |
| | | 110 | CCAAATTTCCCCTTGCCCAG | chr19:57232069-57232092_− |
| AXL | ENST00000301178.8 | 111 | GAGTAGGTCCACGGGCTCTG | chr19:41221963-41221986_− |
| | | 112 | TGCCACACACACTGTCAGAT | chr19:41239227-41239250_− |
| | | 113 | GCCTAGCCGAAGCTGATGGG | chr19:41238028-41238051_− |
| | | 114 | CCACCTCCAGCTCCGTGGGT | chr19:41231222-41231245_− |
| | | 115 | AGTAGGTCCACGGGCTCTGG | chr19:41221962-41221985_− |
| B4GALNT1 | ENST00000341156.8 | 116 | CCAGTACCCCCTACAGGGTG | chr12:57631012-57631035_− |
| | | 117 | CCCACGGCGCAAGAGGTAGC | chr12:57632011-57632034_+ |
| | | 118 | GCAGTTGTGAGTCCAGTGGG | chr12:57631321-57631344_− |
| | | 119 | TGGCAGGGGCTATGAGCAGC | chr12:57631045-57631068_+ |
| | | 120 | GGGGGCGCCCACGGCGCAAG | chr12:57632004-57632027_+ |
| BAX | ENST00000345358.11 | 121 | ATGATCTGCTCAGAGCTGGT | chr19:48955549-48955572_− |
| | | 122 | GCAGCTGACATGTTTTCTGA | chr19:48956249-48956272_+ |
| | | 123 | GTTTCATCCAGGATCGAGCA | chr19:48955685-48955708_+ |
| | | 124 | TCTGACGGCAACTTCAACTG | chr19:48956264-48956287_+ |
| | | 125 | GGCGGTGATGGACGGGTCCG | chr19:48954921-48954944_+ |
| BCL2 | ENST00000398117.1 | 126 | CCATTATAAGCTGTCGCAGA | chr18:63318587-63318610_− |
| | | 127 | TCCAGCCGCATCCCGGGACC | chr18:63318470-63318493_− |
| | | 128 | CGCGCGGGGACGCTTTGCCA | chr18:63318269-63318292_− |
| | | 129 | GCGGCGAGGTCCTGGCGACC | chr18:63318452-63318475_+ |
| | | 130 | CACACCTGGATCCAGGATAA | chr18:63318088-63318111_− |
| BCL2L1 | ENST00000307677.4 | 131 | TCCCAGCTCCACATCACCCC | chr20:31721868-31721891_− |
| | | 132 | AGCAGTAAAGCAAGCGCTGA | chr20:31721944-31721967_− |
| | | 133 | TGGCAACCCATCCTGGCACC | chr20:31722040-31722063_− |
| | | 134 | TCCTACAAGCTTTCCCAGAA | chr20:31722156-31722179_− |
| | | 135 | CAGCAGCAGTTTGGATGCCC | chr20:31721983-31722006_− |
| BCL2L2 | ENST00000250405.9 | 136 | CTGAGCCGCCAGATCAGAGA | chr14:23307945-23307968_− |
| | | 137 | GACCTGGGTGAAGCGTTGTT | chr14:23307990-23308013_− |
| | | 138 | AGGCAGAAGGGTTATGTCTG | chr14:23307833-23307856_+ |
| | | 139 | GGTTATAAGCTGAGGCAGAA | chr14:23307821-23307844_+ |
| | | 140 | GCGTTGTTGGGCTGAGCCTG | chr14:23307978-23308001_− |
| BIRC5 | ENST00000350051.7 | 141 | CCAGGCAGGGGGCAACGTCG | chr17:78214323-78214346_− |
| | | 142 | ATGCGGTGGTCCTTGAGAAA | chr17:78214349-78214372_− |
| | | 143 | GCTGCGCCTGCACCCCGGAG | chr17:78214404-78214427_+ |
| | | 144 | GAACATAAAAAGCATTCGTC | chr17:78216667-78216690_+ |
| | | 145 | CAGGCGCAGCCCTCCAAGAA | chr17:78214391-78214414_− |
| BLK | ENST00000259089.8 | 146 | CTGGCCAGGTCACTCGTCAC | chr8:11549039-11549062_+ |
| | | 147 | GAGAAGCTACAGGTCCTGAA | chr8:11548102-11548125_+ |
| | | 148 | CTTTAGATCACAGGGTCGGA | chr8:11550164-11550187_+ |
| | | 149 | AGGTGGTTCTTTAGATCACA | chr8:11550156-11550179_+ |
| | | 150 | GAAGGTCAGCGCCCAAGACA | chr8:11543301-11543324_+ |
| BMX | ENST00000357607.6 | 151 | TGTCATATTCATAGTAGGAA | chrX:15508463-15508486_− |
| | | 152 | ACTGCATGTTGAAGTTTGGC | chrX:15522512-15522535_− |
| | | 153 | TGGTACTTGAAGATGGTGGC | chrX:15522446-15522469_− |
| | | 154 | GGTACTTGAAGATGGTGGCT | chrX:15522445-15522468_− |
| | | 155 | TGGTACTTGACCAGCAGGTG | chrX:15516125-15516148_− |
| BRAF | ENST00000646891.1 | 156 | AGAGAAGAAACCAATTGGTT | chr7:140808039-140808062_− |
| | | 157 | GAGAGAAGAAACCAATTGGT | chr7:140808040-140808063_− |
| | | 158 | CAACAGTTATTGGAATCTCT | chr7:140834801-140834824_− |
| | | 159 | GTGCTTTCTTTAGACTGTCT | chr7:140808946-140808969_+ |
| | | 160 | TGGGTGGTGTTCAAAGAACT | chr7:140800444-140800467_+ |
| BRD2 | ENST00000374825.8 | 161 | GCAGGCACCGAAGCCATTGT | chr6:32974567-32974590_− |
| | | 162 | TGGACATGGGTACTATTAAG | chr6:32975411-32975434_+ |
| | | 163 | AAGAGACGGCAGGCACCTGA | chr6:32976274-32976297_− |
| | | 164 | GGGCACTGGTAACACTGCCC | chr6:32976253-32976276_− |
| | | 165 | GTGTCCAATCCCAAAAAGCC | chr6:32974627-32974650_+ |
| BRD3 | ENST00000303407.11 | 166 | TCGTGGCGGTGGACATCCTC | chr9:134053461-134053484_+ |
| | | 167 | ATTTGATTGCGTCCACGGGC | chr9:134053275-134053298_+ |
| | | 168 | ACCTTTGCCCTTTGGAGCAG | chr9:134051592-134051615_+ |
| | | 169 | GAGTGCAAGCGAATGTATGC | chr9:134052346-134052369_− |
| | | 170 | CGCCACGACAGTCGCCCCCG | chr9:134053446-134053469_− |
| BRD4 | ENST00000263377.6 | 171 | GGGTGGCCGCGATGATGGGT | chr19:15265367-15265390_+ |
| | | 172 | TCTTTGGAGGTTTCACAGGC | chr19:15264618-15264641_+ |
| | | 173 | GAGCAGGTATTGCAGTTGGT | chr19:15272898-15272921_+ |
| | | 174 | TTCAGCTTGACGGCATCCAC | chr19:15272821-15272844_+ |
| | | 175 | TGGCTCGTGAATGGGGTCAA | chr19:15264697-15264720_+ |
| BTK | ENST00000308731.7 | 176 | CTTCCTTAGTTCTTCAGTTG | chrX:101370022-101370045_+ |
| | | 177 | TCTCCCCAACTGAAGAACTA | chrX:101370025-101370048_− |
| | | 178 | GAACCAGATCACTGTTGTAC | chrX:101362662-101362685_+ |
| | | 179 | GCCCTTCATCATATACAACC | chrX:101370060-101370083_+ |
| | | 180 | AATCCGGTACAACAGTGATC | chrX:101362665-101362688_− |
| CCND1 | ENST00000227507.2 | 181 | TGGTTTCCACTTCGCAGCAC | chr11:69641327-69641350_− |
| | | 182 | ATGCCAACCTCCTCAACGAC | chr11:69641368-69641391_+ |
| | | 183 | GCACAGGAGCTGGTGTTCCA | chr11:69641311-69641334_− |
| | | 184 | GCGACGATCTTCCGCATGGA | chr11:69641472-69641495_− |
| | | 185 | GGTTGGCATCGGGGTACGCG | chr11:69641354-69641377_− |
| CCND2 | ENST00000261254.7 | 186 | TGCAGATGGGACTTCGGAGT | chr12:4276082-4276105_− |
| | | 187 | CTCGTGGCACAGCAGCTCCA | chr12:4274038-4274061_− |
| | | 188 | CCAGGTAATTCATGGCCAGA | chr12:4276039-4276062_− |
| | | 189 | CAGCTCCATGGCCAGCCCGG | chr12:4274026-4274049_− |
| | | 190 | GCAGAACCTGCTCACCATCG | chr12:4274120-4274143_+ |
| CCND3 | ENST00000372991.8 | 191 | TACTCGGGCAGCGAACAGGC | chr6:41941648-41941671_+ |
| | | 192 | GGGGTACGTAGCGCTCCTCC | chr6:41941528-41941551_+ |
| | | 193 | AACACAGCAGCTCCATACTC | chr6:41941633-41941656_+ |
| | | 194 | GTCTTGCGTCCCCACCCGAA | chr6:41940494-41940517_− |
| | | 195 | ATGGAGCTGCTGTGTTGCGA | chr6:41941626-41941649_− |
| CD19 | ENST00000324662.7 | 196 | ATTACCCACATATCTCTGGC | chr16:28933376-28933399_− |
| | | 197 | CTGGACCCATGTGCACCCCA | chr16:28933312-28933335_+ |
| | | 198 | GTGACGCCTCCCCCAGGAAG | chr16:28936521-28936544_+ |
| | | 199 | AGAGCTGAAGGACGATCGCC | chr16:28933357-28933380_+ |
| | | 200 | CGGGCCACAGCTCAAGACGC | chr16:28933421-28933444_+ |
| CD274 | ENST00000381577.3 | 201 | TCTGAAGTGCAGCATTTCCC | chr9:5457304-5457327_− |
| | | 202 | TTGAAGGACCAGCTCTCCCT | chr9:5457287-5457310_+ |
| | | 203 | TACCGCTGCATGATCAGCTA | chr9:5457359-5457382_+ |
| | | 204 | TGAACATGAACTGACATGTC | chr9:5462885-5462908_+ |
| | | 205 | TGAACTGACATGTCAGGCTG | chr9:5462891-5462914_+ |
| CD38 | ENST00000226279.7 | 206 | TATCAGCCACTAATGAAGTT | chr4:15816592-15816615_+ |
| | | 207 | TGAAAGCATCCCATACACTT | chr4:15816522-15816545_− |
| | | 208 | CCGGGGACAAACCCTGCTGC | chr4:15778442-15778465_+ |
| | | 209 | CTCCTAGAGAGCCGGCAGCA | chr4:15778453-15778476_− |
| | | 210 | CTGGACCTGTGTGAACTGAT | chr4:15824911-15824934_− |
| CDK1 | ENST00000395284.7 | 211 | CCATAGTTAGTCAATGGGTA | chr10:60780146-60780169_− |
| | | 212 | AAGGGTAGACACAAAACTAC | chr10:60784724-60784747_+ |
| | | 213 | ACACAAAACTACAGGTCAAG | chr10:60784732-60784755_+ |
| | | 214 | TATCCCTCCTGGTCAGTACA | chr10:60785747-60785770_+ |
| | | 215 | AGATCTCCAGAAGTATTGCT | chr10:60791907-60791930_+ |
| CDK2 | ENST00000266970.8 | 216 | CCTTAAGCAGAGAGATCTCT | chr12:55967886-55967909_− |
| | | 217 | AAGCAGAGAGATCTCTCGGA | chr12:55967882-55967905_− |
| | | 218 | TGGAATAATATTTGCAGCCC | chr12:55969509-55969532_− |
| | | 219 | CGAGCTCCTGAAATCCTCCT | chr12:55969492-55969515_+ |
| | | 220 | AATAATATTTGCAGCCCAGG | chr12:55969506-55969529_− |
| CDK4 | ENST00000257904.10 | 221 | CTTGCCAGCCGAAACGATCA | chr12:57751205-57751228_− |
| | | 222 | ACCTCACGAACTGTGCTGAT | chr12:57751547-57751570_+ |
| | | 223 | TGCCTATGGGACAGTGTACA | chr12:57751650-57751673_− |
| | | 224 | CACGAACTGTGCTGATGGGA | chr12:57751551-57751574_+ |
| | | 225 | AGCATGTAGACCAGGACCTA | chr12:57751257-57751280_− |
| CDK5 | ENST00000485972.5 | 226 | ATCTCCCGGAGGGCGGAACT | chr7:151056946-151056969_+ |
| | | 227 | TCTGCATCGCGGCGGCCGCG | chr7:151057841-151057864_+ |
| | | 228 | GAGGCTGGATGACGATGATG | chr7:151057070-151057093_− |
| | | 229 | TTTCTCGTATTTCTGCATCG | chr7:151057830-151057853_+ |
| | | 230 | TCCATCGACATGTGGTCAGC | chr7:151055290-151055313_− |
| CDK6 | ENST00000265734.8 | 231 | GAAGAACGGAGGCCGTTTCG | chr7:92833202-92833225_− |
| | | 232 | CCACTGAGGTTAGAGCCATC | chr7:92725624-92725647_+ |
| | | 233 | AGTTCAGATGTTGATCAACT | chr7:92623047-92623070_− |
| | | 234 | AACATTCTGGTGACCAGCAG | chr7:92725692-92725715_− |
| | | 235 | CCGCATCTATAGTTTCCAGA | chr7:92725639-92725662_− |
| CDK7 | ENST00000256443.7 | 236 | TCGGGCTTTACGGCGCCGGA | chr5:69234956-69234979_+ |
| | | 237 | ATTTATGTCCAAAAGCATCA | chr5:69255461-69255484_− |
| | | 238 | ACTTCACGTCCAGAGCCATC | chr5:69234971-69234994_− |
| | | 239 | CAATAGAGCTTATACACATC | chr5:69259903-69259926_+ |
| | | 240 | AACTTTGGGCACACCAACTG | chr5:69269259-69269282_+ |
| CDK9 | ENST00000373264.4 | 241 | GCTTCTAAAACACGAGAATG | chr9:127787555-127787578_+ |
| | | 242 | CGAGCAACAGCTCCGGGGGC | chr9:127788362-127788385_− |
| | | 243 | TCTGCGAGCATGACCTTGCT | chr9:127787994-127788017_+ |
| | | 244 | CGGCCCCCGGAGCTGTTGCT | chr9:127788363-127788386_+ |
| | | 245 | TACCCTTGCAGCGGTTATAG | chr9:127787950-127787973_− |
| CHD1 | ENST00000614616.4 | 246 | ATAGGATGGCTGCTTCTTCA | chr5:98897269-98897292_+ |
| | | 247 | ATTCCTTGGAGGTCTAAATT | chr5:98893581-98893604_− |
| | | 248 | GGCTTCTCAAATGAATGCTG | chr5:98896257-98896280_− |
| | | 249 | TATTATCTGGTGGTTTAATG | chr5:98889107-98889130_+ |
| | | 250 | GCATTGATGAGTATTTTAGC | chr5:98898291-98898314_− |
| CHEK1 | ENST00000428830.6 | 251 | CCAGTTGATGTTTGGTCCTG | chr11:125633299-125633322_+ |
| | | 252 | CAAAATCTCAGACTTTGGCT | chr11:125633169-125633192_+ |
| | | 253 | TTATTTCTGGAGTACTGTAG | chrll:125627784-125627807_+ |
| | | 254 | GAGATTCTTCCATCAACTCA | chr11:125629265-125629288_+ |
| | | 255 | ATGGTATTGGAATAACTCAC | chr11:125629400-125629423_+ |
| CHEK2 | ENST00000328354.10 | 256 | GTTGAGGCTCAGCAGTCTCA | chr22:28734680-28734703_− |
| | | 257 | CGATTATGGGCCCTTCAGGA | chr22:28734416-28734439_− |
| | | 258 | TGCCTGTGGAGAGGTAAAGC | chr22:28711991-28712014_− |
| | | 259 | TGATCAGTCAGTTTATCCTA | chr22:28719434-28719457_− |
| | | 260 | AATACAGAGCTTGTAGGGAA | chr22:28725035-28725058_− |
| CPT1A | ENST00000265641.9 | 261 | TGCAAAAATCAATCGGACTC | chr11:68812443-68812466_− |
| | | 262 | CATCATCACTGGCGTGTACC | chr11:68812548-68812571_− |
| | | 263 | GTGTCTTTGACAGCCGGGAC | chr11:68804009-68804032_+ |
| | | 264 | GTCGGTGAGGCCTCTTATGA | chr11:68799324-68799347_− |
| | | 265 | TTTAATACTTCCCGGATCCC | chr11:68793325-68793348_− |
| CRBN | ENST00000231948.8 | 266 | GCACGATGACGACAGCTGTC | chr3:3174197-3174220_− |
| | | 267 | GCCACCATTTATATGAACAT | chr3:3167647-3167670_+ |
| | | 268 | TGTATGTGATGTCGGCAGAC | chr3:3175162-3175185_+ |
| | | 269 | CCATGTCTGTTTACCCGCAA | chr3:3179683-3179706_+ |
| | | 270 | CGCACCATACTGACTTCTTG | chr3:3174103-3174126_+ |
| CRTC1 | ENST00000321949.12 | 271 | TGGTGTCCAGGCCCGAGGAT | chr19:18745830-18745853_− |
| | | 272 | CGTCATTGTGCTCTGGTGCA | chr19:18749797-18749820_− |
| | | 273 | TGGTGTCCGCGGGTGGTGAG | chr19:18747081-18747104_− |
| | | 274 | CGCGGGTGGTGAGAGGTACA | chr19:18747074-18747097_− |
| | | 275 | CCTCGGGCCTGGACACCAGC | chr19:18745835-18745858_+ |
| CRTC2 | ENST00000368633.1 | 276 | TAACCAGATTGGCTCTGGCC | chr1:153955075-153955098_− |
| | | 277 | GAGCCAATCTGGTTAACATT | chr1:153955083-153955106_+ |
| | | 278 | CATCCTGCCCAGCCGACGTG | chr1:153953265-153953288_− |
| | | 279 | AGAGCCAATCTGGTTAACAT | chr1:153955082-153955105_+ |
| | | 280 | AGTCCCCAGGATACCTACCC | chr1:153953303-153953326_− |
| CSF1R | ENST00000286301.7 | 281 | GGCCACGCAGGAGTAGTTGC | chr5:150077324-150077347_+ |
| | | 282 | TCCTTCCTGGCCAGAAACCC | chr5:150070493-150070516_− |
| | | 283 | TCCTGTGCTAGCACGTTCCA | chr5:150080302-150080325_+ |
| | | 284 | ATCCCAGACCTGCAGCACTT | chr5:150070029-150070052_+ |
| | | 285 | AACGGTGACCTTGCGATGTG | chr5:150080943-150080966_− |
| CSNK2A1 | ENST00000646561.1 | 286 | GATCATAATTGTCATGTCCA | chr20:489781-489804_+ |
| | | 287 | CATCATATTGGCGCTGCTGA | chr20:486379-486402_+ |
| | | 288 | AGCAGCGCCAATATGATGTC | chr20:486374-486397_− |
| | | 289 | GCTTACTGCAAGAGAGGCAA | chr20:487441-487464_− |
| | | 290 | TGAGGATAGCCAAGGTTCTG | chr20:488751-488774_− |
| CSNK2A2 | ENST00000262506.7 | 291 | TAATCAAGATGATTACCAAC | chr16:58196821-58196844_− |
| | | 292 | GACCAAGTTTTCGAACCAGT | chr16:58196806-58196829_+ |
| | | 293 | TTGTCCTGTCCATGGAAGAA | chr16:58167216-58167239_+ |
| | | 294 | GGAACCATTCTTCCATGGAC | chr16:58167220-58167243_− |
| | | 295 | AGCAAGCATGATCTTTCGAA | chr16:58167241-58167264_− |
| CXCR1 | ENST00000295683.2 | 296 | TATTACAGATCCACAGATGT | chr2:218165180-218165203_− |
| | | 297 | TCATCAAAATCCCACATCTG | chr2:218165170-218165193_+ |
| | | 298 | CAGGCTCAGCAGGAACACTA | chr2:218165049-218165072_+ |
| | | 299 | CAGCAGGTAGACATCAGTGA | chr2:218164974-218164997_+ |
| | | 300 | TCTCAGTTTCTAGCATACAG | chr2:218165105-218165128_+ |
| CXCR2 | ENST00000318507.6 | 301 | GGCGGCATCTAGTAGAAAAG | chr2:218134889-218134912_− |
| | | 302 | GGTCAGGGCAAAGAGTAGGT | chr2:218135078-218135101_− |
| | | 303 | CAGGCTCAGCAGGAATACCA | chr2:218134967-218134990_− |
| | | 304 | CAGCAGGTAGACATCAGTGA | chr2:218135042-218135065_− |
| | | 305 | GCGGCATCTAGTAGAAAAGG | chr2:218134888-218134911_− |
| CXCR4 | ENST00000409817.1 | 306 | CTTCTGGGCAGTTGATGCCG | chr2:136115629-136115652_− |
| | | 307 | AGGGAAGCGTGATGACAAAG | chr2:136115650-136115673_+ |
| | | 308 | GCATTTTCTTCACGGAAACA | chr2:136115826-136115849_+ |
| | | 309 | AGGGGACTATGACTCCATGA | chr2:136115851-136115874_− |
| | | 310 | GAAGCGTGATGACAAAGAGG | chr2:136115653-136115676_+ |
| CYP17A1 | ENST00000369887.3 | 311 | TCGCTGACTCTGGCGCACAC | chr10:102835326-102835349_− |
| | | 312 | TGGGCCAAAACAAATAAGCT | chr10:102837306-102837329_+ |
| | | 313 | GCCTGGTGGACCTAGTCCCC | chr10:102834790-102834813_− |
| | | 314 | GGTATCGCCTTCGCTGACTC | chr10:102835336-102835359_− |
| | | 315 | GATTGTCGGCCACCACCAGC | chr10:102837117-102837140_− |
| CYP19A1 | ENST00000396402.5 | 316 | CCAATTCCCATGCAGTAGCC | chr15:51236984-51237007_+ |
| | | 317 | CATTATGTGGAACATACTTG | chr15:51227908-51227931_+ |
| | | 318 | GCGAGTCTGGATCTCTGGAG | chr15:51236877-51236900_− |
| | | 319 | GTGACCATACGAACAAGGCC | chr15:51222491-51222514_+ |
| | | 320 | TGTGACCATACGAACAAGGC | chr15:51222490-51222513_+ |
| DDR2 | ENST00000367922.7 | 321 | TCTCTTGGCGGAACCGTCAT | chr1:162754826-162754849_+ |
| | | 322 | AGATAAATGATGGAACCTCC | chr1:162755710-162755733_− |
| | | 323 | TGTCTTACAATGCTCCAGCT | chr1:162755672-162755695_+ |
| | | 324 | CGATGCCATGACCTCCTGCA | chr1:162754752-162754775_− |
| | | 325 | TCACTCTGGTGGGGACCCAG | chr1:162754727-162754750_+ |
| DHFR | ENST00000439211.6 | 326 | GTAGACATGGTCTGGATAGT | chr5:80637901-80637924_− |
| | | 327 | CCTCCCGCTGCTGTCATGGT | chr5:80654481-80654504_− |
| | | 328 | GACATGGTCTGGATAGTTGG | chr5:80637898-80637921_− |
| | | 329 | CGAACCAACCATGACAGCAG | chr5:80654477-80654500_+ |
| | | 330 | CCAACCATGACAGCAGCGGG | chr5:80654481-80654504_+ |
| DHH | ENST00000649637.1 | 331 | CCTGGGCGCCAGTGGGCCAG | chr12:49094322-49094345_− |
| | | 332 | AGCGGACCCTGGGCGCCAGT | chr12:49094329-49094352_− |
| | | 333 | TTGTGCCCGGCGTGCCAGAG | chr12:49094347-49094370_− |
| | | 334 | GCCATGGATACAGCGGACCT | chr12:49094507-49094530_+ |
| | | 335 | TAGATTGGTCAGGAGAGCCA | chr12:49094491-49094514_+ |
| DHX9 | ENST00000367549.3 | 336 | CAAGGATTCCAGTTGGATTG | chr1:182858834-182858857_− |
| | | 337 | GTCAGACAACTGTACCATCT | chr1:182858168-182858191_+ |
| | | 338 | TGGTGTTCCTGGGCCCACCT | chr1:182853336-182853359_+ |
| | | 339 | ACTTGGTTTTGTCGCTGAGA | chr1:182858789-182858812_− |
| | | 340 | GCGACAAAACCAAGTGGGTG | chr1:182858797-182858820_+ |
| DNMT1 | ENST00000340748.8 | 341 | CCTGAGGTTTCCGTTTGGCA | chr19:10175595-10175618_+ |
| | | 342 | ATAAATGAATGGTGGATCAC | chr19:10155893-10155916_− |
| | | 343 | ACTGAATGCACTTGGGAGGG | chr19:10159897-10159920_+ |
| | | 344 | CTGAATGCACTTGGGAGGGT | chr19:10159898-10159921_+ |
| | | 345 | GAAGCAGGTCAGTTTGTGCT | chr19:10159659-10159682_+ |
| DOT1L | ENST00000398665.7 | 346 | GTCGATAGTGTGCAGGTAGT | chr19:2207648-2207671_− |
| | | 347 | CGGCAGCGGCCACGGGTAGA | chr19:2164236-2164259_− |
| | | 348 | CAGAGGTGCAAAGGGTTTCG | chr19:2206743-2206766_− |
| | | 349 | AGTTGGTGGCAGCAGCAACC | chr19:2193707-2193730_− |
| | | 350 | ATAGTGATGTTTGCAGTTGG | chr19:2193721-2193744_− |
| DPP4 | ENST00000360534.7 | 351 | AGTTACAGAATCACATGGAC | chr2:162038348-162038371_− |
| | | 352 | GTCCATGTGATTCTGTAACT | chr2:162038351-162038374_+ |
| | | 353 | ATGATGAATCCAGTGGAAGA | chr2:162024815-162024838_− |
| | | 354 | GATTATTCAATATCTCCTGA | chr2:162045565-162045588_− |
| | | 355 | CCCGTGGTTCTGCTGAACAA | chr2:162073400-162073423_− |
| DRD2 | ENST00000362072.7 | 356 | TTCCCGTCTGACCCGTTGAA | chr11:113424568-113424591_+ |
| | | 357 | CTTCAACGGGTCAGACGGGA | chr11:113424566-113424589_− |
| | | 358 | CGGCCCTTCAACGGGTCAGA | chr11:113424571-113424594_− |
| | | 359 | GAGGCTGACGATCAGGTAGT | chr11:113424423-113424446_+ |
| | | 360 | TGTGTGCCATCAGCATCGAC | chr11:113418025-113418048_− |
| EDNRA | ENST00000324300.10 | 361 | TGGGTTGATGAGTGGTAACC | chr4:147485821-147485844_− |
| | | 362 | GAAGTAATTTTAGTCTGCTG | chr4:147485888-147485911_− |
| | | 363 | TCCATCTTGAGGCAAATTTG | chr4:147485663-147485686_− |
| | | 364 | ATTTTCATCGTGGGAATGGT | chr4:147485948-147485971_+ |
| | | 365 | TCTGCGCTCTTAGTGTTGAC | chr4:147519956-147519979_+ |
| EGFR | ENST00000275493.6 | 366 | TAAATGCCACCGGCAGGATG | chr7:55156632-55156655_− |
| | | 367 | ATCCCAAGGATGTTATGTTC | chr7:55160165-55160188_− |
| | | 368 | AGCTGTCGGCCCCACAGGCT | chr7:55155863-55155886_− |
| | | 369 | CCTTGCACGTGGCTTCGTCT | chr7:55154024-55154047_− |
| | | 370 | GCAGCGCCCGGAGCACTGCT | chr7:55152563-55152586_− |
| EHMT1 | ENST00000460843.5 | 371 | TCCCGTTGGATCAAAGCCCT | chr9:137777940-137777963_− |
| | | 372 | CGGTGAGAGATGCTGCTCTC | chr9:137776638-137776661_− |
| | | 373 | TTGATCCAACGGGACCTGCT | chr9:137777949-137777972_+ |
| | | 374 | CTCCAGCACATCTGGACCGT | chr9:137762680-137762703_− |
| | | 375 | AGCACTCCCCTCCCCAAGGG | chr9:137717069-137717092_− |
| EHMT2 | ENST00000375537.8 | 376 | GACCATCCCCCGGGGTGACG | chr6:31888125-31888148_− |
| | | 377 | GAGTGATGATGTCCACTCAC | chr6:31892840-31892863_− |
| | | 378 | CGGGCCAAGATGTCAATGAC | chr6:31896437-31896460_− |
| | | 379 | GCCTCATGGTCTCCCGCTTG | chr6:31888460-31888483_+ |
| | | 380 | GAGGAGTGGGAGACGGTGGT | chr6:31892470-31892493_− |
| EPHA2 | ENST00000358432.7 | 381 | TCGCGGCCCCCGCTGTCCTG | chr1:16138091-16138114_+ |
| | | 382 | GTGTGCAAGGCATCGACGCT | chr1:16138274-16138297_+ |
| | | 383 | GCTCACTGTCACACTGGTGC | chr1:16137964-16137987_+ |
| | | 384 | GAGGTGGCACCCTCAGGGGA | chr1:16138336-16138359_+ |
| | | 385 | CGTCCAGCGCAGCTCCACCT | chr1:16138117-16138140_+ |
| EPHB4 | ENST00000358173.7 | 386 | GTGTTAGAGTGGCTATTGGC | chr7:100820217-100820240_+ |
| | | 387 | CAGGGCTCCACCAGGTCCCG | chr7:100819684-100819707_+ |
| | | 388 | TCCTGCAGTGTCTGACATCC | chr7:100818617-100818640_− |
| | | 389 | TCCGGGGTTCGAGGCAGCTG | chr7:100822288-100822311_− |
| | | 390 | GCTGTGATGTTCCTGGCCGA | chr7:100817207-100817230_+ |
| ERBB2 | ENST00000269571.9 | 391 | AGCTCTCCGGCAGAAATGCC | chr17:39712418-39712441_− |
| | | 392 | GCCGAATGTATACCGGCCCT | chr17:39710433-39710456_− |
| | | 393 | CCAGAACCTGCAAGTAATCC | chr17:39715497-39715520_+ |
| | | 394 | GGAGCACTTGCGAGAGGTGA | chr17:39712340-39712363_+ |
| | | 395 | TGCGCCCGAGGGCACTGCTG | chr17:39716329-39716352_+ |
| ERBB3 | ENST00000267101.7 | 396 | GTGGTAGCAGAGCTGCCTAT | chr12:56093442-56093465_− |
| | | 397 | GGCCTGTCCTCCTGACAAGA | chr12:56088576-56088599_+ |
| | | 398 | ACTGTCATTGAAGTGCCGGC | chr12:56088021-56088044_− |
| | | 399 | CGTAGGCCCCCGAAGCACCT | chr12:56093481-56093504_− |
| | | 400 | CCAACCTCCGCGTGGTGCGA | chr12:56085058-56085081_+ |
| ERBB4 | ENST00000342788.8 | 401 | TGTGTTCCAGTGATGGCTGT | chr2:211679134-211679157_− |
| | | 402 | TTTTCTAACCTGGTGACCAT | chr2:211704121-211704144_− |
| | | 403 | TTCTAGTCACTGGTATTCAT | chr2:211712048-211712071_− |
| | | 404 | TGGAGGCTGCTCAGGACCTA | chr2:211725089-211725112_− |
| | | 405 | GCTTGTGATGGCATTGGCAC | chr2:211712154-211712177_− |
| ESR1 | ENST00000440973.5 | 406 | CCCCTACGGCCCCGGGTCTG | chr6:151808145-151808168_+ |
| | | 407 | AGCCTCAGACCCGGGGCCGT | chr6:151808147-151808170_− |
| | | 408 | GCTGCGGCGTTCGGCTCCAA | chr6:151808167-151808190_+ |
| | | 409 | AAATTCAGATAATCGACGCC | chr6:151842599-151842622_+ |
| | | 410 | CCCTTGGATCTGATGCAGTA | chr6:151807949-151807972_− |
| ESR2 | ENST00000341099.5 | 411 | AACTGGCGATGGACCACTAA | chr14:64282701-64282724_+ |
| | | 412 | TAGCGATCTTGCTTCACACC | chr14:64282646-64282669_+ |
| | | 413 | AGTGTACAATCGATAAAAAC | chr14:64268855-64268878_− |
| | | 414 | AATGTGTTGTGGCCAACACC | chr14:64282734-64282757_− |
| | | 415 | CAGTAACAGGGCTGGCGCAA | chr14:64280100-64280123_+ |
| EZH2 | ENST00000320356.6 | 416 | GCAATGAGCTCACAGAAGTC | chr7:148846483-148846506_+ |
| | | 417 | TTTAATGGGATGACTTGTGT | chr7:148832700-148832723_+ |
| | | 418 | CTCACCGAACAGCAGCTCCC | chr7:148826599-148826622_− |
| | | 419 | TGTTGGAAAATCCAAGTCAC | chr7:148832717-148832740_+ |
| | | 420 | ACTTCTGTGAGCTCATTGCG | chr7:148846479-148846502_− |
| F2R | ENST00000319211.4 | 421 | GCTGTTGTCTGCCCGCACCC | chr5:76716360-76716383_+ |
| | | 422 | CCATTGTCCCGGGCTCTGCG | chr5:76716288-76716311_− |
| | | 423 | CGGGTGCGGGCAGACAACAG | chr5:76716358-76716381_− |
| | | 424 | GCCGCCTGCTTCAGTCTGTG | chr5:76716334-76716357_+ |
| | | 425 | CCGGGACAATGGGGCCGCGG | chr5:76716299-76716322_+ |
| FCGR1A | ENST00000369168.4 | 426 | CTGGGAGCAGCTCTACACAG | chr1:149784095-149784118_+ |
| | | 427 | TAGAGCTGCTCCCAGGCAGA | chr1:149784087-149784110_− |
| | | 428 | ACCAGCTTGGAGACAACATG | chr1:149782726-149782749_+ |
| | | 429 | AACCGTAACCTTGCACTGTG | chr1:149784060-149784083_+ |
| | | 430 | GCATGACACCTCAAGGCCAG | chr1:149788412-149788435_− |
| FGF1 | ENST00000621536.4 | 431 | TTCCGGATGGCACAGTGGAT | chr5:142613983-142614006_− |
| | | 432 | ACTTGGCCATGGACACCGAC | chr5:142600716-142600739_− |
| | | 433 | CAGATTAAACTTCTCGGTCA | chr5:142614073-142614096_+ |
| | | 434 | TACTTGGCCATGGACACCGA | chr5:142600717-142600740_− |
| | | 435 | GGCCCCCGTTGCTACAGTAG | chr5:142614021-142614044_+ |
| FGF2 | ENST00000644866.2 | 436 | CTGCCCGCCTTGCCCGAGGA | chr4:122827198-122827221_+ |
| | | 437 | GCCGCTTGGGGTCCTTGAAG | chr4:122827245-122827268_− |
| | | 438 | CGGCTGCCATGGTCCCTGCG | chr4:122827161-122827184_− |
| | | 439 | TAACCGTTACCTGGCTATGA | chr4:122876378-122876401_+ |
| | | 440 | TTGGGGTCCTTGAAGTGGCC | chr4:122827240-122827263_− |
| FGFR1 | ENST00000447712.6 | 441 | AAGCACCTCCATCTCTTTGT | chr8:38421899-38421922_+ |
| | | 442 | GGTTTGGGGTCCCACTGGAA | chr8:38427985-38428008_+ |
| | | 443 | ACCCTGCTTGCAGGATGGGC | chr8:38424663-38424686_+ |
| | | 444 | GACTCCGTGCCCGCAGACTC | chr8:38429749-38429772_− |
| | | 445 | CAAGGTCGGGGACGGCCTAG | chr8:38457365-38457388_+ |
| FGFR2 | ENST00000457416.6 | 446 | CAAGTTTCGCTGCCCAGCCG | chr10:121551366-121551389_− |
| | | 447 | CGCACCTCTAGCGACTCCCC | chr10:121565631-121565654_+ |
| | | 448 | ACCCCAGCTGACCATGGTTA | chr10:121593802-121593825_+ |
| | | 449 | GGTTGGCATTGGGTTCCCCC | chr10:121551349-121551372_+ |
| | | 450 | CACCGGCCCATCCTCCAAGC | chr10:121520135-121520158_− |
| FGFR3 | ENST00000440486.7 | 451 | GGGGACGGAGCAGCGCGTCG | chr4:1794008-1794031_+ |
| | | 452 | GCGGAAGCGGACGGTGTTGG | chr4:1801423-1801446_− |
| | | 453 | AATAGAATTGCCCGCCAGGC | chr4:1803773-1803796_− |
| | | 454 | CTTCGGCAGCGGGGATGCTG | chr4:1799293-1799316_+ |
| | | 455 | ACTCCGGGGCCTACAGCTGC | chr4:1799451-1799474_+ |
| FGFR4 | ENST00000292408.8 | 456 | GATGGAGAGCGTGGTGCCCT | chr5:177091710-177091733_+ |
| | | 457 | CGCTACCTCTGCCTGGCACG | chr5:177090589-177090612_+ |
| | | 458 | GCCAGCGGATGGTGGGCGTG | chr5:177091031-177091054_− |
| | | 459 | AGCTGGACAGCGGAACTTGA | chr5:177091000-177091023_− |
| | | 460 | GTACACGGCCAGCAGGTGCC | chr5:177090513-177090536_− |
| FGR | ENST00000374005.7 | 461 | GAGAGGCAGCTGCTTTCACC | chr1:27617239-27617262_− |
| | | 462 | CTTTCACCAGGCAACCCCCA | chr1:27617227-27617250_− |
| | | 463 | ATGTGGGCAAATGAGGATGC | chr1:27623767-27623790_+ |
| | | 464 | CTCGCTTTCCCGAATGAGAA | chr1:27617202-27617225_+ |
| | | 465 | GAGGCTCGGTCTCTCAGCTC | chr1:27621618-27621641_− |
| FKBP1A | ENST00000400137.8 | 466 | GGGCGCACCTTCCCCAAGCG | chr20:1392859-1392882_− |
| | | 467 | TCAGCGTCCGCCGCCGCCAT | chr20:1392993-1393016_− |
| | | 468 | CGCAGGTCTGGCCGCGCTTG | chr20:1392848-1392871_+ |
| | | 469 | CACCTGCACTCCCATGGCGG | chr20:1392983-1393006_+ |
| | | 470 | CAAGCGCGGCCAGACCTGCG | chr20:1392845-1392868_− |
| FLT1 | ENST00000282397.8 | 471 | GTAAGACCGCTTGCCAGCTA | chr13:28430096-28430119_+ |
| | | 472 | TCCCCGAGCCTCAGATCACT | chr13:28384918-28384941_− |
| | | 473 | GTTAGGTGACGTAACCCGGC | chr13:28438241-28438264_+ |
| | | 474 | CTTGACACTTTGATCCCTGA | chr13:28434191-28434214_− |
| | | 475 | CAGGTGCTTGAAACCGTAGC | chr13:28430109-28430132_− |
| FLT3 | ENST00000241453.11 | 476 | GTCCACGTACATCTGATTTG | chr13:28048328-28048351_+ |
| | | 477 | GGTAACCAAAGCTGATTGAC | chr13:28049390-28049413_+ |
| | | 478 | TCCACGTACATCTGATTTGT | chr13:28048329-28048352_+ |
| | | 479 | CCCAGGTGAGCCCGAATCCA | chr13:28049660-28049683_+ |
| | | 480 | GAGCAAAAGGGTCTTGATAA | chr13:28048280-28048303_− |
| FLT4 | ENST00000261937.10 | 481 | GACGTAGCTGCCTGTGTCGT | chr5:180630624-180630647_+ |
| | | 482 | GTCAAGTTCTGCGTGAGCCG | chr5:180621218-180621241_+ |
| | | 483 | CCCGTAGGCCGTGCAGGTGA | chr5:180625942-180625965_+ |
| | | 484 | GGCATGTGGACTGTGGCGCC | chr5:180626219-180626242_+ |
| | | 485 | TCTTTGTACCACACGATGCT | chr5:180621131-180621154_+ |
| FNTA | ENST00000302279.7 | 486 | GTGACTTCAAAAGAACTCTC | chr8:43069560-43069583_− |
| | | 487 | CTACATCACTGCAATAATTG | chr8:43069611-43069634_+ |
| | | 488 | TGTGGACCAACTTCTGAAAG | chr8:43077247-43077270_+ |
| | | 489 | GGGTCGGGGAGGCTGCGCAA | chr8:43056362-43056385_+ |
| | | 490 | TCTTTTGAAGTCACTTCAGA | chr8:43069569-43069592_+ |
| FOLH1 | ENST00000256999.6 | 491 | CCCGCGCTGGCTGTGCGCTG | chr11:49208336-49208359_− |
| | | 492 | TCACGAAACCGACTCGGCTG | chr11:49208372-49208395_− |
| | | 493 | GTGCTAGCTCAACAGAATCC | chr11:49200331-49200354_+ |
| | | 494 | TCTCCTTCACGAAACCGACT | chr11:49208378-49208401_− |
| | | 495 | CAGCCGAGTCGGTTTCGTGA | chr11:49208375-49208398_+ |
| FOLR1 | ENST00000312293.9 | 496 | AGTGTGGGTGGCTGTAGTAG | chr11:72192211-72192234_+ |
| | | 497 | ATGAAATGCCGTTTGCAGGC | chr11:72195381-72195404_− |
| | | 498 | CCAGTTGAATCTATATAGGT | chr11:72195337-72195360_− |
| | | 499 | CTGCAAACGGCATTTCATCC | chr11:72195386-72195409_+ |
| | | 500 | GACATTGAGAAGCTCAGTCC | chr11:72192258-72192281_− |
| FOLR2 | ENST00000298223.10 | 501 | CTGGGACCACTGCGGCAAGA | chr11:72220955-72220978_+ |
| | | 502 | CGGGCTCCATCTTGCCGCAG | chr11:72220961-72220984_− |
| | | 503 | TGGCATCCATACAGACATTG | chr11:72218664-72218687_− |
| | | 504 | GTCCTGGGCACTGCACATGG | chr11:72218630-72218653_− |
| | | 505 | TGATCTCCTCAATGTCTGTA | chr11:72218658-72218681_+ |
| FOLR3 | ENST00000611028.2 | 506 | CCTGGGGCCCTGGATCCGGC | chr11:72139127-72139150_+ |
| | | 507 | ATAAAGTGGCGCTTGCAGGT | chr11:72139071-72139094_− |
| | | 508 | GGCCATACAGCTCGTCCTCG | chr11:72136095-72136118_− |
| | | 509 | TGGCTTTGGTGACTGCTGCG | chr11:72135989-72136012_+ |
| | | 510 | GTTAAAGTTGTACAGGCGGG | chr11:72139024-72139047_− |
| FRK | ENST00000606080.1 | 511 | AGATGTTGCTCATTGTGCCT | chr6:116060298-116060321_+ |
| | | 512 | GGCTGAGGACAGAAGCCTAC | chr6:116059974-116059997_− |
| | | 513 | TCAACCGTGATTGAAAATCC | chr6:116060213-116060236_− |
| | | 514 | GTTGCTCATTGTGCCTTGGT | chr6:116060302-116060325_+ |
| | | 515 | GGCTCCAGTCAGCAACTACA | chr6:116060018-116060041_− |
| FYN | ENST00000354650.7 | 516 | CGAAGCTGGGGTAGTGCTGA | chr6:111719924-111719947_+ |
| | | 517 | TTACATTCCCAGCAATTATG | chr6:111707949-111707972_− |
| | | 518 | GTCCTTGGCCCCCGGCTGCG | chr6:111719870-111719893_+ |
| | | 519 | CCGAAGCTGGGGTAGTGCTG | chr6:111719923-111719946_+ |
| | | 520 | AATGGGCTGTGTGCAATGTA | chr6:111720029-111720052_− |
| FZD8 | ENST00000374694.2 | 521 | GCGCCGCTCATGCGCCAGTA | chr10:35641052-35641075_− |
| | | 522 | GCAGCGCTCTAGCGGCGCTG | chr10:35641350-35641373_− |
| | | 523 | GAGGCGCACAGCATGGAGTG | chr10:35641418-35641441_− |
| | | 524 | GATGCCCTTACACAGCGGCA | chr10:35641291-35641314_+ |
| | | 525 | TAGCCGATGCCCTTACACAG | chr10:35641286-35641309_+ |
| GART | ENST00000381839.7 | 526 | TCGCTTATGGTCCTGTGCTG | chr21:33530821-33530844_+ |
| | | 527 | CACCGCCCTAACTGTTGTCA | chr21:33528214-33528237_− |
| | | 528 | GGAATAATGCTGACCAAGAA | chr21:33528567-33528590_− |
| | | 529 | AAACAAGTGTTGGTTGCCCC | chr21:33539211-33539234_− |
| | | 530 | CGCTTATGGTCCTGTGCTGG | chr21:33530822-33530845_+ |
| GNRH1 | ENST00000276414.4 | 531 | TCCTCCAGGGCGCAGTCCAT | chr8:25423228-25423251_+ |
| | | 532 | CTACTGACTTGGTGCGTGGA | chr8:25423271-25423294_− |
| | | 533 | TATTCTACTGACTTGGTGCG | chr8:25423275-25423298_− |
| | | 534 | AGCTCCTTTCAGGTCTCGGA | chr8:25421575-25421598_+ |
| | | 535 | GGGAGAACGTGGCTGGTGCG | chr8:25421596-25421619_+ |
| GNRHR | ENST00000226413.4 | 536 | CTGATTGTCATGCCACTGGA | chr4:67754039-67754062_− |
| | | 537 | TCATGCCACTGGATGGGATG | chr4:67754032-67754055_− |
| | | 538 | GTAACTCTCCAGCATACCAT | chr4:67753998-67754021_+ |
| | | 539 | TGATTGTCATGCCACTGGAT | chr4:67754038-67754061_− |
| | | 540 | AAAGACACTACTGAGGATCC | chr4:67753825-67753848_+ |
| GSK3A | ENST00000222330.7 | 541 | TACACGGACATCAAAGTGAT | chr19:42240048-42240071_− |
| | | 542 | CTCTGGGCCTTGGCCTAGAG | chr19:42240089-42240112_+ |
| | | 543 | CACTAGCTTCCCGCCGCCCG | chr19:42242194-42242217_− |
| | | 544 | TTGGGGTCGTGTACCAGGCA | chr19:42240014-42240037_− |
| | | 545 | TCACGGCCCAGCTTCACCCC | chr19:42242178-42242201_+ |
| GSK3B | ENST00000316626.5 | 546 | CGTTATTTCTTCTACTCCAG | chr3:119947274-119947297_− |
| | | 547 | CGGCTTGCAGCTCTCCGCAA | chr3:120093386-120093409_+ |
| | | 548 | CTCCTGGGCAGGGTCCAGAC | chr3:120002177-120002200_− |
| | | 549 | TTCATGCTGCCAAAAGCTGA | chr3:120093354-120093377_+ |
| | | 550 | CAACAGTGGTGGCAACTCCT | chr3:120002192-120002215_− |
| HCK | ENST00000534862.6 | 551 | GCTGCCCGCGAGACGAGGAG | chr20:32052455-32052478_+ |
| | | 552 | AGGACAGTGTGGGCTGGCGC | chr20:32071723-32071746_− |
| | | 553 | AGGCTCGATCCCTGGCCACC | chr20:32074639-32074662_+ |
| | | 554 | AGAATGTATTGCCTCCGACC | chr20:32071688-32071711_− |
| | | 555 | GCGCCCGCTCCTCGTCTCGC | chr20:32052459-32052482_− |
| HDAC1 | ENST00000373548.7 | 556 | TTCGGTGAGGCTTCATTGGG | chr1:32302651-32302674_− |
| | | 557 | CCCAGGAACTGGGGACCTAC | chr1:32327655-32327678_+ |
| | | 558 | AGTAGTAACAGACTTTCCTC | chr1:32292189-32292212_− |
| | | 559 | GTACTCTCCATACTTATGAA | chr1:32327630-32327653_− |
| | | 560 | TTACGTCAATGATATCGTCT | chr1:32327035-32327058_+ |
| HDAC10 | ENST00000216271.9 | 561 | GTGTAGCCCGTGTTTCTGCT | chr22:50249861-50249884_+ |
| | | 562 | CTCCCGGGCACCATGGCCAG | chr22:50249939-50249962_− |
| | | 563 | TGTGCAAAATGGGCTTGCCC | chr22:50250066-50250089_− |
| | | 564 | GAGTCAGATGCAGACGCAGT | chr22:50249373-50249396_− |
| | | 565 | CAGCGTTTCCCATCCCAACC | chr22:50249149-50249172_+ |
| HDAC11 | ENST00000295757.7 | 566 | CAACATCACCTTCATGGGCC | chr3:13481314-13481337_+ |
| | | 567 | CAAAGGGATGCAGCTTCTCC | chr3:13481332-13481355_− |
| | | 568 | CCCTTTGATGCCGGAAAATG; | chr3:13481348-13481371_+ |
| | | 569 | AAGCTGCATCCCTTTGATGC | chr3:13481339-13481362_+ |
| | | 570 | AACGGGGGGGATTTCTGTGA | chr3:13496754-13496777_− |
| HDAC2 | ENST00000519065.5 | 571 | GATATGGCTGTTAATTGGGC | chr6:113956096-113956119_− |
| | | 572 | GAGCCCATGGCGTACAGTCA | chr6:113970891-113970914_− |
| | | 573 | GGGGAATACTTTCCTGGCAC | chr6:113953286-113953309_− |
| | | 574 | TCTTCGGCAGTGGCTTTATG | chr6:113958740-113958763_+ |
| | | 575 | TTCCTGGCACAGGAGACTTG | chr6:113953276-113953299_− |
| HDAC3 | ENST00000305264.7 | 576 | CGAGCAGAACTCAAAGAGCC | chr5:141630091-141630114_+ |
| | | 577 | TCATGTTGGGAGGCCTGGTA | chr5:141634921-141634944_+ |
| | | 578 | TGGTGGGGCTGACTCTCTGC | chr5:141634862-141634885_+ |
| | | 579 | CCAGGCGATGGGGCTTCATA | chr5:141636597-141636620_+ |
| | | 580 | GATATTGCCATTAACTGGGC | chr5:141629887-141629910_− |
| HDAC4 | ENST00000345617.7 | 581 | GTCGACACTCCGCTCTGGGG | chr2:239156716-239156739_+ |
| | | 582 | GCCTGGGGCGCTGCTGCACG | chr2:239139770-239139793_+ |
| | | 583 | GTGACGAGGGGTGCTTGTGC | chr2:239134246-239134269_+ |
| | | 584 | CAAGGGCGAGGTGCTCAGGT | chr2:239134332-239134355_+ |
| | | 585 | CTCCACGCACAGTCCTTGGT | chr2:239126639-239126662_− |
| HDAC5 | ENST00000225983.10 | 586 | TGTGCAGAGAAGTCCGCGGC | chr17:44110743-44110766_+ |
| | | 587 | AGCAGGGAGGCATGCCCGTG | chr17:44091322-44091345_+ |
| | | 588 | CGGGCAGTCCCCACTAGTGA | chr17:44088565-44088588_− |
| | | 589 | GCCCTCCAGTCCCTGCGGCA | chr17:44091427-44091450_− |
| | | 590 | CACGTTCACCCGTCACTAGT | chr17:44088556-44088579_+ |
| HDAC6 | ENST00000334136.10 | 591 | GTTAGCTGGGCGAACCCTGC | chrX:48814979-48815002_− |
| | | 592 | CTGGTTCCAAGGCACATTGA | chrX:48814543-48814566_− |
| | | 593 | CTGAGTCGTAGGTGTCTGCT | chrX:48806427-48806450_− |
| | | 594 | GATATACCATCAATGTGCCT | chrX:48814537-48814560_+ |
| | | 595 | CTTGAGGCTGAAGCACTGGC | chrX:48803136-48803159_+ |
| HDAC7 | ENST00000080059.11 | 596 | TGACCACCGAGCGGCTCTCT | chr12:47795312-47795335_− |
| | | 597 | GTGGGCACCCGGGCTCACCT | chr12:47802245-47802268_+ |
| | | 598 | TAAGGACTGGGCAAAGTGGA | chr12:47795336-47795359_+ |
| | | 599 | GGCTGCAGTAGTGGGCACCC | chr12:47802235-47802258_+ |
| | | 600 | CAGCGGGGCATGAGAGCCTG | chr12:47795593-47795616_+ |
| HDAC8 | ENST00000647594.1 | 601 | GCTGCCCAATGCCTGATTGA | chrX:72567939-72567962_− |
| | | 602 | ACATTCCGTCAATCAGGCAT | chrX:72567934-72567957_+ |
| | | 603 | CATTCCGTCAATCAGGCATT | chrX:72567935-72567958_+ |
| | | 604 | ATCCGGACTCCATAGAATAT | chrX:72568757-72568780_− |
| | | 605 | CCTGGCCAAGATCCCCAAAC | chrX:72572649-72572672_− |
| HDAC9 | ENST00000417496.6 | 606 | AGGTCTGTCCTTAGGTCTAA | chr7:18585324-18585347_− |
| | | 607 | CTAAAGGTGAGATGGGCTCC | chr7:18585308-18585331_− |
| | | 608 | CTTAGGTCTAAAGGTGAGAT | chr7:18585315-18585338_− |
| | | 609 | CCATCTCACCTTTAGACCTA | chr7:18585316-18585339_+ |
| | | 610 | TCAGCTTCAGGAGCATATCA | chr7:18585500-18585523_+ |
| HPRT1 | ENST00000298556.7 | 611 | GTCGCCATAACGGAGCCGGC | chrX:134460296-134460319_− |
| | | 612 | GCGGGTCGCCATAACGGAGC | chrX:134460300-134460323_− |
| | | 613 | CTCATGGACTAATTATGGAC | chrX:134473443-134473466_+ |
| | | 614 | AAAATCTACAGTCATAGGAA | chrX:134475320-134475343_− |
| | | 615 | GCCCCCCTTGAGCACACAGA | chrX:134475236-134475259_− |
| HPSE | ENST00000405413.6 | 616 | CTGGCAATCTCAAGTCAACC | chr4:83322218-83322241_− |
| | | 617 | CCTTGGAAGAGCAGTAGTCC | chr4:83313143-83313166_+ |
| | | 618 | TGGCGTCAATGGTGACGGAC | chr4:83334592-83334615_+ |
| | | 619 | ACGACGTCCTGTGCTTGCGC | chr4:83334666-83334689_+ |
| | | 620 | CATTGACGCCAACCTGGCCA | chr4:83334580-83334603_− |
| HSP90AA1 | ENST00000216281.12 | 621 | ACGATGATGAGCAGTACGCT | chr14:102085800-102085823_− |
| | | 622 | GATCAAAAGGAGCACGTCGT | chr14:102084494-102084517_+ |
| | | 623 | TCTCACGGGATATGTTTAGA | chr14:102083926-102083949_+ |
| | | 624 | AGATCAAAAGGAGCACGTCG | chr14:102084493-102084516_+ |
| | | 625 | CGATGATGAGCAGTACGCTT | chr14:102085799-102085822_− |
| HSPA1A | ENST00000375651.6 | 626 | CACCACCTACTCCTGCGTGG | chr6:31815791-31815814_+ |
| | | 627 | GTGTTGGAACACCCCCACGC | chr6:31815802-31815825_− |
| | | 628 | GACCAAGGCATTCTACCCCG | chr6:31816085-31816108_+ |
| | | 629 | CAACGACGGAGACAAGCCCA | chr6:31816040-31816063_+ |
| | | 630 | GGACACCGAGCGGCTCATCG | chr6:31815890-31815913_+ |
| HSPA1B | ENST00000375650.4 | 631 | CAGGTCGATGCCGATCGCCG | chr6:31827960-31827983_− |
| | | 632 | CACCACCTACTCCTGCGTGG | chr6:31827985-31828008_+ |
| | | 633 | GTGTTGGAACACCCCCACGC | chr6:31827996-31828019_− |
| | | 634 | CAACGACGGAGACAAGCCCA | chr6:31828234-31828257_+ |
| | | 635 | GGACACCGAGCGGCTCATCG | chr6:31828084-31828107_+ |
| HSPB1 | ENST00000248553.6 | 636 | GCATAGCCGCCTCTTCGACC | chr7:76302783-76302806_+ |
| | | 637 | GAGTGGTCGCAGTGGTTAGG | chr7:76302832-76302855_+ |
| | | 638 | TGCCGGAGGAGTGGTCGCAG | chr7:76302824-76302847_+ |
| | | 639 | CAGCCGGCAACTCAGCAGCG | chr7:76302942-76302965_+ |
| | | 640 | CGGGCTGCCCCGGCTGCCGG | chr7:76302810-76302833_+ |
| IDH1 | ENST00000345146.6 | 641 | TGGGTAAAACCTATCATCAT | chr2:208248390-208248413_− |
| | | 642 | ACCCATCCACTCACAAGCCG | chr2:208248408-208248431_+ |
| | | 643 | AATATCCCCCGGCTTGTGAG | chr2:208248414-208248437_− |
| | | 644 | CCCCCGGCTTGTGAGTGGAT | chr2:208248409-208248432_− |
| | | 645 | CCCATCCACTCACAAGCCGG | chr2:208248409-208248432_+ |
| IDH2 | ENST00000330062.7 | 646 | GATGTTCCGGATAGTTCCAT | chr15:90088694-90088717_+ |
| | | 647 | CCAAGCCCATCACCATTGGC | chr15:90088604-90088627_− |
| | | 648 | GTTCGCTCTCCAGCTTGGGA | chr15:90102386-90102409_− |
| | | 649 | TTGGGATGGCCGGCTACCTG | chr15:90102372-90102395_− |
| | | 650 | AACATCCCACGCCTAGTCCC | chr15:90088632-90088655_− |
| IDO1 | ENST00000522495.5 | 651 | CACACGCTATGGAAAACTCC | chr8:39913926-39913949_+ |
| | | 652 | ATGGCATATGTGTGGGGCAA | chr8:39918165-39918188_+ |
| | | 653 | CTTTGCTCTGCCAAATCCAC | chr8:39913987-39914010_+ |
| | | 654 | GCATCACCATGGCATATGTG | chr8:39918157-39918180_+ |
| | | 655 | CATCACCATGGCATATGTGT | chr8:39918158-39918181_+ |
| IFNAR1 | ENST00000270139.7 | 656 | TGGGTGTTGTCCGCAGCCGC | chr21:33325109-33325132_+ |
| | | 657 | AGTGTTATGTGGGCTTTGGA | chr21:33343347-33343370_+ |
| | | 658 | ATAGTGATACACATCTCTCC | chr21:33343314-33343337_+ |
| | | 659 | GAACAAAAGATAGTGTTATG | chr21:33343336-33343359_+ |
| | | 660 | AAGCAGCACTACTTACGTCA | chr21:33343610-33343633_+ |
| IFNAR2 | ENST00000342136.8 | 661 | TACAGCAATGTATAGTGAGT | chr21:33245039-33245062_− |
| | | 662 | TGTATAGTGAGTTGGTACAA | chr21:33245031-33245054_− |
| | | 663 | AGTGAGTTGGTACAATGGAG | chr21:33245026-33245049_− |
| | | 664 | CATATGAAATACCAAACACG | chr21:33243682-33243705_− |
| | | 665 | AAACCAACAATCTCAAACTC | chr21:33248722-33248745_− |
| IGF1R | ENST00000650285.1 | 666 | CTCTCGCTCTGGCCGACGAG | chr15:98649647-98649670_+ |
| | | 667 | CAGCCCATGTAGTAAGATGC | chr15:98913144-98913167_− |
| | | 668 | GTTTTGGGGCAGGCGCAGCA | chr15:98916766-98916789_− |
| | | 669 | GTAAACGGCGTACTGAGTCC | chr15:98913171-98913194_− |
| | | 670 | ACACATCGGCTTCTCCTCCA | chr15:98708020-98708043_− |
| IHH | ENST00000295731.6 | 671 | CTGAACTGCTTGTAGGCGAG | chr2:219060309-219060332_+ |
| | | 672 | GCAGCTTCACACCGGGCCAC | chr2:219057627-219057650_+ |
| | | 673 | CCGCAATAAGTATGGACTGC | chr2:219057510-219057533_− |
| | | 674 | TGTGAAGCTGCGGGTGACCG | chr2:219057615-219057638_− |
| | | 675 | GTGAAGCTGCGGGTGACCGA | chr2:219057614-219057637_− |
| IKBKB | ENST00000520810.6 | 676 | AGTCTTTGCACATCATTCGT | chr8:42306393-42306416_+ |
| | | 677 | ACTGCCAAGGAGGAGATCTC | chr8:42290247-42290270_+ |
| | | 678 | ATCTCGGGCAGCCACCACAT | chr8:42290166-42290189_− |
| | | 679 | TGAAAGAGCGCCTTGGGACA | chr8:42272149-42272172_+ |
| | | 680 | GGGCAGCCACCACATTGGGG | chr8:42290161-42290184_− |
| IL1B | ENST00000263341.6 | 681 | GATCACTGAACTGCACGCTC | chr2:112832746-112832769_− |
| | | 682 | GCAGGCCGCGTCAGTTGTTG | chr2:112833466-112833489_− |
| | | 683 | TCCCATGTGTCGAAGAAGAT | chr2:112832801-112832824_+ |
| | | 684 | CTACAGCAAGGGCTTCAGGC | chr2:112833484-112833507_− |
| | | 685 | GTGCAGTTCAGTGATCGTAC | chr2:112832753-112832776_+ |
| IL2RA | ENST00000379959.7 | 686 | CTCACGTTCATCATGGTGCC | chr10:6062098-6062121_− |
| | | 687 | TCACTCTATATGCTCTGTAC | chr10:6025883-6025906_− |
| | | 688 | GGGACTGCTCACGTTCATCA | chr10:6062105-6062128_− |
| | | 689 | ATGGCTTTGAATGTGGCGTG | chr10:6025973-6025996_+ |
| | | 690 | GCAAAGTCCAATGCAGCCAG | chr10:6024261-6024284_− |
| 1L2RB | ENST00000216223.9 | 691 | CGGCCAGGCATGGACTTGGC | chr22:37143528-37143551_+ |
| | | 692 | GGCCCAGGATGCTTGACTCA | chr22:37142460-37142483_+ |
| | | 693 | CCCATCTTGGCTCCAGACAC | chr22:37143567-37143590_+ |
| | | 694 | ACTCACGGGGAGCAGCTCAC | chr22:37142475-37142498_+ |
| | | 695 | CTGTGTCTGGAGCCAAGATG | chr22:37143566-37143589_− |
| 1L2RG | ENST00000374202.6 | 696 | TTGTTCAGCTCCAGGACCCA | chrX:71110549-71110572_− |
| | | 697 | AAACACTGAACCTCTGGGAG | chrX:71110977-71111000_+ |
| | | 698 | CCTGTCTCCTGGGTTCCCGT | chrX:71110533-71110556_+ |
| | | 699 | AAAACACTGAACCTCTGGGA | chrX:71110976-71110999_+ |
| | | 700 | TTTCTTCAGAGAATAGATAG | chrX:71110626-71110649_+ |
| IL6 | ENST00000404625.5 | 701 | GTTCATAGCTGGGCTCCTGG | chr7:22727245-22727268_− |
| | | 702 | TGGAGAAGGAGTTCATAGCT | chr7:22727255-22727278_− |
| | | 703 | GGAAGGCAGCAGGCAACACC | chr7:22727480-22727503_− |
| | | 704 | TTTGTCAATTCGTTCTGAAG | chr7:22727566-22727589_− |
| | | 705 | CTTCCAATCTGGATTCAATG | chr7:22728784-22728807_+ |
| IL6R | ENST00000368485.7 | 706 | CAGCAATGTTGTTTGTGAGT | chr1:154430528-154430551_+ |
| | | 707 | GCACGGCTCCTGGAAGTCTT | chr1:154434532-154434555_− |
| | | 708 | AGCCTTTGTCGTCAGGGATG | chr1:154430563-154430586_− |
| | | 709 | AGCAATGTTGTTTGTGAGTG | chr1:154430529-154430552_+ |
| | | 710 | TTGTTTGTGAGTGGGGTCCT | chr1:154430536-154430559_+ |
| INHA | ENST00000243786.2 | 711 | GGGGCCCCCCGCGGTGACCA | chr2:219572496-219572519_+ |
| | | 712 | GGGGCAGCCGCCTGACTCCA | chr2:219572529-219572552_− |
| | | 713 | GTGCAGCACCATAGCTCACC | chr2:219572363-219572386_− |
| | | 714 | TAGCAGGGCCAGGTGAGCTA | chr2:219572355-219572378_+ |
| | | 715 | CCTGTGTGTGAAGCCCCCCA | chr2:219572561-219572584_− |
| INHBA | ENST00000242208.4 | 716 | TTCCCCCACCCCAGGATCCG | chr7:41700292-41700315_− |
| | | 717 | TTGGCTGAGAGGATTTCTGT | chr7:41700340-41700363_− |
| | | 718 | AGTCGGGGAGAACGGGTATG | chr7:41700070-41700093_− |
| | | 719 | AGATAGAGGATGACATTGGA | chr7:41700047-41700070_− |
| | | 720 | GTGAGGAGTTCCCCCACCCC | chr7:41700300-41700323_− |
| INHBB | ENST00000295228.3 | 721 | CGGCGTGCGTGATGTTGGGC | chr2:120346457-120346480_− |
| | | 722 | TCGTCGCCAGCGGCGCACCA | chr2:120346169-120346192_+ |
| | | 723 | CGTCGTGCGGCGGCTTCCGG | chr2:120346354-120346377_+ |
| | | 724 | GACACCTGTACGTCGTGCGG | chr2:120346344-120346367_+ |
| | | 725 | CAGGACACCTGTACGTCGTG | chr2:120346341-120346364_+ |
| INHBC | ENST00000309668.2 | 726 | CTCAAAGCAGCTCTGGACAC | chr12:57435081-57435104_− |
| | | 727 | CCGCTGGCTCTCCAGTTCCA | chr12:57434983-57435006_− |
| | | 728 | GGTCAGTGTCCAGCATGTGG | chr12:57434955-57434978_+ |
| | | 729 | TGGCTCTCCAGTTCCAAGGT | chr12:57434979-57435002_− |
| | | 730 | GTCAGTGTCCAGCATGTGGG | chr12:57434956-57434979_+ |
| INHBE | ENST00000266646.2 | 731 | TTATTCTGGGACGACTGGTC | chr12:57455703-57455726_− |
| | | 732 | TGGTGCGAGCACAGGGGACA | chr12:57455582-57455605_+ |
| | | 733 | GCCCTCCGGAGACTACAGCC | chr12:57455758-57455781_+ |
| | | 734 | ATGAGTTATTCTGGGACGAC | chr12:57455708-57455731_− |
| | | 735 | GGCCATTCACCAGGAGCATG | chr12:57455519-57455542_+ |
| ITK | ENST00000422843.7 | 736 | GAAGCGGACTTTAAAGTTCG | chr5:157181044-157181067_− |
| | | 737 | CTACCAAACCAATGATCCTC | chr5:157222909-157222932_+ |
| | | 738 | GACGATCTTCAAAGTATGCC | chr5:157181087-157181110_− |
| | | 739 | TGGACAGTTCTGAGATTCAC | chr5:157222968-157222991_+ |
| | | 740 | ATCTTCAAAGTATGCCAGGC | chr5:157181083-157181106_− |
| JAK1 | ENST00000342505.4 | 741 | GTCCATCCTGCTCGGTCTTG | chr1:64869410-64869433_+ |
| | | 742 | TGGGGTCTCGAATAGGAGCC | chr1:64869428-64869451_+ |
| | | 743 | CAGCTGGCTCATGGGGTAGA | chr1:64850839-64850862_+ |
| | | 744 | TCGAAACTCAGCTGGCTCAT | chr1:64850831-64850854_+ |
| | | 745 | GATATTGAGAACGAGTGTCT | chr1:64869385-64869408_− |
| JAK2 | ENST00000381652.3 | 746 | TGTATATTTTCAAGCACGGC | chr9:5064993-5065016_− |
| | | 747 | TCTTTTGAATTGTTACCAGA | chr9:5069122-5069145_+ |
| | | 748 | GTTCTGAAAAAGACTCTGCA | chr9:5021968-5021991_+ |
| | | 749 | AAGAAAGCAGGTAATCAGAC | chr9:5066702-5066725_+ |
| | | 750 | TGTACTTCGATGCAGTCCTA | chr9:5066731-5066754_+ |
| JAK3 | ENST00000458235.5 | 751 | CGGCGGCATCGCCTGGACCC | chr19:17842326-17842349_− |
| | | 752 | CAGCCCACCCACATCATCCT | chr19:17838333-17838356_− |
| | | 753 | CATGTGCTGCTGCCCGCTCG | chr19:17844316-17844339_− |
| | | 754 | GGCGGCATCGCCTGGACCCA | chr19:17842325-17842348_− |
| | | 755 | GCAAAGAGGGAGTGGTACAC | chr19:17843869-17843892_+ |
| KDM1A | ENST00000356634.7 | 756 | TGTCCGTTGGCTTCATAAAG | chr1:23059140-23059163_− |
| | | 757 | GCTGGGCCCGACAGGCCCGC | chr1:23019716-23019739_− |
| | | 758 | GCGGTTCCGCCAGGCCCCCG | chr1:23019805-23019828_− |
| | | 759 | CGGAACCGCCGGGGTCCGCA | chr1:23019819-23019842_+ |
| | | 760 | CTGCTTCTTGAGAAGTCATC | chr1:23050426-23050449_− |
| KDR | ENST00000645273.1 | 761 | ATGTCTGCCTTGCTCAAGAC | chr4:55104688-55104711_− |
| | | 762 | GTATCCAAGTTCTTGCAAAC | chr4:55104806-55104829_+ |
| | | 763 | CTGCACAGGTGTACAATCCT | chr4:55113354-55113377_+ |
| | | 764 | ACCATTTTATTTCTGGGGGT | chr4:55110651-55110674_+ |
| | | 765 | TACTTGTCGTCTGATTCTCC | chr4:55102457-55102480_+ |
| KIF11 | ENST00000260731.4 | 766 | TCCTGCATCTCTCAATCTTG | chr10:92613597-92613620_+ |
| | | 767 | ACGTGGAATTATACCAGCCA | chr10:92609023-92609046_− |
| | | 768 | TAGTGTACGAACTGGAGGAT | chr10:92606336-92606359_+ |
| | | 769 | AATCCCTGTTGACTTTGGGA | chr10:92613455-92613478_+ |
| | | 770 | TGAAGAGTATACCTGGGAAG | chr10:92607215-92607238_+ |
| KIT | ENST00000288135.5 | 771 | ACCAATCTATTGTGGGCTCT | chr4:54723650-54723673_− |
| | | 772 | CTCATCGCGGTAGCTGCGAT | chr4:54657996-54658019_− |
| | | 773 | TGACTTCAATTATGAACGTC | chr4:54703761-54703784_+ |
| | | 774 | CTACTGCTTCGCGTCCAGAC | chr4:54658059-54658082_+ |
| | | 775 | CAGAACGCAGAGAAAATCCC | chr4:54658033-54658056_− |
| KRAS | ENST00000256078.8 | 776 | AGGGACCAGTACATGAGGAC | chr12:25227299-25227322_− |
| | | 777 | TCTCGACACAGCAGGTCAAG | chr12:25227336-25227359_− |
| | | 778 | CAATGAGGGACCAGTACATG | chr12:25227304-25227327_− |
| | | 779 | GGACCAGTACATGAGGACTG | chr12:25227297-25227320_− |
| | | 780 | GGACTCTGAAGATGTACCTA | chr12:25225729-25225752_− |
| LAP3 | ENST00000226299.8 | 781 | CCTCCACAGACGAGAGCTCC | chr4:17583504-17583527_− |
| | | 782 | ACGACTACTCGCCCCGCAGC | chr4:17577483-17577506_− |
| | | 783 | GCAAGAACATCTTGTCGGCT | chr4:17577452-17577475_− |
| | | 784 | CTGGACCACCTCTGAAGGCA | chr4:17581761-17581784_+ |
| | | 785 | GAACAGGAAAACTGGCATGA | chr4:17582341-17582364_+ |
| LCK | ENST00000336890.9 | 786 | TTGCCATCCAGTGGGACTAT | chr1:32274404-32274427_− |
| | | 787 | TCCCTGACCACGGGCCAGGA | chr1:32275345-32275368_+ |
| | | 788 | TGTGGCCAAAGCGAACAGCC | chr1:32275386-32275409_+ |
| | | 789 | CGGGAGAGCGAGAGCACCGC | chr1:32275650-32275673_+ |
| | | 790 | AAGGCGCAGTCCCTGACCAC | chr1:32275336-32275359_+ |
| LDLR | ENST00000558518.5 | 791 | CGCGGCGAGGAGCAAGGCGA | chr19:11089579-11089602_− |
| | | 792 | CCCCGCCGCGGCGAGGAGCA | chr19:11089585-11089608_− |
| | | 793 | AGGGGTCTTTACGTGTTCCA | chr19:11105458-11105481_+ |
| | | 794 | CCTGGGGCTGGAAATTGCGC | chr19:11089555-11089578_+ |
| | | 795 | GCGGGACCACAGGTGAGCAC | chr19:11105335-11105358_− |
| LHCGR | ENST00000294954.11 | 796 | GCTGCCACGAGCGCTGCGCG | chr2:48755589-48755612_− |
| | | 797 | AGCTTTCAGAGGACTTAATG | chr2:48731236-48731259_− |
| | | 798 | GGAAGATTTATAAATGCTCC | chr2:48725690-48725713_+ |
| | | 799 | AGTCGAGTGAGACCGGCCGT | chr2:48755510-48755533_+ |
| | | 800 | GATCACTTTGACAGGGAGGT | chr2:48731267-48731290_+ |
| LIMK1 | ENST00000336180.6 | 801 | TCTCATAGTACTGGTGCGAC | chr7:74096642-74096665_− |
| | | 802 | CGCTTGCCATGAGATGAGGC | chr7:74099134-74099157_− |
| | | 803 | TGACGGGGGTCACCACAGTC | chr7:74099046-74099069_− |
| | | 804 | GCCCATCCGAAATGTGCCCC | chr7:74105952-74105975_+ |
| | | 805 | GATCCGGTCTCCGACGTGGA | chr7:74105912-74105935_− |
| LYN | ENST00000519728.5 | 806 | TAACCAGGAAGGACGCAGAA | chr8:55950697-55950720_+ |
| | | 807 | CTTTGCTGTTTATTGGACGT | chr8:55941964-55941987_− |
| | | 808 | TTTCAAGGATATAACCAGGA | chr8:55950686-55950709_+ |
| | | 809 | AAAGACAGCTTGAGTGACGA | chr8:55941883-55941906_+ |
| | | 810 | CAACGTCCAATAAACAGCAA | chr8:55941965-55941988_+ |
| MAP1A | ENST00000300231.5 | 811 | CTCTAGCAGTTACAGCGACT | chr15:43521820-43521843_+ |
| | | 812 | TCCACTGGGGACTGATGAAA | chr15:43524497-43524520_− |
| | | 813 | CTCCCGTACTGAAGCTACGC | chr15:43524103-43524126_+ |
| | | 814 | AGAAATGGGGCATCTGATGC | chr15:43525177-43525200_+ |
| | | 815 | TGAGGCAGGTCGTGGCAGAT | chr15:43528206-43528229_− |
| MAP2 | ENST00000360351.8 | 816 | AAAGTTAAATCCAAGGGCTA | chr2:209694301-209694324_− |
| | | 817 | AATTAGTGACTTTGGACAGA | chr2:209695316-209695339_+ |
| | | 818 | TATTTCCACTGACAATTTGA | chr2:209695666-209695689_− |
| | | 819 | ACTTTGGACAGATGGCTTCA | chr2:209695324-209695347_+ |
| | | 820 | TGTCTCTGGCTGAGAAACTA | chr2:209692756-209692779_− |
| MAP2K1 | ENST00000307102.9 | 821 | CGTTAACTGCAGAGCCGTCG | chr15:66387388-66387411_− |
| | | 822 | GACGCCCATCCAGCTGAACC | chr15:66387364-66387387_+ |
| | | 823 | GTTAACTGCAGAGCCGTCGG | chr15:66387387-66387410_− |
| | | 824 | CAGCCGCATCTCCTTCCACC | chr15:66485126-66485149_− |
| | | 825 | TGGAGATCAAACCCGCAATC | chr15:66436755-66436778_+ |
| MAP2K2 | ENST00000262948.9 | 826 | GGGCCAGCATCGGGGCTCCG | chr19:4123865-4123888_+ |
| | | 827 | CAGCATTTGCATGGAACACA | chr19:4110507-4110530_− |
| | | 828 | CGAGCAGCAGAAGAAGCGGC | chr19:4117558-4117581_− |
| | | 829 | CGGGGAGATCAGCATTTGCA | chr19:4110516-4110539_− |
| | | 830 | CTTCCTCCGGGCCAGCATCG | chr19:4123857-4123880_+ |
| MAP3K7 | ENST00000369329.7 | 831 | TGTGAAGATAAGCCACTCCT | chr6:90560121-90560144_+ |
| | | 832 | CGAGTCATCAGGCTCTCAAT | chr6:90552121-90552144_+ |
| | | 833 | TTTACAGTGTTCCCAAGGAG | chr6:90560133-90560156_− |
| | | 834 | GTGAAGATAAGCCACTCCTT | chr6:90560122-90560145_+ |
| | | 835 | TCCATTGAAGGGCGCTGGGA | chr6:90552082-90552105_+ |
| MAP3K8 | ENST00000263056.5 | 836 | TCCTCGGGGCGCCTTTGGAA | chr10:30447876-30447899_+ |
| | | 837 | TTCCTGTAAGTCAGCTTCCA | chr10:30447835-30447858_− |
| | | 838 | ATGACAACCATGGTACCTCT | chr10:30439138-30439161_− |
| | | 839 | CCGATGTTCTCCTGATCCCC | chr10:30447818-30447841_+ |
| | | 840 | GTAATGGAGTACATGAGCAC | chr10:30438935-30438958_+ |
| MAPK1 | ENST00000215832.10 | 841 | CGGGCCCGGAGATGGTCCGC | chr22:21867392-21867415_− |
| | | 842 | GCGGGCCCGGAGATGGTCCG | chr22:21867393-21867416_− |
| | | 843 | CCGCGGGCAGGTGTTCGACG | chr22:21867376-21867399_− |
| | | 844 | TTCTCTACCAGATCCTCAGA | chr22:21805933-21805956_− |
| | | 845 | GTGTGGCCACATATTCTGTC | chr22:21799049-21799072_+ |
| MAPK11 | ENST00000330651.10 | 846 | GACATGTCGGGCCCTCGCGC | chr22:50270272-50270295_− |
| | | 847 | GCCGGTAGAAGCCGGCGCGA | chr22:50270261-50270284_+ |
| | | 848 | ACTCGGCCGGGATCATCCAC | chr22:50267256-50267279_− |
| | | 849 | CGGTCTTGTTCAGCTCCTGC | chr22:50270243-50270266_+ |
| | | 850 | CTCGGCCGGGATCATCCACC | chr22:50267255-50267278_− |
| MAPK14 | ENST00000229794.8 | 851 | GCATGAATGATGGACTGAAA | chr6:36052753-36052776_− |
| | | 852 | AAGTAACCGCAGTTCTCTGT | chr6:36052784-36052807_− |
| | | 853 | ATTCAGCTGACATAATTCAC | chr6:36073697-36073720_+ |
| | | 854 | TACACCTGCAAGGTCTCTGG | chr6:36059311-36059334_+ |
| | | 855 | TACCAGAACCTGTCTCCAGT | chr6:36028226-36028249_+ |
| MAPK3 | ENST00000263025.8 | 856 | TTTGTGATTTCGGCCTGGCC | chr16:30118139-30118162_− |
| | | 857 | GGGGAGCCCCGTAGAACCGA | chr16:30123153-30123176_− |
| | | 858 | CACATACTCCGTCAGGAAGC | chr16:30118091-30118114_+ |
| | | 859 | GCGTAGCCACATACTCCGTC | chr16:30118084-30118107_+ |
| | | 860 | CCACGCGAGTCTTGCGCACG | chr16:30121971-30121994_+ |
| MAPK8 | ENST00000374176.8 | 861 | TAGTAGCGAGTCACTACATA | chr10:48420253-48420276_− |
| | | 862 | GAATCAGACTCATGCCAAGC | chr10:48404914-48404937_+ |
| | | 863 | ACTTTGAAGATTCTTGACTT | chr10:48420193-48420216_+ |
| | | 864 | GCCCATGCCAAGGATGACCT | chr10:48420284-48420307_− |
| | | 865 | GAGTCTGATTCTGAAATGGT | chr10:48404902-48404925_− |
| MAPK9 | ENST00000452135.6 | 866 | TCAGTTTTATAGTGTGCAAG | chr5:180280515-180280538_− |
| | | 867 | AAGCATCTGGTAAAGAAGGT | chr5:180261725-180261748_+ |
| | | 868 | GTAACGTTTTAGGACAGTGA | chr5:180280483-180280506_+ |
| | | 869 | GCTGAAACCAATTGGCTCTG | chr5:180280455-180280478_− |
| | | 870 | GTCACCCATACATCACTGTT | chr5:180241054-180241077_− |
| MCL1 | ENST00000369026.2 | 871 | CTGGAGACCTTACGACGGGT | chr1:150578880-150578903_− |
| | | 872 | CCACCCTCACGCCAGACTCC | chr1:150579308-150579331_− |
| | | 873 | GGGACCTCGGCGCCAATGGG | chr1:150579270-150579293_+ |
| | | 874 | ACCTTACGACGGGTTGGGGA | chr1:150578874-150578897_− |
| | | 875 | GCCATCCCCAACCCGTCGTA | chr1:150578873-150578896_+ |
| MDM2 | ENST00000539479.6 | 876 | TTGAAGTTATTAAAGTCTGT | chr12:68813574-68813597_+ |
| | | 877 | AGACACTTATACTATGAAAG | chr12:68813606-68813629_+ |
| | | 878 | TACCATGATCTACAGGAACT | chr12:68820333-68820356_+ |
| | | 879 | ATCAGTAGGTACAGACATGT | chr12:68809221-68809244_− |
| | | 880 | GCTTCTCTGTGAAAGAGCAC | chr12:68816923-68816946_+ |
| MET | ENST00000397752.7 | 881 | TCCTGTTTACCTTGGTGCAG | chr7:116699124-116699147_+ |
| | | 882 | TTACTTCTTGACGGTCCAAA | chr7:116699860-116699883_+ |
| | | 883 | CAAGGCTGACCATATGTGGC | chr7:116755388-116755411_+ |
| | | 884 | AAGAAAACTAGAGTTCTCCT | chr7:116755447-116755470_+ |
| | | 885 | CTACAAAGAAGTTGATGAAC | chr7:116699653-116699676_− |
| MGMT | ENST00000306010.7 | 886 | TGCGCACCGCGAGGACCTGC | chr10:129467259-129467282_− |
| | | 887 | CTTTGCGTCCCGACGCCCGC | chr10:129467244-129467267_+ |
| | | 888 | GAAATAAAGCTCCTGGGCAA | chr10:129536339-129536362_+ |
| | | 889 | GTGCGCACCGCGAGGACCTG | chr10:129467260-129467283_− |
| | | 890 | GCAAAGCGTTCTAGGGGCGC | chr10:129467227-129467250_− |
| MRE11 | ENST00000323929.7 | 891 | TTTTCTTAATAACTCGAGGC | chr11:94485994-94486017_+ |
| | | 892 | TTCATGAAAATAAGCCCTCA | chr11:94486030-94486053_− |
| | | 893 | CTGTTTTATATCTCACAACC | chr11:94471618-94471641_− |
| | | 894 | GGCAATCATGACGATCCCAC | chr11:94479674-94479697_− |
| | | 895 | CTTTGGACGTTCAATGTCTG | chr11:94478800-94478823_− |
| MS4A1 | ENST00000345732.8 | 896 | GTCCAAAACCACTCTTCAGG | chr11:60462450-60462473_+ |
| | | 897 | TGTAACAGTATTGGGTAGAT | chr11:60466114-60466137_− |
| | | 898 | CGGATCACTCCTGGCAGCAA | chr11:60464298-60464321_+ |
| | | 899 | CTCATGAAGAAGCTTTGCGT | chr11:60462491-60462514_− |
| | | 900 | TGAGGGAATCTAAGACTTTG | chr11:60462510-60462533_+ |
| MSLN | ENST00000382862.7 | 901 | CAATGTGGACCTGCTCCCGA | chr16:764966-764989_+ |
| | | 902 | GGGGCCCCGAGAACGCATCT | chr16:764906-764929_− |
| | | 903 | AAGAAACGGGTGCAGGCCTG | chr16:764925-764948_− |
| | | 904 | GACTCGGCCACAAAGCGCCC | chr16:765175-765198_− |
| | | 905 | GACCCCTGTTGGGGTCCTGT | chr16:762699-762722_+ |
| MST1R | ENST00000296474.7 | 906 | AAGTCGCGAGAGGCCGCGTA | chr3:49903490-49903513_+ |
| | | 907 | ACAAAGTCTTTCCGGGGCAC | chr3:49897661-49897684_+ |
| | | 908 | CCGTGCCAGGCGTGTGTGCA | chr3:49902763-49902786_+ |
| | | 909 | TTTGAGCTGTCCTTGGGCAG | chr3:49898056-49898079_+ |
| | | 910 | CTAAGGGCATGGCATTTCAT | chr3:49898605-49898628_− |
| MTOR | ENST00000361445.8 | 911 | CCAATTCTCCTATTGTTGCC | chr1:11233402-11233425_+ |
| | | 912 | ACAGCTTAGGACATGGTTCA | chr1:11243256-11243279_+ |
| | | 913 | CAAGTACTGCAAAGATCTCA | chr1:11248021-11248044_− |
| | | 914 | TCGCCACCCAGGCATGCCCA | chr1:11240410-11240433_− |
| | | 915 | GCAGCTCCTTGATATCCTGC | chr1:11241580-11241603_+ |
| MUC5AC | ENST00000621226.2 | 916 | CATGGGAAGCTGAGCTGCAT | chr11:1175227-1175250_+ |
| | | 917 | TGGGCTGGCAGGTGCTGACA | chr11:1174924-1174947_− |
| | | 918 | GCCGGCTGCTTCTGCCCTGA | chr11:1164510-1164533_+ |
| | | 919 | AAGGCCATCAAGATTTTCCT | chr11:1177320-1177343_+ |
| | | 920 | GCGTGACACTGAGCCTGGAT | chr11:1167955-1167978_+ |
| NAE1 | ENST00000290810.7 | 921 | TTCCTTCAAAGAAGCAGTAT | chr16:66824858-66824881_− |
| | | 922 | CTTAAAAACTTGGTACTACC | chr16:66826675-66826698_− |
| | | 923 | GAACCGAGCTGAAGCTGCCA | chr16:66823578-66823601_− |
| | | 924 | TATGTCCTACAGATCAAAAG | chr16:66821493-66821516_+ |
| | | 925 | AGGACCACAGTCATACTCCA | chr16:66817466-66817489_− |
| NAMPT | ENST00000222553.7 | 926 | TCTGCCGCAGGATTCATCTC | chr7:106284867-106284890_+ |
| | | 927 | CATCTCGGGCCGGAGGACAG | chr7:106284881-106284904_+ |
| | | 928 | CCGCAGGATTCATCTCGGGC | chr7:106284871-106284894_+ |
| | | 929 | CCGGCCCGAGATGAATCCTG | chr7:106284871-106284894_− |
| | | 930 | GCTCACTTGGTTAACTTCAA | chr7:106268556-106268579_− |
| NCSTN | ENST00000294785.9 | 931 | TCTTTCTGCGTCCTACTAGC | chr1:160343459-160343482_+ |
| | | 932 | AGAAATACAGTGGAATTCGC | chr1:160350147-160350170_+ |
| | | 933 | TCCACACATTGTAATCAGAC | chr1:160351711-160351734_− |
| | | 934 | GGACATTAAAGCCTGACGAC | chr1:160351761-160351784_+ |
| | | 935 | CAGTGGAATTCGCTGGGCAA | chr1:160350154-160350177_+ |
| NEK11 | ENST00000383366.8 | 936 | ATTCAGGAATATAAACAAGC | chr3:131109823-131109846_+ |
| | | 937 | TTCCAGTTAAAGTTGTGGCC | chr3:131133869-131133892_− |
| | | 938 | TGGTTTATCCAGCTGCTGCT | chr3:131109877-131109900_+ |
| | | 939 | CAAGAAAGCCAAACGAGGAG | chr3:131029851-131029874_+ |
| | | 940 | CCGACTTTGTGTCATAGCCT | chr3:131133926-131133949_− |
| NOTCH1 | ENST00000277541.7 | 941 | TCCAGGTTGATCTCGCAGTT | chr9:136515375-136515398_+ |
| | | 942 | GTTGATGTTGGTCTGGCAGT | chr9:136513108-136513131_+ |
| | | 943 | CGCAGGGGTTGGAGGCGCAC | chr9:136523143-136523166_+ |
| | | 944 | TTGATGTTGGTCTGGCAGTT | chr9:136513109-136513132_+ |
| | | 945 | GGCCCGCGATGCTCCCAGCC | chr9:136544080-136544103_− |
| NOTCH2 | ENST00000256646.6 | 946 | AGTACAGTTTCCATGGATGC | chr1:119955053-119955076_+ |
| | | 947 | ATGCTCACAAGGATTGCTAT | chr1:119967598-119967621_+ |
| | | 948 | GCTACCTGTCTGGATAAGAT | chr1:119967458-119967481_− |
| | | 949 | CAGGTGCTCCCTTCAAAACC | chr1:119987063-119987086_+ |
| | | 950 | TCAGAATGGAGGGGTTTGTG | chr1:119987004-119987027_− |
| NOTCH3 | ENST00000263388.6 | 951 | CATGTCCCACCGGCCCTGCA | chr19:15185306-15185329_+ |
| | | 952 | CAAATGGCCCGGCCGTTCAC | chr19:15189334-15189357_+ |
| | | 953 | GGTCGCGGCCGGCCGCCATG | chr19:15200899-15200922_− |
| | | 954 | GCACCTGCCCAAGTGCTCGC | chr19:15189142-15189165_+ |
| | | 955 | GTGAACGTGGACGACTGTCC | chr19:15191821-15191844_− |
| NOTCH4 | ENST00000375023.3 | 956 | CAGAGGCAAAAGAAGGCTCC | chr6:32216961-32216984_+ |
| | | 957 | TCCTAGGGGCTGTTCGAATG | chr6:32221037-32221060_− |
| | | 958 | TATGGCAGGAGGTGCCTTTG | chr6:32221139-32221162_+ |
| | | 959 | CCACGTTGTGAGCTGCGGGC | chr6:32221069-32221092_− |
| | | 960 | GCACAGGTTGGGAGCACACA | chr6:32215344-32215367_+ |
| NPEPPS | ENST00000322157.8 | 961 | TGTAGACATAACAGGTGTGC | chr17:47585557-47585580_− |
| | | 962 | TCGCTCCATACAGTATAATT | chr17:47605395-47605418_− |
| | | 963 | AATACCTGGACCAAACAAAT | chr17:47596408-47596431_+ |
| | | 964 | CAGCATGGCAGAGCTGTACT | chr17:47603945-47603968_− |
| | | 965 | AGAGAAAGGTCACGAATGCC | chr17:47603980-47604003_− |
| NR3C1 | ENST00000343796.6 | 966 | CAGTGTGCTTGCTCAGGAGA | chr5:143400769-143400792_− |
| | | 967 | GACTTCTATAAAACCCTAAG | chr5:143400735-143400758_− |
| | | 968 | TTTGGAAGGAAACTCGAATG | chr5:143400109-143400132_− |
| | | 969 | TCATCGAACTCTGCACCCCT | chr5:143399969-143399992_− |
| | | 970 | TCCAAAGAATCATTAACTCC | chr5:143400810-143400833_− |
| NRAS | ENST00000369535.4 | 971 | TGAAATGACTGAGTACAAAC | chr1:114716141-114716164_− |
| | | 972 | AGAGACCAATACATGAGGAC | chr1:114713865-114713888_− |
| | | 973 | TGAATATGATCCCACCATAG | chr1:114716048-114716071_− |
| | | 974 | GATCATATTCATCTACAAAG | chr1:114716060-114716083_+ |
| | | 975 | GAGTACAAACTGGTGGTGGT | chr1:114716131-114716154_− |
| NTRK1 | ENST00000524377.5 | 976 | GTGTGCAGCTGCACACTGGC | chr1:156873634-156873657_− |
| | | 977 | GCATCCCCTTCTCTGTGGAT | chr1:156873680-156873703_+ |
| | | 978 | CTCTCCTGGAAAACTGTGCA | chr1:156866937-156866960_+ |
| | | 979 | CGGCGGTGGAGATGCACCAC | chr1:156873656-156873679_+ |
| | | 980 | CCAGCGCTGTAGCCAGCGCA | chr1:156868138-156868161_− |
| NTRK2 | ENST00000277120.7 | 981 | TTATAGAACCACTGAAGCGC | chr9:84727736-84727759_− |
| | | 982 | GGATTCTGGATTAAAATTTG | chr9:84702356-84702379_+ |
| | | 983 | CCCGCCATGGCGCGGCTCTG | chr9:84670775-84670798_+ |
| | | 984 | GAAGCCCCAGAGCCGCGCCA | chr9:84670779-84670802_− |
| | | 985 | GCCGTGGTACTCCGTGTGAT | chr9:84727809-84727832_− |
| NTRK3 | ENST00000394480.6 | 986 | CACCCCTTCCTGATGTGGAC | chr15:88136502-88136525_− |
| | | 987 | GCAAGACTGAGATCAATTGC | chr15:88256015-88256038_− |
| | | 988 | ACTGCAGCTGTGACATCCGC | chr15:88137515-88137538_− |
| | | 989 | GCCAGAAACTACACTTGGCT | chr15:88256113-88256136_+ |
| | | 990 | CTGATGTTCATGCGGAAGAG | chr15:88137411-88137434_+ |
| PARP1 | ENST00000366794.9 | 991 | GAACAACTCCTGAAGGCTCT | chr1:226380045-226380068_+ |
| | | 992 | CCACCTCAACGTCAGGGTGC | chr1:226402285-226402308_+ |
| | | 993 | ACGGAGGCGCTGGTTTCTGG | chr1:226383100-226383123_+ |
| | | 994 | GCAAGTGCCTTCTGGGGAGT | chr1:226386327-226386350_− |
| | | 995 | GTAGCTGATGGCATGGTGTT | chr1:226385645-226385668_− |
| PARP2 | ENST00000250416.9 | 996 | AACTCGTAGATGCCAGAGAC | chr14:20344998-20345021_+ |
| | | 997 | ATGGTATGCCAGGAAGGTCA | chr14:20345084-20345107_+ |
| | | 998 | ATCTACGAGTTTTCTTGGCA | chr14:20344986-20345009_− |
| | | 999 | AGCTTTGCCCTTTAACAGCA | chr14:20345405-20345428_− |
| | | 1000 | AGCAAGATGGTATGCCAGGA | chr14:20345078-20345101_+ |
| PARP3 | ENST00000431474.5 | 1001 | CATCTTCTAGCCTTGTGAAG | chr3:51944415-51944438_− |
| | | 1002 | GGACCACTTTGTGTCTCACC | chr3:51944496-51944519_+ |
| | | 1003 | TGATGAGCTTCTGCGTGGCT | chr3:51944833-51944856_− |
| | | 1004 | TGTCCACTCAGCAGCAACCC | chr3:51943508-51943531_+ |
| | | 1005 | CGATAAGTGTGTACTTGCCC | chr3:51944514-51944537_− |
| PDE5A | ENST00000264805.9 | 1006 | GAAATGCACCATTTTCATAG | chr4:119562846-119562869_− |
| | | 1007 | TCTTAGACCCATTGTTGTCA | chr4:119607100-119607123_− |
| | | 1008 | TATGCCAATTAAGAATCATA | chr4:119596527-119596550_− |
| | | 1009 | GCACGAGGACTCTGCTGCAA | chr4:119607183-119607206_+ |
| | | 1010 | CAAGGGACAAGAGCAAGATT | chr4:119607200-119607223_+ |
| PDGFRA | ENST00000257290.9 | 1011 | CGGAGATCCACTCCCGAGAC | chr4:54273592-54273615_+ |
| | | 1012 | TGCGGGCCTACCCACCTCCC | chr4:54267635-54267658_+ |
| | | 1013 | TGCCTCCTACGACAGCAGAC | chr4:54263805-54263828_+ |
| | | 1014 | GTGCCTCGGCGGCCCACACA | chr4:54261310-54261333_+ |
| | | 1015 | ATGATCACCATGGCTCAACT | chr4:54272420-54272443_+ |
| PDGFRB | ENST00000261799.8 | 1016 | CGCGCAACGTGTCGGAGACC | chr5:150132748-150132771_− |
| | | 1017 | CAGGACAGTGGGCGGTGGGT | chr5:150132829-150132852_+ |
| | | 1018 | GCATCGGAGCCGGACACTGC | chr5:150132864-150132887_− |
| | | 1019 | CTACTATGTCTACAGACTCC | chr5:150134749-150134772_− |
| | | 1020 | GTGACACTGCACGAGAAGAA | chr5:150134886-150134909_− |
| PGF | ENST00000555567.5 | 1021 | TGCTGGGAACGGCTCGTCAG | chr14:74953909-74953932_− |
| | | 1022 | CGAGCCGTTCCCAGCAGACA | chr14:74953916-74953939_+ |
| | | 1023 | TAAAGATCCGTTCTGGGGAC | chr14:74948556-74948579_− |
| | | 1024 | GGCTTCATCTTCTCCCGCAG | chr14:74946384-74946407_+ |
| | | 1025 | TCAGCTCCACGTAGGAGGGC | chr14:74948537-74948560_+ |
| PIGF | ENST00000281382.10 | 1026 | GGTCCTAACAAACATAAGCA | chr2:46612266-46612289_+ |
| | | 1027 | GGATTGGGAAAGACCATGGC | chr2:46592473-46592496_− |
| | | 1028 | CACTGGATTGGGAAAGACCA | chr2:46592477-46592500_− |
| | | 1029 | CATGGTCTTTCCCAATCCAG | chr2:46592478-46592501_+ |
| | | 1030 | AAGTTCTCCAAGAAGAGTGA | chr2:46615063-46615086_+ |
| PIK3CA | ENST00000263967.3 | 1031 | ATGCCTCCACGACCATCATC | chr3:179198825-179198848_+ |
| | | 1032 | CTTCAATCACTGACATATCT | chr3:179210483-179210506_− |
| | | 1033 | TGCACTATTTATAACCCAAA | chr3:179203706-179203729_− |
| | | 1034 | GAGTACCTTGTTCCAATCCC | chr3:179204566-179204589_+ |
| | | 1035 | GGTTAAAGATCCAGAAGTAC | chr3:179199726-179199749_+ |
| PIK3CB | ENST00000477593.5 | 1036 | CCTGCCCCATTTTATACTTG | chr3:138734752-138734775_− |
| | | 1037 | ATCGTATTTACCCACGCTAC | chr3:138712275-138712298_+ |
| | | 1038 | ACTTGTGGGATTGTCTTGGA | chr3:138742670-138742693_− |
| | | 1039 | TATAGGAGTCAATATCCATA | chr3:138755915-138755938_+ |
| | | 1040 | CACTGGGATTTATATCCAGT | chr3:138759210-138759233_− |
| PIK3CD | ENST00000377346.8 | 1041 | TACAAGGACCAGCTTAAGAC | chr1:9719959-9719982_+ |
| | | 1042 | CCGCAGGGTACCAGGCCCCC | chr1:9716007-9716030_− |
| | | 1043 | ATGGGAGGTGGTTTGGCACG | chr1:9717072-9717095_− |
| | | 1044 | ACGCAGGATGCCCCCTGGGG | chr1:9710448-9710471_+ |
| | | 1045 | GATGCGGAACGGCTGCTCCA | chr1:9717558-9717581_− |
| PIK3CG | ENST00000496166.5 | 1046 | CCTCCTCTGTGAAGGGTTTG | chr7:106868760-106868783_− |
| | | 1047 | CAGAGAGCGATTTAATATCA | chr7:106872888-106872911_− |
| | | 1048 | GATGACTGCACGGGAGTCAC | chr7:106868539-106868562_+ |
| | | 1049 | TTTAGAGTTCCATATGATCC | chr7:106874758-106874781_+ |
| | | 1050 | GGACGTCACCCACGGGTGCA | chr7:106868150-106868173_− |
| PIM3 | ENST00000360612.4 | 1051 | GCCGCTCGAACCAGTCCAGC | chr22:49961518-49961541_− |
| | | 1052 | AAGGAGCGGGTGACCGAGTG | chr22:49961338-49961361_+ |
| | | 1053 | GAAGGAGCGGGTGACCGAGT | chr22:49961337-49961360_+ |
| | | 1054 | TGAAGGAGCGGGTGACCGAG | chr22:49961336-49961359_+ |
| | | 1055 | GGTGGTGCTGCTGCGCAAGG | chr22:49961464-49961487_+ |
| PLK1 | ENST00000300093.8 | 1056 | CGTAGGTAGTATCGGGCCTC | chr16:23680125-23680148_− |
| | | 1057 | CAACCAAAGTCGAATATGAC | chr16:23680928-23680951_+ |
| | | 1058 | TGGTGTTGGAGCTCTGCCGC | chr16:23679314-23679337_+ |
| | | 1059 | AGGTGGATGTGTGGTCCATT | chr16:23681027-23681050_+ |
| | | 1060 | AAGTCGAATATGACGGGGAG | chr16:23680934-23680957_+ |
| PLK2 | ENST00000274289.7 | 1061 | GCAGTAGCGCTTCCCAGTCG | chr5:58459710-58459733_+ |
| | | 1062 | GAGTAGCTAAACCTCATCAA | chr5:58458990-58459013_− |
| | | 1063 | CAGAAGTTCGATACTACCTC | chr5:58458462-58458485_− |
| | | 1064 | AGTAGCTAAACCTCATCAAA | chr5:58458989-58459012_− |
| | | 1065 | ACTCGGGGCCGGAGATCTCG | chr5:58459746-58459769_− |
| PLK3 | ENST00000372201.4 | 1066 | AGGCCCGAAGGATGGCGGCC | chr1:44803074-44803097_− |
| | | 1067 | CTTGCACCGGGACCTCAAGT | chr1:44801728-44801751_+ |
| | | 1068 | CTGCTCCGGAGGCTCCAACC | chr1:44801901-44801924_− |
| | | 1069 | GGCTTGGCGACGCGGCTCTG | chr1:44800908-44800931_− |
| | | 1070 | CTTCAGGTCAGCCGTCTCAA | chr1:44802980-44803003_− |
| POLA1 | ENST00000379068.7 | 1071 | TCACAGTCGTCGCCGTGCAC | chrX:24693967-24693990_− |
| | | 1072 | GTCACCTAGCAGACCATCCT | chrX:24715156-24715179_− |
| | | 1073 | TGGGACCAACACATCTAGCC | chrX:24726988-24727011_+ |
| | | 1074 | TCAACTTTACACCAACTGAC | chrX:24727795-24727818_− |
| | | 1075 | GTCGCCGTGCACAGGTGCCA | chrX:24693959-24693982_− |
| PORCN | ENST00000326194.10 | 1076 | CATCCTCATCTACCTACTCA | chrX:48511463-48511486_+ |
| | | 1077 | CATGCAAGCACCGTGGCAGG | chrX:48511314-48511337_+ |
| | | 1078 | GGCGGCCTTGGACAGCTTGT | chrX:48512479-48512502_− |
| | | 1079 | CCATCCGTGGGGGTCTGCAA | chrX:48509801-48509824_+ |
| | | 1080 | AATGGCCACCTTTAGCCGCC | chrX:48509819-48509842_+ |
| PPARG | ENST00000287820.10 | 1081 | CCCATAACAGCATGGAATAG | chr3:12351574-12351597_− |
| | | 1082 | ACGACATTCAATTGCCATGA | chr3:12381408-12381431_− |
| | | 1083 | AGAGCCTTCCAACTCCCTCA | chr3:12381394-12381417_+ |
| | | 1084 | TCATGCTTGTGAAGGATGCA | chr3:12381469-12381492_+ |
| | | 1085 | AGTGAAGGGCTTGATATCAA | chr3:12379796-12379819_− |
| PPP2CA | ENST00000481195.5 | 1086 | AAAAGAATCCAACGTGCAAG | chr5:134206091-134206114_− |
| | | 1087 | AACGCATCACCATTCTTCGA | chr5:134201985-134202008_− |
| | | 1088 | ACATCGAACCTCTTGCACGT | chr5:134206083-134206106_+ |
| | | 1089 | GACCACAGCAAGTCACACAT | chr5:134200470-134200493_+ |
| | | 1090 | AGCTCACCAGCTAGTGATGG | chr5:134200333-134200356_− |
| PRKCA | ENST00000413366.7 | 1091 | AGTCGTCGGTCTTTGTCTGA | chr17:66688311-66688334_− |
| | | 1092 | TGTCCCCAGCCTCTGCGGAA | chr17:66645419-66645442_+ |
| | | 1093 | CTTGTGAACGTTCATATCGC | chr17:66645382-66645405_− |
| | | 1094 | CAACCGCTTCGCCCGCAAAG | chr17:66302901-66302924_+ |
| | | 1095 | GAGGGGGCGGATTTACCTAA | chr17:66645455-66645478_+ |
| PRKCB | ENST00000643927.1 | 1096 | GGGTCAGCCATCTTGCGCGC | chr16:23836163-23836186_− |
| | | 1097 | GTGCTCTCCTCGCCCTCGCT | chr16:23836202-23836225_− |
| | | 1098 | TGGTCCGTGCCACACAGGCT | chr16:24035459-24035482_− |
| | | 1099 | GGTCAGCCATCTTGCGCGCG | chr16:23836162-23836185_− |
| | | 1100 | CCACGTTTTGTGACCACTGT | chr16:24032181-24032204_+ |
| PRKCE | ENST00000306156.7 | 1101 | CGACTCGCGCATCGGCCAAA | chr2:45652240-45652263_+ |
| | | 1102 | TGGCGCAGCGACCAGGCTGT | chr2:45652157-45652180_− |
| | | 1103 | ATGCGGCCGAGGAAGCGGCA | chr2:45976466-45976489_+ |
| | | 1104 | GAACGGGAGCCGCCACTTCG | chr2:45652420-45652443_+ |
| | | 1105 | ACACTACCATGGTCGGGGCG | chr2:45652087-45652110_− |
| PRKCG | ENST00000263431.3 | 1106 | TTTCTGCAAAACAGGGGCCG | chr19:53882536-53882559_− |
| | | 1107 | GCGATTCAGAGGGGGGACCC | chr19:53882519-53882542_+ |
| | | 1108 | GGAGCTGCTCAAGGCGCCCG | chr19:53892613-53892636_+ |
| | | 1109 | CGGCGTAGGCGATTCAGAGG | chr19:53882511-53882534_+ |
| | | 1110 | TACGTGGATCTCATCTGCTG | chr19:53889990-53890013_− |
| PRKCI | ENST00000295797.4 | 1111 | CTCATTGCAAAGGCCCTCAA | chr3:170235264-170235287_− |
| | | 1112 | AAACTCGTCACAATTGAATG | chr3:170270519-170270542_+ |
| | | 1113 | AACTCGTCACAATTGAATGT | chr3:170270520-170270543_+ |
| | | 1114 | CACCATGAAATGGATAGATG | chr3:170235325-170235348_+ |
| | | 1115 | TTAAATTATCTTCATGAGCG | chr3:170284488-170284511_+ |
| PRKDC | ENST00000314191.6 | 1116 | CGCTTATAGAGCTGGTACAT | chr8:47912452-47912475_+ |
| | | 1117 | CTGTGAACTTTTACATAGCA | chr8:47912531-47912554_− |
| | | 1118 | AAGCCACGCAGATGCCAGAA | chr8:47912490-47912513_− |
| | | 1119 | TGTAGCACTCCAACGCGGCC | chr8:47893183-47893206_+ |
| | | 1120 | TGATGAAGAGCTATGTGGCC | chr8:47914023-47914046_− |
| PRLR | ENST00000618457.4 | 1121 | TGTCCCAGGCCTCCACCAGC | chr5:35086254-35086277_+ |
| | | 1122 | GGAAGTCCTCCATCTGTCCC | chr5:35086240-35086263_+ |
| | | 1123 | ATTATTCACTGACTTACCAC | chr5:35086212-35086235_− |
| | | 1124 | ATAAGGAAACATTCACCTGC | chr5:35086269-35086292_− |
| | | 1125 | AAACATTCACCTGCTGGTGG | chr5:35086263-35086286_− |
| PSEN1 | ENST00000324501.9 | 1126 | ATTATCTAATGGACGACCCC | chr14:73170855-73170878_+ |
| | | 1127 | AAAGAGCATGATCACATGCT | chr14:73170944-73170967_− |
| | | 1128 | GGCAGGAGCACAACGACAGA | chr14:73170812-73170835_+ |
| | | 1129 | GCTTTTATACCCGGAAGGAT | chr14:73171019-73171042_+ |
| | | 1130 | ACCCCAGGGTAACTCCCGGC | chr14:73170870-73170893_+ |
| PSENEN | ENST00000587708.6 | 1131 | CCAGGTTCATAGCTGCGCTG | chr19:35745917-35745940_− |
| | | 1132 | CTGTGCCGGAAGTACTACCT | chr19:35745969-35745992_+ |
| | | 1133 | ATGTTGACCAACCAGAGAAA | chr19:35746435-35746458_− |
| | | 1134 | GATTTGGCTCTGTTCTGTGT | chr19:35746493-35746516_− |
| | | 1135 | GTTCTGTGTAGGCTGGGACA | chr19:35746482-35746505_− |
| PSMB1 | ENST00000262193.6 | 1136 | ACAGCCATGTATTCGGCTCC | chr6:170553207-170553230_− |
| | | 1137 | GGCGAAAATCGCAGCTGCAA | chr6:170553147-170553170_+ |
| | | 1138 | TCGCAGCTGCAAAGGGCCCG | chr6:170553155-170553178_+ |
| | | 1139 | GATGGAACCGCACAGAGCCG | chr6:170553172-170553195_− |
| | | 1140 | TCTGATACTCGATTGAGTGA | chr6:170549047-170549070_− |
| PSMB10 | ENST00000358514.8 | 1141 | GTCCCGGTCTTGCGTGCGTG | chr16:67936422-67936445_+ |
| | | 1142 | ACGCGTCCTCCCGGGGCTCA | chr16:67936447-67936470_− |
| | | 1143 | TCTCGAAGGAGAAGCCCCCT | chr16:67936703-67936726_+ |
| | | 1144 | GGGCTGGCTTCAGCATCTTG | chr16:67936733-67936756_+ |
| | | 1145 | TCCCGGTCTTGCGTGCGTGA | chr16:67936423-67936446_+ |
| PSMB2 | ENST00000373237.3 | 1146 | GGAGGCGACAAGAACATAGT | chr1:35641381-35641404_+ |
| | | 1147 | AAGATATTACTCCTGTGTGT | chr1:35636380-35636403_− |
| | | 1148 | GCCACCATGGAGTACCTCAT | chr1:35641415-35641438_− |
| | | 1149 | AGGTACTCCATGGTGGCGGA | chr1:35641421-35641444_+ |
| | | 1150 | GATACCGATGAGGTACTCCA | chr1:35641411-35641434_+ |
| PSMB5 | ENST00000361611.10 | 1151 | AAAAACCCGCGCTGGTTCAC | chr14:23034825-23034848_+ |
| | | 1152 | CGCTACCGGTGAACCAGCGC | chr14:23034830-23034853_− |
| | | 1153 | TGGGACACCCCAGCCTGGCG | chr14:23034734-23034757_+ |
| | | 1154 | CAAGTCCGAAAAACCCGCGC | chr14:23034817-23034840_+ |
| | | 1155 | GCTTCATGGAACAACCACCC | chr14:23034691-23034714_− |
| PSMB8 | ENST00000374882.7 | 1156 | ATTCTTCCAGTCCCTGGGTG | chr6:32843058-32843081_− |
| | | 1157 | GATTCCGGCCGCTGCCCTCG | chr6:32843946-32843969_+ |
| | | 1158 | CCCCGGGGTAAAGCGAGCTC | chr6:32843856-32843879_+ |
| | | 1159 | ACAGAATTCTTCCAGTCCCT | chr6:32843063-32843086_− |
| | | 1160 | ATTCCGGCCGCTGCCCTCGG | chr6:32843947-32843970_+ |
| PSMB9 | ENST00000374859.2 | 1161 | CCTTGCAGGGATGCTGCGGG | chr6:32854219-32854242_+ |
| | | 1162 | CCGCCCGCAGCATCCCTGCA | chr6:32854219-32854242_− |
| | | 1163 | GCCCGGGGTAAGTCCCCGGT | chr6:32854247-32854270_− |
| | | 1164 | GTGTGGACTTCTCCCGCCCG | chr6:32854262-32854285_− |
| | | 1165 | GCGGGCGGGAGCACCAACCG | chr6:32854234-32854257_+ |
| PSMD1 | ENST00000308696.10 | 1166 | GAGGTTTTATACGAAGATGA | chr2:231062506-231062529_+ |
| | | 1167 | GGCTATAAGCTAACATTCCT | chr2:231070032-231070055_− |
| | | 1168 | AAGATGAAGGTTTCCGGAGT | chr2:231062519-231062542_+ |
| | | 1169 | AGGCTGCAAACTGCCGACTC | chr2:231062532-231062555_− |
| | | 1170 | CAATGATAACTCTGAATATG | chr2:231062640-231062663_+ |
| PSMD2 | ENST00000310118.8 | 1171 | GGACGAGAAGCCGAGCGGCA | chr3:184299337-184299360_+ |
| | | 1172 | ATGCCAATCTCAGAGCTTCA | chr3:184302450-184302473_− |
| | | 1173 | CTTCTCGTCCGTGCCGCCGG | chr3:184299324-184299347_− |
| | | 1174 | GTATCGGCTAGTGGGCTCCC | chr3:184301601-184301624_+ |
| | | 1175 | GCTTCTCGTCCGTGCCGCCG | chr3:184299325-184299348_− |
| PTCH1 | ENST00000331920.10 | 1176 | CAGAGACTCTTATTTAAACT | chr9:95506528-95506551_− |
| | | 1177 | GCCTCCAGCCGGCCGTCCCG | chr9:95508271-95508294_+ |
| | | 1178 | ATTCTGTCCTGTTTCACTGA | chr9:95476776-95476799_+ |
| | | 1179 | TATCACAGAAACAGGTTACA | chr9:95482138-95482161_− |
| | | 1180 | CTGGCAGGAGGAGTTGATTG | chr9:95480005-95480028_− |
| PTGS2 | ENST00000367468.9 | 1181 | GGGTACAATCGCACTTATAC | chr1:186679354-186679377_+ |
| | | 1182 | TTTCTCCATAGAATCCTGTC | chr1:186679333-186679356_+ |
| | | 1183 | TTCTCCATAGAATCCTGTCC | chr1:186679334-186679357_+ |
| | | 1184 | CCGACTCCCTTGGGTGTCAA | chr1:186678259-186678282_− |
| | | 1185 | CCATAGTCAGCATTGTAAGT | chr1:186678355-186678378_+ |
| PTK2 | ENST00000522684.5 | 1186 | GAATGCTTCAAGTGTGCTCT | chr8:140818880-140818903_− |
| | | 1187 | ATTTGGTGTGTGATTCAAGT | chr8:140890692-140890715_+ |
| | | 1188 | GGGTACTGCCGGCTGGTGAA | chr8:140800493-140800516_− |
| | | 1189 | CAAATCTGTAGACTGGAGAC | chr8:140818908-140818931_+ |
| | | 1190 | GGCGATCATACTGGGAGATG | chr8:140846300-140846323_− |
| PTK6 | ENST00000542869.2 | 1191 | CCGCCTCGTTCAGGTGCAGC | chr20:63534240-63534263_+ |
| | | 1192 | AAGATCTGGCGGCGTGCCGG | chr20:63534263-63534286_− |
| | | 1193 | TCCCGGGACACCATGGCGGG | chr20:63537300-63537323_+ |
| | | 1194 | CCGACGCACAGCTTCCGAGC | chr20:63535016-63535039_+ |
| | | 1195 | GGGCCGGCTGCACCTGAACG | chr20:63534243-63534266_− |
| RAC1 | ENST00000356142.4 | 1196 | CGGTAAGGATATAACCTCCC | chr7:6398675-6398698_+ |
| | | 1197 | ACGGTAAGGATATAACCTCC | chr7:6398674-6398697_+ |
| | | 1198 | CGGCTTGTCTTTGCCCCGGG | chr7:6398689-6398712_− |
| | | 1199 | GGTAAGGATATAACCTCCCG | chr7:6398676-6398699_+ |
| | | 1200 | AATCGGCTTGTCTTTGCCCC | chr7:6398692-6398715_− |
| RAD50 | ENST00000378823.7 | 1201 | GATGAATTAACCTCACTGTT | chr5:132591921-132591944_+ |
| | | 1202 | TGATGAATTAACCTCACTGT | chr5:132591920-132591943_+ |
| | | 1203 | TCTAATTGGCCTTTAAGTGA | chr5:132579434-132579457_+ |
| | | 1204 | TTGCTTCTTTCGGCTATCCA | chr5:132587625-132587648_− |
| | | 1205 | AAACGTTTGCAAACATGTCC | chr5:132557310-132557333_+ |
| RAF1 | ENST00000251849.8 | 1206 | TGGAGCACATACAGGGAGCT | chr3:12618697-12618720_− |
| | | 1207 | GCAGTTTGGCTATCAGCGCC | chr3:12618597-12618620_− |
| | | 1208 | GGATGTCGACCTCTGCCTCT | chr3:12604192-12604215_+ |
| | | 1209 | GTATGCGAGAGTCTGTTTCC | chr3:12606211-12606234_− |
| | | 1210 | GTGTTAAAGGTGAAGGCGTG | chr3:12604244-12604267_+ |
| RARA | ENST00000254066.9 | 1211 | CGGGCACCTCAATGGGTACC | chr17:40331256-40331279_+ |
| | | 1212 | GGGGAGAGTCCACCCAGCAT | chr17:40331305-40331328_− |
| | | 1213 | GCAGCTCCTGCCCGACACCT | chr17:40331231-40331254_+ |
| | | 1214 | GGGGGGAAGAAGAAGGCGTA | chr17:40331284-40331307_− |
| | | 1215 | AAAGCAAGGCTTGTAGATGC | chr17:40348381-40348404_− |
| RARB | ENST00000330688.8 | 1216 | GACTCGCAGTGTAGAAATCC | chr3:25428775-25428798_− |
| | | 1217 | GCTCTCAAAGCATGCTTCAG | chr3:25428824-25428847_+ |
| | | 1218 | CAAACCCTGCTTCGTCTGCC | chr3:25461268-25461291_+ |
| | | 1219 | AGCTTTCTCCTGGAGCATGC | chr3:25428804-25428827_− |
| | | 1220 | TTGTCCTGGCAGACGAAGCA | chr3:25461272-25461295_− |
| RARG | ENST00000425354.6 | 1221 | CCTTCCCAGGGGCACTCAGG | chr12:53227440-53227463_− |
| | | 1222 | TTGTCATTGCACACGAAGCA | chr12:53215691-53215714_+ |
| | | 1223 | GCCTTCCCAGGGGCACTCAG | chr12:53227441-53227464_− |
| | | 1224 | CTAGGGCTCAGCATCTCGAA | chr12:53227411-53227434_+ |
| | | 1225 | AAGCATGGCTTGTAGACCCG | chr12:53215706-53215729_+ |
| RET | ENST00000355710.7 | 1226 | TGCAGTCAGCAAGAGACGGC | chr10:43112132-43112155_+ |
| | | 1227 | AAGTATACGCGGGCACAGCC | chr10:43102421-43102444_− |
| | | 1228 | AGCAGAGCTCCCGGGGCTTG | chr10:43102480-43102503_− |
| | | 1229 | GACGTACAGCAAGGGCGTGC | chr10:43100521-43100544_− |
| | | 1230 | AGGCCAACGGCAGCTTCGTG | chr10:43106529-43106552_+ |
| RICTOR | ENST00000357387.7 | 1231 | AAATAATTATCCATGAGGTC | chr5:38967203-38967226_+ |
| | | 1232 | TCCTCTACCTGTTGTGACTG | chr5:38967969-38967992_− |
| | | 1233 | CGGCGAGGAGAACGTCCCGC | chr5:39074122-39074145_− |
| | | 1234 | CCCGTCAATATGGCGGCGAT | chr5:39074363-39074386_− |
| | | 1235 | GACAGTTGGAGGCTTTCAGA | chr5:38967387-38967410_− |
| ROCK1 | ENST00000399799.2 | 1236 | TGCAGAAGTAGTTCTTGCAT | chr18:21045322-21045345_− |
| | | 1237 | TGTAGAGATAACGATCATCT | chr18:21045430-21045453_+ |
| | | 1238 | TTTGTGCCTTCCTTACTGAC | chr18:21042095-21042118_− |
| | | 1239 | GAATGTGACTGGTGGTCGGT | chr18:21042590-21042613_− |
| | | 1240 | TTGGATGCAATCCATTCCAT | chr18:21045303-21045326_− |
| ROCK2 | ENST00000315872.10 | 1241 | TTTTGGCACGTGTATGAAGA | chr2:11235706-11235729_− |
| | | 1242 | GAAGCCTGACAACATGCTCT | chr2:11235757-11235780_− |
| | | 1243 | AATCAGAGGTCTACAGATGA | chr2:11286597-11286620_− |
| | | 1244 | TCCACGTTGATGGGGGAGCG | chr2:11344005-11344028_+ |
| | | 1245 | CGCCCCCGAGACCGCGCCGG | chr2:11344078-11344101_− |
| ROS1 | ENST00000368508.7 | 1246 | GCTGGGTCACTTTCTCTACT | chr6:117385699-117385722_− |
| | | 1247 | GTCCTGTAACTGGGACTTGG | chr6:117403152-117403175_+ |
| | | 1248 | CCCTGGTCAGAGCCCTCAGT | chr6:117387796-117387819_− |
| | | 1249 | GTAGATAGTATTTGGTAAAG | chr6:117396916-117396939_+ |
| | | 1250 | CAGCAAAGGGGATGCGAGGT | chr6:117389616-117389639_+ |
| RPL3 | ENST00000216146.8 | 1251 | ATCACGCCATCAAATCCCGC | chr22:39319595-39319618_+ |
| | | 1252 | CCACCGAGGCCTGCGCAAGG | chr22:39314780-39314803_− |
| | | 1253 | ACAGAGAAGGCTACACGAGC | chr22:39314737-39314760_+ |
| | | 1254 | CATGGGTGGTGTCTCTACAA | chr22:39317573-39317596_+ |
| | | 1255 | TGAGATGATCGACGTCATCG | chr22:39315392-39315415_− |
| RPS6KB1 | ENST00000225577.8 | 1256 | CATGAGGCGACGAAGGAGGC | chr17:59893183-59893206_+ |
| | | 1257 | TAAATGAAAGCATGGACCAT | chr17:59910568-59910591_+ |
| | | 1258 | GCGGCGGGTCCGGGCCCATG | chr17:59893167-59893190_+ |
| | | 1259 | AAGTAACAGGAGCAAATACT | chr17:59914650-59914673_+ |
| | | 1260 | AGGCGACGAAGGAGGCGGGA | chr17:59893187-59893210_+ |
| RPTOR | ENST00000306801.7 | 1261 | AGGCACTGTGAGAAAATTGA | chr17:80545728-80545751_+ |
| | | 1262 | ATAGGTCAGCCGGGAGGTCG | chr17:80754119-80754142_− |
| | | 1263 | CTTCTGTAAATTTGCACCGA | chr17:80643763-80643786_− |
| | | 1264 | GGGGATCATGGGCAGCAGCT | chr17:80754100-80754123_− |
| | | 1265 | GATGGGTCCTCAGAAAGCTC | chr17:80643737-80643760_+ |
| RRM1 | ENST00000300738.9 | 1266 | GTGAGTTGTATTCGGGCTAC | chr11:4118422-4118445_+ |
| | | 1267 | AATGTTGACTTGGCCACCAT | chr11:4107487-4107510_− |
| | | 1268 | CAATGTTGACTTGGCCACCA | chr11:4107488-4107511_− |
| | | 1269 | ACAGCTCGATATGTGGATCA | chr11:4119895-4119918_+ |
| | | 1270 | CTCGATATGTGGATCAAGGT | chr11:4119899-4119922_+ |
| RXRA | ENST00000481739.1 | 1271 | CAGGGACGGGTGCAGCGAGG | chr9:134401691-134401714_− |
| | | 1272 | GGAGCCGATGCCAGGCCCCA | chr9:134401709-134401732_− |
| | | 1273 | CCTCGCTGCACCCGTCCCTG | chr9:134401694-134401717_+ |
| | | 1274 | AGGAAGCCATGTTTCCTGAG | chr9:134408234-134408257_− |
| | | 1275 | TGGCGCCCACCCCTGCGCTG | chr9:134429207-134429230_− |
| RXRB | ENST00000374680.3 | 1276 | ATTTCTTTTCGCACCCCCAC | chr6:33200393-33200416_+ |
| | | 1277 | CCCATGGAAGAACTGATGAC | chr6:33199229-33199252_+ |
| | | 1278 | AGGCCCCGGACCCCTAAGAC | chr6:33198381-33198404_+ |
| | | 1279 | CTCCCCTGGCTCCGGCTCCG | chr6:33200275-33200298_+ |
| | | 1280 | CCTGGCCACTGGCATGAAGA | chr6:33197764-33197787_− |
| RXRG | ENST00000359842.9 | 1281 | TTTCCAATCCCGGGAAGCCC | chr1:165419943-165419966_+ |
| | | 1282 | CTGCAAGTGCTCCTGAGGGT | chr1:165428759-165428782_+ |
| | | 1283 | CTGTGGACAAGGCTGCTGAT | chr1:165428909-165428932_+ |
| | | 1284 | CCCTCTTCATGCCCATGACA | chr1:165417043-165417066_+ |
| | | 1285 | GAGAGCCCAGGGCATTGAGG | chr1:165428810-165428833_+ |
| S1PR1 | ENST00000305352.6 | 1286 | CAATAAAATAGTACATGGGT | chr1:101239214-101239237_− |
| | | 1287 | TAAAATAGTACATGGGTCGG | chr1:101239211-101239234_− |
| | | 1288 | CGTCCGGCATTACAACTACA | chr1:101239058-101239081_+ |
| | | 1289 | GTCCGGCATTACAACTACAC | chr1:101239059-101239082_+ |
| | | 1290 | TTGCCAATAAAATAGTACAT | chr1:101239218-101239241_− |
| SH2B3 | ENST00000341259.6 | 1291 | GCTCCAGCATCCAGGAGGTC | chr12:111446780-111446803_+ |
| | | 1292 | CTCGGCCGGGGAGCTGCCAG | chr12:111418591-111418614_+ |
| | | 1293 | GTGTCCCGGTAGTCGCGGCC | chr12:111418397-111418420_− |
| | | 1294 | CTGGCAGCTCCCCGGCCGAG | chr12:111418588-111418611_− |
| | | 1295 | CTGTGAGTTGCACGCCGTAG | chr12:111418231-111418254_+ |
| SHH | ENST00000297261.6 | 1296 | AAGAAAACACCGGAGCGGAC | chr7:155811834-155811857_− |
| | | 1297 | AGACCCTAGGCGCCAGCGGA | chr7:155811939-155811962_− |
| | | 1298 | GGTGAGTTCCTTAAATCGCT | chr7:155811891-155811914_+ |
| | | 1299 | GTATGCTCGGGACTGGCGTG | chr7:155812048-155812071_− |
| | | 1300 | GGATGAAGAAAACACCGGAG | chr7:155811839-155811862_− |
| SIK1 | ENST00000270162.7 | 1301 | TGGCGGGGGCCTGGCACACC | chr21:43417666-43417689_+ |
| | | 1302 | GTGCCAGGCCCCCGCCAGCC | chr21:43417660-43417683_− |
| | | 1303 | CAGGAGAGCCTCTGTCCACG | chr21:43421317-43421340_− |
| | | 1304 | CCCCTCAAAGACTTCCGGGG | chr21:43421275-43421298_+ |
| | | 1305 | AGTCGTCTCCCCCTCCACCA | chr21:43418514-43418537_− |
| SIRT1 | ENST00000212015.10 | 1306 | CGCAAGAGGCCGCGGAGAGA | chr10:67884820-67884843_+ |
| | | 1307 | TGTCGGCCCCCGCCGCCGAG | chr10:67884762-67884785_− |
| | | 1308 | CACATGCAAGCTCTAGTGAC | chr10:67887491-67887514_+ |
| | | 1309 | GGCCGCGTCGTCCCCCGCCG | chr10:67884789-67884812_+ |
| | | 1310 | TGGGGCGGCCCCAGAGCGTG | chr10:67884876-67884899_+ |
| SLAMF7 | ENST00000368043.7 | 1311 | GAACCAGCCATATTGCTCTC | chr1:160739289-160739312_− |
| | | 1312 | GCTGGTCGGTTCCGTTGGTG | chr1:160748221-160748244_+ |
| | | 1313 | TATATCCTTTGGCAGCTCAC | chr1:160739334-160739357_+ |
| | | 1314 | CTTCCAGAGAGCAATATGGC | chr1:160739286-160739309_+ |
| | | 1315 | GCTTTACTTTGGACTTCAGG | chr1:160748254-160748277_− |
| SLC2A2 | ENST00000314251.7 | 1316 | CTCAACTAATCACCATGCTC | chr3:171014548-171014571_− |
| | | 1317 | GGCCTGGTTCCTATGTATAT | chr3:171007229-171007252_− |
| | | 1318 | CAGTCTCTTCCTCAGCCCAA | chr3:171014580-171014603_+ |
| | | 1319 | GTTCATTGAGTATGAGATTG | chr3:171014614-171014637_+ |
| | | 1320 | CTGCAAAGCTGGATACAGAC | chr3:171014523-171014546_+ |
| SMO | ENST00000249373.7 | 1321 | GCCCCTGTGCCATCGTGGAG | chr7:129203506-129203529_+ |
| | | 1322 | CCGCTGCCCGCCCAGCGCGG | chr7:129189155-129189178_+ |
| | | 1323 | TGGCTGGCCCAGTTCATGGA | chr7:129205702-129205725_+ |
| | | 1324 | CCCCTGTGCCATCGTGGAGA | chr7:129203507-129203530_+ |
| | | 1325 | AGCAGCGGGGGGCATTCCGG | chr7:129203384-129203407_− |
| SRC | ENST00000373578.6 | 1326 | CGTCACCTCCCCGCAGAGGG | chr20:37384368-37384391_+ |
| | | 1327 | GGCGCCCTCCGACTCCATCC | chr20:37393963-37393986_+ |
| | | 1328 | CAGCGGGCCCGCCCTCTGCG | chr20:37384376-37384399_− |
| | | 1329 | CTGGCCCACTCGCTCAGCAC | chr20:37393910-37393933_+ |
| | | 1330 | TCCTAGACTCATAGTCATAG | chr20:37386096-37386119_− |
| SSTR2 | ENST00000357585.3 | 1331 | TTGACACCACAGAGCCATTG | chr17:73169378-73169401_− |
| | | 1332 | TTGCTTGTCAGGTCATAGTA | chr17:73169424-73169447_− |
| | | 1333 | ATTTGACCTCAATGGCTCTG | chr17:73169372-73169395_+ |
| | | 1334 | CACTCAATGGAAGCCACACA | chr17:73169338-73169361_+ |
| | | 1335 | AAAAGCAGCCATGGACATGG | chr17:73169309-73169332_+ |
| SSTR5 | ENST00000293897.5 | 1336 | GCTGGAACGCCTCCTCCCCG | chr16:1078899-1078922_+ |
| | | 1337 | GCCTCCAGAGGCAGCCCCCG | chr16:1078914-1078937_− |
| | | 1338 | AGGCGGTGACAACAGGACGC | chr16:1078933-1078956_+ |
| | | 1339 | GCTGTACCTGCTGGTGTGTG | chr16:1079002-1079025_+ |
| | | 1340 | CTGGAACGCCTCCTCCCCGG | chr16:1078900-1078923_+ |
| STAT3 | ENST00000264657.9 | 1341 | CTGCTGTAGCTGATTCCATT | chr17:42348489-42348512_+ |
| | | 1342 | GAAACTGCCGCAGCTCCATT | chr17:42348416-42348439_+ |
| | | 1343 | AGCCGATCTAGGCAGATGTT | chr17:42337443-42337466_+ |
| | | 1344 | TCCAGTTCACTACTAAAGTC | chr17:42333671-42333694_− |
| | | 1345 | GACCCCTGATTTTAGCAGGA | chr17:42348512-42348535_− |
| SYK | ENST00000375754.8 | 1346 | CCCTTCGTGCAGCAGGCACA | chr9:90862237-90862260_− |
| | | 1347 | AGCCGTTGTTGTCTCTGGCT | chr9:90862208-90862231_− |
| | | 1348 | GCCATGCTTCAGGGGCCGGA | chr9:90843880-90843903_− |
| | | 1349 | CAGAAGATTACCTGGTCCAG | chr9:90843971-90843994_+ |
| | | 1350 | GGTTCCATGGAAAAATCTCT | chr9:90845518-90845541_+ |
| TBK1 | ENST00000331710.9 | 1351 | ATGTCTCCACTCCAGTCAAT | chr12:64480075-64480098_− |
| | | 1352 | CAAATTATTTGCTATTGAAG | chr12:64460304-64460327_+ |
| | | 1353 | CGACGCTTAGTCTTAGAACC | chr12:64484378-64484401_+ |
| | | 1354 | ATCAAGAACTTATCTACGAA | chr12:64484355-64484378_+ |
| | | 1355 | ATGCGTGTTATAGGGGAAGA | chr12:64466965-64466988_+ |
| TEK | ENST00000380036.8 | 1356 | AGGGGGGAGGATCCGGTGGA | chr9:27185530-27185553_− |
| | | 1357 | TCTTCACCTCGGCCTTCACC | chr9:27169593-27169616_+ |
| | | 1358 | ACAGTCATAGTTAAAGTAGC | chr9:27168505-27168528_− |
| | | 1359 | ATGCTGGAGTGTACTCGGCC | chr9:27169554-27169577_+ |
| | | 1360 | CTGGAGAGCAGAGACATCCT | chr9:27180315-27180338_− |
| TERT | ENST00000310581.9 | 1361 | CGAAGAAGCCACCTCTTTGG | chr5:1294026-1294049_− |
| | | 1362 | CCCGCGCCTCCTCGCACCCG | chr5:1294210-1294233_+ |
| | | 1363 | CTGGCGGCTGGTGCAGCGCG | chr5:1294862-1294885_− |
| | | 1364 | AGCAGCCGGTGTCTGTGCCC | chr5:1293600-1293623_− |
| | | 1365 | GGTCCACTAGCGTGTGGCGG | chr5:1294307-1294330_+ |
| TGFB1 | ENST00000221930.5 | 1366 | TGGATAGTCCCGCGGCCGGC | chr19:41352950-41352973_+ |
| | | 1367 | TGGTTATCTTTTGATGTCAC | chr19:41344775-41344798_− |
| | | 1368 | TCACCGGAGTTGTGCGGCAG | chr19:41344759-41344782_− |
| | | 1369 | TACTGGTGCTGACGCCTGGC | chr19:41352969-41352992_− |
| | | 1370 | CAACCACTGCCGCACAACTC | chr19:41344756-41344779_+ |
| TGFBR1 | ENST00000374994.8 | 1371 | GTTACGTCATGAAAACATCC | chr9:99138042-99138065_+ |
| | | 1372 | CCTCTTCATTTGGCACTCGA | chr9:99132629-99132652_− |
| | | 1373 | GTTTGGAGAGGAAAGTGGCG | chr9:99137938-99137961_+ |
| | | 1374 | TGTCTGCTGCTATAAATCCC | chr9:99138060-99138083_− |
| | | 1375 | TTACGTCATGAAAACATCCT | chr9:99138043-99138066_+ |
| TLR5 | ENST00000642603.1 | 1376 | CGGCCATCAAAGGAGCAGGA | chr1:223112945-223112968_+ |
| | | 1377 | ATGAGCTCGAGCCCCTACAA | chr1:223112446-223112469_− |
| | | 1378 | CCTCCTTGTCAATAGTCAAG | chr1:223112763-223112786_+ |
| | | 1379 | CTCCTTGTCAATAGTCAAGG | chr1:223112764-223112787_+ |
| | | 1380 | TATACAAGCTATTAGCTGCG | chr1:223112403-223112426_+ |
| TLR7 | ENST00000380659.3 | 1381 | ATGTTCTTAAACCATCTTGG | chrX:12886423-12886446_− |
| | | 1382 | ACATCCAGAGTGACATCACA | chrX:12885610-12885633_− |
| | | 1383 | CAGTCTGTGAAAGGACGCTG | chrX:12885749-12885772_− |
| | | 1384 | CAGCTACTAGAGATACCGCA | chrX:12885919-12885942_+ |
| | | 1385 | ACTGTGTACCTATTCCACTG | chrX:12885803-12885826_+ |
| TLR8 | ENST00000311912.5 | 1386 | CAAAATAGCTCCTGCAGCCT | chrX:12910403-12910426_+ |
| | | 1387 | ATTGAAGCACCACCATCACA | chrX:12919844-12919867_− |
| | | 1388 | AAAGACTCTGGCAAACCAGA | chrX:12919460-12919483_− |
| | | 1389 | ACAAGGCACGCATGGAAATG | chrX:12919827-12919850_− |
| | | 1390 | AAGTCAAGTATTTCTAAGCG | chrX:12920048-12920071_− |
| TNF | ENST00000449264.2 | 1391 | TGGAGTGATCGGCCCCCAGA | chr6:31575899-31575922_+ |
| | | 1392 | GCTCATGGTGTCCTTTCCAG | chr6:31575724-31575747_− |
| | | 1393 | AAGCACCGCCTGGAGCCCTG | chr6:31575810-31575833_− |
| | | 1394 | TTGGAGTGATCGGCCCCCAG | chr6:31575898-31575921_+ |
| | | 1395 | TGGGCTACAGGCTTGTCACT | chr6:31576783-31576806_− |
| TNFRSF8 | ENST00000263932.6 | 1396 | TAGAAGCAGCTTCCTGGGCG | chr1:12110124-12110147_− |
| | | 1397 | ACTACTATGACAAGGCTGTC | chr1:12084503-12084526_+ |
| | | 1398 | GCCTCATCCAGGTAGTAGTC | chr1:12097159-12097182_− |
| | | 1399 | CCAGCACCATGCCTGTAAGA | chr1:12110093-12110116_+ |
| | | 1400 | TCTGTGAGCCGGCTTCCCCA | chr1:12109586-12109609_+ |
| TNFSF11 | ENST00000398795.6 | 1401 | CTGCGTGGCTCGGAGGAGAT | chr13:42574336-42574359_+ |
| | | 1402 | GGCCACGAACATGGAGCGGG | chr13:42574433-42574456_− |
| | | 1403 | CCTAATAGAATATCAGAAGA | chr13:42581131-42581154_+ |
| | | 1404 | AATTAATACCTGATTCATGT | chr13:42581234-42581257_+ |
| | | 1405 | GACTACACCAAGTACCTGCG | chr13:42574321-42574344_+ |
| TNFSF13B | ENST00000375887.8 | 1406 | GCTTTCCGTCTTTGGAGGAT | chr13:108270011-108270034_− |
| | | 1407 | GAAGCTCCAGCTGTCACCGC | chr13:108270204-108270227_+ |
| | | 1408 | TCTTTGGAGGATCGGACAGA | chr13:108270003-108270026_− |
| | | 1409 | TGTTTCTTCTGGACCCTGAA | chr13:108270400-108270423_− |
| | | 1410 | GTCTTTGGAGGATCGGACAG | chr13:108270004-108270027_− |
| TNKS | ENST00000310430.10 | 1411 | CAGCGCTAGGCCGTGCCGCG | chr8:9556111-9556134_− |
| | | 1412 | CCCTTCGCCTCCCCGCGGCA | chr8:9556101-9556124_+ |
| | | 1413 | ACGGCCTAGCGCTGCCGGAG | chr8:9556120-9556143_+ |
| | | 1414 | GTAACTCAGCAGGAGGCGGC | chr8:9720440-9720463_− |
| | | 1415 | TCCCGACTGCCATCCCCCTC | chr8:9556134-9556157_− |
| TOP1 | ENST00000361337.2 | 1416 | CCCACTCATGTCGGCCCGGA | chr20:41029053-41029076_− |
| | | 1417 | GGTCCCCACTCATGTCGGCC | chr20:41029057-41029080_− |
| | | 1418 | TAGCTACTTCCTCTGCTTTG | chr20:41097238-41097261_− |
| | | 1419 | CCCCACTCATGTCGGCCCGG | chr20:41029054-41029077_− |
| | | 1420 | GAAGAAGAGCGCTATCCTGA | chr20:41092475-41092498_+ |
| TOP1MT | ENST00000329245.8 | 1421 | GGGCTCGCGCAGGACGCAGA | chr8:143334752-143334775_− |
| | | 1422 | CGATAACACCGTCACGTGGC | chr8:143324552-143324575_− |
| | | 1423 | CGTGGCGACCATCCCAAGAT | chr8:143325396-143325419_− |
| | | 1424 | GCCGGCGGGGCACCAGTGGA | chr8:143324585-143324608_− |
| | | 1425 | CCACGGCCACGGAACAAGCC | chr8:143325414-143325437_+ |
| TOP2A | ENST00000423485.5 | 1426 | GATAATGATTATGACAGATC | chr17:40407547-40407570_− |
| | | 1427 | CTCCGCCCAGACACCTACAT | chr17:40416761-40416784_− |
| | | 1428 | CAGAACCAATGTAGGTGTCT | chr17:40416756-40416779_+ |
| | | 1429 | TTTGGGCACCAGCACATCAA | chr17:40406469-40406492_− |
| | | 1430 | AGACACCTACATTGGTTCTG | chr17:40416753-40416776_− |
| TOP2B | ENST00000435706.6 | 1431 | CGGCGTGGGCGGCGGCAACG | chr3:25664242-25664265_− |
| | | 1432 | CGGAGACAGCGTAGGCTACA | chr3:25626781-25626804_− |
| | | 1433 | CATAATCTTTCCATAGCGTA | chr3:25630043-25630066_+ |
| | | 1434 | CATGTAGCCTACGCTGTCTC | chr3:25626782-25626805_+ |
| | | 1435 | GATTTGGCTGGTTCGTGTAG | chr3:25635969-25635992_− |
| TUBA1A | ENST00000301071.11 | 1436 | TGCCTGTGATAAGTTGCTCA | chr12:49186398-49186421_+ |
| | | 1437 | CACCTTCTTCAGTGAGACGG | chr12:49186664-49186687_− |
| | | 1438 | ACACCTTCTTCAGTGAGACG | chr12:49186665-49186688_− |
| | | 1439 | CACCCTGAGCAACTTATCAC | chr12:49186400-49186423_− |
| | | 1440 | GGAGATCATTGACCTCGTGT | chr12:49186326-49186349_− |
| TUBA4A | ENST00000248437.8 | 1441 | GAGCTCTATTGCTTGGAACA | chr2:219252147-219252170_− |
| | | 1442 | CAGATCCACAAAAACTGCCC | chr2:219252023-219252046_+ |
| | | 1443 | GTGCTGGAAAACACGTACCC | chr2:219252041-219252064_− |
| | | 1444 | CTGCTGGGAGCTCTATTGCT | chr2:219252154-219252177_− |
| | | 1445 | ACCCAGAGCAGCTCATCACT | chr2:219251654-219251677_− |
| TUBB | ENST00000327892.12 | 1446 | ATATGTTCCTCGTGCCATCC | chr6:30722924-30722947_+ |
| | | 1447 | CATTGTAGTACACAGAGATG | chr6:30722613-30722636_− |
| | | 1448 | TGTTCCTCGTGCCATCCTGG | chr6:30722927-30722950_+ |
| | | 1449 | AGGTTCTAGATCCACCAGGA | chr6:30722938-30722961_− |
| | | 1450 | AGATCCACCAGGATGGCACG | chr6:30722931-30722954_− |
| TUBB1 | ENST00000217133.1 | 1451 | CGAATGCTGTCCATCGTCCC | chr20:59023530-59023553_− |
| | | 1452 | GACTTGGCTGGGAGCGACCG | chr20:59022877-59022900_+ |
| | | 1453 | GGACCTAGAACCTGGGACGA | chr20:59023520-59023543_+ |
| | | 1454 | GCTGCAAGGCCGAGGCCCCG | chr20:59022894-59022917_− |
| | | 1455 | GCTGATTCTCTCCAGCTGCA | chr20:59022908-59022931_− |
| TUBB3 | ENST00000315491.11 | 1456 | TACTGGGCGCGTCTGGCGGG | chr16:89923379-89923402_− |
| | | 1457 | ATTCTGGTGGACCTGGAACC | chr16:89933490-89933513_+ |
| | | 1458 | AGGCCTGAAGAGATGTCCAA | chr16:89933539-89933562_− |
| | | 1459 | GATGTCCAAAGGCCCCTGAG | chr16:89933528-89933551_− |
| | | 1460 | CCATGGACAGTGTCCGCTCA | chr16:89933515-89933538_+ |
| TUBD1 | ENST00000325752.7 | 1461 | TAGTGACTCACACAGTTCCC | chr17:59890908-59890931_− |
| | | 1462 | CATCATAATGAGTATGGCTG | chr17:59880997-59881020_− |
| | | 1463 | TCATCATAATGAGTATGGCT | chr17:59880998-59881021_− |
| | | 1464 | ATATTTCCATTGGCCAGACT | chr17:59886138-59886161_+ |
| | | 1465 | TCAATTGTAACAGTGCAACT | chr17:59890976-59890999_− |
| TUBE1 | ENST00000368662.9 | 1466 | TACGACCACCGACTGGGTCA | chr6:112087413-112087436_+ |
| | | 1467 | GACCACCGACTGGGTCATGG | chr6:112087416-112087439_+ |
| | | 1468 | GCGCACCACCATGACCCAGT | chr6:112087421-112087444_− |
| | | 1469 | GACACGAGATAATGAAGTTG | chr6:112074760-112074783_+ |
| | | 1470 | CATTAGCAAAATCGACCTCA | chr6:112076075-112076098_− |
| TUBG1 | ENST00000251413.7 | 1471 | GCCGCACTGGCCCAACTGTA | chr17:42609756-42609779_− |
| | | 1472 | TCTTAGAACGGCTGAATGAC | chr17:42612484-42612507_+ |
| | | 1473 | TGTCATTCAGCCGTTCTAAG | chr17:42612482-42612505_− |
| | | 1474 | TGCCGCACTGGCCCAACTGT | chr17:42609757-42609780_− |
| | | 1475 | CGGCATCGCTCCTCAGGCAC | chr17:42609720-42609743_− |
| TXN | ENST00000374517.5 | 1476 | TACTTCAAGGAATATCACGT | chr9:110250837-110250860_+ |
| | | 1477 | TTTATCACCTGCAGCGTCCA | chr9:110251423-110251446_+ |
| | | 1478 | TCAGACTCCAGCAGCCAAGA | chr9:110256431-110256454_− |
| | | 1479 | TTTGCAAGGCCCACACCACG | chr9:110251378-110251401_+ |
| | | 1480 | TAGTTGACTTCTCAGCCACG | chr9:110251393-110251416_− |
| TYK2 | ENST00000525621.5 | 1481 | TCTCCGCCATGGCATCCCCC | chr19:10366438-10366461_− |
| | | 1482 | CGAAGGACAGCGCCGCAGCC | chr19:10364731-10364754_+ |
| | | 1483 | CCCCAGCGTTCGGGAACTTG | chr19:10362341-10362364_− |
| | | 1484 | GGGCTGGGCTCCATCCCCAA | chr19:10378343-10378366_+ |
| | | 1485 | CTCTGGGGATCTCTAGGATG | chr19:10368325-10368348_+ |
| TYMS | ENST00000323274.14 | 1486 | AGCCGGCCACAGGCATGGCG | chr18:657735-657758_− |
| | | 1487 | CTTCCAGTGGAGGCATTTTG | chr18:662273-662296_+ |
| | | 1488 | CTGCCCCAAAATGCCTCCAC | chr18:662276-662299_− |
| | | 1489 | CACGTTTGGTTGTCAGCAGA | chr18:659647-659670_− |
| | | 1490 | CGGCCACAGGCATGGCGCGG | chr18:657732-657755_− |
| VDR | ENST00000395324.6 | 1491 | GGAACGTGCCCCGGATCTGT | chr12:47879038-47879061_− |
| | | 1492 | CCCCGGATCTGTGGGGTGTG | chr12:47879030-47879053_− |
| | | 1493 | CTGACCCTGGAGACTTTGAC | chr12:47879059-47879082_− |
| | | 1494 | CAGGCGAAGCATGAAGCGGA | chr12:47865157-47865180_− |
| | | 1495 | ACTTTGACCGGAACGTGCCC | chr12:47879047-47879070_− |
| VEGFA | ENST00000372055.8 | 1496 | GAGAAGTGCTAGCTCGGGCC | chr6:43770945-43770968_+ |
| | | 1497 | AGTAGCTCGCCGAGGCGCCG | chr6:43771149-43771172_+ |
| | | 1498 | GGCTTCCCCCGCGCGGACCA | chr6:43770905-43770928_− |
| | | 1499 | GCCGCGAGAAGTGCTAGCTC | chr6:43770940-43770963_+ |
| | | 1500 | CCTCTCCGGCTCGGGCTGTG | chr6:43771183-43771206_− |
| VEGFB | ENST00000309422.6 | 1501 | GCCCAGTGGGCACACACTCC | chr11:64235966-64235989_− |
| | | 1502 | TGCGTGACTGTGCAGCGCTG | chr11:64235922-64235945_+ |
| | | 1503 | AGGGCTCATGGTGCCCGCCG | chr11:64234819-64234842_− |
| | | 1504 | ACAGTGCTGTGAAGCCAGAC | chr11:64237200-64237223_+ |
| | | 1505 | CGGACTTGGTGCTGCCCAGT | chr11:64235979-64236002_− |
| WEE1 | ENST00000450114.6 | 1506 | TTGCGGAAGGTCTTGTGTGG | chr11:9574452-9574475_− |
| | | 1507 | ACTCGCCGCTGCCGCCCGCG | chr11:9574108-9574131_+ |
| | | 1508 | TCCTCCTCACAGTCGCTGCA | chr11:9574017-9574040_− |
| | | 1509 | GAGGAGGACCTGTTGCTGCC | chr11:9574200-9574223_+ |
| | | 1510 | CGATCAAAAAAGCCATTGGC | chr11:9576624-9576647_+ |
| XIAP | ENST00000434753.7 | 1511 | CTATCTTTTGAGAACTGGGC | chrX:123886075-123886098_+ |
| | | 1512 | GCTTCATAATCTGCCATGGA | chrX:123886439-123886462_− |
| | | 1513 | CCAAATTGCAGATTTATCAA | chrX:123885923-123885946_+ |
| | | 1514 | CTTCTTCACTATACATGGCA | chrX:123886132-123886155_− |
| | | 1515 | ATAGTCTGGCCAGTTCTGAA | chrX:123886167-123886190_− |
| XPO1 | ENST00000401558.6 | 1516 | TAAAGGAGCATCCTGATGCT | chr2:61526467-61526490_− |
| | | 1517 | AACAAGAATGGCCCAAACAT | chr2:61499864-61499887_− |
| | | 1518 | TTAAATGCTTAGATTTGACT | chr2:61499719-61499742_+ |
| | | 1519 | GGCAAATATAGCTGTCTCCT | chr2:61493906-61493929_+ |
| | | 1520 | TGGTCTCAAAAATATATCCC | chr2:61498698-61498721_+ |
| YES1 | ENST00000584307.5 | 1521 | GAAGCAAGATCAATCGCTAC | chr18:747979-748002_− |
| | | 1522 | GGATCCTCCAAATGGTGTCA | chr18:756632-756655_+ |
| | | 1523 | TTGAATCCTGGAAATCAACG | chr18:745982-746005_− |
| | | 1524 | ATGTTTCGGCGAAGTGTGGA | chr18:743259-743282_− |
| | | 1525 | CAGAGTATCAAATTGTGCTC | chr18:745732-745755_+ |

TABLE 6B
Library of gene modulatory reagents.

| | SEQ ID | | |
|---|---|---|---|
| Target gene | NO | gRNA # | gRNA Seq |
| ANPEP | 1526 | 1 | CGTTCAGGGCATAATCGCCG |
| ANPEP | 1527 | 2 | TCACGGTGGATACCAGCACG |
| ANPEP | 1528 | 3 | CATCACGCTTATCCACCCCA |
| ANPEP | 1529 | 4 | CCTTGGACCAAAGTAAAGCG |
| HDAC11 | 1530 | 1 | GGCGAGCGTGATGTCCGCAT |
| HDAC11 | 1531 | 2 | CGAGGCTGGGCCATCAACGT |
| HDAC11 | 1532 | 3 | GCAACAGCAAAGGACCACTG |
| HDAC11 | 1533 | 4 | ACAACCGCCACATCTACCCA |
| ROCK1 | 1534 | 1 | GCAAAGTCTGTGGCAATGTG |
| ROCK1 | 1535 | 2 | AGTCATACCTGAACAACCCA |
| ROCK1 | 1536 | 3 | TTACATATTATAGCAATCGT |
| ROCK1 | 1537 | 4 | CATGGTACGATGTGATACAG |
| BRAF | 1538 | 1 | ATACCCAATAGAGTCCGAGG |
| BRAF | 1539 | 2 | TCATAATTAACACACATCAG |
| BRAF | 1540 | 3 | ACAAATGATTAAGTTGACAC |
| BRAF | 1541 | 4 | GGGGGTAGCAGACAAACCTG |
| NRAS | 1542 | 1 | CCATGAGAGACCAATACATG |
| NRAS | 1543 | 2 | TGAATATGATCCCACCATAG |
| NRAS | 1544 | 3 | TGATGTACCTATGGTGCTAG |
| NRAS | 1545 | 4 | TTGCGGATATTAACCTCTAC |
| PPARG | 1546 | 1 | CACGACATTCAATTGCCATG |
| PPARG | 1547 | 2 | AGTGAAGGGCTTGATATCAA |
| PPARG | 1548 | 3 | TGGCATCTCTGTGTCAACCA |
| PPARG | 1549 | 4 | ACAGATGTGATCTTAACTGT |
| INIIBB | 1550 | 1 | GCATACCTTCCCACTCACGG |
| INIIBB | 1551 | 2 | CAGGACACCTGTACGTCGTG |
| INIIBB | 1552 | 3 | GAAGAAGTATAGGCGGACCC |
| INIIBB | 1553 | 4 | TCGCCTGGGTCCACGAACAC |
| TOP2A | 1554 | 1 | TCCCGTCAGAACATGGACCC |
| TOP2A | 1555 | 2 | AGCATTGTAAAGATGTATCG |
| TOP2A | 1556 | 3 | TGTACGCTTATCCTGACTGA |
| TOP2A | 1557 | 4 | ATTCAGTACCAAATTTACTG |
| ADA | 1558 | 1 | TCACCGTACTGTCCACGCCG |
| ADA | 1559 | 2 | TGGACATACTCAAGACAGAG |
| ADA | 1560 | 3 | CACAGACTGGTCCCCCAAGG |
| ADA | 1561 | 4 | TCCCAGCTAACACAGCAGAG |
| SRC | 1562 | 1 | GACCTGGAACGGTACCACCA |
| SRC | 1563 | 2 | TCAATGCAGAGAACCCGAGA |
| SRC | 1564 | 3 | TGTCCTTCAAGAAAGGCGAG |
| SRC | 1565 | 4 | GTCACGGAGTACATGAGCAA |
| PIM1 | 1566 | 1 | GGCGAGTCGGAGGACAACTG |
| PIM1 | 1567 | 2 | GTCCAGGAGCCTAATGACGC |
| PIM1 | 1568 | 3 | AAGGACCGGATTTCCGACTG |
| PIM1 | 1569 | 4 | TCTTCGACTTCATCACGGAA |
| RPTOR | 1570 | 1 | GATCTGGCAGAACTTCGACT |
| RPTOR | 1571 | 2 | CTTACGTCGCAACGCCAAGG |
| RPTOR | 1572 | 3 | TGGTGTGGACCCTCCCGATG |
| RPTOR | 1573 | 4 | CACATGGCGTGCATGTACGT |
| PTK6 | 1574 | 1 | CGCACCCGACAGGACGTAGT |
| PTK6 | 1575 | 2 | CGTGGAAGACGTCCCCCGCG |
| PTK6 | 1576 | 3 | CTCTCCCAGTCATCCCAATG |
| PTK6 | 1577 | 4 | GCCGACGCACAGCTTCCGAG |
| CDK7 | 1578 | 1 | AGCTCCAAATAGTAACTCGG |
| CDK7 | 1579 | 2 | ATCTCTGGCCTTGTAAACGG |
| CDK7 | 1580 | 3 | TTTCCATAAAATCAAAGACA |
| CDK7 | 1581 | 4 | TTAAAAACCTTACCCTATGT |
| PSMB10 | 1582 | 1 | CGATCAGCGATGCACCCACG |
| PSMB10 | 1583 | 2 | CACTAACGATTCGGTCGTGG |
| PSMB10 | 1584 | 3 | GAGCTACACGCGTTATCTAC |
| PSMB10 | 1585 | 4 | TCTCCTTCGAGAACTGCCAA |
| MAP2K1 | 1586 | 1 | CATCCTAGTCAACTCCCGTG |
| MAP2K1 | 1587 | 2 | GCAGCAGCGAAAGCGCCTTG |
| MAP2K1 | 1588 | 3 | GGGCACAAGGTCCTACATGT |
| MAP2K1 | 1589 | 4 | TATGGTGCGTTCTACAGCGA |
| VEGFB | 1590 | 1 | CACACTCCAGGCCATCGTCA |
| VEGFB | 1591 | 2 | CATCTATCCATGACACCACT |
| VEGFB | 1592 | 3 | GCAGCACCAAGTCCGGATGC |
| VEGFB | 1593 | 4 | CGGTACCCGAGCAGTCAGCT |
| SLC2A2 | 1594 | 1 | GTGCCACTAGAATAGGCTGT |
| SLC2A2 | 1595 | 2 | CCTTTACATCAAGTTAGATG |
| SLC2A2 | 1596 | 3 | CACCGATATACATAGGAACC |
| SLC2A2 | 1597 | 4 | TAGTTGGAGCTCTCTTGATG |
| EHMT1 | 1598 | 1 | GGGCCGGTGCACAAACAGCG |
| EHMT1 | 1599 | 2 | TTCGGCTGCTTCCATCAACG |
| EHMT1 | 1600 | 3 | ACTTATACGACTCAGAACCT |
| EHMT1 | 1601 | 4 | CAACACACTAACTCGGATAG |
| CDK5 | 1602 | 1 | TAGCCGCAATGTGCTACACA |
| CDK5 | 1603 | 2 | CCTTTACAATCTCAGGATCG |
| CDK5 | 1604 | 3 | CGTCCGCTGTTACTCAGCTG |
| CDK5 | 1605 | 4 | CCGGGAGACTCATGAGATCG |
| CXCR2 | 1606 | 1 | GGCGGCATCTAGTAGAAAAG |
| CXCR2 | 1607 | 2 | TAAGATGACCAGCATCACGA |
| CXCR2 | 1608 | 3 | CAGTGGCACGATGAAGCCAA |
| CXCR2 | 1609 | 4 | CCAGCCTGCTATGAGGACAT |
| IFNAR1 | 1610 | 1 | GTACATTGTATAAAGACCAC |
| IFNAR1 | 1611 | 2 | ATAATTGGATAAAATTGTCT |
| IFNAR1 | 1612 | 3 | CTCCGCGTACAAGCATCTGA |
| IFNAR1 | 1613 | 4 | TAGATGACAACTTTATCCTG |
| EHMT2 | 1614 | 1 | CAAGAGGTGACCATCCCCCG |
| EHMT2 | 1615 | 2 | CTCCAGGTGGTTGTTCACCA |
| EHMT2 | 1616 | 3 | CGGACAGGTACAACTGCCGA |
| EHMT2 | 1617 | 4 | CTCTCCGTCCACACTCTCAG |
| DHX9 | 1618 | 1 | CAAAACATTATACTGGCATG |
| DHX9 | 1619 | 2 | TCAATCACAGCATCCAAAGG |
| DHX9 | 1620 | 3 | TGGATGTGGGAAAACCACAC |
| DHX9 | 1621 | 4 | GGAGATTTACCAACAACCAT |
| RAC1 | 1622 | 1 | TCACATCTAGTGGTATCCTG |
| RAC1 | 1623 | 2 | CTGTTTGCGGATAGGATAGG |
| RAC1 | 1624 | 3 | ATTTAAGATACTTACACAGT |
| RAC1 | 1625 | 4 | CTGGGCTTATGGGATACAGC |
| KIF11 | 1626 | 1 | TCTTGTGTAGGAGTATACGG |
| KIF11 | 1627 | 2 | GACTGAATTACCTTGTTACG |
| KIF11 | 1628 | 3 | GAAGTTAGTGTACGAACTGG |
| KIF11 | 1629 | 4 | ACCTAATGAAGAGTATACCT |
| TUBD1 | 1630 | 1 | AATGAATTCTCAGACATGTG |
| TUBD1 | 1631 | 2 | ATAGAGGGACAAATACCTAG |
| TUBD1 | 1632 | 3 | AGTGACTCACACAGTTCCCA |
| TUBD1 | 1633 | 4 | GCATGCTTCTGTCAAAAACA |
| PSMD1 | 1634 | 1 | CCTTAGATCGTTAGACACGG |
| PSMD1 | 1635 | 2 | TGAATCTCTCTGTCGTGACA |
| PSMD1 | 1636 | 3 | GTTAGCCAGAGCCACTAACT |
| PSMD1 | 1637 | 4 | TCACTACACCAAACAATGTG |
| HDAC9 | 1638 | 1 | AGCTTTGATCCAATGATGTG |
| HDAC9 | 1639 | 2 | AACAGCATGAGAACTTGACA |
| HDAC9 | 1640 | 3 | TTTCCCTCTAAAGTAACATG |
| HDAC9 | 1641 | 4 | GAGAGCGCACGTGTGTGCGT |
| PIGF | 1642 | 1 | GGTCCTAACAAACATAAGCA |
| PIGF | 1643 | 2 | TTTCAAACTTACTCTATCAG |
| PIGF | 1644 | 3 | AAGAAGTTCATTATCACACA |
| PIGF | 1645 | 4 | TATTGGAAACACACTTGACA |
| TUBA4A | 1646 | 1 | GCTCGATGTCTAGGTTGCGG |
| TUBA4A | 1647 | 2 | TTCCTCTCACCAATGACCGT |
| TUBA4A | 1648 | 3 | GAGCCGCTCCATCAGGAGTG |
| TUBA4A | 1649 | 4 | GTGACAAGACCATTGGTGGA |
| TOP1MT | 1650 | 1 | GCTCCGATAACACCGTCACG |
| TOP1MT | 1651 | 2 | TCATGAATACACAACAAAGG |
| TOP1MT | 1652 | 3 | CCATACGAGCCCCTTCCCGA |
| TOP1MT | 1653 | 4 | GTGGCGACCATCCCAAGATG |
| NTRK3 | 1654 | 1 | TCTTCACACGCTCAACGCCG |
| NTRK3 | 1655 | 2 | CGTCAACCTGACCGTACGAG |
| NTRK3 | 1656 | 3 | CATGTGGAATACTACCAAGA |
| NTRK3 | 1657 | 4 | TCATGCCATCAACTTGACGC |
| RXRA | 1658 | 1 | CCTACGTGGAGGCAAACATG |
| RXRA | 1659 | 2 | AGGACTGCCTGATTGACAAG |
| RXRA | 1660 | 3 | AGGAAGCCATGTTTCCTGAG |
| RXRA | 1661 | 4 | CAAGGACCGGAACGAGAATG |
| MCL1 | 1662 | 1 | AGGCGCTGGAGACCTTACGA |
| MCL1 | 1663 | 2 | GTAATAACACCAGTACGGAC |
| MCL1 | 1664 | 3 | AGTCGCTGGAGATTATCTCT |
| MCL1 | 1665 | 4 | CCAAAAGTCGCCCTCCCGGG |
| MTOR | 1666 | 1 | TCAGGAAATGATCCGCACAG |
| MTOR | 1667 | 2 | GGTGATGGCCTGGACAACCA |
| MTOR | 1668 | 3 | CAGCATCGGATGCTTAGGAG |
| MTOR | 1669 | 4 | GTGAAGGGGGTAATGTGACG |
| HDAC7 | 1670 | 1 | TGCAGTCGGTCCACTCTGAG |
| HDAC7 | 1671 | 2 | GTTACCTGTAGGGAATGCCG |
| HDAC7 | 1672 | 3 | AAGGACTGGGCAAAGTGGAA |
| HDAC7 | 1673 | 4 | GACCTGGAGACAGATGGCGG |
| CHEK1 | 1674 | 1 | ACACCACCTGAAGTGACTCG |
| CHEK1 | 1675 | 2 | TGGTATTGGAATAACTCACA |
| CHEK1 | 1676 | 3 | CTTACTGCAATGCTCGCTGG |
| CHEK1 | 1677 | 4 | TTTCTGGAGTACTGTAGTGG |
| PSEN1 | 1678 | 1 | GCCACGCAGTCCATTCAGGG |
| PSEN1 | 1679 | 2 | TAAAACCTATAACGTTGCTG |
| PSEN1 | 1680 | 3 | ACCTGCCGGGAGTTACCCTG |
| PSEN1 | 1681 | 4 | TGTATTTATACAGAACCACC |
| FOLH1 | 1682 | 1 | TTATAGGCGTGGAATTGCAG |
| FOLH1 | 1683 | 2 | GGAGAGAAAGCACTGAAAGG |
| FOLH1 | 1684 | 3 | GAGGCGCCCCCCTACCGAAG |
| FOLH1 | 1685 | 4 | AAGATTCCAACCATCTGGAT |
| FKBP1A | 1686 | 1 | GGGCGCACCTTCCCCAAGCG |
| FKBP1A | 1687 | 2 | ACCGGTGTAGTGCACCACGC |
| FKBP1A | 1688 | 3 | GGCAAGCAGGAGGTGATCCG |
| FKBP1A | 1689 | 4 | GAAACCATCTCCCCAGGAGA |
| TUBG1 | 1690 | 1 | GACATGATGGTAGACACCTG |
| TUBG1 | 1691 | 2 | CGTGGAGGAGTTCGCCACCG |
| TUBG1 | 1692 | 3 | AGATGGTAGTGACAGTCTAG |
| TUBG1 | 1693 | 4 | TGACTGGTCCGTAGTGAGAG |
| GNRHR | 1694 | 1 | GTCCTGCAAAGACACTACTG |
| GNRHR | 1695 | 2 | GGAAGAAAGTAACCGTCACT |
| GNRHR | 1696 | 3 | TGTGGAACATTACAGTCCAA |
| GNRHR | 1697 | 4 | AGTCTCCAACAGGTTGGCTA |
| PLK2 | 1698 | 1 | ATCACCACCATTCGCACTCG |
| PLK2 | 1699 | 2 | AGCCATGGAACTAAAAGTTG |
| PLK2 | 1700 | 3 | TATGTTGTCCAAAAACCCAG |
| PLK2 | 1701 | 4 | GTCCTCAACAAACAAGGACA |
| PDGFRB | 1702 | 1 | TGTGGTAAGGCATATCCAAG |
| PDGFRB | 1703 | 2 | GACTAACGTGACGTACTGGG |
| PDGFRB | 1704 | 3 | GTCCCCTATGATCACCAACG |
| PDGFRB | 1705 | 4 | CTCCCGTGTCTAGCCCAGTG |
| FOLR1 | 1706 | 1 | AGTTGGGGGAGCACTCGTAG |
| FOLR1 | 1707 | 2 | CCATCCAGTGTCGACCCTGG |
| FOLR1 | 1708 | 3 | CATGAACGCCAAGCACCACA |
| FOLR1 | 1709 | 4 | GCTGCTCCTTCTAGTGTGGG |
| PLK3 | 1710 | 1 | GCTGATAGGGAGTCGATCGG |
| PLK3 | 1711 | 2 | GGTGCGAAAAACGCACGATG |
| PLK3 | 1712 | 3 | GTGACCATACATCCGCCTCA |
| PLK3 | 1713 | 4 | CTCAAGTACTTGCACCAGCG |
| MST1R | 1714 | 1 | CTGCCCACCTAAGCTTACTG |
| MST1R | 1715 | 2 | GTGGCATGTTAGTCACGGTG |
| MST1R | 1716 | 3 | GTGGGTATCAACGTGACCGT |
| MST1R | 1717 | 4 | CTCGGACCACATATTCAGGA |
| TUBA1A | 1718 | 1 | CTGTGATAAGTTGCTCAGGG |
| TUBA1A | 1719 | 2 | AGGTTGGACGCTCAATATCG |
| TUBA1A | 1720 | 3 | CTGGAGACCCGTGCACTGGT |
| TUBA1A | 1721 | 4 | TACAGAAAGCTGTTCATGGT |
| POLA1 | 1722 | 1 | CATGACACAACAGCTCACAT |
| POLA1 | 1723 | 2 | CAACAAGAACTCGTTACGCT |
| POLA1 | 1724 | 3 | AAGCACGCAATAAAGACAAG |
| POLA1 | 1725 | 4 | CTCTACACACTTTACCGTGG |
| DYRK1A | 1726 | 1 | TTCAACCAAAATACACCCGA |
| DYRK1A | 1727 | 2 | TCAGCAACCTCTAACTAACC |
| DYRK1A | 1728 | 3 | TGAGAAACACCAATTTCCGA |
| DYRK1A | 1729 | 4 | TTACAGGAGTACAAACCACC |
| ERBB4 | 1730 | 1 | CTGGTGTGTCCAGATAGCTA |
| ERBB4 | 1731 | 2 | AGCGGCGACACGACAGACAT |
| ERBB4 | 1732 | 3 | ATGTCCAGATGGCTTACAGG |
| ERBB4 | 1733 | 4 | ATAGAGTACTCTTCCACCAA |
| NCSTN | 1734 | 1 | TAAAGCCTATAAATACAACT |
| NCSTN | 1735 | 2 | CCAGCAGAACCATGTAAGGG |
| NCSTN | 1736 | 3 | ATGGTCTACGATATGGAGAA |
| NCSTN | 1737 | 4 | CAGTGCCCAAATGATGGGTT |
| ANGPT2 | 1738 | 1 | ACCGAGTCATCGTATTCGAG |
| ANGPT2 | 1739 | 2 | ACCAAACAGCGGAGCAAACG |
| ANGPT2 | 1740 | 3 | TAACGTGTAGATGCCATTCG |
| ANGPT2 | 1741 | 4 | AAGAACTGAATTATTCACCG |
| AR | 1742 | 1 | AGGGTACCACACATCAGGTG |
| AR | 1743 | 2 | CCTTAAAGACATCCTGAGCG |
| AR | 1744 | 3 | GGACGCAACCTCTCTCGGGG |
| AR | 1745 | 4 | TCCAGCTTGATGCGAGCGTG |
| SLAMF7 | 1746 | 1 | TCAGGGAGTAGCCTCCATCT |
| SLAMF7 | 1747 | 2 | ACTCAGGGATCTACTATGTG |
| SLAMF7 | 1748 | 3 | GACCAATCTGACATGCTGCA |
| SLAMF7 | 1749 | 4 | TGGCTGTATGGTGACAAGAG |
| DHFR | 1750 | 1 | CGGCCCGGCAGATACCTGAG |
| DHFR | 1751 | 2 | AACCTTAGGGAACCTCCACA |
| DHFR | 1752 | 3 | GTCGCTGTGTCCCAGAACAT |
| DHFR | 1753 | 4 | GACATGGTCTGGATAGTTGG |
| PDGFRA | 1754 | 1 | TAAGTCAGGGGAAACGATTG |
| PDGFRA | 1755 | 2 | CCTGCGTTCTGAACTCACGG |
| PDGFRA | 1756 | 3 | GACTTGGTCGATGATCACCA |
| PDGFRA | 1757 | 4 | AAATAATCCGTCATTCCTAG |
| TUBB3 | 1758 | 1 | CTGGCCCGGGAAGCGCAAGG |
| TUBB3 | 1759 | 2 | CATGGACAGTGTCCGCTCAG |
| TUBB3 | 1760 | 3 | CAGCTGGTGGATGGACAGCG |
| TUBB3 | 1761 | 4 | CTGGGCCAAGGGTCACTACA |
| IGF1R | 1762 | 1 | GGAGAACGACCATATCCGTG |
| IGF1R | 1763 | 2 | TTCCGAAATTTACCGCATGG |
| IGF1R | 1764 | 3 | GGTACAATGTGAAAGGCCGA |
| IGF1R | 1765 | 4 | TGTGGGGAATAAGCCCCCAA |
| RAF1 | 1766 | 1 | GACCATGTGGACATTAGGTG |
| RAF1 | 1767 | 2 | AGACTTCTCCACGAACACAA |
| RAF1 | 1768 | 3 | GCCGAACAAGCAAAGAACAG |
| RAF1 | 1769 | 4 | TGTTGCAGTAAAGATCCTAA |
| SYK | 1770 | 1 | GGTGTACGAGAGCCCCTACG |
| SYK | 1771 | 2 | CACACCACTACACCATCGAG |
| SYK | 1772 | 3 | ATCCGAGCCAGAGACAACAA |
| SYK | 1773 | 4 | TAATAACTCATCTTTAAGAG |
| MAP2 | 1774 | 1 | TTTGTCACCAGAGATATGCG |
| MAP2 | 1775 | 2 | CCTGATAAAAAGGACATGCA |
| MAP2 | 1776 | 3 | CATGCCAAGTCCCTTTCAAG |
| MAP2 | 1777 | 4 | GATTGCCGTCAAATTGTCAG |
| FOLR2 | 1778 | 1 | AACTTTAACTGGGACCACTG |
| FOLR2 | 1779 | 2 | AGAGGACTGTCAGCGCTGGT |
| FOLR2 | 1780 | 3 | ACTGGCACAGAGGATGGGAC |
| FOLR2 | 1781 | 4 | AAGCACCACAAGACAAAGCC |
| CDK9 | 1782 | 1 | CCAGAGTGTCACCACACGGT |
| CDK9 | 1783 | 2 | TCTCCCGCAAGGCTGTAATG |
| CDK9 | 1784 | 3 | GCGGTTATAGGGGGAAGCTG |
| CDK9 | 1785 | 4 | GCTGACTGATGAGGGCGAGT |
| PRKCG | 1786 | 1 | ACTCGAAGGTCACAAATTCG |
| PRKCG | 1787 | 2 | ATTCCACACAGGGTTTAGCG |
| PRKCG | 1788 | 3 | CGAGTATTACAATGTGCCGG |
| PRKCG | 1789 | 4 | CTCTACGGGCTTGTGCACCA |
| MS4A1 | 1790 | 1 | TGGGTGCATAGATCCCTGCT |
| MS4A1 | 1791 | 2 | TCATGAAGAAGCTTTGCGTG |
| MS4A1 | 1792 | 3 | GTAACAGTATTGGGTAGATG |
| MS4A1 | 1793 | 4 | TATTATTTCCGGATCACTCC |
| INHA | 1794 | 1 | TGCCCCGAAGACATGCCCTG |
| INHA | 1795 | 2 | GAGGACAAGTCAGCTGCCAG |
| INHA | 1796 | 3 | CGGACCAGACCACCCAGTGG |
| INHA | 1797 | 4 | CAGCAGCACCAGGACGGGGT |
| F2R | 1798 | 1 | GGAGCTGGTCAAATATCCGG |
| F2R | 1799 | 2 | AAATGACCGGGGATCTAAGG |
| F2R | 1800 | 3 | TGGCCATGATGTTTAGTGGG |
| F2R | 1801 | 4 | CACAAACAGCACATCTGCCG |
| MAPK11 | 1802 | 1 | GCTTCTGGACGTCTTCACGC |
| MAPK11 | 1803 | 2 | CTGCGGTCGCACCTACCCGG |
| MAPK11 | 1804 | 3 | TGCGCGCGTGGATCAGCGAC |
| MAPK11 | 1805 | 4 | CCAGACGGAGCCGTAGGCGC |
| PLK1 | 1806 | 1 | AACCAAAGTCGAATATGACG |
| PLK1 | 1807 | 2 | CCTGCCTGACCATTCCACCA |
| PLK1 | 1808 | 3 | AGCCAAGCACAATTTGCCGT |
| PLK1 | 1809 | 4 | CGTTGTCCTCGAAAAAGCCG |
| TUBB | 1810 | 1 | GCTGACCACACCAACCTACG |
| TUBB | 1811 | 2 | CCCCACCGGCACCTACCACG |
| TUBB | 1812 | 3 | AGATCCACCAGGATGGCACG |
| TUBB | 1813 | 4 | CTGCATTCCAGGTCAGTCTG |
| HDAC8 | 1814 | 1 | ATTTGAGCGTATTCTCTACG |
| HDAC8 | 1815 | 2 | ATAGTCAAATATCCCTTCAG |
| HDAC8 | 1816 | 3 | GTAAATGTGCCCATTCAGGA |
| HDAC8 | 1817 | 4 | GCTGCAGATAAGCATCAGTG |
| PTCH1 | 1818 | 1 | TGATGTCGATGGGCTTATCG |
| PTCH1 | 1819 | 2 | AGCGGGAATTGGGATTAACG |
| PTCH1 | 1820 | 3 | AAATGTACGAGCACTTCAAG |
| PTCH1 | 1821 | 4 | AAGGTGTAATAATCAAACAA |
| PARP3 | 1822 | 1 | GTGGACATGTTGGATCCACG |
| PARP3 | 1823 | 2 | AGCAAGCAACAGATTGCACG |
| PARP3 | 1824 | 3 | ACTTATCGAAGTACAGGCAG |
| PARP3 | 1825 | 4 | GATGACGGTGTAAAAGTGTG |
| HDAC2 | 1826 | 1 | GATGTATCAACCTAGTGCTG |
| HDAC2 | 1827 | 2 | TACAACAGATCGTGTAATGA |
| HDAC2 | 1828 | 3 | CCTCCTCCAAGCATCAGTAA |
| HDAC2 | 1829 | 4 | TCAAAGAGTCCATCAAACAC |
| APH1A | 1830 | 1 | GACACCACTGATGATACCGA |
| APH1A | 1831 | 2 | GCGTTATCATCCTGGTCGCA |
| APH1A | 1832 | 3 | AGTGATCAAGAAAAGCGCGA |
| APH1A | 1833 | 4 | GCTCACCATAGGCCATCTGG |
| PSMB1 | 1834 | 1 | ATAATAAGGCCATGACTACG |
| PSMB1 | 1835 | 2 | GTATACAGCTTTGATCCAGT |
| PSMB1 | 1836 | 3 | TCTGATACTCGATTGAGTGA |
| PSMB1 | 1837 | 4 | TTACCCTCCGTTGAAAACGT |
| DYRK3 | 1838 | 1 | CCACGGGCGCAGTTCAACCA |
| DYRK3 | 1839 | 2 | TTGGTGGTCCCAATAATGGA |
| DYRK3 | 1840 | 3 | GGCATCCAAAGATTGCAAGA |
| DYRK3 | 1841 | 4 | TGCAGAGTACATTTGAACAG |
| JAK3 | 1842 | 1 | TGACGCGGAGGCGTATTCGG |
| JAK3 | 1843 | 2 | ACTCTCCAGGCTTAACACAG |
| JAK3 | 1844 | 3 | GTGTACAAATTCCTGCACCA |
| JAK3 | 1845 | 4 | AGCTCTCGAAGACTGCTGTG |
| ATR | 1846 | 1 | TGACGTGCGAAAACAAGATG |
| ATR | 1847 | 2 | GCCCAGGTCACCAATTGTGG |
| ATR | 1848 | 3 | CTGTGTGAGATGGTCAAGCA |
| ATR | 1849 | 4 | GATGCTTTGATTTATATGCA |
| FGFR4 | 1850 | 1 | GAGGTAGATCTAGACTCACG |
| FGFR4 | 1851 | 2 | TTGCACATAGGGGAAACCGT |
| FGFR4 | 1852 | 3 | GGTAACTGTGCCTATTCGAG |
| FGFR4 | 1853 | 4 | TGGTGGCCACTGGTACAAGG |
| MAPK1 | 1854 | 1 | ATCCAGACCATGATCACACA |
| MAPK1 | 1855 | 2 | CAACCTCTCGTACATCGGCG |
| MAPK1 | 1856 | 3 | GCTGACCTTGAGATCACAGG |
| MAPK1 | 1857 | 4 | CCTACTGCCAGAGAACCCTG |
| HPSE | 1858 | 1 | TGGCAATCTCAAGTCAACCA |
| HPSE | 1859 | 2 | TGGTGACGGACAGGAACGAG |
| HPSE | 1860 | 3 | CCACCAAACCTCAGGTACGC |
| HPSE | 1861 | 4 | TAAAAATGTCCAATACATCA |
| IL2RB | 1862 | 1 | GCTGGGAAAAGAACTTCGAG |
| IL2RB | 1863 | 2 | CCACAGATGCAACATAAGCT |
| IL2RB | 1864 | 3 | TTGGGAAGGACACCATTCCG |
| IL2RB | 1865 | 4 | CAGGGTGACGATGTCAACTG |
| BTK | 1866 | 1 | TATGAGTATGACTTTGAACG |
| BTK | 1867 | 2 | GATGGTAGTTAATGAGCTCA |
| BTK | 1868 | 3 | ATAAGGAGTTACCGTATCCC |
| BTK | 1869 | 4 | CTGTGTTTGCTAAATCCACA |
| IHH | 1870 | 1 | CTTGACGGAGCAATGCACGT |
| IHH | 1871 | 2 | CCAGCAGTCCATACTTATTG |
| IHH | 1872 | 3 | GATGTCTGGATTGTAATTGG |
| IHH | 1873 | 4 | GCGAGCGGCACGAGTTTGCG |
| RARG | 1874 | 1 | TGGGCAAGTATACCACGGTG |
| RARG | 1875 | 2 | GGGCTCAGCATCTCGAAAGG |
| RARG | 1876 | 3 | AAGCATGGCTTGTAGACCCG |
| RARG | 1877 | 4 | GCTACAGAAGTGCTTCGAAG |
| EDNRA | 1878 | 1 | TGTCAACACTAAGAGCGCAG |
| EDNRA | 1879 | 2 | CACATAGATAAGGTCTCCAA |
| EDNRA | 1880 | 3 | TGAAGCGATTGGCTTCGTCA |
| EDNRA | 1881 | 4 | CAACATCTCACAAGTCATGA |
| HSPA1B | 1882 | 1 | TCCATCCTGACGATCGACGA |
| HSPA1B | 1883 | 2 | GAGCTACAAGGGGGAGACCA |
| HSPA1B | 1884 | 3 | ACACCGTGTTTGACGCGAAG |
| HSPA1B | 1885 | 4 | AGGATGCGGGTGTGATCGCG |
| BIRC5 | 1886 | 1 | ATGCGGTGGTCCTTGAGAAA |
| BIRC5 | 1887 | 2 | GAACATAAAAAGCATTCGTC |
| BIRC5 | 1888 | 3 | GCTGCGCCTGCACCCCGGAG |
| BIRC5 | 1889 | 4 | CAAGTCTGGCTCGTTCTCAG |
| PDE5A | 1890 | 1 | TGTTGCTGAAGGTTCAACAC |
| PDE5A | 1891 | 2 | GGGGCACTGTTATCTGCACG |
| PDE5A | 1892 | 3 | AAGAGAGCTACAGTCGTTAG |
| PDE5A | 1893 | 4 | AATTAAGAATCATAGGGAAG |
| MAP3K7 | 1894 | 1 | ACCCAAAGCGCTAATTCACA |
| MAP3K7 | 1895 | 2 | AATATTAGGATGGTTCACAC |
| MAP3K7 | 1896 | 3 | CCCAGCTTTCCGAATCATGT |
| MAP3K7 | 1897 | 4 | CACACATGACCAATAACAAG |
| JAK1 | 1898 | 1 | CCGGAAGTAGCCATCTACCA |
| JAK1 | 1899 | 2 | GCCTAGACAGCACCGTAATG |
| JAK1 | 1900 | 3 | TGGTTTCATTCGAATGACGG |
| JAK1 | 1901 | 4 | CACACTTACTCTCCACGTCG |
| PARP1 | 1902 | 1 | CGATGCCTATTACTGCACTG |
| PARP1 | 1903 | 2 | TACCGATCACCGTACCCACA |
| PARP1 | 1904 | 3 | AGCTAGGCATGATTGACCGC |
| PARP1 | 1905 | 4 | GGCCATGATTGAGAAACTCG |
| NR3C1 | 1906 | 1 | TAGAAAAAACTGTTCGACCA |
| NR3C1 | 1907 | 2 | CATCGAACTCTGCACCCCTG |
| NR3C1 | 1908 | 3 | ATCAACAGGTCTGATCTCCA |
| NR3C1 | 1909 | 4 | CTTTAAGTCTGTTTCCCCCG |
| IL6 | 1910 | 1 | CAAATTCGGTACATCCTCGA |
| IL6 | 1911 | 2 | TGCCTGGTGAAAATCATCAC |
| IL6 | 1912 | 3 | TTTGTCAATTCGTTCTGAAG |
| IL6 | 1913 | 4 | TACTCTCAAATCTGTTCTGG |
| MSLN | 1914 | 1 | GCCCCGCAGAGCGTCCATCG |
| MSLN | 1915 | 2 | GGTGCTGGATCACAGACTCG |
| MSLN | 1916 | 3 | ACCCACCTAACATTTCCAGG |
| MSLN | 1917 | 4 | CAGCCGGGGTAGCAGCACTT |
| IKBKB | 1918 | 1 | GCTGGTTCATATCTTGAACA |
| IKBKB | 1919 | 2 | GCCATGGAGTACTGCCAAGG |
| IKBKB | 1920 | 3 | TTTGCAGGCATTCAAAAGTG |
| IKBKB | 1921 | 4 | TGAGGGCCACACATTGGACA |
| LIMK1 | 1922 | 1 | GTGAAGAATTCCATCCACGT |
| LIMK1 | 1923 | 2 | TCCGGCTTATACTCCCAGCG |
| LIMK1 | 1924 | 3 | CGATAAAGGTCCCACACGTG |
| LIMK1 | 1925 | 4 | GGTGTGGCCGGCAGACTACG |
| AURKC | 1926 | 1 | CTAGGAGGAAGACAATGTGT |
| AURKC | 1927 | 2 | AGATGAACAGCGCACAGCCA |
| AURKC | 1928 | 3 | ATTCTGGAATATGCTCCAAG |
| AURKC | 1929 | 4 | GGAAATAGTTATACAGGCGC |
| BRD2 | 1930 | 1 | CCAGCTGCAATACCTACACA |
| BRD2 | 1931 | 2 | ACCACTCTCTCTACGCATAG |
| BRD2 | 1932 | 3 | ACAGTGGTAGGTATCTCAGG |
| BRD2 | 1933 | 4 | CTTGTTGTAAATGTAACAGT |
| ROS1 | 1934 | 1 | TACACCCCAGTCTACCGCAG |
| ROS1 | 1935 | 2 | CTGGGCTGGAAAGACATATG |
| ROS1 | 1936 | 3 | TGGTGATGCCATACCATGTG |
| ROS1 | 1937 | 4 | GTGCACACCATACCTCCATG |
| PIK3CD | 1938 | 1 | CAAGATGTGCCAATTCTGCG |
| PIK3CD | 1939 | 2 | TGTGCGCAGTAACCCCAACA |
| PIK3CD | 1940 | 3 | CAGCGGCTGCCGGAACACTG |
| PIK3CD | 1941 | 4 | TGATGGCGAAGGAGCCTACG |
| TLR5 | 1942 | 1 | TATACAAGCTATTAGCTGCG |
| TLR5 | 1943 | 2 | TTTGTCATATAACCTTCTGG |
| TLR5 | 1944 | 3 | GACACCTGGATCTTTCACAT |
| TLR5 | 1945 | 4 | TACCCCCTTGACTATTGACA |
| TNKS | 1946 | 1 | CCTACATTAGTCAACTGCCA |
| TNKS | 1947 | 2 | GGTACTCTACAACAGACACG |
| TNKS | 1948 | 3 | TGGTGCTGTGCTGCTAACTC |
| TNKS | 1949 | 4 | GATATTCAGGACTTACTGAG |
| BCL2 | 1950 | 1 | TGTCGCAGAGGGGCTACGAG |
| BCL2 | 1951 | 2 | CTGACGCCCTTCACCGCGCG |
| BCL2 | 1952 | 3 | GGCCTTCTTTGAGTTCGGTG |
| BCL2 | 1953 | 4 | TGGACATCTCGGCGAAGTCG |
| ITK | 1954 | 1 | ATACTTTGAAGATCGTCATG |
| ITK | 1955 | 2 | AACTATCACCAACATAATGG |
| ITK | 1956 | 3 | ATCCTCAGGAACTCGCACTG |
| ITK | 1957 | 4 | GGAAGGGGCTATGTCAGAAG |
| CDK1 | 1958 | 1 | GACAAAACACAATCCCCTGT |
| CDK1 | 1959 | 2 | GTATTCCAAAAGCTCTGGCA |
| CDK1 | 1960 | 3 | ACCCTTATACACAACTCCAT |
| CDK1 | 1961 | 4 | GATCTCCAGAAGTATTGCTG |
| MET | 1962 | 1 | CCGATCGCACACATTTGTCG |
| MET | 1963 | 2 | AGCTGTGGCAGCGTCAACAG |
| MET | 1964 | 3 | CTCACTGATATCGAATGCAA |
| MET | 1965 | 4 | TACTGTATTGTGTTGTCCCG |
| PIM2 | 1966 | 1 | ATCCTGATAGACCTACGCCG |
| PIM2 | 1967 | 2 | ATAGCAGTGCGACTTCGAGT |
| PIM2 | 1968 | 3 | CAAGCAGGCGGATCACGCCA |
| PIM2 | 1969 | 4 | TGTCACGATGGACAACTCCA |
| TBK1 | 1970 | 1 | TCCACGTTATGATTTAGACG |
| TBK1 | 1971 | 2 | ACAGTGTATAAACTCCCACA |
| TBK1 | 1972 | 3 | AATCAAGAACTTATCTACGA |
| TBK1 | 1973 | 4 | AGTTGATCTTTGGAGCATTG |
| CDK2 | 1974 | 1 | CATGGGTGTAAGTACGAACA |
| CDK2 | 1975 | 2 | CAAATATTATTCCACAGCTG |
| CDK2 | 1976 | 3 | TCTGAGGTTTAAGGTCTCGG |
| CDK2 | 1977 | 4 | AAGCAGAGAGATCTCTCGGA |
| TLR8 | 1978 | 1 | CATCGTTAAAAATGCCCCAG |
| TLR8 | 1979 | 2 | ATTTAAGCGGGAACTGTCCG |
| TLR8 | 1980 | 3 | TCTTACTGAATTGTCCGACT |
| TLR8 | 1981 | 4 | AACTTATCGACTATCAACTT |
| CPT1A | 1982 | 1 | CACATCGTCGTGTACCATCG |
| CPT1A | 1983 | 2 | GCTCAGTGAACATCCACCCG |
| CPT1A | 1984 | 3 | TACGCCAAATCTCTACTACA |
| CPT1A | 1985 | 4 | ACATCTACCTCCGAGGACGA |
| HSPA1A | 1986 | 1 | GACCAAGGCATTCTACCCCG |
| HSPA1A | 1987 | 2 | CCGCAAGTTCGGCGACCCGG |
| HSPA1A | 1988 | 3 | GCCGTCGTCGATCGTCAGGA |
| HSPA1A | 1989 | 4 | CTGCTGCAGGACTTCTTCAA |
| MGMT | 1990 | 1 | CGCAAACGGTGCGCACCGCG |
| MGMT | 1991 | 2 | GGTACTTGGAAAAATGGACA |
| MGMT | 1992 | 3 | CTGCACGAAATAAAGCTCCT |
| MGMT | 1993 | 4 | ACTCTTCGATAGCCTCGGGC |
| VEGFA | 1994 | 1 | TGGTTTCGGAGGCCCGACCG |
| VEGFA | 1995 | 2 | GGAGGGCAGAATCATCACGA |
| VEGFA | 1996 | 3 | GGAGGAAGAGTAGCTCGCCG |
| VEGFA | 1997 | 4 | AGATGTACTCGATCTCATCA |
| PIK3CA | 1998 | 1 | GTTCGAACAGGTATCTACCA |
| PIK3CA | 1999 | 2 | AACCTCGAACCATAGGATCT |
| PIK3CA | 2000 | 3 | GGATTTAGCTATTCCCACGC |
| PIK3CA | 2001 | 4 | GAAGCTGTATAATGCTTGGG |
| PTGS2 | 2002 | 1 | GGGCTCTAGTATAATAGGAG |
| PTGS2 | 2003 | 2 | GTGGCATACATCATCAGACC |
| PTGS2 | 2004 | 3 | TCAAGACAGATCATAAGCGA |
| PTGS2 | 2005 | 4 | AGTATAAGTGCGATTGTACC |
| IL2RG | 2006 | 1 | CATACCAATAATGCAGAGTG |
| IL2RG | 2007 | 2 | CTGCCCATCCACACTAGGCA |
| IL2RG | 2008 | 3 | GGTGCAGTACCGGACTGACT |
| IL2RG | 2009 | 4 | CATATCTCCAGTGATCCCCT |
| INHBA | 2010 | 1 | CGAGGAAGTGGGCTTAAAGG |
| INHBA | 2011 | 2 | ACAGGCAATCCGAACGTCCA |
| INHBA | 2012 | 3 | GCTGCACTTCGAGATTTCCA |
| INHBA | 2013 | 4 | GCGATCAGAAAGCTTCATGT |
| JAK2 | 2014 | 1 | CTGCCACTGCAATACCAACG |
| JAK2 | 2015 | 2 | AATGAAGAGTACAACCTCAG |
| JAK2 | 2016 | 3 | AGAAAACGATCAAACCCCAC |
| JAK2 | 2017 | 4 | TCTTCAGGAGAGAATACCAT |
| NEK11 | 2018 | 1 | CATTATCACGGAGTACTGTG |
| NEK11 | 2019 | 2 | CCAAGGCTATGACACAAAGT |
| NEK11 | 2020 | 3 | GAGTTGACTACATGCATGAG |
| NEK11 | 2021 | 4 | TCAGACAAGAAAGCCAAACG |
| GART | 2022 | 1 | GTCTTCGGGAAGTACCTGAG |
| GART | 2023 | 2 | GCAGAGCAGTCCATCTAAGG |
| GART | 2024 | 3 | TGGCCTTCACAACCAAAGCA |
| GART | 2025 | 4 | AGCTAGGTCATACTCTCCAG |
| PSENEN | 2026 | 1 | CTGTGCCGGAAGTACTACCT |
| PSENEN | 2027 | 2 | CCTGGAGCGAGTGTCCAATG |
| PSENEN | 2028 | 3 | ACAGAACAGAGCCAAATCAA |
| PSENEN | 2029 | 4 | TGAGCACTATCACCCAGAAG |
| PRKDC | 2030 | 1 | GTTCTCAGAAACGATCAACA |
| PRKDC | 2031 | 2 | CTCCATAATCCGGACCACAA |
| PRKDC | 2032 | 3 | GCACATCATCATGCACCGTG |
| PRKDC | 2033 | 4 | GCAACATCAGAATACTATGG |
| LDLR | 2034 | 1 | CTTAAGGTCATTGCAGACGT |
| LDLR | 2035 | 2 | CAGAGCACTGGAATTCGTCA |
| LDLR | 2036 | 3 | GACAACGGCTCAGACGAGCA |
| LDLR | 2037 | 4 | ATGAACAGGATCCACCACGA |
| FGR | 2038 | 1 | CGAGTTGAACTGAACCCGTG |
| FGR | 2039 | 2 | AGGGGACTTCAGAAGCTACG |
| FGR | 2040 | 3 | AGCTTGGATTGAGTCAACAG |
| FGR | 2041 | 4 | GTGACCGAGTTCATGTGTCA |
| DOT1L | 2042 | 1 | GGAGCGAATCGCCAACACG |
| DOT1L | 2043 | 2 | GCTGGAGGACTCATCCAAGG |
| DOT1L | 2044 | 3 | GCAGCAGGTCTACAACCACT |
| DOT1L | 2045 | 4 | TCAGACCGTGTCTCAGACGG |
| DRD2 | 2046 | 1 | TAGCGCGTATTGTACAGCAT |
| DRD2 | 2047 | 2 | CCTGATCGTCAGCCTCGCAG |
| DRD2 | 2048 | 3 | CTCTTCGGACTCAATAACGC |
| DRD2 | 2049 | 4 | GTGGCATAGTAGTTGTAGTG |
| PRKCA | 2050 | 1 | GCTCCACACTAAATCCGCAG |
| PRKCA | 2051 | 2 | CCTTGACCGAGTGAAACTCA |
| PRKCA | 2052 | 3 | AGGAAGGAAACATGGAACTC |
| PRKCA | 2053 | 4 | AGGTGGGGCTTCCGTAAGTG |
| LHCGR | 2054 | 1 | GTGGCTGGGGTAAGTCAACG |
| LHCGR | 2055 | 2 | GCACAATGGAGCCTTCCGTG |
| LHCGR | 2056 | 3 | CTTGGTTTGGGAATCAACTG |
| LHCGR | 2057 | 4 | TAGCCCATAATATCTTCACA |
| ADORA3 | 2058 | 1 | GATGCCCAGGCTGACAACAA |
| ADORA3 | 2059 | 2 | ATGGCGCACATGACAACCAG |
| ADORA3 | 2060 | 3 | GACATTTCTGTGGTACTCTG |
| ADORA3 | 2061 | 4 | ATTGGACTCTGCGCCATAGT |
| TERT | 2062 | 1 | CACACGCTAGTGGACCCCGA |
| TERT | 2063 | 2 | GTGACACCACACAGAAACCA |
| TERT | 2064 | 3 | CTCACGCAGACGGTGCTCTG |
| TERT | 2065 | 4 | GGCCCGCACACGCAGCACGA |
| HDAC10 | 2066 | 1 | AGTCAGATGCAGACGCAGTG |
| HDAC10 | 2067 | 2 | AGGATTTGACTCAGCCATCG |
| HDAC10 | 2068 | 3 | CCGCAGCCCTGGATCGCCTG |
| HDAC10 | 2069 | 4 | GACAACGCCGGATATCACAT |
| PRKCB | 2070 | 1 | TGACGTGGAGTGCACTATGG |
| PRKCB | 2071 | 2 | CAGAGGGCCAAGATCAGTCA |
| PRKCB | 2072 | 3 | CTTGCTGGATGTGATACATG |
| PRKCB | 2073 | 4 | CCACAGTGGTCACAAAACGT |
| GNRH1 | 2074 | 1 | GCAGTCCATAGGACCAGTGC |
| GNRH1 | 2075 | 2 | CCAATTCAAAAACTCCTAGC |
| GNRH1 | 2076 | 3 | GCGTGGTGCATTCGAAGCGT |
| GNRH1 | 2077 | 4 | CTACTGACTTGGTGCGTGGA |
| STAT3 | 2078 | 1 | ACGCCGGTCTTGATGACGAG |
| STAT3 | 2079 | 2 | GAGACCGAGGTGTATCACCA |
| STAT3 | 2080 | 3 | AACATGGAAGAATCCAACAA |
| STAT3 | 2081 | 4 | TCGGCCGGTGCTGTACAATG |
| FGFR2 | 2082 | 1 | CTTAGTCCAACTGATCACGG |
| FGFR2 | 2083 | 2 | TGTGTCTGTTCTAGCACTCG |
| FGFR2 | 2084 | 3 | GCCGGCAAATGCCTCCACAG |
| FGFR2 | 2085 | 4 | GATAGCCATTTACTGCATAG |
| LCK | 2086 | 1 | GACCCACTGGTTACCTACGA |
| LCK | 2087 | 2 | GCCGGGAAAAGTGATTCGAG |
| LCK | 2088 | 3 | CTACAACGGGCACACGAAGG |
| LCK | 2089 | 4 | GCTGGTTCGGCTCTACGCTG |
| FOLR3 | 2090 | 1 | GAAGAAGAATGCCTGCTGCA |
| FOLR3 | 2091 | 2 | TGGCTTTGGTGACTGCTGCG |
| FOLR3 | 2092 | 3 | ACTCATACCTGCCGGATCCA |
| FOLR3 | 2093 | 4 | AACTTTAACTGGGATCACTG |
| PORCN | 2094 | 1 | TGCCGACATTCCTCCCATCG |
| PORCN | 2095 | 2 | GTGACATGGCACAAGATGCG |
| PORCN | 2096 | 3 | GGAGCTGCCTCGGTCAATGG |
| PORCN | 2097 | 4 | TAGCTGTGGAAGGATATCCA |
| APH1B | 2098 | 1 | GGTTTGAAGAGTATAAACCC |
| APH1B | 2099 | 2 | TCTTGCCATGAACCAAACAA |
| APH1B | 2100 | 3 | GAAGATGATACGCAACGGCT |
| APH1B | 2101 | 4 | TTGGGCTTTGGAATCATGAG |
| PARP2 | 2102 | 1 | CATGCAATGAATTCTACACC |
| PARP2 | 2103 | 2 | AATACCAAGAAAGCCCCACT |
| PARP2 | 2104 | 3 | GGGGGCGCAAGGCACAATGT |
| PARP2 | 2105 | 4 | TTGTTCAGGCAATCTCAACA |
| BCL2L2 | 2106 | 1 | GTGTGCTGAGAGTGTCAACA |
| BCL2L2 | 2107 | 2 | AGCCCAACAACGCTTCACCC |
| BCL2L2 | 2108 | 3 | ACTTTGTAGGTTATAAGCTG |
| BCL2L2 | 2109 | 4 | AGACAAAGAAGGCTACAAGG |
| CYP17A1 | 2110 | 1 | CCATACGAACCGAATAGATG |
| CYP17A1 | 2111 | 2 | ATCGCGTCCAACAACCGTAA |
| CYP17A1 | 2112 | 3 | TATGGACTGTCCGTTGTGGG |
| CYP17A1 | 2113 | 4 | CAATACCTCCTACAAGAATG |
| EIF4E | 2114 | 1 | AAAGCTTACCTGTTCTGTAG |
| EIF4E | 2115 | 2 | GAAGATGAGAAAAACAAACG |
| EIF4E | 2116 | 3 | TTCTCCTCTTCTGTAGTCGG |
| EIF4E | 2117 | 4 | AAACTTGGCAAGCAAACCTG |
| ESR1 | 2118 | 1 | TCAGATAATCGACGCCAGGG |
| ESR1 | 2119 | 2 | CTGACCGTAGACCTGCGCGT |
| ESR1 | 2120 | 3 | TACTCGGAATAGAGTATCGG |
| ESR1 | 2121 | 4 | TCCAGGTACACCTCGCCCAG |
| IDH2 | 2122 | 1 | GTCCTTGAAACGCCCATCGT |
| IDH2 | 2123 | 2 | CTTTAGCTGGATGTCCACGT |
| IDH2 | 2124 | 3 | GGGCATGTACAACACCGACG |
| IDH2 | 2125 | 4 | ACTTCGACAAGAATAAGATC |
| IDO1 | 2126 | 1 | ATCCCAGAACTAGACGTGCA |
| IDO1 | 2127 | 2 | ACCAGACCGTCTGATAGCTG |
| IDO1 | 2128 | 3 | GATACTTACTCATAAGTCAG |
| IDO1 | 2129 | 4 | AGAACGGGACACTTTGCTAA |
| NOTCH3 | 2130 | 1 | CACGCTGTGTGATCGCAACG |
| NOTCH3 | 2131 | 2 | GACACCGATGTCTCAATGGG |
| NOTCH3 | 2132 | 3 | GGTTGGTGCAGATACCATGA |
| NOTCH3 | 2133 | 4 | GACAGGACAGTCTGACAGCG |
| AKT2 | 2134 | 1 | TCTCGTCTGGAGAATCCACG |
| AKT2 | 2135 | 2 | GACCCCATGGACTACAAGTG |
| AKT2 | 2136 | 3 | GGGGGGTAGAGTCTGATCAG |
| AKT2 | 2137 | 4 | CTCTTGAGTACTTGCACTCG |
| AURKA | 2138 | 1 | CTTCGAATGACAGTAAGACA |
| AURKA | 2139 | 2 | CCATATAGAAAATAATCCTG |
| AURKA | 2140 | 3 | CCTGAAAACTCACCGAAGGT |
| AURKA | 2141 | 4 | TGCTTGCAAAGGAATGCGCT |
| PPP2CA | 2142 | 1 | AACGCATCACCATTCTTCGA |
| PPP2CA | 2143 | 2 | AAAAGAATCCAACGTGCAAG |
| PPP2CA | 2144 | 3 | AATAAAAGTCATACCTCATG |
| PPP2CA | 2145 | 4 | CTCGCCATCTATAGATACAC |
| DNMT1 | 2146 | 1 | GATTTCTGATGAAAAAGACG |
| DNMT1 | 2147 | 2 | GCTCTACTGGAGCGACGAGG |
| DNMT1 | 2148 | 3 | GAGGCAAAAAGAAATCCCCA |
| DNMT1 | 2149 | 4 | TCACCCAAAAAAATGCACCA |
| NTRK1 | 2150 | 1 | CCCTTTCGAGTTCAACCCCG |
| NTRK1 | 2151 | 2 | GCGCAGACACCCGTGCCGCA |
| NTRK1 | 2152 | 3 | AGGGCACAAGAACAGTGCAG |
| NTRK1 | 2153 | 4 | CTGGAGCTCCGTGATCTGAG |
| IFNAR2 | 2154 | 1 | TGAAAGTGATAGCGATACTG |
| IFNAR2 | 2155 | 2 | TGAGTGGAGAAGCACACACG |
| IFNAR2 | 2156 | 3 | CGTCATTGAAGAACAGTCAG |
| IFNAR2 | 2157 | 4 | ATATCCATGGCTTCCAACGG |
| MAPK8 | 2158 | 1 | TAGTGGATTTATGGTCTGTG |
| MAPK8 | 2159 | 2 | AGAATCAGACTCATGCCAAG |
| MAPK8 | 2160 | 3 | TGATATTAGATATTGATCAG |
| MAPK8 | 2161 | 4 | AGAAACTGCAACCAACAGTA |
| SMO | 2162 | 1 | CAAGAACTACCGATACCGTG |
| SMO | 2163 | 2 | GATTCTTGATCTCACAGTCA |
| SMO | 2164 | 3 | CCACATTCGTGGCTGACTGG |
| SMO | 2165 | 4 | CAAGTGTGAGAATGACCGGG |
| MAP3K8 | 2166 | 1 | ATCAGTCAGATATGGAACTG |
| MAP3K8 | 2167 | 2 | CTTCGGTCATTTGAACACTT |
| MAP3K8 | 2168 | 3 | CCAGGGGATCAGGAGAACAT |
| MAP3K8 | 2169 | 4 | TGACACATGGTCATTAGACT |
| TXN | 2170 | 1 | TACTTCAAGGAATATCACGT |
| TXN | 2171 | 2 | CACACTCTGAAGCAACATCC |
| TXN | 2172 | 3 | TAGTTGACTTCTCAGCCACG |
| TXN | 2173 | 4 | GGTGAAGCAGATCGAGAGCA |
| IDH1 | 2174 | 1 | ATGTAGATCCAATTCCACGT |
| IDH1 | 2175 | 2 | CCCATCCACTCACAAGCCGG |
| IDH1 | 2176 | 3 | CAAGCTATGAAATCAGAGGG |
| IDH1 | 2177 | 4 | TACCTTCAAAGTTATGTACC |
| GSK3A | 2178 | 1 | CCTCCCATAACTCTGACCGA |
| GSK3A | 2179 | 2 | GCCCGAGACAGTGTACCGGG |
| GSK3A | 2180 | 3 | TCAGCCTCACAATATTGCAG |
| GSK3A | 2181 | 4 | AGGAGACATTGGGCTCCCCT |
| PSMB9 | 2182 | 1 | GTAGATGCGCTCGTGCAGCG |
| PSMB9 | 2183 | 2 | CCAGACCCATTACCCCGGTG |
| PSMB9 | 2184 | 3 | TATCAGCTATAAATATCGAG |
| PSMB9 | 2185 | 4 | TCCCAGGGTTCCATATACCT |
| CD19 | 2186 | 1 | CTAGGTCCGAAACATTCCAC |
| CD19 | 2187 | 2 | GGAAAGTATTATTGTCACCG |
| CD19 | 2188 | 3 | ATGAAAAGCCAGATGGCCAG |
| CD19 | 2189 | 4 | AAGATGAAGAATGCCCACAA |
| ESR2 | 2190 | 1 | CCAGTTATCACATCTGTATG |
| ESR2 | 2191 | 2 | TCAGCCTGTTCGACCAAGTG |
| ESR2 | 2192 | 3 | CCCCAGTGCGCCCTTCACCG |
| ESR2 | 2193 | 4 | AGCAGGGCTATAGAATGTCA |
| FLT3 | 2194 | 1 | AAAGCTGTTCATGTGAACCA |
| FLT3 | 2195 | 2 | GGTGCTTTGCGATTCACAGG |
| FLT3 | 2196 | 3 | GTAACCAAAGCTGATTGACT |
| FLT3 | 2197 | 4 | GGGGTCTCAACGCACACCCG |
| MAP2K2 | 2198 | 1 | AAGCACCAGATCATGCACCG |
| MAP2K2 | 2199 | 2 | ACGGCGAGTTGCATTCGTGC |
| MAP2K2 | 2200 | 3 | GGCCCATCCCCTACCAGCGA |
| MAP2K2 | 2201 | 4 | GGATTCCCGAGGAGATCCTG |
| PGF | 2202 | 1 | CGTGTCCGAGTACCCCAGCG |
| PGF | 2203 | 2 | CCAGCGCCCGGCAGTAGCTG |
| PGF | 2204 | 3 | TGGGAACGGCTCGTCAGAGG |
| PGF | 2205 | 4 | GCTCCTAAAGATCCGTTCTG |
| ADORA2A | 2206 | 1 | GCGGCGGCCGACATCGCAGT |
| ADORA2A | 2207 | 2 | TGGCTTGGTGACCGGCACGA |
| ADORA2A | 2208 | 3 | ATGCTAGGTTGGAACAACTG |
| ADORA2A | 2209 | 4 | AAGCAGTTGATGATGTGTAG |
| CXCR1 | 2210 | 1 | CAGAACAGCATGACAAACAG |
| CXCR1 | 2211 | 2 | GAAATGACACAGCAAAATGG |
| CXCR1 | 2212 | 3 | CAGGCTCAGCAGGAACACTA |
| CXCR1 | 2213 | 4 | ACTGACCCAGAAGCGTCACT |
| AKT3 | 2214 | 1 | CTGCACCATAGAAACGTGTG |
| AKT3 | 2215 | 2 | ATTTCATGTAGATACTCCAG |
| AKT3 | 2216 | 3 | ACAAATTGATAATATAGGAG |
| AKT3 | 2217 | 4 | TATTTGAAACTACTAGGTAA |
| DPP4 | 2218 | 1 | GGATTCCAAACAACACACAG |
| DPP4 | 2219 | 2 | CTGCTGTGTAGAGTATAGAG |
| DPP4 | 2220 | 3 | CTACTTGTGTGATGTGACAT |
| DPP4 | 2221 | 4 | GAATATAAAGGAATGCCAGG |
| RET | 2222 | 1 | CGGCACAGCTCGTCGCACAG |
| RET | 2223 | 2 | CTAGATCGGGAAAGTCTGTG |
| RET | 2224 | 3 | TGCCGAACTTCACTACATGG |
| RET | 2225 | 4 | CCCGGTGACCGTGTACGACG |
| CHEK2 | 2226 | 1 | GGGCCCATAATCGAGCCCAG |
| CHEK2 | 2227 | 2 | AGGTAAAGCTGGCTTTCGAG |
| CHEK2 | 2228 | 3 | GCATACATAGAAGATCACAG |
| CHEK2 | 2229 | 4 | AGAGCTGTTTGACAAAGTGG |
| NAE1 | 2230 | 1 | TCAAAGAAGCAGTATCGGCA |
| NAE1 | 2231 | 2 | TAGCTAAATATTTAGCACAG |
| NAE1 | 2232 | 3 | CACAGCACTAAATACAACTC |
| NAE1 | 2233 | 4 | ATCATTATAAAAGAACATCC |
| HSP90AA1 | 2234 | 1 | GATCTGTCAAGCTTTCATAC |
| HSP90AA1 | 2235 | 2 | TCTCACGGGATATGTTTAGA |
| HSP90AA1 | 2236 | 3 | CAGTGAGGACAGACACAGGT |
| HSP90AA1 | 2237 | 4 | GATCAAAAGGAGCACGTCGT |
| TYK2 | 2238 | 1 | TGAATGACGTGGCATCACTG |
| TYK2 | 2239 | 2 | CAGGCGGCCCTCATACACGT |
| TYK2 | 2240 | 3 | AATACCTAGCCACACTCGAG |
| TYK2 | 2241 | 4 | TTGGGCCTGAGCATCGAAGA |
| ABL2 | 2242 | 1 | AACCTCTGTAATGACGACGG |
| ABL2 | 2243 | 2 | TGTACACCATCACTCCACAG |
| ABL2 | 2244 | 3 | TATCGAATGGAACAGCCTGA |
| ABL2 | 2245 | 4 | GGTTCAACATCACAACCATA |
| FGF1 | 2246 | 1 | TGAGCCGTATAAAAGCCCGT |
| FGF1 | 2247 | 2 | CTCCTCTACTGTAGCAACGG |
| FGF1 | 2248 | 3 | TTCCTGAGGATCCTTCCGGA |
| FGF1 | 2249 | 4 | AGAAGTTTAATCTGCCTCCA |
| ERBB3 | 2250 | 1 | ATGAGGCGATACTTGGAACG |
| ERBB3 | 2251 | 2 | TGTCGAAATTATAGCCGAGG |
| ERBB3 | 2252 | 3 | ATCATGTGAGACAACACCGG |
| ERBB3 | 2253 | 4 | ACCATTGCCCAACCTCCGCG |
| NTRK2 | 2254 | 1 | TGAATGGAATGCACCAGTGG |
| NTRK2 | 2255 | 2 | ACGTCACTGATAAAACCGGT |
| NTRK2 | 2256 | 3 | AACCTGCAGATACCCAATTG |
| NTRK2 | 2257 | 4 | TTGGTGATGCCAAAGTACTG |
| HDAC3 | 2258 | 1 | TCATCAATGCCATCCCGCAG |
| HDAC3 | 2259 | 2 | ACCTGGAGCACAATGCACGT |
| HDAC3 | 2260 | 3 | TGGGTCAATGCCAGGCGATG |
| HDAC3 | 2261 | 4 | GTCAGCCCCACCAATATGCA |
| B4GALNT1 | 2262 | 1 | CCCGTAGCCGATCATAACGG |
| B4GALNT1 | 2263 | 2 | GGATCAAGGAGCAAGTAGTG |
| B4GALNT1 | 2264 | 3 | TGGAGTTACTCTCACTGGAG |
| B4GALNT1 | 2265 | 4 | GCGCGTTAGTGGCCCCTACG |
| RBM39 | 2266 | 1 | TGGCGGCAAGAATTCGACCA |
| RBM39 | 2267 | 2 | TTGGCACGCCTAAAACTCGT |
| RBM39 | 2268 | 3 | GGACCTATGAGGCTTTATGT |
| RBM39 | 2269 | 4 | AAATTTAACAGTGCCATCCG |
| HDAC1 | 2270 | 1 | CATCCGTCCAGATAACATGT |
| HDAC1 | 2271 | 2 | TGAGTCATGCGGATTCGGTG |
| HDAC1 | 2272 | 3 | GCACCGGGCAACGTTACGAA |
| HDAC1 | 2273 | 4 | GGAGATGTTCCAGCCTAGTG |
| IL6R | 2274 | 1 | TGGAAACTATTCATGCTACC |
| IL6R | 2275 | 2 | ACTCACAAACAACATTGCTG |
| IL6R | 2276 | 3 | CCGTGGCCAGAAACCCCCGC |
| IL6R | 2277 | 4 | CCTGAGCACCCAGTGAACAG |
| BAX | 2278 | 1 | TCGGAAAAAGACCTCTCGGG |
| BAX | 2279 | 2 | GTTTCATCCAGGATCGAGCA |
| BAX | 2280 | 3 | AGTAGAAAAGGGCGACAACC |
| BAX | 2281 | 4 | GGACGAACTGGACAGTAACA |
| TOP1 | 2282 | 1 | CGACCATGAATATACTACCA |
| TOP1 | 2283 | 2 | TGGAAGAGGCTCATATGGTG |
| TOP1 | 2284 | 3 | ACTCACTCATCCTCATCTCG |
| TOP1 | 2285 | 4 | CAAACATAAAGACAGAGACA |
| EGFR | 2286 | 1 | TGTCACCACATAATTACCTG |
| EGFR | 2287 | 2 | GTGGAGCCTCTTACACCCAG |
| EGFR | 2288 | 3 | GTCTGCGTACTTCCAGACCA |
| EGFR | 2289 | 4 | TCTTGCCGGAATGTCAGCCG |
| RAD50 | 2290 | 1 | CTAGGAACGTGAGTTAAGCA |
| RAD50 | 2291 | 2 | AAGCGGCGTGATGAAATGCT |
| RAD50 | 2292 | 3 | AAACAGCACAAGTTAGACAC |
| RAD50 | 2293 | 4 | AAAAACTGCCAACCAACTGA |
| BLK | 2294 | 1 | CAGGTCCCGATCATTCATAG |
| BLK | 2295 | 2 | GCTGGTCCGACTCTACGCAG |
| BLK | 2296 | 3 | GCTTCTTGCTCCAATCAACA |
| BLK | 2297 | 4 | ACTCGGGCCACAAAGTTACT |
| GSK3B | 2298 | 1 | ATACCTTGACATAAATCACA |
| GSK3B | 2299 | 2 | CAGTATCAGGATCCAACAAG |
| GSK3B | 2300 | 3 | GTGGCTCCAAAGATCAACTC |
| GSK3B | 2301 | 4 | AGGTCCTGGGAACTCCAACA |
| MAPK3 | 2302 | 1 | GCAGTTGCAGTACATCGGCG |
| MAPK3 | 2303 | 2 | AGTAGGTCTGATGTTCGAAG |
| MAPK3 | 2304 | 3 | TTCCGCCATGAGAATGTCAT |
| MAPK3 | 2305 | 4 | TGGAGGGCTTTAGATCTCGG |
| MUC5AC | 2306 | 1 | GAAGCCGGGAACCTACTACT |
| MUC5AC | 2307 | 2 | CAAGATGTGCCTCAACTACG |
| MUC5AC | 2308 | 3 | CCGTTAATGACTTTGCCACG |
| MUC5AC | 2309 | 4 | GAGGTGAGCATTGAACACCT |
| NBN | 2310 | 1 | TTCCCGAACTTTGAAGTCGG |
| NBN | 2311 | 2 | AGAATGCACTCACCTTGTCA |
| NBN | 2312 | 3 | CCAGGACCAAGCCTTTCACA |
| NBN | 2313 | 4 | AGCAGCCTCCACAAATTGAA |
| DDR2 | 2314 | 1 | CCGTGACAAACCGAGCACTG |
| DDR2 | 2315 | 2 | GGGCTAGGCCAATTGACCGA |
| DDR2 | 2316 | 3 | GTAATTGATCTTGTACATGG |
| DDR2 | 2317 | 4 | AAGTTGATGACAGCAACACT |
| NOTCH2 | 2318 | 1 | TTGATGTCCATCTCACAACG |
| NOTCH2 | 2319 | 2 | CTGGGGACACACATCGACGA |
| NOTCH2 | 2320 | 3 | TTGTTGATATAATCCCAGCA |
| NOTCH2 | 2321 | 4 | CATTGGTGGATACAGATGCG |
| VDR | 2322 | 1 | ACAGCTCTAGGGTCACAGAA |
| VDR | 2323 | 2 | CCACACACCCCACAGATCCG |
| VDR | 2324 | 3 | CTGCCGGCTCAAACGCTGTG |
| VDR | 2325 | 4 | CCATCATTCACACGAACTGG |
| BMX | 2326 | 1 | GGTTAGAAATTCGAGCCAAG |
| BMX | 2327 | 2 | GGGAAGACTTCCCTGACTGG |
| BMX | 2328 | 3 | TGTTGGCCTTTGTTGACACA |
| BMX | 2329 | 4 | GGGGTTACCCCTTATCTCTG |
| ABL1 | 2330 | 1 | TCAGTGATGATATAGAACGG |
| ABL1 | 2331 | 2 | GGTTCATCATCATTCAACGG |
| ABL1 | 2332 | 3 | TTGCTCCCTCGAAAAGAGCG |
| ABL1 | 2333 | 4 | CTTAGGCTATAATCACAATG |
| ANGPT1 | 2334 | 1 | TTGCAATATGGATGTCAATG |
| ANGPT1 | 2335 | 2 | GCAGCTTGAGAATTACATTG |
| ANGPT1 | 2336 | 3 | AGATATAACCGGATTCAACA |
| ANGPT1 | 2337 | 4 | GCAGAGAGATGCTCCACACG |
| CRTC2 | 2338 | 1 | AGGGCCACTTACCCCCACGT |
| CRTC2 | 2339 | 2 | CTTCGCCAGCTAGACTCTGG |
| CRTC2 | 2340 | 3 | GAGGTTGGGATTGCTTAGGG |
| CRTC2 | 2341 | 4 | CCACCTGCCATGAACACGGG |
| CRBN | 2342 | 1 | ACCAATGTTCATATAAATGG |
| CRBN | 2343 | 2 | CTGACTGTGTTCTTAGCTCA |
| CRBN | 2344 | 3 | TGTATGTGATGTCGGCAGAC |
| CRBN | 2345 | 4 | TGAAGAGGTAATGTCTGTCC |
| IL1B | 2346 | 1 | CTTCGACACATGGGATAACG |
| IL1B | 2347 | 2 | GGTGGTCGGAGATTCGTAGC |
| IL1B | 2348 | 3 | CATGGCCACAACAACTGACG |
| IL1B | 2349 | 4 | CTGAAAGCTCTCCACCTCCA |
| TNFSF11 | 2350 | 1 | ACCCCGATCATGGTACCAAG |
| TNFSF11 | 2351 | 2 | ATTCTATTAGGATCCATCTG |
| TNFSF11 | 2352 | 3 | CATGAGCCATCCACCATCGC |
| TNFSF11 | 2353 | 4 | GGAGGGCCACGAACATGGAG |
| FZD8 | 2354 | 1 | CGGCGGTGGTTAGGTCGGTG |
| FZD8 | 2355 | 2 | GGCCTGCTACCTCTTCGTGT |
| FZD8 | 2356 | 3 | TAGCCGATGCCCTTACACAG |
| FZD8 | 2357 | 4 | ACACGCTCACCATAGGCGCG |
| TGFBR1 | 2358 | 1 | AGAACGTTCGTGGTTCCGTG |
| TGFBR1 | 2359 | 2 | TAAAAGGGCGATCTAATGAA |
| TGFBR1 | 2360 | 3 | GTTGTGTATAACTTTGTCTG |
| TGFBR1 | 2361 | 4 | ATGGGCAAGACCGCTCGCCG |
| FGF2 | 2362 | 1 | TCTCCCGGACCCCGTCAACT |
| FGF2 | 2363 | 2 | GCCACTTCAAGGACCCCAAG |
| FGF2 | 2364 | 3 | GGGTGCCAGATTAGCGGACG |
| FGF2 | 2365 | 4 | TTCACGGATGGGTGTCTCCG |
| DYRK4 | 2366 | 1 | TTGGATGTACGTGTATACTG |
| DYRK4 | 2367 | 2 | CAAAGACAACACCTACAATG |
| DYRK4 | 2368 | 3 | CTCCAGAACTTCATAGCGGT |
| DYRK4 | 2369 | 4 | TGAAGCCAAGAAGCTCGACA |
| IL2RA | 2370 | 1 | GGATACAGGGCTCTACACAG |
| IL2RA | 2371 | 2 | TGGCTTTGAATGTGGCGTGT |
| IL2RA | 2372 | 3 | TTGTTTCGTTGTGTTCCGAG |
| IL2RA | 2373 | 4 | CTGCAGGGAACCTCCACCAT |
| ARAF | 2374 | 1 | GCCCAACAAGCAACGCACGG |
| ARAF | 2375 | 2 | GTAGTGATGGAACCCCCCGG |
| ARAF | 2376 | 3 | TGGTCTACCGACTCATCAAG |
| ARAF | 2377 | 4 | AGTGTCCAGGATTTGTCCGG |
| MAPK14 | 2378 | 1 | TGATGAAATGACAGGCTACG |
| MAPK14 | 2379 | 2 | CACAAAAACGGGGTTACGTG |
| MAPK14 | 2380 | 3 | AAGTAACCGCAGTTCTCTGT |
| MAPK14 | 2381 | 4 | CAAGGCGAGTAATACCTGTC |
| CSF1R | 2382 | 1 | ACGCTACCTTCCAAAACACG |
| CSF1R | 2383 | 2 | GTTGGAAATCTACTTGATCG |
| CSF1R | 2384 | 3 | GCTGCCTTACAACGAGAAGT |
| CSF1R | 2385 | 4 | GCTTGCTAATGCTACCACCA |
| ROCK2 | 2386 | 1 | TGTTTAGGGAGGTACGACTT |
| ROCK2 | 2387 | 2 | ACCGGATTATATATCACCTG |
| ROCK2 | 2388 | 3 | AGCTGAACATAAGGCCACAA |
| ROCK2 | 2389 | 4 | TAGTAGGTAAATCCGATGAA |
| PRMT5 | 2390 | 1 | GGAGAAAAACCCAAATGCCG |
| PRMT5 | 2391 | 2 | GGTACTGAGAGTATTTGATG |
| PRMT5 | 2392 | 3 | GAAGATTCGCAGGAACTCCG |
| PRMT5 | 2393 | 4 | TGCACCAACTACACACACAG |
| FGFR3 | 2394 | 1 | CATCCGGCAGACGTACACGC |
| FGFR3 | 2395 | 2 | GGTGCTGAATGCCTCCCACG |
| FGFR3 | 2396 | 3 | AAGAACGGCAGGGAGTTCCG |
| FGFR3 | 2397 | 4 | CCCGAGACAGCTCCCATTTG |
| CSNK2A2 | 2398 | 1 | AGTTTACCTGATAGTCCACG |
| CSNK2A2 | 2399 | 2 | TGATATTTGTTCCACCACGA |
| CSNK2A2 | 2400 | 3 | TAGCAAGCATGATCTTTCGA |
| CSNK2A2 | 2401 | 4 | TGTGCATGATTCCCTTGCTG |
| SIRT1 | 2402 | 1 | CTCTGAGCCATACCTATCCG |
| SIRT1 | 2403 | 2 | GCGGCGGCGATTGGGTACCG |
| SIRT1 | 2404 | 3 | TCTGGTTTCATGATAGCAAG |
| SIRT1 | 2405 | 4 | ATAGCCTTGTCAGATAAGGA |
| FGFR1 | 2406 | 1 | GTTGCCCGCCAACAAAACAG |
| FGFR1 | 2407 | 2 | CTGGTCTTAGGCAAACCCCT |
| FGFR1 | 2408 | 3 | AGTTCAAATGCCCTTCCAGT |
| FGFR1 | 2409 | 4 | ACAGTGTGTACCTTCCAGAA |
| FRK | 2410 | 1 | CTATATTCCTTCTAACTACG |
| FRK | 2411 | 2 | AGTTGCGGTCTATCTCCCAT |
| FRK | 2412 | 3 | GCAGTGAAAACATTAAAACC |
| FRK | 2413 | 4 | CACTACACCAAGACAAGTGA |
| NAMPT | 2414 | 1 | GGGATGGAACTACATTCTTG |
| NAMPT | 2415 | 2 | CTGTTCCAGCAGCAGAACAC |
| NAMPT | 2416 | 3 | AATAGTATCAAGAAGTACAC |
| NAMPT | 2417 | 4 | CAGAGGAGTCTCTTCCCAAG |
| ERBB2 | 2418 | 1 | AACTACCTTTCTACGGACGT |
| ERBB2 | 2419 | 2 | TTGGGATCCTCATCAAGCGA |
| ERBB2 | 2420 | 3 | TCATCGCTCACAACCAAGTG |
| ERBB2 | 2421 | 4 | GTTACCTATACATCTCAGCA |
| CHD1 | 2422 | 1 | TTAATTCGCCTAAGAGAACG |
| CHD1 | 2423 | 2 | TTCCGATGACTCATCAAGTG |
| CHD1 | 2424 | 3 | AAGCAGCCATCCTATATTGG |
| CHD1 | 2425 | 4 | ATGCCCAATTTAGACCTCCA |
| CD38 | 2426 | 1 | TCTGGCCCATCAGTTCACAC |
| CD38 | 2427 | 2 | TCAGACCGTACCTTGCAACA |
| CD38 | 2428 | 3 | CTTGACGCATCGCGCCAGGA |
| CD38 | 2429 | 4 | TCGCGGTGGTCGTCCCGAGG |
| EPHA2 | 2430 | 1 | GAAGCCCCTGAAGACATACG |
| EPHA2 | 2431 | 2 | CACACACCCGTATGGCAAAG |
| EPHA2 | 2432 | 3 | TCACGGAGAAACCCTCGGTG |
| EPHA2 | 2433 | 4 | CTGGTGCGGGTCAGTCCGTG |
| TLR7 | 2434 | 1 | AATGGGGCATTATAACAACG |
| TLR7 | 2435 | 2 | CAGCTACTAGAGATACCGCA |
| TLR7 | 2436 | 3 | CAGTCTGTGAAAGGACGCTG |
| TLR7 | 2437 | 4 | GAAGATTATGTAATGGCGAG |
| PRKCE | 2438 | 1 | GCACCGCTTGTGGACCACGC |
| PRKCE | 2439 | 2 | GCCTTGTCATTTGACAACCG |
| PRKCE | 2440 | 3 | CCACGTTGGTCTCACATCGA |
| PRKCE | 2441 | 4 | ATGTGATCATCGATCTCTCA |
| EPHB4 | 2442 | 1 | GTGCCCGGAAGTACCCGACG |
| EPHB4 | 2443 | 2 | CTTCCGGGTGAGATGCTCCG |
| EPHB4 | 2444 | 3 | CTGCACGTCACACACTTCGT |
| EPHB4 | 2445 | 4 | TGTCTGACATCCGGGTGACG |
| TNFSF13B | 2446 | 1 | GCTGTCTTGCTGCCTCACGG |
| TNFSF13B | 2447 | 2 | CAAACTCACTTTCAGTCCCG |
| TNFSF13B | 2448 | 3 | TGTTTCCATCCTCCCACGGA |
| TNFSF13B | 2449 | 4 | TGGCTTCTCAGCTTTAAAAG |
| PSMB5 | 2450 | 1 | TTTGTACTGATACACCATGT |
| PSMB5 | 2451 | 2 | GCTTCATGGAACAACCACCC |
| PSMB5 | 2452 | 3 | CCGCTACCGGTGAACCAGCG |
| PSMB5 | 2453 | 4 | ATCTGTGGCTGGGATAAGAG |
| TNF | 2454 | 1 | TTGGAGTGATCGGCCCCCAG |
| TNF | 2455 | 2 | AGAGCTCTTACCTACAACAT |
| TNF | 2456 | 3 | GGAGCTGAGAGATAACCAGC |
| TNF | 2457 | 4 | GAGACACTTACTGACTGCCT |
| NOTCH4 | 2458 | 1 | ACACCCCTCCATTAACACAT |
| NOTCH4 | 2459 | 2 | GCTGCCATTGTATAGGCATG |
| NOTCH4 | 2460 | 3 | TGTGTCAGCCTGGCTATTCG |
| NOTCH4 | 2461 | 4 | GGAGGCCTCTGTGTCGACAG |
| FCGR1A | 2462 | 1 | CTGGGAGCAGCTCTACACAG |
| FCGR1A | 2463 | 2 | ATGGGCAGCAAGACCCTGCG |
| FCGR1A | 2464 | 3 | TGTGACATCCCCACTCCTGG |
| FCGR1A | 2465 | 4 | TCCAGCAGAGTCTTCACGGA |
| TUBB1 | 2466 | 1 | GCTGATCGAGAATGTCCTAG |
| TUBB1 | 2467 | 2 | GCTGACGACACCCACCTATG |
| TUBB1 | 2468 | 3 | TGTGGTGGAGCCCTACAACG |
| TUBB1 | 2469 | 4 | GCTCATGAACAAGATTAGAG |
| CCND3 | 2470 | 1 | CACTGGAAGTAGGAGGCGCG |
| CCND3 | 2471 | 2 | ACCAAGGCCTGTCGGTCACG |
| CCND3 | 2472 | 3 | ACACACGCACCCGCAACTGG |
| CCND3 | 2473 | 4 | CATGTGCAATCACAGCAGCC |
| SIK1 | 2474 | 1 | ATGGTCGTGACAGTACTCCA |
| SIK1 | 2475 | 2 | GCATCGAGTCACCAAAACGC |
| SIK1 | 2476 | 3 | CCGGGGACACTTACATGGCG |
| SIK1 | 2477 | 4 | CCCCTCAAAGACTTCCGGGG |
| PIK3CB | 2478 | 1 | AAAAATGCGCAAATTCAGCG |
| PIK3CB | 2479 | 2 | TGTAGCGTGGGTAAATACGA |
| PIK3CB | 2480 | 3 | AAAGAGCACTTGGTAATCGG |
| PIK3CB | 2481 | 4 | TAAAACCATCGTAAGCTCAG |
| HDAC5 | 2482 | 1 | ACGTTCACCCGTCACTAGTG |
| HDAC5 | 2483 | 2 | TATGCCCTGTACTTACAGTG |
| HDAC5 | 2484 | 3 | GCCGGGTGCGCTGTTACACA |
| HDAC5 | 2485 | 4 | AGGCCTGCTTAGCAAGTGCG |
| S1PR1 | 2486 | 1 | GTCCGGCATTACAACTACAC |
| S1PR1 | 2487 | 2 | ACAAGCTCACTCCCGCCCAG |
| S1PR1 | 2488 | 3 | TCTGCAGTACAGAATGACGA |
| S1PR1 | 2489 | 4 | CAATAAAATAGTACATGGGT |
| PRKCI | 2490 | 1 | TCAGAATCCATCTACCGTAG |
| PRKCI | 2491 | 2 | TGTCTCGAACCTCATTGCAA |
| PRKCI | 2492 | 3 | ATCTGCACAGACCGAATATG |
| PRKCI | 2493 | 4 | AGCAAGAATGCAGCCCAACA |
| RPL3 | 2494 | 1 | GGCTACGTGGAAACCCCTCG |
| RPL3 | 2495 | 2 | TGAGATGATCGACGTCATCG |
| RPL3 | 2496 | 3 | CCGTGTCATTGCCCACACCC |
| RPL3 | 2497 | 4 | CCACCGAGGCCTGCGCAAGG |
| PSMD2 | 2498 | 1 | AGCCCACTAGCCGATACTTG |
| PSMD2 | 2499 | 2 | GATGCTCGTGGAACGACTAG |
| PSMD2 | 2500 | 3 | GGAAATCGTCCCCTATAACA |
| PSMD2 | 2501 | 4 | CCTGTGGAATGATAGCAGTA |
| HDAC6 | 2502 | 1 | TGTGCTGAGTTCCATTACCG |
| HDAC6 | 2503 | 2 | AGGACACGCAGCGATCTAGG |
| HDAC6 | 2504 | 3 | GCTTCCAGTGCTGAGTACGT |
| HDAC6 | 2505 | 4 | CCTCTAGGATAAGGATAATG |
| RRM1 | 2506 | 1 | CTTGTACCCCAATTCCAATG |
| RRM1 | 2507 | 2 | CCTACCTAGAAAGTTGTGGG |
| RRM1 | 2508 | 3 | TGGCAAACACTCTCCCATGG |
| RRM1 | 2509 | 4 | GGATCTCTTCATGAAACGAG |
| CSNK2A1 | 2510 | 1 | CTAGTTGGTGAGGATAGCCA |
| CSNK2A1 | 2511 | 2 | AGTCACATGTGGTGGAATGG |
| CSNK2A1 | 2512 | 3 | AGACATTGTAAAAGACCCTG |
| CSNK2A1 | 2513 | 4 | GATTGATCATGAGCACAGAA |
| HCK | 2514 | 1 | ATCCGGACCCTGGACAACGG |
| HCK | 2515 | 2 | AATGGCCTCGTAATCATACA |
| HCK | 2516 | 3 | ATGTATTGCCTCCGACCTGG |
| HCK | 2517 | 4 | CCAGCTTGAGGGATTCCCGA |
| HDAC4 | 2518 | 1 | CTTACCCGTACCAGTAGCGA |
| HDAC4 | 2519 | 2 | GCATCAGCGTGTCATACACG |
| HDAC4 | 2520 | 3 | GGGGCTGACTTACCGCAGAG |
| HDAC4 | 2521 | 4 | GGAGCCCATTGAGAGCGATG |
| RXRG | 2522 | 1 | TGTGTTTAACCAGAGATCCG |
| RXRG | 2523 | 2 | GAGGCAGAATGTGCTACCAG |
| RXRG | 2524 | 3 | TACGCTTGGCCCATTCAACG |
| RXRG | 2525 | 4 | CTTCAAGAGGACGATAAGGA |
| RARB | 2526 | 1 | AAGCAGGGTTTGTACACTCG |
| RARB | 2527 | 2 | GTGGATTGACCCAAACCGAA |
| RARB | 2528 | 3 | AAGGCCGTCTGAGAAAGTCA |
| RARB | 2529 | 4 | GTGTTATTAATAAAGTCACC |
| KRAS | 2530 | 1 | AAGAGGAGTACAGTGCAATG |
| KRAS | 2531 | 2 | AGATATTCACCATTATAGGT |
| KRAS | 2532 | 3 | CTGAATTAGCTGTATCGTCA |
| KRAS | 2533 | 4 | GATGTACCTATGGTCCTAGT |
| TGFB1 | 2534 | 1 | GGTTTCCACCATTAGCACGC |
| TGFB1 | 2535 | 2 | TTGATGTCACCGGAGTTGTG |
| TGFB1 | 2536 | 3 | GGTGAAGCGGAAGCGCATCG |
| TGFB1 | 2537 | 4 | GAATGGTGGCCAGGTCACCT |
| INHBC | 2538 | 1 | TCTCACTTTCAAGGTCCAAG |
| INHBC | 2539 | 2 | CAGAGCTCAGTCATCCTGGG |
| INHBC | 2540 | 3 | AAGACGAGTCTGGTTGATGG |
| INHBC | 2541 | 4 | ACCTCCACGGGGTCCCACAG |
| CD274 | 2542 | 1 | GGTTCCCAAGGACCTATATG |
| CD274 | 2543 | 2 | ACTGCTTGTCCAGATGACTT |
| CD274 | 2544 | 3 | ACATGTCAGTTCATGTTCAG |
| CD274 | 2545 | 4 | CACCACCAATTCCAAGAGAG |
| KDM1A | 2546 | 1 | TGGAATAGCAGAGACTCCGG |
| KDM1A | 2547 | 2 | CTAAATAACTGTGAACTCGG |
| KDM1A | 2548 | 3 | TTTCTGAAACAGGATCGTGT |
| KDM1A | 2549 | 4 | TGAGAAGTCATCCGGTCATG |
| XPO1 | 2550 | 1 | AGTGAGCTCTCAAAAAACGT |
| XPO1 | 2551 | 2 | TAGTCGAATGGCTAAACCAG |
| XPO1 | 2552 | 3 | TCTCAGGGAAACTCTTATGG |
| XPO1 | 2553 | 4 | TCACACCAGCAATCTCAGTG |
| PIK3CG | 2554 | 1 | ACTTAACCCTCTCACAGCAG |
| PIK3CG | 2555 | 2 | TGGCGGCGGACTTCTACCAC |
| PIK3CG | 2556 | 3 | GAGAATACGTCCTCCACATG |
| PIK3CG | 2557 | 4 | TTGCCTCTACAAAAACTGTG |
| IL6ST | 2558 | 1 | TGAGTTGCATTGTGAACGAG |
| IL6ST | 2559 | 2 | TCTTAAATAGGTGCGATGCA |
| IL6ST | 2560 | 3 | AAACGAAGCTGTCTTAGAGT |
| IL6ST | 2561 | 4 | GATCTGATGTAACCTTCCCA |
| TYMS | 2562 | 1 | ATGTGCGCTTGGAATCCAAG |
| TYMS | 2563 | 2 | TCTACAGATTATTCAGGACA |
| TYMS | 2564 | 3 | TTCCAAGGGAGTGAAAATCT |
| TYMS | 2565 | 4 | ACCAAACGTGTGTTCTGGAA |
| ACPP (ACP3) | 2566 | 1 | ACTCCTTGGCTAGTACACTT |
| ACPP (ACP3) | 2567 | 2 | AAAGGCAGGTATAGCAACTG |
| ACPP (ACP3) | 2568 | 3 | CTACGACCCTTTATATTGTG |
| ACPP (ACP3) | 2569 | 4 | GCCATGAGGATTCCTTTATG |
| PIM3 | 2570 | 1 | GCTCCGTGATAAAGTCGAAG |
| PIM3 | 2571 | 2 | GGCTTCGGCACGGTCTACGC |
| PIM3 | 2572 | 3 | GACTGGTTCGAGCGGCCCGA |
| PIM3 | 2573 | 4 | ACGGTCTACACCGACTTCGA |
| MAP1A | 2574 | 1 | CAGGCTGCTTAGGAATAACG |
| MAP1A | 2575 | 2 | TGGAACAGGACACATACTGG |
| MAP1A | 2576 | 3 | ACACAAAGACCGTTGGCCAG |
| MAP1A | 2577 | 4 | TGTTGAACATAAGGCTCCGG |
| DYRK1B | 2578 | 1 | AGGCTCGCAAGTACTTTGAA |
| DYRK1B | 2579 | 2 | GATGAAGTACTATATAGGTG |
| DYRK1B | 2580 | 3 | CACAGAGAGCTTACGCAGCG |
| DYRK1B | 2581 | 4 | CATGACTACATCGTGCGCAG |
| BCL2L1 | 2582 | 1 | CAGGCGACGAGTTTGAACTG |
| BCL2L1 | 2583 | 2 | GACCCCAGTTTACCCCATCC |
| BCL2L1 | 2584 | 3 | CAGTGGCTCCATTCACCGCG |
| BCL2L1 | 2585 | 4 | CTCCGATTCAGTCCCTTCTG |
| FLT1 | 2586 | 1 | ACAGCCACAGTCCGGCACGT |
| FLT1 | 2587 | 2 | AGGTTGAGGGATACCATATG |
| FLT1 | 2588 | 3 | CTTACCATATATATGCACTG |
| FLT1 | 2589 | 4 | TGGCCACTGTGTGATCACTG |
| PSMB2 | 2590 | 1 | GAGGAGGTTCACATGATATG |
| PSMB2 | 2591 | 2 | TTTATAAGATGCGAAATGGT |
| PSMB2 | 2592 | 3 | AAGATATTACTCCTGTGTGT |
| PSMB2 | 2593 | 4 | AGGGCCAGCGCTGTATTACA |
| CCND2 | 2594 | 1 | ATGTGCTCAATGAAGTCATG |
| CCND2 | 2595 | 2 | TGAAGGTCTGAGCATGCTTG |
| CCND2 | 2596 | 3 | ACTCCCATTATAGGTCTGTG |
| CCND2 | 2597 | 4 | CACTTGAAGTAGGAGCACTG |
| CDK6 | 2598 | 1 | GCCCGCGACTTGAAGAACGG |
| CDK6 | 2599 | 2 | AACACTCCAGAGATCCACGG |
| CDK6 | 2600 | 3 | TGGCTCACCTGACCACGTTG |
| CDK6 | 2601 | 4 | CATTGCAGGTCGTCACGCTG |
| KDR | 2602 | 1 | TAATGTACACGACTCCATGT |
| KDR | 2603 | 2 | CCAATCACACAATTAAAGCG |
| KDR | 2604 | 3 | CAGCCTCTGCCAATCCATGT |
| KDR | 2605 | 4 | CAAGAACTGAACTAAATGTG |
| BRD4 | 2606 | 1 | AGTCGATTTCAATCTCGTCG |
| BRD4 | 2607 | 2 | AGTCGAACTGTCACTGTCCG |
| BRD4 | 2608 | 3 | CCAGACCCCTGTCATGACAG |
| BRD4 | 2609 | 4 | CACCAAACTCCTGAGCATCA |
| CCND1 | 2610 | 1 | GTGTTCAATGAAATCGTGCG |
| CCND1 | 2611 | 2 | GGTTGGCATCGGGGTACGCG |
| CCND1 | 2612 | 3 | CGTGCCTCCGTAGGTCTGCG |
| CCND1 | 2613 | 4 | AGAGGCCACGAACATGCAAG |
| LAP3 | 2614 | 1 | CTCCACCGCAGACATGACGA |
| LAP3 | 2615 | 2 | AAGTGCTAGTAGTAAAACCG |
| LAP3 | 2616 | 3 | TGTCGGCAAAGCTCTATGGA |
| LAP3 | 2617 | 4 | GACCTCATGAGGGCTGACAT |
| PSMB8 | 2618 | 1 | CCAGAGCTCGCTTTACCCCG |
| PSMB8 | 2619 | 2 | AAGGAACGTTCAGATTGAGA |
| PSMB8 | 2620 | 3 | ACTAATGTAGGACCCAGCTG |
| PSMB8 | 2621 | 4 | TGGAGAACGTATTTCAGTGT |
| EZH2 | 2622 | 1 | ATGTTGGGGGTACATTCAGG |
| EZH2 | 2623 | 2 | TTATCAGAAGGAAATTTCCG |
| EZH2 | 2624 | 3 | TTATGATGGGAAAGTACACG |
| EZH2 | 2625 | 4 | CTTCTGTGAGCTCATTGCGC |
| HPRT1 | 2626 | 1 | AATAAATCAAGGTCATAACC |
| HPRT1 | 2627 | 2 | CTGTCCATAATTAGTCCATG |
| HPRT1 | 2628 | 3 | ACTAGAATGACCAGTCAACA |
| HPRT1 | 2629 | 4 | CACAGAGGGCTACAATGTGA |
| NPEPPS | 2630 | 1 | GTGATAGGGACCATCCACTG |
| NPEPPS | 2631 | 2 | GTCCGTGTTTACACTCCTGT |
| NPEPPS | 2632 | 3 | GAAAGTTTAGAAAATGCTAG |
| NPEPPS | 2633 | 4 | GCAAAGGCTGTAGTTGATGG |
| CDK4 | 2634 | 1 | CCAGATGGCACTTACACCCG |
| CDK4 | 2635 | 2 | AGTGTGAGAGTCCCCAATGG |
| CDK4 | 2636 | 3 | GTCCACATATGCAACACCTG |
| CDK4 | 2637 | 4 | GTCTACATGCTCAAACACCA |
| FYN | 2638 | 1 | ACGGGGACCTTGCGTACGAG |
| FYN | 2639 | 2 | TGGATACTACATTACCACCC |
| FYN | 2640 | 3 | GTCCCCCGAATCATTCCTTG |
| FYN | 2641 | 4 | TTGTCCTTTGGAAACCCAAG |
| RPS6KB1 | 2642 | 1 | CTCTTAGCCCCCATTCACTG |
| RPS6KB1 | 2643 | 2 | AATGAAAGCATGGACCATGG |
| RPS6KB1 | 2644 | 3 | CTTCGGGTACTTGGTAAAGG |
| RPS6KB1 | 2645 | 4 | AGCAGAACGGAATATTCTGG |
| CRTC1 | 2646 | 1 | TGTCAGTGGACAAACACGGA |
| CRTC1 | 2647 | 2 | AAATCCCAGTACCTGCAACT |
| CRTC1 | 2648 | 3 | GGTACCGTGACTGCAAGCGG |
| CRTC1 | 2649 | 4 | GCGGCCAACCTGACGCACCT |
| PTK2 | 2650 | 1 | TCTGATGATAAATGACTGCG |
| PTK2 | 2651 | 2 | ATGTGGGAGATACTGATGCA |
| PTK2 | 2652 | 3 | ACTTAAAGCTCAGCTCAGGT |
| PTK2 | 2653 | 4 | AGAGCAAAAGATTTGTACAC |
| FNTA | 2654 | 1 | TCACAAACCCGTCGTCCATG |
| FNTA | 2655 | 2 | ATGCAGCCAATTATACAGTG |
| FNTA | 2656 | 3 | ATAGGCGAGTATTAGTGGAA |
| FNTA | 2657 | 4 | TGTGGACCAACTTCTGAAAG |
| RARA | 2658 | 1 | GTGTAGCTCTCAGAGCACTC |
| RARA | 2659 | 2 | CTTCAAAGCACTTCTGCAGT |
| RARA | 2660 | 3 | AGAGTCCACCCAGCATAGGG |
| RARA | 2661 | 4 | AAGCAAGGCTTGTAGATGCG |
| PRLR | 2662 | 1 | CCATGAATGATACAACCGTG |
| PRLR | 2663 | 2 | TGTCCAGACTACATAACCGG |
| PRLR | 2664 | 3 | AAGACAGAAAACCCTACCTG |
| PRLR | 2665 | 4 | AATGGACTGACATTAGATGC |
| ALK | 2666 | 1 | CCATACCTTAAATACGTAGG |
| ALK | 2667 | 2 | CTGTAGCACTTTCAGAAGCG |
| ALK | 2668 | 3 | TCCAGACAACCCATTTCGAG |
| ALK | 2669 | 4 | CTCTATTGCAGTTAGCGGAG |
| CXCR4 | 2670 | 1 | TGACATGGACTGCCTTGCAT |
| CXCR4 | 2671 | 2 | TCTTCTGGTAACCCATGACC |
| CXCR4 | 2672 | 3 | CATCTTTGCCAACGTCAGTG |
| CXCR4 | 2673 | 4 | ACACCGAGGAAATGGGCTCA |
| RICTOR | 2674 | 1 | GGCATAGTCGCAAACATCTG |
| RICTOR | 2675 | 2 | TCTCCAAGGTGGGATAACGC |
| RICTOR | 2676 | 3 | GTGCCAAATAATTATCCATG |
| RICTOR | 2677 | 4 | TGTATAGGCAGGTAGACGTG |
| MDM2 | 2678 | 1 | GAGAACATTACCGGATTCGA |
| MDM2 | 2679 | 2 | TACCATGATCTACAGGAACT |
| MDM2 | 2680 | 3 | AGACACTTATACTATGAAAG |
| MDM2 | 2681 | 4 | CAACATCTGTTGCAATGTGA |
| SHH | 2682 | 1 | CTCACCCGCAGAGAACTCGG |
| SHH | 2683 | 2 | AGTGGCCAGGAGTGAAACTG |
| SHH | 2684 | 3 | GCGCCAGCGGAAGGTATGAA |
| SHH | 2685 | 4 | TGTGGCGCCGCACAACGACT |
| SSTR5 | 2686 | 1 | AGGCGGTGACAACAGGACGC |
| SSTR5 | 2687 | 2 | ACGTCCGCGAACACCAGGAG |
| SSTR5 | 2688 | 3 | GAAGGACGCGGCGTTCTGCG |
| SSTR5 | 2689 | 4 | GAGAATGTAGATGTTGGTGA |
| TUBE1 | 2690 | 1 | ATTTGCTCCTCAACCTAACG |
| TUBE1 | 2691 | 2 | ACTTTCCAGAATTCACCATG |
| TUBE1 | 2692 | 3 | ACATCTGTCAGTGTATACAG |
| TUBE1 | 2693 | 4 | TTTATAATACATTCCATGGG |
| KIT | 2694 | 1 | TCAGACTTAATAGTCCGCGT |
| KIT | 2695 | 2 | GAAAGAAGACAACGACACGC |
| KIT | 2696 | 3 | GAATGGCATGCTCCAATGTG |
| KIT | 2697 | 4 | TCTAGTGCATTCAAGCACAA |
| AGXT | 2698 | 1 | GCTGCTGTTCTTAACCCACG |
| AGXT | 2699 | 2 | CCCCTTTACATGGACCGGCA |
| AGXT | 2700 | 3 | CCGATGACCAAGGACCCTGG |
| AGXT | 2701 | 4 | GTCACTGAAGGAGATGAGCG |
| NOTCH1 | 2702 | 1 | TGCAGGTCAGTACTGTACCG |
| NOTCH1 | 2703 | 2 | TCCTGCCAGAACACCCACGG |
| NOTCH1 | 2704 | 3 | TCGCACGCCTCCTCGATCAG |
| NOTCH1 | 2705 | 4 | TTGACGTCGATCTCGCATCG |
| YES1 | 2706 | 1 | CTAGTCGCAAAGATTCTCGA |
| YES1 | 2707 | 2 | TCCAAAAGGCGTTACCCCTG |
| YES1 | 2708 | 3 | AAATTGGTGAAACACTACAC |
| YES1 | 2709 | 4 | AGAGAGAGTGAAACAACTAA |
| CYP19A1 | 2710 | 1 | TGATAGCAGAAAAAAGACGC |
| CYP19A1 | 2711 | 2 | CAGCATGACACGACGCAGAA |
| CYP19A1 | 2712 | 3 | GAGGGCACATCCTCAATACC |
| CYP19A1 | 2713 | 4 | GCATGAATTCTCCATATACC |
| TEK | 2714 | 1 | TGGCACAGGAACACCCATAG |
| TEK | 2715 | 2 | AGACCACTCTAAATTTGACC |
| TEK | 2716 | 3 | GCCTGAAACAGCATACCAGG |
| TEK | 2717 | 4 | TACTCGGCCAGGTATATAGG |
| TNFRSF8 | 2718 | 1 | GTGTCCCTTAGACGACCTCG |
| TNFRSF8 | 2719 | 2 | AGCTGCTTCTAAACTGACGA |
| TNFRSF8 | 2720 | 3 | ATCCAGAACGGGCTTCCCCG |
| TNFRSF8 | 2721 | 4 | GATGTGGCACAGATCATGCC |
| HSPB1 | 2722 | 1 | TGCCGGAGGAGTGGTCGCAG |
| HSPB1 | 2723 | 2 | CTCGGAGATCCGGCACACTG |
| HSPB1 | 2724 | 3 | TGGTCGAAGAGGCGGCTATG |
| HSPB1 | 2725 | 4 | CACTGCGGGGCTCTCGATGG |
| WEE1 | 2726 | 1 | TCATCAACAGAGCCCGCCAA |
| WEE1 | 2727 | 2 | CCATGAAGAGAGAACTACCC |
| WEE1 | 2728 | 3 | CCAGGAGATGCGTCGCCGCG |
| WEE1 | 2729 | 4 | TCTACGACGACACTGTCCTG |
| RXRB | 2730 | 1 | GGACAACAAAGACTGCACAG |
| RXRB | 2731 | 2 | ACGGCTATGTGCAATCTGCG |
| RXRB | 2732 | 3 | GCCCTGGCTGGATCCCGCAG |
| RXRB | 2733 | 4 | GTGGCTTCACATCTTCAGGG |
| INHBE | 2734 | 1 | GCACAGTTACTGGACAACCG |
| INHBE | 2735 | 2 | CTCACCCCTCAAGCCACTAG |
| INHBE | 2736 | 3 | GGGCACTGGTGCGAGCACAG |
| INHBE | 2737 | 4 | CCCTCCGGAGACTACAGCCA |
| AURKB | 2738 | 1 | ATTCTAGAGTATGCCCCCCG |
| AURKB | 2739 | 2 | TCTTTCCGGAGGACTCGCTG |
| AURKB | 2740 | 3 | CATCAACCCATACTGCAGGT |
| AURKB | 2741 | 4 | TGACGAGCAGCGAACAGCCA |
| AKT1 | 2742 | 1 | GAAGGTGCGTTCGATGACAG |
| AKT1 | 2743 | 2 | CCTGCACTCGGAGAAGAACG |
| AKT1 | 2744 | 3 | TGTTGAGGGGAGCCTCACGT |
| AKT1 | 2745 | 4 | TGTCATGGAGTACGCCAACG |
| LYN | 2746 | 1 | TGAAAGACAAGTCGTCCGGG |
| LYN | 2747 | 2 | GCTCGTGAGGCTCTACGCTG |
| LYN | 2748 | 3 | TTACTATAACAACAGTACCA |
| LYN | 2749 | 4 | TAATAACATCACCATGCACA |
| SSTR2 | 2750 | 1 | GGAGCCCACTCGGATTCCAG |
| SSTR2 | 2751 | 2 | CATCGACCGATACCTGGCTG |
| SSTR2 | 2752 | 3 | TGGTCTTCATCTTGGCATAG |
| SSTR2 | 2753 | 4 | AGCCCAGCATATATCATGAT |
| MAPK9 | 2754 | 1 | AGTACCGTGTCACCACGTAA |
| MAPK9 | 2755 | 2 | CCGGGAACAGGACTTTATGG |
| MAPK9 | 2756 | 3 | AGAAACTTCAGCCAACTGTG |
| MAPK9 | 2757 | 4 | CTTATGTCAGGTTATTCACA |
| AXL | 2758 | 1 | CTGAGAACATTAGTGCTACG |
| AXL | 2759 | 2 | CGAAGCCCATAACGCCAAGG |
| AXL | 2760 | 3 | CCTAGCAGTACATACCACCA |
| AXL | 2761 | 4 | CCCGAAGCCAATGTACCTCG |
| XIAP | 2762 | 1 | ATGACAACTAAAGCACCGCA |
| XIAP | 2763 | 2 | ATGGATATACTCAGTTAACA |
| XIAP | 2764 | 3 | TCTGACCAGGCACGATCACA |
| XIAP | 2765 | 4 | TATCAGACACCATATACCCG |
| BRD3 | 2766 | 1 | CATCACTGCAAACGTCACGT |
| BRD3 | 2767 | 2 | GATGCTATCCAAGAAGCACG |
| BRD3 | 2768 | 3 | ATACAATCCCCCAGACCACG |
| BRD3 | 2769 | 4 | TGAAGGTACACAGCAAGTGG |
| TOP2B | 2770 | 1 | TAGGCTACATGGCTTACCAG |
| TOP2B | 2771 | 2 | GTGTACACTGATATTAACAG |
| TOP2B | 2772 | 3 | ATGATTATGACCGATCAGGT |
| TOP2B | 2773 | 4 | ATCAACGTGTAGAGCCTGAG |
| SH2B3 | 2774 | 1 | ACTACCGGGACACAGGCCGT |
| SH2B3 | 2775 | 2 | GGAGCTCTTCGACCCACCCA |
| SH2B3 | 2776 | 3 | TGAGTTGCACGCCGTAGCGG |
| SH2B3 | 2777 | 4 | GCAGCAGCTGAATTCATGGA |
| DHH | 2778 | 1 | CCGCAACCACGTCCACGTGT |
| DHH | 2779 | 2 | CAGGATTCACTCCACTACGA |
| DHH | 2780 | 3 | AGGAAGAGCAGCACCGGCGT |
| DHH | 2781 | 4 | GAAATGCAACTGTGCGCCTG |
| DYRK2 | 2782 | 1 | TTGAGGATAACAGTAACAAG |
| DYRK2 | 2783 | 2 | CTAAATGCTAAGAAGCGCCA |
| DYRK2 | 2784 | 3 | ACAGCATTCATAGACGGCAG |
| DYRK2 | 2785 | 4 | CAAGCACTGCAGAATCGAGT |
| FLT4 | 2786 | 1 | GCCCTCCAGTCACGGCACTG |
| FLT4 | 2787 | 2 | CTCACCTCTCACGAACACGT |
| FLT4 | 2788 | 3 | CATCGAATCCAAGCCATCCG |
| FLT4 | 2789 | 4 | CATACCATGCACAATGACCT |

TABLE 6C
Library of gene modulatory reagents.

| Target gene | SEQ ID NO | gRNA # | gRNA Seq |
|---|---|---|---|
| CTRL-non-1 | 2790 | 1 | CCCGATGGACTATACCGAAC |
| CTRL-non-2 | 2791 | 1 | TCAATTCTCACTCACGACCA |
| CTRL-non-3 | 2792 | 1 | GTTGATCGAAAATGGGAGAA |
| CTRL-non-4 | 2793 | 1 | CGTCCCTTCGTCTCTGCTTA |
| CTRL-non-5 | 2794 | 1 | AATCGACTCGAACTTCGTGT |
| CTRL-non-6 | 2795 | 1 | AGCTCGCCATGTCGGTTCTC |
| CTRL-non-7 | 2796 | 1 | CAGAGACAATGACATGTAGA |
| CTRL-non-8 | 2797 | 1 | AACCACGGCATTGAGAGGTG |
| CTRL-non-9 | 2798 | 1 | CAAATACAATTACTTATAGC |
| CTRL-non-10 | 2799 | 1 | CGACTAACCGGAAACTTTTT |
| CTRL-non-11 | 2800 | 1 | CAAAAGTCTCGCTTGGTCCT |
| CTRL-non-12 | 2801 | 1 | CAGTAGCGATCGAATGTCAA |
| CTRL-non-13 | 2802 | 1 | AGTATTAGGTACCTGCCCTA |
| CTRL-non-14 | 2803 | 1 | CTGGCTTATTAGCTATAAAG |
| CTRL-non-15 | 2804 | 1 | AATATTTGGCTCGGCTGCGC |
| CTRL-non-16 | 2805 | 1 | GCTTTCAATTGCAAAAATAC |
| CTRL-non-17 | 2806 | 1 | ATACTCTCACAGGTACATAA |
| CTRL-non-18 | 2807 | 1 | GCAAATTCAGACAGCTAATT |
| CTRL-non-19 | 2808 | 1 | TCGCGCTTGGGTTATACGCT |
| CTRL-non-20 | 2809 | 1 | CCTACGCGGTAGGGAACTTT |
| CTRL-non-21 | 2810 | 1 | GACCGCAAAGTGGTCCGAAG |
| CTRL-non-22 | 2811 | 1 | GCTGTTCCGAAGTTGAGAAT |
| CTRL-non-23 | 2812 | 1 | CGCGTGTAGCTGGAGACAAG |
| CTRL-non-24 | 2813 | 1 | CGCGGCCCACGCGTCATCGC |
| CTRL-non-25 | 2814 | 1 | TCCTGCCAAGAAACACCCTT |
| CTRL-non-26 | 2815 | 1 | AAATTGGCTTTCGTTCGTGC |
| CTRL-non-27 | 2816 | 1 | TAAGGCGACCTGCGCTTGTG |
| CTRL-non-28 | 2817 | 1 | TCGCGGACATAGGGCTCTAA |
| CTRL-non-29 | 2818 | 1 | CGGTTTACATCTGCCCATCG |
| CTRL-non-30 | 2819 | 1 | CGATGGATCCCTAGTTCCTG |
| CTRL-non-31 | 2820 | 1 | CGAACTTAATCCCGTGGCAA |
| CTRL-non-32 | 2821 | 1 | GTTCATTTCCAAGTCCGCTG |
| CTRL-non-33 | 2822 | 1 | GTTTTCAGTTGCCCAACAGC |
| CTRL-non-34 | 2823 | 1 | CCTGCGGTGCACGGCTAGCC |
| CTRL-non-35 | 2824 | 1 | GGGCGCTAAGATATATGCCC |
| CTRL-non-36 | 2825 | 1 | CAAACAGTCTAAGGCGACGA |
| CTRL-non-37 | 2826 | 1 | TGCCCACTTAGCAACACTCT |
| CTRL-non-38 | 2827 | 1 | AACGCTGTCGTACGTGTATA |
| CTRL-non-39 | 2828 | 1 | GCCGCCGATTTCATAAGTAA |
| CTRL-non-40 | 2829 | 1 | ACGCATGCTTCCCAAAGCGT |
| CTRL-non-41 | 2830 | 1 | AAGCACTAGTCCGTATGATG |
| CTRL-non-42 | 2831 | 1 | TAGTCTCACCTGATGGCGTG |
| CTRL-non-43 | 2832 | 1 | AGAAGAAAAAAATGTCTACG |
| CTRL-non-44 | 2833 | 1 | TCTGGCTTGACACGACCGTT |
| CTRL-non-45 | 2834 | 1 | TCTATTTTGTCTGCGCAGAA |
| CTRL-non-46 | 2835 | 1 | CGAGCAAAGATTGTTGGATA |
| CTRL-non-47 | 2836 | 1 | TTCTTAGAAGTTGCTCCACG |
| CTRL-non-48 | 2837 | 1 | TAATCACATTGCTTAACCGG |
| CTRL-non-49 | 2838 | 1 | GGAGAGGGCCCGCGAACTCA |
| CTRL-non-50 | 2839 | 1 | ACCCATATATGCTGCCGCAC |
| CTRL-non-51 | 2840 | 1 | CGGGATGCAGCTGGAGAGGA |
| CTRL-non-52 | 2841 | 1 | GTCCTCATCCGGTCAGGCTG |
| CTRL-non-53 | 2842 | 1 | AAATACAAGCTATAGCGATA |
| CTRL-non-54 | 2843 | 1 | GCGATCGGAGTGCCACGATA |
| CTRL-non-55 | 2844 | 1 | AGCGATTCACGTATTAGATG |
| CTRL-non-56 | 2845 | 1 | GAGTAATTTCGAACGTATTG |
| CTRL-non-57 | 2846 | 1 | TTCTTCGGCCTACACCCGGT |
| CTRL-non-58 | 2847 | 1 | GTACCCCTATGGCCGTTCTA |
| CTRL-non-59 | 2848 | 1 | ATAGCGGATGTCCTTGGAAA |
| CTRL-non-60 | 2849 | 1 | CGTGTTTGGAATTTGCCGCG |
| CTRL-non-61 | 2850 | 1 | GCCACACGAATCATAAAGAG |
| CTRL-non-62 | 2851 | 1 | ACTTACGGCACTCGCATGCC |
| CTRL-non-63 | 2852 | 1 | GAGACCACTTTCGTGCAAGC |
| CTRL-non-64 | 2853 | 1 | TGCCGCTATACTAAAACCTT |
| CTRL-non-65 | 2854 | 1 | ATCTCTATACTGTCACTCGC |
| CTRL-non-66 | 2855 | 1 | TCATCTTACATCTGGGAGAC |
| CTRL-non-67 | 2856 | 1 | TGAACGGTGAAGAGATAGGG |
| CTRL-non-68 | 2857 | 1 | CTTCCTGCGTGGCTTTAAAC |
| CTRL-non-69 | 2858 | 1 | AAACGAGATCGAGAAAGGTA |
| CTRL-non-70 | 2859 | 1 | GAGCAGCTGTCAGGTCTTGT |
| CTRL-non-71 | 2860 | 1 | GGTACTGGAAGTCCGAAACC |
| CTRL-non-72 | 2861 | 1 | AGGCCACAAATTGTATACAG |
| CTRL-non-73 | 2862 | 1 | CCCTTCTGGCGGGCCAAACA |
| CTRL-non-74 | 2863 | 1 | TCAACCCCAGCGCACCGTTG |
| CTRL-non-75 | 2864 | 1 | TCGAGAGGAAAAACACACTG |
| CTRL-non-76 | 2865 | 1 | GGCGCATTAAAGTCGAGAGC |
| CTRL-non-77 | 2866 | 1 | GGAGATGCGGCCTTCTCAAA |
| CTRL-non-78 | 2867 | 1 | CCGCTGTCTCACTAATCTCA |
| CTRL-non-79 | 2868 | 1 | AGCATTCTCACCAAGACCGA |
| CTRL-non-80 | 2869 | 1 | GACTTCTAGAATATAAAAGA |
| CTRL-non-81 | 2870 | 1 | CTGGTGACCGACAATTACAC |
| CTRL-non-82 | 2871 | 1 | ATAGCCGCCGCTCATTACTT |
| CTRL-non-83 | 2872 | 1 | TACCCTCCGGATACGGACTG |
| CTRL-non-84 | 2873 | 1 | CTGCCCCAGGCGTAATCCTC |
| CTRL-non-85 | 2874 | 1 | TACGTAAGTGACGACAGGAA |
| CTRL-non-86 | 2875 | 1 | CAGCGCCGAAACTCTTTCCG |
| CTRL-non-87 | 2876 | 1 | CGCTTCCGCGGCCCGTTCAA |
| CTRL-non-88 | 2877 | 1 | TAAGCCTCATGAAGGAGGGG |
| CTRL-non-89 | 2878 | 1 | ACAGCGCTCTCGTGTACTAT |
| CTRL-non-90 | 2879 | 1 | AGTCTTAAAGACCCTAAGCT |
| CTRL-non-91 | 2880 | 1 | CTTAAGTCATGAGCAAAGAT |
| CTRL-non-92 | 2881 | 1 | GTTACGTACCCTCCAACTTC |
| CTRL-non-93 | 2882 | 1 | GGATATTGAGTAAACCCGAT |
| CTRL-non-94 | 2883 | 1 | CGACGCTAGGTAACGTAGAG |
| CTRL-non-95 | 2884 | 1 | GAGAAGGATGGAAATTAGAA |
| CTRL-non-96 | 2885 | 1 | CTCCGTTATGTGGCATGAGA |
| CTRL-non-97 | 2886 | 1 | GGGCCTACGATCAGAGGTGT |
| CTRL-non-98 | 2887 | 1 | TCTAAAGCCGTCCTGATGTT |
| CTRL-non-99 | 2888 | 1 | AAGGCGCGCGAATGTGGCAG |
| CTRL-non-100 | 2889 | 1 | GAACGTAGAAATTCCCATTT |
| Ctrl-hg38-1 | 2890 | 1 | GCCCAGTGGGTCCTCGTGAC |
| Ctrl-hg38-2 | 2891 | 1 | TTGACATCAGAAACAGACGT |
| Ctrl-hg38-3 | 2892 | 1 | TGTCATGGTTTGACTGTCCC |
| Ctrl-hg38-4 | 2893 | 1 | GGCTCGGGATCCCTAGCAGG |
| Ctrl-hg38-5 | 2894 | 1 | GTGTTGGATAAGTAATGGGG |
| Ctrl-hg38-6 | 2895 | 1 | GGATATTTGAGCTCATCTCT |
| Ctrl-hg38-7 | 2896 | 1 | GATACATTATAAGGAGCATT |
| Ctrl-hg38-8 | 2897 | 1 | ATAATTAGTTTTGAGAATAG |
| Ctrl-hg38-9 | 2898 | 1 | TGAGGGACATTCATCCGGCC |
| Ctrl-hg38-10 | 2899 | 1 | GAGAACCAAGGATGGCTTAT |
| Ctrl-hg38-11 | 2900 | 1 | TGCACTATTTCATGGTGATA |
| Ctrl-hg38-12 | 2901 | 1 | CGTTTTAAACTTGCAAGGTA |
| Ctrl-hg38-13 | 2902 | 1 | ACCGGCTTCAACTCCTTCAG |
| Ctrl-hg38-14 | 2903 | 1 | CATAGACCAATCAGCACTTT |
| Ctrl-hg38-15 | 2904 | 1 | CTTGGGCATTTATGTCTCCA |
| Ctrl-hg38-16 | 2905 | 1 | AATAATATTTCCCTGCACTC |
| Ctrl-hg38-17 | 2906 | 1 | CTGGAAAAGTTTAGCAATCA |
| Ctrl-hg38-18 | 2907 | 1 | TTAGTCACAGTCCCCTACCT |
| Ctrl-hg38-19 | 2908 | 1 | ACTTATACCACATGGTAACC |
| Ctrl-hg38-20 | 2909 | 1 | CTGCTATGGGGGGAGTTATT |
| Ctrl-hg38-21 | 2910 | 1 | AAGTCAACATGTCTAACTCA |
| Ctrl-hg38-22 | 2911 | 1 | CCTAGCTCACTTAAGTTTGC |
| Ctrl-hg38-23 | 2912 | 1 | GGGAGGTATTATTGGATTCT |
| Ctrl-hg38-24 | 2913 | 1 | TTTATCAATTTACTGAAGTA |
| Ctrl-hg38-25 | 2914 | 1 | CTTACCCTATAGGGGATATT |
| Ctrl-hg38-26 | 2915 | 1 | CATGAGCCCCAAGGGATCTT |
| Ctrl-hg38-27 | 2916 | 1 | AGGCCTGAGACTCACATTTC |
| Ctrl-hg38-28 | 2917 | 1 | TCAATCTCAGCCCAGGACCT |
| Ctrl-hg38-29 | 2918 | 1 | AGATACGTTTTAGACCTCAA |
| Ctrl-hg38-30 | 2919 | 1 | TAGAGCCTCAACCATGTTTT |
| Ctrl-hg38-31 | 2920 | 1 | CCAGGGGACCTGAACGCGTG |
| Ctrl-hg38-32 | 2921 | 1 | ACACAGAGACATCTCCTGTC |
| Ctrl-hg38-33 | 2922 | 1 | GGTAAGAATGAATAGTTCCC |
| Ctrl-hg38-34 | 2923 | 1 | TGTCCTCATTCCAATTCTTG |
| Ctrl-hg38-35 | 2924 | 1 | ATACGACTTAGCAGCCTTGT |
| Ctrl-hg38-36 | 2925 | 1 | AAGGCCAATGATTGAAGTCA |
| Ctrl-hg38-37 | 2926 | 1 | AGCACTTTAACTAGACAATA |
| Ctrl-hg38-38 | 2927 | 1 | AGCAACACCAGCATCATGGC |
| Ctrl-hg38-39 | 2928 | 1 | TAATTAAACACTTGCAACAT |
| Ctrl-hg38-40 | 2929 | 1 | GAAGCACACGTGGTGTATTT |
| Ctrl-hg38-41 | 2930 | 1 | GGAGCTTGAAGTTCTAAATG |
| Ctrl-hg38-42 | 2931 | 1 | AATCAGCATTAACAGAAAGG |
| Ctrl-hg38-43 | 2932 | 1 | CATGCTTAAGAGTTTCCTAT |
| Ctrl-hg38-44 | 2933 | 1 | ATCATACCCCATGAGACTTT |
| Ctrl-hg38-45 | 2934 | 1 | ATACTCACATCATGTTAAGA |
| Ctrl-hg38-46 | 2935 | 1 | CGCCCATGTTGGCCATAAAC |
| Ctrl-hg38-47 | 2936 | 1 | ATCAGTCTGTCTTGTAGGCA |
| Ctrl-hg38-48 | 2937 | 1 | TTGCTGGGCTGACCCAAGCT |
| Ctrl-hg38-49 | 2938 | 1 | GTTTAGGTAAAAATACCTTG |
| Ctrl-hg38-50 | 2939 | 1 | TTCTACCCCACCACATCCCA |
| Ctrl-hg38-51 | 2940 | 1 | TCGTGGAAACATACAAGTCT |
| Ctrl-hg38-52 | 2941 | 1 | TCATGGTCGGATGAGTTGGG |
| Ctrl-hg38-53 | 2942 | 1 | AAGCATTTGCATCCAGACAA |
| Ctrl-hg38-54 | 2943 | 1 | CCTGCTTCAGCCACTAAGCA |
| Ctrl-hg38-55 | 2944 | 1 | TGGTCTAGCGTGTCTCGCTC |
| Ctrl-hg38-56 | 2945 | 1 | CCAGCCAAACTACACCCCAG |
| Ctrl-hg38-57 | 2946 | 1 | CAACGACTGCATTAGGGCAA |
| Ctrl-hg38-58 | 2947 | 1 | TTCCCTGGGATAGCTGATAT |
| Ctrl-hg38-59 | 2948 | 1 | TGTTGTGTGCATACCCTAGT |
| Ctrl-hg38-60 | 2949 | 1 | GATGAATATGAATGTATGAC |
| Ctrl-hg38-61 | 2950 | 1 | TAAGTGCATTTCTATGTCCT |
| Ctrl-hg38-62 | 2951 | 1 | GATTATCTATCCTGGTTCCC |
| Ctrl-hg38-63 | 2952 | 1 | AATAAATAGCATGACTTATG |
| Ctrl-hg38-64 | 2953 | 1 | TTTGCAATCCCCGAGGTGAC |
| Ctrl-hg38-65 | 2954 | 1 | GCTGCCACAAAGGTTGTCAA |
| Ctrl-hg38-66 | 2955 | 1 | GCAATAATTAGGACCACCCA |
| Ctrl-hg38-67 | 2956 | 1 | AGATGACTGGAGTGCTACAA |
| Ctrl-hg38-68 | 2957 | 1 | CTCATTGACCCACATAAAAT |
| Ctrl-hg38-69 | 2958 | 1 | AACTCTTCTGTCGATGAGCA |
| Ctrl-hg38-70 | 2959 | 1 | GGGTAAACAGTCATGCTGCA |
| CTRL-non | 2960 | 1 | AAACTGTGCGACGGTAAGCG |
| CTRL-non | 2961 | 2 | AAAGATTCACCTCGCTACGG |
| CTRL-non | 2962 | 3 | AACATGTCATCGTTTACGCC |
| CTRL-non | 2963 | 4 | AACCGATTTCAATCGCGTGG |
| CTRL-non | 2964 | 5 | AACGAAAGCTCGTTAACTCG |
| CTRL-non | 2965 | 6 | AACGATGCGGGCGACGTGCT |
| CTRL-non | 2966 | 7 | AACTCCCCCGACTCCGTTCG |
| CTRL-non | 2967 | 8 | AAGCGATGGTCCGTATACTA |
| CTRL-non | 2968 | 9 | AATATTTGGCTCGGCTGCGC |
| CTRL-non | 2969 | 10 | AATCCGGAGTAATCCGACCC |
| CTRL-non | 2970 | 11 | AATCGACTCGAACTTCGTGT |
| CTRL-non | 2971 | 12 | ACACCATATCGGCGGGACGC |
| B2M | 2972 | 1 | GGCCGAGATGTCTCGCTCCG |
| B2M | 2973 | 1 | GGCCGAGATGTCTCGCTCCG |
| B2M | 2974 | 2 | GGCCACGGAGCGAGACATCT |
| B2M | 2975 | 2 | GGCCACGGAGCGAGACATCT |
| B2M | 2976 | 1 | GGCCGAGATGTCTCGCTCCG |
| B2M | 2977 | 1 | GGCCGAGATGTCTCGCTCCG |
| B2M | 2978 | 2 | GGCCACGGAGCGAGACATCT |
| B2M | 2979 | 2 | GGCCACGGAGCGAGACATCT |

TABLE 6D
Library of gene modulatory reagents.

| Target | SEQ | | |
|---|---|---|---|
| gene | ID NO | gRNA # | gRNA Seq |
| BIRC5 | 2980 | 1 | AGTTCTTGAATGTAGAGATG |
| BIRC5 | 2981 | 2 | GGGCAGTCTCACCCGCTCCG |
| BIRC5 | 2982 | 3 | TCTTGAATGTAGAGATGCGG |
| BIRC5 | 2983 | 4 | CAAGTCTGGCTCGTTCTCAG |
| BRAF | 2984 | 1 | GGGCCAGGCTCTGTTCAACG |
| BRAF | 2985 | 2 | ATACCCAATAGAGTCCGAGG |
| BRAF | 2986 | 3 | GCCCAACAAACAGAGGACAG |
| BRAF | 2987 | 4 | TCATAATTAACACACATCAG |
| CDK4 | 2988 | 1 | AAGGCCCGTGATCCCCACAG |
| CDK4 | 2989 | 2 | GTCTACATGCTCAAACACCA |
| CDK4 | 2990 | 3 | CCAGTGGCTGAAATTGGTGT |
| CDK4 | 2991 | 4 | AGCCACTGGCTCATATCGAG |
| CDK6 | 2992 | 1 | GCCCGCGACTTGAAGAACGG |
| CDK6 | 2993 | 2 | CCAGCAGTACGAATGCGTGG |
| CDK6 | 2994 | 3 | GACCTTCGAGCACCCCAACG |
| CDK6 | 2995 | 4 | AATGAAGAAAGTCCAGACCT |
| CYP11B1 | 2996 | 1 | CACTGTCCTGGGGACCCGGG |
| CYP11B1 | 2997 | 2 | AATGGGCCCTAGTTCCTGGA |
| CYP11B1 | 2998 | 3 | CAAAGGGCAGCACTGTCCTG |
| CYP11B1 | 2999 | 4 | CCCCACAGGTACGACTTGGG |
| CYP11B2 | 3000 | 1 | CATCGGGAGGAACCTCTGCA |
| CYP11B2 | 3001 | 2 | AAACGGCAGCACCGTCCTAG |
| CYP11B2 | 3002 | 3 | CCCCACAGGTACAACTTGGG |
| CYP11B2 | 3003 | 4 | CAACTTGGGAGGACCACGCA |
| EGFR | 3004 | 1 | GAGAACCTAGAAATCATACG |
| EGFR | 3005 | 2 | TGTCACCACATAATTACCTG |
| EGFR | 3006 | 3 | CCAGATGGATGTGAACCCCG |
| EGFR | 3007 | 4 | ATGGACTTCCAGAACCACCT |
| FLT3 | 3008 | 1 | AGATACATCCACTTCCACAG |
| FLT3 | 3009 | 2 | GAGACAGGAAATGTTCCCTG |
| FLT3 | 3010 | 3 | AGAACAATGATTCATCAGTG |
| FLT3 | 3011 | 4 | AAAGCTGTTCATGTGAACCA |
| GART | 3012 | 1 | TTATCCTGGAGACTACACCA |
| GART | 3013 | 2 | AGAATTGAGCAAGGGCAGTG |
| GART | 3014 | 3 | TATCCTGGAGACTACACCAA |
| GART | 3015 | 4 | GTCTTCGGGAAGTACCTGAG |
| JAK1 | 3016 | 1 | ACAAAGAGGAGAGATACCTG |
| JAK1 | 3017 | 2 | GAGGAGCTCCAAGAAGACTG |
| JAK1 | 3018 | 3 | CAAAGGACAAGGCCTCCTCG |
| JAK1 | 3019 | 4 | GAAGACTGAGGTGAACCTGG |
| JAK2 | 3020 | 1 | AGAGTTATAGATGGCCAGTG |
| JAK2 | 3021 | 2 | CTGCCACTGCAATACCAACG |
| JAK2 | 3022 | 3 | CAACCTCACCAACATTACAG |
| JAK2 | 3023 | 4 | AATGAAGAGTACAACCTCAG |
| KIF11 | 3024 | 1 | TCTTGTGTAGGAGTATACGG |
| KIF11 | 3025 | 2 | GAAGGGGAAGAACATCCAGG |
| KIF11 | 3026 | 3 | GCCCTCCAAGAGAATCCTGG |
| KIF11 | 3027 | 4 | TTCGTCTGCGAAGAAGAAAG |
| KIT | 3028 | 1 | CTGGGTCTGTGAGAGGACAG |
| KIT | 3029 | 2 | GCCTAATCTCGTCGCCCACG |
| KIT | 3030 | 3 | TCAACCATCTGTGAGTCCAG |
| KIT | 3031 | 4 | ACCACTAGCTTTCCAAACGG |
| MAP2K1 | 3032 | 1 | AAGCACAAGATCATGCACAG |
| MAP2K1 | 3033 | 2 | GCAGCAGCGAAAGCGCCTTG |
| MAP2K1 | 3034 | 3 | GAGTTGACTAGGATGTTGGA |
| MAP2K1 | 3035 | 4 | CTTACCCAGAAGCAGAAGGT |
| MAP2K2 | 3036 | 1 | AAGCACCAGATCATGCACCG |
| MAP2K2 | 3037 | 2 | CCTGGGGAAAGTCAGCATCG |
| MAP2K2 | 3038 | 3 | AGGTGCTGAAAGAGGCCAAG |
| MAP2K2 | 3039 | 4 | GGATTCCCGAGGAGATCCTG |
| MDM2 | 3040 | 1 | TTCCGAAGCTGGAATCTGTG |
| MDM2 | 3041 | 2 | CGAAGCTGGAATCTGTGAGG |
| MDM2 | 3042 | 3 | ACAGGTGTCACCTTGAAGGT |
| MDM2 | 3043 | 4 | GTGGTTACAGCACCATCAGT |
| MET | 3044 | 1 | TCAGCTTCCCAACTTCACCG |
| MET | 3045 | 2 | CTTGGTGCAGAGGAGCAATG |
| MET | 3046 | 3 | ACTCTGTTCGATATTCATCA |
| MET | 3047 | 4 | ATGTCAGCAGTATGATTGTG |
| MTOR | 3048 | 1 | GCTGATGCAAGTGAAGACTG |
| MTOR | 3049 | 2 | TCAGGAAATGATCCGCACAG |
| MTOR | 3050 | 3 | AATAGGGTGAATGATCCGGG |
| MTOR | 3051 | 4 | CGTGCTGGACATCATCCGAG |
| PIK3CA | 3052 | 1 | TTATTAATGTAGCCTCACGG |
| PIK3CA | 3053 | 2 | CCATCATCAGGTGAACTGTG |
| PIK3CA | 3054 | 3 | GTTTGACTGGTTCAGCAGTG |
| PIK3CA | 3055 | 4 | AGCAAATCTTCTAATCCATG |
| PSMB5 | 3056 | 1 | CTGCAACTATGACTCCATGG |
| PSMB5 | 3057 | 2 | CGTGTTGGAGAGACCGCTAC |
| PSMB5 | 3058 | 3 | CCGCTACCGGTGAACCAGCG |
| PSMB5 | 3059 | 4 | GAGATCAACCCATACCTGCT |
| RPL3 | 3060 | 1 | GGCTACGTGGAAACCCCTCG |
| RPL3 | 3061 | 2 | CATTGTAGAGACACCACCCA |
| RPL3 | 3062 | 3 | TGTAGAGACACCACCCATGG |
| RPL3 | 3063 | 4 | TGTGGGCATTGTGGGCTACG |
| TOP2A | 3064 | 1 | ATATAATGACTTCATCAACA |
| TOP2A | 3065 | 2 | TGTTGTGAAGAAGAAGAACA |
| TOP2A | 3066 | 3 | GTGTACGCTTATCCTGACTG |
| TOP2A | 3067 | 4 | AACCAATGTAGGTGTCTGGG |
| TUBG1 | 3068 | 1 | CCAGGACGAGATGAGCGATG |
| TUBG1 | 3069 | 2 | TGTGTCACTCCATTGCTGGG |
| TUBG1 | 3070 | 3 | TTTTGACATCATAGACCGGG |
| TUBG1 | 3071 | 4 | TGTAGGGTGATGATTTCCCT |

### EXAMPLES

### Example 1

### Genetic Pharmacopeia

A drug library comprising molecularly targeted oncology drugs of Table 2B was generated. The drug library is updated periodically to include additional targeted oncology drugs as they are identified. A genetic pharmacopeia was generated to represent the genetic targets of the drug library (Table 5B).

A library of gene modulatory reagents comprising guide RNA (gRNA) sequences associated with each gene target was designed. As shown in Table 6A, five potential gRNA sequences were designed for each oncology drug target to generate gRNA sequences having SEQ ID NOS: 1-1525. The library of gene modulatory reagents is constructed to comprise at least one gRNA sequence selected from SEQ ID NOS: 1-1525. The library is constructed in a format compatible with use in primary cancer cells using a viral delivery method (adenovirus for Cas nuclease delivery, lentivirus for gRNA delivery).

### Example 2

### Cancer Functional Susceptibility Profiling

A method is performed to determine the functional susceptibility of a patient's cancer cells to one or more perturbagens which model the action of the targeted oncology drugs identified in Example 1. The library comprising at least one gRNA sequence selected from SEQ ID NOS: 1-1525 and associated gene editing agent(s) (e.g., RNA-guided nuclease) are delivered to primary cancer cells derived from the patient in order to genetically modify the cancer cells. The Cas nuclease and gRNA are delivered by lentivirus. In this example, genetic modification occurs via gene editing using a CRISPR-based method. The modified cancer cells are propagated in vivo, however, the method may be employed in in vitro environments that mimic the in vivo context. The effect of each gene edit is evaluated by screening the modified cancer cells in a pooled or array format. Next-generation sequencing is performed to determine the effect of the individual perturbations on the viability of the patient's cancer cells. Oncology drug(s) associated with the perturbagens that reduce viability of the cancer cells are selected as a putative therapeutic for the patient.

### Example 3

### CRISPR-Based Method for Personalized Functional Genomics

Methods for the identification of patient-specific tumor therapeutic vulnerabilities were performed utilizing function genomics as outlined in FIG. 2. Patient-derived samples, obtained directly from the patient or after passage in mice (PDX), were dissociated and infected with a gRNA library corresponding to the desired therapeutic drug collection. Cells were viably maintained in vitro, using 3D and/or organoid approaches, allowing gRNA which target essential tumor regulators to be gradually depleted from the population (“drop-out). This approach leveraged the insight that the effect of each clinically used targeted oncology drug can be modeled by CRISPR-mediated mutation of the corresponding gene encoding the drug target (FIG. 3).

Guide-RNA Library Cloning

A library of guide RNAs (gRNA) with 1685 elements having 1585 gRNAs directed against drug target genes and 100 control gRNAs was designed (FIG. 4). The library comprises the target gRNAs of Table 6B and control gRNAs having SEQ ID NOS: 2790-2959 of Table 6C. Guide RNAs targeting the ubiquitously expressed but not essential cell surface molecule beta-2 microglobulin (B2M) were also included. The 20 nt gRNA sequence was flanked on either side by a sequence containing a recognition site for the Type-IIS restriction enzyme Bbs-I, and outside of the Bbs-I elements flanked by primer binding sites that could be used for PCR amplification of the library. The upstream and downstream Bbs-I elements were designed such that Bbs-I digestion of the PCR product releases the 20 bp gRNA encoding sequence flanked with 4 bp overhangs compatible with the corresponding overhangs in the destination vector for gRNA expression. Using primers complementary to the primer binding sites in the library oligonucleotide pool, the library was amplified by PCR for 10 cycles using Q5 DNA polymerase. PCR products were purified using Zymo Clean&Concentrate kit and then included in a GoldenGate cloning reaction using 20 cycles of 37° C. digestion with Bbs-I followed by 16° C. ligation with T4-DNA ligase to introduce the library into the destination vector for gRNA expression. The GoldenGate cloning reaction was further cleaned using Zymo Clean&Concentrate kit and then used in multiple reactions for electroporation into electrocompetent Stbl-4 bacteria. The entire transformation reaction from 3-5 electroporations was inoculated into 600 ml of LB with appropriate antibiotic selection and grown for 18 hours at 30° C. to avoid recombination. Bacterial cells were harvested and DNA isolated using Zymo Maxiprep kit. Barcode readcount distribution was measured by next generation sequencing of the pooled plasmid DNA or transduced cells (FIG. 5), demonstrating near-complete barcode representation and broadly equal readcount distribution.

Another library of gRNAs directed against drug target genes was prepared comprising the gRNAs of Table 6D. The library also includes gRNAs having SEQ ID NOS: 2972-2979 directed to B2M, and control gRNAs having SEQ ID NOS: 2890-2905 and 2960-2971.

Virus Production

Lentiviral particles containing viral genome encoding expression units for the gRNA library and Cas9 were generated by transfecting 293FT cells with transfer vector and 2^{nd} generation lentiviral packaging plasmids (DR8.9 and pCMV-VSVG) in a ratio of 4:3:1 using Lipofectamine-3000 (Thermo) according to the manufacturer's instructions. Six hours after transfection, medium was changed to DMEM harvest medium containing 10% FCS. Virus containing supernatant was harvested at 30 and 54 hours after transfection, centrifuged for 5 minutes at 2500 rpm to remove debris and filtered through a 45 μm filter before pooling. Virus was precipitated from culture supernatants by incubation with PEG-8000 at 10% final concentration for >4 hours. PEG-precipitate was centrifuged for 1 hour at 4000 rpm and the pellet resuspended in ˜1/100 the original volume. Aliquots were stored at −80° C. until use.

Tumor Processing

Tumor pieces were finely chopped using sterile razor blades in 0.5 ml digestion mix (DMEM/F12 with 1 mg/ml collagenase IV, 10 uM Y27632 and 20 ug/ml DNase). These were digested for 30 min at 37C, triturated with a 10 ml pipette, then digested for an additional 15 min at 37 C. The mixture was strained through a 100 uM strainer. Cells were washed once with FACS buffer (PBS with 0.% BSA, 1 mM EDTA) and resuspended in organoid medium (Advanced DMEM/F12 with 10 uM SB202190, 1× HEPES, 1.25 mM N-acetylcysteine, 10 mM nicotinamide, 1X Glutamax, 1X Primocin, 5% Knockout Serum Replacement, 1× B27 supplement, 0.1 nM cholera toxin, 0.5 uM A83-01, 10 uM Y27632, 1 uM PGE2, 10 nM [Leu15]-Gastrin I, 10 ng/ml rhFGF10, 10 ng/ml rhFGF2, 50 ng/ml EGF, 0.3 ug/ml hydrocortisone). For FACS analysis, 10 ul of cell sample was diluted with 190u1 FACS buffer containing 5 nM ToPro-3.

Tumor Cell Infection and Culture

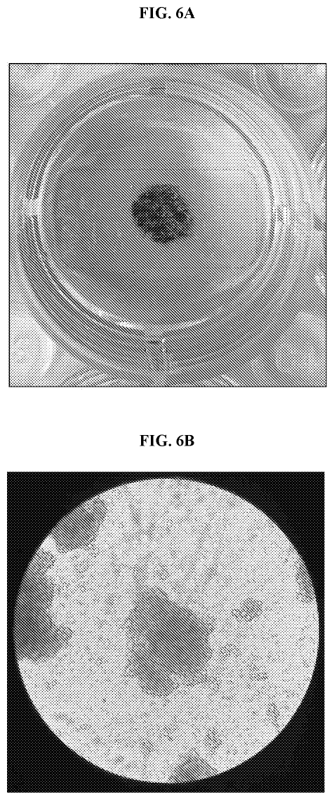

Cells were mixed in organoid medium with lentivirus at a target MOI of <1 in the presence 4 ug/ml polybrene, and incubated for 1 hour at room temperature. The suspension was then spun, the pellet resuspended in a minimal volume of organoid medium, and then plated onto collagen sponges (Ethicon) for 3D culture (FIG. 6A). Cells were grown at 37C with 5% CO2. Medium was changed every 2 days.

Sponge Harvest

Sponges were digested for 15 min at 37C with lmg/ml Collagenase IV in DMEM-F12. Analysis of the re-isolated cells demonstrated the outgrowth of the small tumor-derived tumoroids/organoids (FIG. 6B). A small sample was retained for FACS (as described above), and the remainder was spun at 1200 rpm for 5 min. The supernatant was discarded and the pellet frozen at −80 C for DNA isolation. Expression of beta-2 microglobulin protein was analyzed using directly conjugated anti-B2M antibodies (FIG. 7), demonstrating loss of B2M protein expression at the precise frequency expected based on the relative abundance of B2M-targeting gRNAs in the gRNA library.

Cancer Cell Line Culture

A549 lung carcinoma cells (American Type Culture Collection) were grown in Dulbecco's Modified Eagle Medium (Gibco) supplemented with 10% (v/v) fetal bovine serum, 1× Glutamax, and 1× antibiotic/antimycotic.

DNA Preparation, PCR, and Next-Generation Sequencing (NGS)

Genomic DNA was isolated using the Zymo Quick-DNA Miniprep Plus kit. 5 ug of purified genomic DNA was used as input for first round PCR amplification using the Q5 2X Master Mix and primers specific to the lentiviral vector. 10% of the resulting first round reaction products was then used as input for the second round of PCR amplification, utilizing barcoding primers to allow multiplex NGS readout. Samples were analyzed on the Illumina MiSeq using standard Illumina sequencing primers (Admera).

Sequence Analysis

Read1 sequences corresponding to the PCR barcodes were used for de-multiplexing, generating single-sample FASTA files containing gRNA readcounts. Sequencing data was analyzed using the CRISPRCloud2 platform, generating both CPM-normalized readcounts as well as statistical analysis of gRNA abundance based beta-binomial modeling. Data were visualized as ‘volcano’ plots (DataGraph), describing the relationship between statistical significance and fold-change in gRNA abundance. Typically, comparison was made between gRNA abundance immediately following lentiviral transduction and at the end of the in vitro culture period.

Analysis of dropout frequency using library screening in the A549 lung cancer cell line demonstrated clear loss of gRNAs corresponding to known essential genes (e.g. TOP2A, TUBG1 and others), while non-targeting control gRNAs demonstrated no corresponding decrease in abundance (FIG. 8). The library utilized in this experiment comprised the gRNAs of Tables 6B-6C (SEQ ID NOS: 1526-2959) as described above.

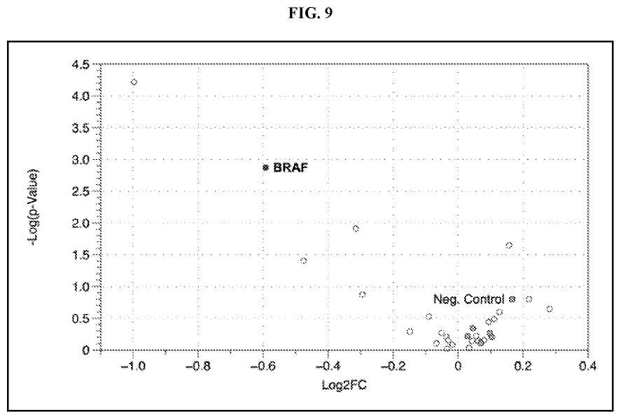

Analysis of dropout frequency using library screening in primary human melanoma tumor sample demonstrated clear loss of gRNAs corresponding to a known melanoma therapeutic vulnerability (e.g. BRAF), while non-targeting control gRNAs demonstrated no corresponding decrease in abundance (FIG. 9). Additional hits corresponding to presumptive cancer therapeutic vulnerabilities were also identified. The library utilized in this experiment comprised the gRNAs of Table 6D (SEQ ID NOS: 2980-3071) and SEQ ID NOS: 2890-2905 and 2960-2979 of Table 6C, as described above.

### Example 4

### In Vivo Validation of Personalized Genomic Profiling

Oncology drugs targeting presumptive cancer therapeutic vulnerabilities identified in Example 3, are tested in an in vivo animal model of the patient's cancer. Drugs that show efficacy for treating the cancer in the animal model are selected for treating the patient's cancer.

While preferred embodiments have been shown and described herein, it will be obvious to those skilled in the art that such embodiments are provided by way of example only. Numerous variations, changes, and substitutions will occur to those skilled in the art without departing from the scope of this application. Various alternatives to the embodiments described herein may be employed in practicing the scope of this application.

What is claimed is:

1. A method of treating cancer in a subject in need thereof, the method comprising administering to the subject a therapeutic molecule selected from a library of therapeutic molecules; wherein the therapeutic molecule has been selected by a method comprising: modifying cancer cells from the subject to knock down or knock out the function of a plurality of genes, each gene in the plurality of genes encoding for a protein target of a therapeutic molecule in the library of therapeutic molecules, whereby the therapeutic molecule has been selected if knocking down or knocking out the function of the gene that encodes for the protein target of the selected therapeutic molecule impairs cancer cell viability.

2. The method of claim 1, wherein the library of therapeutic molecules comprises at least 1, at least 2, at least 3, at least 4, at least 5, at least 10, or at least 20 therapeutic agents of Tables 2-3.

3. The method of claim 1 or claim 2, wherein one or more of the plurality of genes encode for a protein of Tables 3-5D.

4. A method of treating cancer in a subject in need thereof, the method comprising administering to the subject a therapeutic molecule selected from a library of therapeutic molecules; wherein the cancer of the subject has been determined to be susceptible to the selected therapeutic molecule by a method comprising:
(a) contacting a sample of cancer cells from the subject with a library of gene modulatory reagents to generate a plurality of modified cancer cells, wherein each modified cancer cell harbors one or more of the gene modulatory reagents, and each gene modulatory reagent is capable of knocking down or knocking out the function of a gene that encodes a protein target of a therapeutic molecule in the library of therapeutic molecules, and
(b) sequencing the plurality of modified cancer cells, wherein a gene modulatory reagent that impairs cell viability will have fewer sequence reads than a gene modulatory reagent that does not impair cell viability, and the gene that is knocked down or knocked out by the gene modulatory reagent that impairs cell viability encodes for the protein targeted by the selected therapeutic molecule.

5. The method of claim 4, wherein prior to sequencing, one or more of the plurality of modified cancer cells have been propagated.

6. The method of claim 4 or claim 5, wherein the cancer cells are primary cancer cells.

7. The method of any one of claims 4-6, wherein contacting comprises introducing the one or more gene modulatory reagents into each cancer cell by a viral or non-viral delivery method.

8. The method of claim 7, wherein one or more of the gene modulatory reagents in the library are encoded on a viral vector.

9. The method of any one of claims 4-8, wherein the library comprises from about 10 to about 2,000, from about 10 to about 500, from about 10 to about 200, from about 10 to about 150, from about 50 to about 500, from about 50 to about 200, from about 50 to about 2,000, from about 100 to about 2,000, or from about 500 to about 2,000 different gene modulatory reagents.

10. The method of any one of claims 4-9, wherein one or more gene modulatory reagents from the library of gene modulatory reagents comprises a nucleic acid sequence homologous to at least about 15 contiguous nucleotides of a gene encoding a protein of Tables 3-5D.

11. The method of any one of claims 4-10, wherein the library of therapeutic molecules comprises at least 1, at least 2, at least 3, at least 4, at least 5, at least 10, or at least 20 therapeutic agents of Tables 2-3.

12. The method of any one of claims 4-11, wherein one or more of the gene modulatory reagents each comprise a guide RNA (gRNA) sequence comprising at least about 90% homology to a sequence selected from SEQ ID NOS: 1-2789, 2980-3071.

13. The method of any one of claims 4-12, wherein one or more of the gene modulatory reagents comprise a guide RNA (gRNA) sequence comprising homology to at least a portion of the gene that encodes a protein target of a therapeutic molecule in the library of therapeutic molecules.

14. The method of any one of claims 4-13, wherein the sample of cancer cells is contacted with an endonuclease.

15. The method of claim 14, wherein the endonuclease comprises a Cas9 or Cas12a endonuclease.

16. The method of any one of claims 13-15, wherein the gRNA is positioned within a viral vector.

17. A method of generating a plurality of modified cancer cells from a subject having cancer, the method comprising delivering a library of gene modulatory reagents to a sample of cancer cells from the subject to generate the plurality of modified cancer cells; wherein each modified cancer cell harbors one or more of the gene modulatory reagents, and each gene modulatory reagent is capable of knocking down or knocking out the function of a gene that encodes a protein target from a library of protein targets.

18. The method of claim 17, wherein one or more of the gene modulatory reagents comprises a guide RNA (gRNA) sequence comprising homology to at least a portion of the gene whose function is knocked down or knocked out in the modified cancer cell.

19. The method of claim 17, wherein one or more of the gene modulatory reagents each comprise a guide RNA (gRNA) sequence comprising at least about 90% homology to a sequence selected from SEQ ID NOS: 1-2789, 2980-3071.

20. The method of any one of claims 17-19, wherein the sample of cancer cells is contacted with an endonuclease.

21. The method of claim 20, wherein the endonuclease comprises a Cas9 or Cas12a endonuclease.

22. The method of any one of claims 18-21, wherein the gRNA is positioned within a viral vector.

23. The method of any one of claims 17-22, wherein the sample of cancer cells comprises primary cancer cells.

24. The method of any one of claims 17-23, wherein the library comprises from about 10 to about 2,000, from about 10 to about 500, from about 10 to about 200, from about 10 to about 150, from about 50 to about 500, from about 50 to about 200, from about 50 to about 2,000, from about 100 to about 2,000, or from about 500 to about 2,000 different gene modulatory reagents.

25. The method of any one of claims 17-24, wherein the library of protein targets comprises at least 1, at least 2, at least 3, at least 4, at least 5, at least 10, or at least 20 protein targets of Tables 3-5D.

26. The method of any one of claims 17-25, further comprising propagating the modified cancer cells.

27. A compilation comprising a plurality of modified cancer cells, wherein each modified cancer cell harbors one or more gene modulatory reagents, and each gene modulatory reagent is capable of knocking down or knocking out the function of a gene that encodes a protein target from a library of protein targets.

28. The compilation of claim 27, wherein at least one of the one or more of gene modulatory reagents comprises a sequence at least about 90% homologous to a sequence selected from SEQ ID NOS: 1-2789, 2980-3071.

29. The compilation of claim 27 or claim 28, wherein the library of protein targets comprises at least 1, at least 2, at least 3, at least 4, at least 5, at least 10, or at least 20 protein targets of Tables 3-5D.

30. The compilation of any of claims 27-29, wherein one of more of the gene modulatory reagents comprise a guide RNA (gRNA) sequence comprising homology to at least a portion of the gene whose function is knocked down or knocked out in the modified cancer cell.

31. The compilation of claim 30, wherein the gRNA comprises homology to about 10 to about 50 contiguous nucleotides of the gene.

32. The compilation of claim 30 or claim 31, further comprising an endonuclease.

33. The compilation of claim 32, wherein the endonuclease comprises a Cas9 or Cas12a endonuclease.

34. The compilation of any one of claims 30-33, wherein the gRNA is positioned within a viral vector.

35. The compilation of any one of claims 27-34, wherein the modified cancer cells are modified primary cancer cells.

36. The compilation of any one of claims 27-35, comprising from about 10 to about 2,000, from about 10 to about 500, from about 10 to about 200, from about 10 to about 150, from about 50 to about 500, from about 50 to about 200, from about 50 to about 2,000, from about 100 to about 2,000, or from about 500 to about 2,000 different gene modulatory reagents.

37. The compilation of any one of claims 27-36, comprising from about 10 to about 2,000, from about 10 to about 500, from about 10 to about 200, from about 10 to about 150, from about 50 to about 500, from about 50 to about 200, from about 50 to about 2,000, from about 100 to about 2,000, or from about 500 to about 2,000 different populations of modified cancer cells.

38. A method of evaluating the functional effect of genetically modifying cancer cells from a subject, the method comprising: sequencing a plurality of modified cancer cells, wherein each modified cancer cell harbors one or more gene modulatory reagents, each gene modulatory reagent capable of knocking down or knocking out the function of a gene that encodes a protein target in a library of protein targets; and wherein a gene modulatory reagent that impairs cell viability will have fewer sequence reads than a gene modulatory reagent that does not impair cell viability.

39. The method of claim 38, further comprising determining which gene modulatory regents have fewer than a threshold number of sequence reads.

40. The method of claim 39, wherein the threshold number of sequence reads is an expected number of sequence reads if the gene modulatory reagent did not impair cell viability.

41. The method of claim 39, wherein the threshold number of sequence reads is an average number of sequence reads for each gene modulatory reagent in the plurality of modified cancer cells.

42. The method of any one of claims 38-41, further comprising correlating each gene modulatory reagent that has fewer than the threshold number of sequence reads to its corresponding protein target in the library of protein targets.

43. The method of claim 42, further comprising correlating the corresponding protein target to a therapeutic molecule.

44. The method of any one of claims 38-43, wherein the library of protein targets comprises at least 1, at least 2, at least 3, at least 4, at least 5, at least 10, or at least 20 protein targets of Tables 3-5D.

45. The method of any one of claims 38-44, wherein one or more of the gene modulatory reagents each comprise a guide RNA (gRNA) sequence comprising at least about 90% homology to a sequence selected from SEQ ID NOS: 1-2789, 2980-3071.

46. A library comprising a plurality of gene modulatory reagents, each gene modulatory reagent capable of knocking down or knocking out the function of a gene that encodes a protein target from a library of protein targets.

47. The library of claim 46, wherein the plurality of gene modulatory reagents is capable of knocking down or knocking out the function of at least about 50% of the genes that encode for the protein targets in the library.

48. The library of claim 46 or claim 47, wherein the library of protein targets comprises at least 1, at least 2, at least 3, at least 4, at least 5, at least 10, or at least 20 protein targets of Tables 3-5D.

49. The library of any one of claims 46-48, wherein one or more of the plurality of gene modulatory reagents each comprise a guide RNA (gRNA) sequence comprising at least about 90% homology to a sequence selected from SEQ ID NOS: 1-2789, 2980-3071.

50. The library of any one of claims 46-49, wherein the plurality of gene modulatory reagents is capable of knocking down or knocking out the function of about 10 to about 2,000, from about 10 to about 500, from about 10 to about 200, from about 10 to about 150, from about 50 to about 500, from about 50 to about 200, about 50 to about 2,000, or about 100 to about 2,000 genes.

51. The library of any one of claims 46-50, wherein the library comprises about 10 to about 2,000, from about 10 to about 500, from about 10 to about 200, from about 10 to about 150, from about 50 to about 500, from about 50 to about 200, about 50 to about 2,000, or about 100 to about 2,000 gene modulatory reagents.

52. The library of any one of claims 46-51, wherein at least one of the gene modulatory reagents is capable of knocking out the function of a gene.

53. The library of claim 52, wherein at least one of the gene modulatory reagents comprise a gRNA sequence having homology to at least a portion of the gene whose function is knocked out by the gene modulatory reagent.

54. The library of any one of claims 46-53, wherein at least one of the gene modulatory reagents is positioned within a viral vector.
